(19) United States
(12) Patent Application Publication
(10) Pub. No.: US 2005/0165218 A1
(43) Pub. Date: Jul. 28, 2005

(54) Crystal structure of the ligand binding domain of the retinoic acid-related orphan receptor alpha (ror-alpha)

(76) Inventors: Rene Beerli, Binningen (CH); Brigitte Fournier, Riedisheim (FR); Jorg Kallen, Basel (CH); Jean-Marc Schlaeppi, Allschwil (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/509,316

(22) PCT Filed: Apr. 28, 2003
(86) PCT No.: PCT/EP03/04433

(30) Foreign Application Priority Data
Apr. 29, 2002 (US) 60376427

(51) Int. Cl. A61K 38/47; C07K 14/705; G06F 19/00; G01N 33/48; G01N 33/50

(52) U.S. Cl. 530/350; 702/19

(57) ABSTRACT

The present invention relates RORa in crystallized form and methods for the preparation thereof. The invention also provides a three-dimensional model of RORa and means for the design of RORa modulators. Ligands binding to RORa are also provided.

### FIELD OF THE INVENTION

The present invention relates RORα in crystallized form and methods for the preparation thereof. The invention further provides a three-dimensional model of RORα and means for the design of RORα modulators.

### BACKGROUND OF THE INVENTION

The retinoic acid-related orphan receptor α (RORα) is an orphan member of nuclear receptor protein family to which belong receptors such as retinoic acid receptor (RAR), peroxisome proliferator-activated receptor (PPAR), estrogen receptor (ER), vitamin D receptor (VDR) and thyroid receptor (TR). Like other members of the nuclear receptor family, RORα exhibits a modular structure composed of several domains, among them a DNA-binding domain (DBD) and a ligand-binding domain (LBD). The latter displays low degree of homology with the LBD of T3Rβ (25%), VDR (24%), RARα (24%), PPARα (24%) and RXRα (20%) from which X-ray structures have been solved. However, attempts to crystallize the LBD of RORα have failed so far and no X-ray structure of RORα was available. In addition, to this point, no ligand has been identified until now. Our understaniding of the physiological role of the receptor would be greatly enhanced by the discovery of a natural ligand. Further, provision of the spatial organization would assist in the designing of agonists and antagonists of RORα.

### SUMMARY OF THE INVENTION

In one aspect, the present invention provides crystalline LBD of RORα. In a related aspect the invention provides crystalline LBD of RORα associated with a ligand.

In another aspect, the invention provides a set of co-ordinates representing the spatial organization of the LBD of RORα. In a related aspect the invention provides a model of the LBD of RORα comprising a set of co-ordinates embodying the structure of the LBD of RORα. In another related aspect, this invention provides for a set of co-ordinates useful in drug design. In yet another related aspect, the invention provides for a method for identifying a substance binding to the LBD of RORα, comprising providing a model embodying the structure of the LBD of RORα, assessing the interaction of a candidate substance with said model, and selecting a substance which is predicted to interact with the LBD of RORα Substances identified by this method are also provided.

In a further aspect, the invention provides for a method for identifying a compound acting as agonist or antagonist of RORα that binds to the LBD of RORα comprising selecting a potential compound by performing rational drug design with one or more sets of atomic coordinates embodying the structure of the LBD of RORα, contacting the potential compound with a LBD of RORα and measuring the binding of the compound to the LBD of RORα. Agonists and antagonists identified by this method are also provided.

In another aspect, the present invention provides for a method of screening for compounds interacting with RORα comprising contacting RORα with a candidate compound, measuring interactions between the candidate compound and RORα in the absence of sterols, and selecting said compound if it interacts with RORα in the absence of sterols. Preferred sterols are cholesterol or cholesterol derivatives. Compounds identified by this method are also provided.

In another aspect of the present invention, the use of RORα for the screening of cholesterol related diseases is provided.

In yet another aspect the present invention provides a composition comprising LBD of RORα and a sterol, preferably cholesterol or a cholesterol derivative. In a preferred embodiment, said composition is crystallizable.

### BRIEF DESCRIPTION OF THE TABLES AND FIGURES

Table 1: Native crystal data and X-ray data statistics of LBD of RORα in complex with cholesterol.

Table 2: Hg-derivative crystal data, X-ray data and heavy atom refinement statistics (for complex with cholesterol).

Table 3: Refinement statistics (for complex with cholesterol).

Table 4: shows effects of mutations preventing binding of cholesterol to RORα.

Table 5: shows effects of fluvastatin on RORα transcriptional activity.

Table 6: Effect of cholesterol and cholesterol derivative on ROR alpha transcriptional activity.

Table 7: Native crystal data and refinement statistics of LBD of RORα in complex with cholesterol sulfate.

Table 8: Atomic structure coordinates for a representative structure of the LBD of RORα in complex with cholesterol (numbering according to Swissprot P35398-1).

Table 9: Atomic structure coordinates for a representative structure of the LBD of RORα in complex with cholesterol-sulfate (numbering according to Swissprot P35398-2).

FIG. 1: Sequence of human RORα (Swissprot P35398-1).

FIG. 2 shows a schematic representation of the X-ray structure of the complex between RORα-LBD and cholesterol.

FIG. 3 shows a zoomed in view of the complex between RORα-LBD and cholesterol (numbering according to Swissprot P35398-1).

FIG. 4: Proposal of ligands in order to increase the affinity and to obtain antagonistic activity (numbering according to Swissprot P35398-1).

FIG. 5: Proposal of further derivatives of cholesterol in order to increase the affinity (numbering according to Swissprot P35398-1).

FIG. 6 shows the displacement of cholesterol by 25-OH cholesterol and cholesterol sulfate.

FIG. 7 shows a zoomed view of X-ray structure of ROR(alpha)/cholesterol (numbering according to Swissprot P35398-2).

FIG. 8 Overview of interactions made by cholesterol-sulfate with LBP of ROR(alpha) (numbering according to Swissprot P35398-2).

FIG. 9 Comparison of the X-ray structures of ROR(alpha)/cholesterol-sulfate and ROR(alpha)/cholesterol (numbering according to Swissprot P35398-2).

FIG. 10 Comparison of the X-ray structures of ROR(alpha)/cholesterol (left) and ROR(alpha)/cholesterol-sulfate (right) (numbering according to Swissprot P35398-2).

FIG. 11 Sequence of the construct used in crystallization. The secondary structure elements are shown below the sequence. Amino acids that have a nonhydrogen atom closer than 4 Å to cholesterol are highlighted in red (numbering according to Swissprot P35398-2).

### DETAILED DESCRIPTION OF THE INVENTION

The present invention provides crystals of the LBD of RORα. Moreover, the present invention provides the structural determination of such crystals by X-ray crystallography. In one embodiment, the structure of the crystal has been solved to a resolution of 1.88 Å. Surprisingly, it was found that the crystal contained a ligand associated to RORα. The ligand was identified as cholest-5-en-3beta-ol (cholesterol). Thus the present invention not only provides information on the spatial organization of the LBD of RORα useful for instance for in-silico screening, docking and rational drug design, but also cholesterol as a ligand binding to the RORα which is useful for the identification of amino acids involved in the ligand binding. The information provided in accordance with the present invention can be used as basis for the design of compounds binding to the LBD of RORα, as exemplified below. The crystal LBD of RORα provided by this invention can take any crystalline form, but is preferably a single crystal. In a more preferred embodiment the crystal comprises a unit cell having the of a=55 Å±5 Å, b=50 Å±5 Å, c=60 Å±6 Å and β=98.5°±9° and space group P2₁. Preferably, the unit cell dimensions are a=55.9 Å±2 Å, b=49.9 Å±2 Å, c=60.7 Å±2 Å and β=98.7°±5° or a=54.4 Å±2 Å, b=49.9 Å±2 Å, c=60.7 Å±2 Å, β=97.8°±5°. In another preferred embodiment, the crystalline LBD of RORα is of human origin. The crystalline LBD of RORα according to the present invention is preferably associated with a second chemical substance. Such a substance may be any natural or synthetic chemical molecule, preferred are small molecules, more preferred are small lipophilic molecules. Cholesterol has been identified, in accordance with the present invention, as a ligand fitting into this binding pocket. Thus, in a particularly preferred embodiment such a substance is cholesterol or a cholesterol derivative. As used herein, the term “small molecule” refers to a natural or synthetic compound, preferably an organic molecule, with a molecular weight less than 3000 Da, more preferably less than 1000 Da, most preferably less than 500 Da. The term “lipophilic”, as used herein, refers to compounds that are mainly unpolar and that are not or only slightly soluble in water. Typical examples may include fatty acids, retinoic acids, melatonin, steroid hormones, vitamin D derivatives. Other examples may include lipophilic molecules like tamoxifen or raloxifen. In accordance with the present invention, a particularly preferred lipophilic ligand is cholesterol and derivatives thereof. As used herein the term “cholesterol derivative” means a molecule that possesses similarity to cholesterol, such as the same overall structure, but with different substituents or differences in the location of unsaturated bonds or sterical isomers. Examples for such cholesterol derivatives can for instance be found in http://www.steraloids.com.

Crystals of the LBD of RORα and, optionally a second chemical species can be grown by a number of techniques including batch crystallization, vapor diffusion (either by sitting drop or hanging drop) and by microdialysis. Seeding of the crystals in some instances is required to obtain X-ray quality crystals. Standard micro and/or macro seeding of crystals may therefore be used. An initial crystal can be allowed to grow over several weeksat 4° C. or at room temperature (ca. 20° C.) from a hanging drop. Crystals then can be subsequently grown by macroseeding from the initial crystal. Once a crystal of the present invention is grown, X-ray diffraction data can be collected. A MAR imaging plate detector for X- ray diffraction data collection can be used for example. Crystals can be characterized by using X-rays produced in a conventional source (such as a sealed tube or a rotating anode) or using a synchrotron source.

Methods of characterization and data collection include, but are not limited to, precession photography, oscillation/rotation data collection and diffractometer data collection. As exemplified below, heavy atom derivatives can be obtained by soaking crystals in solution with 4 mM methylmercuric acetate for 1 hour. Data processing and reduction can be carried out using programs (DENZO, and SCALEPACK) of the HKL-suite [Otwinowski and Minor, Meth. Enzymol. 276:307-326 (1997)]. Heavy atom positions can be found using programs such as SnB [Weeks, C. M. & Miller, R. (1999) J.Appl.Cryst.32, 120-124.] or programs (e.g. SHELX and RSPS) of the CCP4 program suite [Collaborative Computational Project, Number4, Acta Cryst. D53: 760-763 (1994)]. Electron density maps can be calculated using programs (e.g. MLPHARE and DM) of the CCP4 program suite [Collaborative Computational Project, Number4, Acta Cryst. D53: 760-763 (1994)] or alternatively using SHARP [La Fortelle, E. D. and Bricogne, G., Methods in Enzymology 276:472-494 1997)] and SOLOMON. Molecular models can be built into this map using O [Jones, T. a. et al., ACTA Crystallogr. A47:110-119 (1991)]. A complete molecular model for the protein can be built on the basis of the experimental electron density map. Model building interspersed with positional and simulated annealing refinement using X-PLOR, (Brunger, X-PLOR v.3.1 Manual, New Haven: Yale University, (1993)] or with CNS, using a maximum likelihood residual [Brunger, A. T. et al., Acta Cryst. D54: 905-921 (1998)] can permit an unambiguous trace and sequence assignment of the LBD of RORα.

Accordingly, the present invention provides for a model of the structure of the LBD of RORα useful for rational drug design comprising a set of co-ordinates embodying the structure of the LBD of RORα. Thus, a preferred embodiment provides for a model embodying the structure of the LBD RORα comprising one or more sets of atomic coordinates in Table 8 or 9. Other preferred embodiments provide a computer system comprising computer hardware or the model of the present invention and a computer readable medium comprising the model of the present invention. The set of co-ordinates is preferably determined by crystallographic analysis of the LBD of RORα, however any available method may be used to construct such a model using data disclosed herein or obtained from independent crystallographic analysis of the LBD of RORα. The term “structure co-ordinates” refers to Cartesian co-ordinates derived from mathematical equations related to the patterns obtained on diffraction of a monochromatic beam of X-rays by the atoms (scattering centers) of a protein or protein-ligand complex in crystal form. The diffraction data are used to calculate an electron density map of the repeating unit of the crystal. The electron density maps are then used to establish the positions of the individual atoms of the enzyme or enzyme complex. Variations in co-ordinates may be generated because of mathematical manipulations of the structure co-ordinates. For example, the structure co-ordinates set forth in Table 8 or 9 could be manipulated by crystallographic permutations of the structure co-ordinates, fractionalization of the structure co-ordinates, integer additions or subtractions to sets of the structure co-ordinates, inversion of the structure co-ordinates or any combination of the above. Alternatively, modifications in the crystal structure due to mutations, additions, substitutions, and/or deletions of amino acids, or other changes in any of the components that make up the crystal could also account for variations in structure co-ordinates. If such variations are within an acceptable standard error as compared to the original co-ordinates, the resulting three-dimensional shape is considered to be the same. Various computational analyses are therefore necessary to determine whether a molecule or molecular complex or a portion thereof is sufficiently similar to all or parts of the structure of the LBD of RORα as to be considered the same. Such analyses may be carried out in current software applications, such as the Molecular Similarity application of QUANTA (Molecular Simulations Inc., San Diego, Calif.) version 4.1, and as described in the accompanying User's Guide. For the purpose of this invention, any molecule or molecular complex that has a root mean square deviation of conserved residue backbone atoms (N, Cα, C, O) of less than 1.5 Å; when superimposed on the relevant backbone atoms described by structure co-ordinates listed in Table 8 or 9 are considered identical. More preferably, the root mean square deviation is less than 1.0 Å. The term “root mean square deviation” means the square root of the arithmetic mean of the squares of the deviations from the mean. It is a way to express the deviation or variation from a trend or object. For purposes of this invention, the “root mean square deviation” defines the variation in the backbone of a protein or protein ligand complex from the relevant portion of the backbone of the LBD of RORα as defined by the structure co-ordinates described herein.

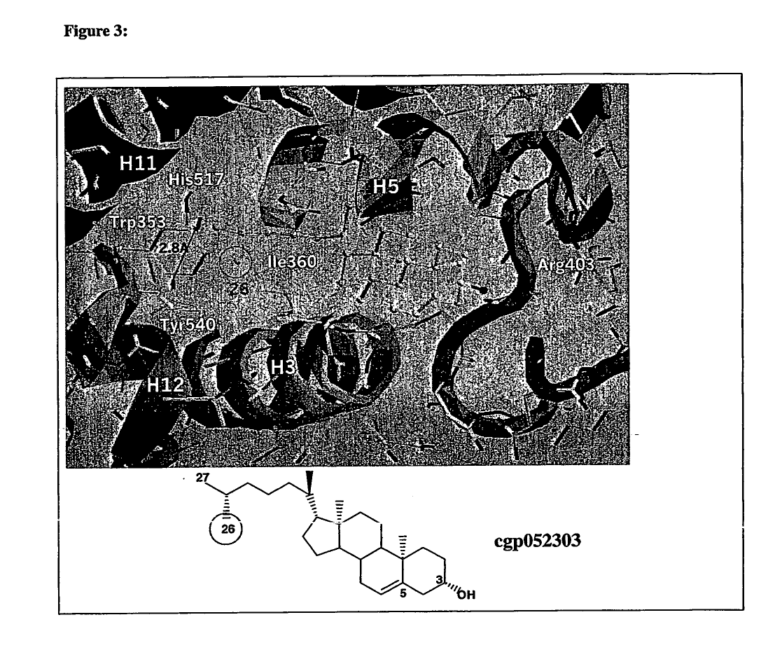

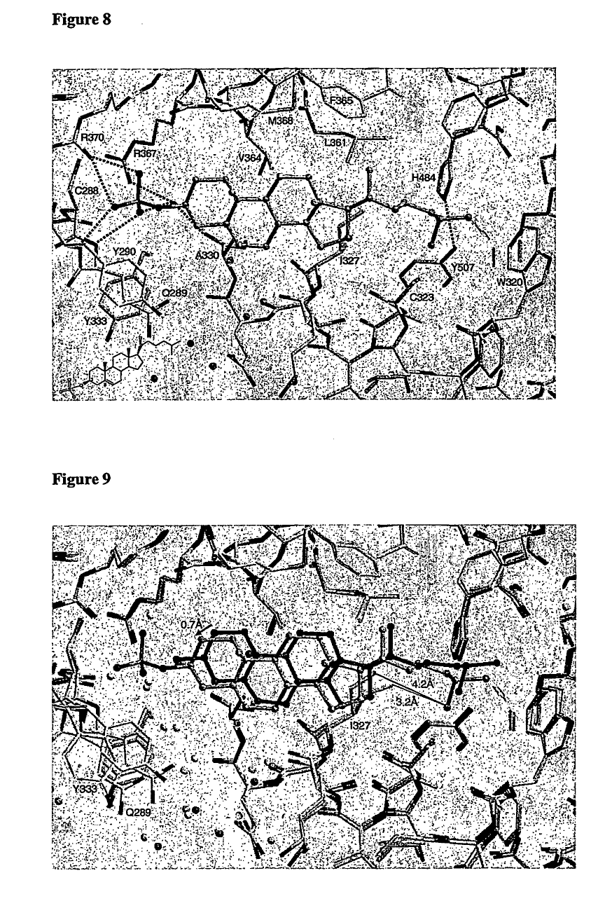

In certain embodiments, the data set embodies a portion of the structure of the LBD of RORα, including without limitation the binding pocket of LBD of RORα. The term “binding pocket”, as used herein, refers to a region of a molecule or molecular complex, that, as a result of its shape, favorably associates with another chemical entity or compound. In accordance with the present invention, a preferred binding pocket includes the amino acids shown in FIGS. 3, 4, 5, 7, 8, 9 or 10 one or more of the following amino acids: Cys321, Gln322, Tyr323, Leu328,Trp353, Cys356, Ala357, Lys359, Ile360, Glu362, Ala363, Val397, Phe398, Arg400, Met4, Arg403, Ala404, Val412, Tyr413, Phe414, Phe424, Leu427, Cys429, Phe432, Ile433, Val436, His517, Lys520 and Tyr540 (numbering according to SWISS-PROT P35398-1).

In one embodiment of the present invention, the model may be used to identify substances that interact with the LBD of RORα. In general, molecular similarity applications in accordance with the present invention permit comparisons between different structures, different conformations of the same structure, and different parts of the same structure. A potential interacting substance is examined through the use of computer modeling using a docking program such as GRAM, DOCK, or AUTODOCK [Dunbrack et al., Folding & Design, 2:27-42 (1997)]. This procedure can include computer fitting of potential ligands to the LBD of RORα, for example to ascertain how well the shape and the chemical structure of the potential ligand will complement with the binding pocked provided by the present application. Computer programs can also be employed to estimate the attraction, repulsion, and steric hindrance of the ligand to the LBD of RORα. Generally the tighter the fit (e.g., the lower the steric hindrance, and/or the greater the attractive force) the more potent the potential drug will be since these properties are consistent with a tighter binding constant. Furthermore, the more specificity in the design of a potential drug the more likely that the drug will not interfere with other properties of the RORα protein or other proteins (particularly proteins present in the nucleus). This will minimize potential side-effects due to unwanted interactions with other proteins. Initially a potential interacting substance could be obtained by screening a chemical library. A ligand selected in this manner could then be systematically modified by computer modeling programs until one or more promising potential ligands are identified. Alternatively, a known ligand of RORα, such as for instance cholesterol as identified in accordance with this invention, may be used as a starting point for systematic modification. Such computer modeling allows the selection of a finite number of rational chemical modifications, as opposed to the countless number of essentially random chemical modifications that could be made, and of which any one might lead to a useful drug. Each chemical modification requires additional chemical steps, which while being reasonable for the synthesis of a finite number of compounds, quickly becomes overwhelming if all possible modifications needed to be synthesized. Thus through the use of the three-dimensional structures disclosed herein and computer modeling, a large number of these compounds can be rapidly screened on the computer monitor screen, and a few likely candidates can be determined without the laborious synthesis of untold numbers of compounds.

Accordingly, methods for identifying substances that bind to the LBD of RORα are provided. Such methods typically include the steps of providing a model embodying the structure of the LBD of RORα, assessing the interaction of a candidate substance with said model, selecting a substance which is predicted to interact with the LBD of RORα, and, optionally, contacting the selected substance with the LBD of RORα. In a preferred embodiment, such a method includes comparing the 3-D structure of candidate compounds with the 3-D molecular model shown in Table 8 or 9 or with the co-ordinates of amino acids which are part of a preferred binding pocket or directly or indirectly involved in binding of a ligand, as herein disclosed for instance in FIGS. 3, 4, 5, 7, 8, 9 or 10. Preferably, said amino acids can form hydrogen bonds with hydrogen bonding functional groups (directly or via water molecules) in a candidate compound or can form favorable vdW-interactions. The interactions are preferably assessed by a computer-assisted method, such as for instance a data processing method in which the structure co-ordinate data as described above is input in a data structure such that the interatomic distances between the atoms of the LBD of RORα are easily retrieved, and the distances between hydrogen-bonding functional groups of different candidate compounds and hydrogen bonding atoms of the amino acids that form the binding pocket in the 3D molecular model are compared (or the distances between groups forming vdW-interactions) thereby allowing the identification of those candidate compounds which would theoretically form the most stable complexes with the 3-D molecular model binding pocket of the LBD of RORα, based on optimal hydrogen bonding and vdW-interactions between the two structures.

In a preferred embodiment the substances are designed to interact via vdW-interactions or via hydrogen bond interactions directly or indirectly (e.g. via water molecules) with atoms of one or more amino acids shown in FIGS. 3, 4, 5, 7, 8, 9 or 10 or selected from the group consisting of Cys321, Gln322, Tyr323, Leu328,Trp353, Cys356, Ala357, Lys359, Ile360, Glu362, Ala363, Val397, Phe398, Arg400, Met401, Arg403, Ala404, Val412, Tyr413, Phe414, Phe424, Leu427, Cys429, Phe432, Ile433, Val436, His517, Lys520 and Tyr540, Gln322, Tyr323, Arg400, Arg403. In a more preferred embodiment the substances interact via vdW-interactions or via hydrogen bond interactions directly or indirectly (e.g. via water molecules) with atoms of one or more amino acids selected from the group consisting of Gln322, Tyr323, Arg400, Arg403 or Trp353, Lys359, Ile360, Ala363, Met401, Phe414, Leu427, Phe432, Val436. Substances identified using the above methods are also provided. Preferred substances are small molecules, more preferred are small lipophilic molecules (possibly with a polar group) and particularly preferred are cholesterol or cholesterol derivatives, such as for instance cholesterol sulfate. In a further preferred embodiment, the binding constant of the substance to RORα is at least 1 μM, preferably at least 100 nM, more preferably at least 10 nM.

In addition, agonists and antagonists of RORα are provided. In one embodiment methods for screening for agonists or antagonists of RORα are provided. Such methods include selecting a potential agonistic or antagonistic compound by performing rational drug design with one or more sets of atomic co-ordinates embodying the structure of the LBD of RORα, contacting the potential compound with a LBD of RORα and measuring the biological activity of RORα. The selection is typically made in conjunction with computer modeling. A potential compound is identified as agonist if it increases the biological activity of RORα or as antagonist if it decreases the biological activity of RORα. Agonists and antagonists identified by such methods are also provided. The agonist or antagonist needs not to bind to the binding pocket used by the natural ligand of RORα, but could also bind at another position and exert its effect allosterically. A preferred embodiment of an agonist according to the present invention is a compound that stabilizes helix 12 (H12) in the agonistic position, i.e. the position in which H12, together with the H3-H4 region, forms the proper interaction surface, i.e. the complete AF-2, for the coactivator (reviewed e.g. in Renaud & Moras, Cell. Mol. Life Sci., 57, 1748-1769, 2000). A preferred embodiment of an antagonist according to the present invention is a compound that destabilizes the agonistic position of H12 for instance by tilting the position of H12 (reviewed e.g. in Renaud & Moras, 2000, supra). Destabilisation of H12 may for instance be achieved by a cholesterol derivative with a bulky substituent at position 26 thus displacing Tyr540 and/or His517. In a preferred embodiment such agonists or antagonists are small molecules. Particularly preferred are lipophilic small molecules. Examples without being limiting are for instance fatty acids, retinoic acids, melatonin, steroid hormones, vitamin D derivatives, but also compounds similar to tamoxifen or raloxifen or derivatives thereof. In one embodiment, such agonists or antagonists may be cholesterol or cholesterol derivatives. In a preferred embodiment of this invention the cholesterol ligand has been modified using the structural information provided by the present invention to a cholesterol derivative binding more strongly to the ligand binding pocked (LBP) of the LBD of RORα provided by the present invention. An example for a more strongly, competitively binding cholesterol derivative that has been designed using the structural information provided by this invention is cholesterol sulfate (see below). In another preferred embodiment, the present invention provides a pharmaceutical composition comprising a therapeutically effective amount of a compound stabilizing H12 of RORα in an agonistic position and a pharmaceutically acceptable carrier. In a related embodiment, the present invention provides a pharmaceutical composition comprising a therapeutically effective amount of a compound destabilizing H12 of RORα in an agonistic position and a pharmaceutically acceptable carrier.

Once a potentially binding substance, such as an agonist or antagonist, is identified it can be either selected from a library of chemicals or alternatively the potential ligand may be synthesized de novo. The de novo synthesis of one or even a relatively small group of specific compounds is reasonable in the art of drug design. The prospective drug can be placed into any standard binding assay to test its effect on any particular RORα function, for instance on the DNA binding of RORα exemplified below. When a suitable drug is identified, a supplemental crystal can be grown which comprises a protein-ligand complex, for instance formed between the binding pocket of the LBD of RORα and the drug. Preferably the crystal effectively diffracts X-rays allowing the determination of the atomic co-ordinates of the protein-ligand complex to a resolution of greater than 5.0 Angstroms, more preferably greater than 3.0 Angstroms or greater than 2.0 Angstroms. The three-dimensional structure of the supplemental crystal can be determined by molecular replacement analysis. Molecular replacement involves using a known three-dimensional structure as a search model to determine the structure of a closely related molecule or protein-ligand complex in a new crystal form. The measured X-ray diffraction properties of the new crystal are compared with the search model structure to compute the position and orientation of the protein in the new crystal. Computer programs that can be used include: programs (AMORE, MOLREP) of the CCP4 program suite [Collaborative Computational Project, Number4, Acta Cryst. D53: 760-763 (1994)] or X-PLOR [Brunger, X-PLOR v.3.1 Manual, New Haven: Yale University, (1993)]. Once the position and orientation are known an electron density map can be calculated using the search model to provide X-ray phases. Thereafter, the electron density is inspected for structural differences and the search model is modified to conform to the new structure. Using this approach, it will be possible to use the claimed structure to solve the three-dimensional structures of any such LBD of RORα complex. For all of the drug screening assays described herein further refinements to the structure of the drug will generally be necessary and can be made by the successive iterations of any and/or all of the steps provided by the particular drug screening assay.

The substances identified by rational design can be further analyzed in drug screening assay. The drug screening assays of the present invention may use any of a number of assays for measuring the functionality of RORα, including for the ability of RORα following ligand binding to transcriptionally regulate a gene, by increasing phosphorylation of RORα, by allowing RORα to dimerize or to heterodimerize with another nuclear receptor, by improving its ability to interact with co-activators, by changing its conformation and by increasing its ability to bind DNA. In one binding assay, a nucleic acid containing a RORα binding site is placed on a coated or onto a solid support. A preferred binding site is a response element (RORE) composed of a 6 bp AT rich motif immediately preceding a half site AGGTCA and the possible variants of this response element that are given in Giguere et al. 1994, Genes & Development 8:538-553, Mc Broom et al. 1995 Mol.Cell. Biol. 15: 796-808, Moraitis & Giguere, 1999; Molecular Endocrinology. 13:431-439. Methods for placing the nucleic acid on the solid support are well known in the art and include linking biotin to the nucleic acid and linking avidin to the solid support. The RORα is allowed to equilibrate with the nucleic acid and drugs are tested to see if they disrupt or enhance the binding.

In another assay, a co-activator protein, such as for instance GRIP or DRIP 205 (Brandon-Atkins et al. 1999, Molecular Endocrinology 13: 1550-1557), or SRC1, NcoA-1, ERAP/P160, SRC2/NcoA-2, ACTR, SRC-3, pCIP, ERAP-140, RIP 140, RIP 160 P/Caf, CBP/P), ARA70, Ada 3, Rap 46, GRIP170, TRIP 1, PGC1 and 2, SPT6, TIFα, SW1/SNUERF, TRAP 100, TRAP 220, DRIP, NSD1 (Robyr et al. 2000, Mol. Endo. 14: 329-347), are placed on a coated or onto a solid support The RORα protein may be labeled. For example, in one embodiment radiolabeled RORα proteins are used to measure the effect of a drug on binding. In another embodiment the natural ultraviolet absorbance of the RORα protein is used. In yet another embodiment, a Biacore chip (Pharmacia) coated with the co-activator peptide is used and the change in surface conductivity can be measured. In yet another embodiment, the effect of a prospective drug (a candidate compound) on interactions between RORα and their DNA binding sites are assayed in living cells that contain or can be induced to contain activated RORα proteins. Cells containing a reporter gene, such as the heterologous gene for luciferase, green fluorescent protein, chloramphenicol acetyl transferase or 3-galactosidase and the like are operably linked to a promoter containing a RORα binding site. A prospective drug is then contacted with the cell. The amount (and/or activity) of reporter produced in the absence and presence of prospective drug is determined and compared. Prospective drugs which reduce the amount (and/or activity) of reporter produced are candidate antagonists of the RORα DNA binding, whereas prospective drugs which increase the amount (and/or activity) of reporter produced are candidate agonists of RORα DNA binding. Assays for detecting the reporter gene products are readily available in the literature. For example, luciferase assays can be performed according to the manufacturer's protocol (Promega), and beta-galactosidase assays can be performed as described by Ausubel et al., [in Current Protocols in Molecular Biology, J. Wiley & Sons, Inc. (1994)]. In one example, the transfection reaction can comprise the transfection of a cell with a plasmid modified to contain a RORα protein.

In one embodiment, the prospective drugs identified by the methods of this invention can be tested for pharmacological activity using assays known in the art. For example, the identified prospective drugs can be tested for activity as potential drugs for the prophylaxis or treatment of a disease or medical condition which involves excessive bone or cartilage loss using a method as disclosed in WO 01/26737. For instance, a reporter assay can be carried out using the bone sialoprotein (BSP) or osteocalcin (OC), which are known modulators of bone mineralization and remodelling. Suitable cells can be transfected with a reporter construct in which a BSP or an OC promoter drive a reporter gene, such as the firefly luciferase gene. A prospective drug is then contacted with the cell. The amount of luciferase activity produced in the absence and presence of prospective drug is determined and compared. In another embodiment, the system for testing prospective drugs according to the present invention can be the use of classical ovariectomized rat model, the loss of ovarian function induces a drop in circulating estrogen promptly followed by decrease of bone mass (Wronski et al., Calcified Tissue International. 45(6):360, 1989). The drug will be tested on ovariectomized animal for a curative treatment of 8 weeks started twelve weeks after ovariectomy and bone mineral density will be monitored. Another type of experiment could be envisaged which is a preventive treatment of intact animals for eight weeks.

Cholesterol has been found to be a ligand of RORα. In accordance with this finding, the present invention provides novel assay methods for the identification of compounds binding to RORα, in particular for the identification of compounds modulating RORα activity, wherein interactions between the candidate compounds and RORα are allowed to take place in a surrounding reduced in cholesterol, preferably free of cholesterol. Such a method typically includes the steps of (a) contacting RORα with a candidate compound, (b) measuring interactions between the candidate compound and RORα in a surrounding essentially free of cholesterol, and (c) selecting said compound if it interacts with RORα. Though not a requirement, it is preferred that all method-steps are carried out in the cholesterol-reduced, or preferably essentially cholesterol-free, surrounding. In a more preferred embodiment, such a method relates to a eukaryotic cellular system. In a yet even more preferred embodiment insect cells are used. Insect cells differ from eukaryotic cells by lacking the capacity for de novo sterol synthesis. It has been shown that these cells can be propagated under cholesterol-free conditions (Cleverley et al. 1997, Exp. Cell Res. 233: 288-296). Thus, such a cell system could for instance provide an appropriate cell background to monitor the activity of a RORα ligand using the RORα cloned in an appropriate insect cell vector and the classical reporter ROREtkluc. In another embodiment, eukaryotic cells, preferably human cells, are used. These cells can for instance be cultured in medium essentially free of cholesterol and in serum essentially free of LDL-cholesterol (the LDL-free serum preparation is described in Goldstein et al 1983, Methods in Enzymology 98:241-260). Mammalian cells are able to produce cholesterol endogenously. The meaning of essentially cholesterol-free surrounding according to the present invention does not include such endogenously produced cholesterol. In a particular embodiment, endogenously cholesterol producing mammalian cells could for instance be used in an assay to screen the ability of a compound to displace endogenous cholesterol.

Nuclear receptors are known to regulate the transcription of specific genes or sets of genes upon ligand binding, which makes them interesting targets for the screening for compounds useful as therapeutics. So far, however, deeper understanding of the molecular mechanism of RORα that could lead to development of therapeutics has been severely hampered by the lack of knowledge of a ligand that binds the LBD of RORα. The identification of cholesterol as ligand of the receptor RORα in accordance with the present invention, now provides new insights into the physiological role of RORα and provides RORα as a target for the screening for compounds useful for the treatment of cholesterol related-diseases. It has been shown that defects in cholesterol biosynthesis lead to a variety of clinical characteristics (Nwokoro et al., Mol Genet Metab 74:1-2 105-19 2001), covering brain damage, skeletal defects, with in some cases osteosclerosis, limb aplasia or vetebral hypoplasia. Thus, cholesterol related diseases may include endocrine disorders, atherosclerosis and cardiovascular diseases, metabolic diseases such as for instance obesity, inflammatory diseases, skin diseases, diseases related to the CNS, such as for instance Alzheimer disease and disorders in cell proliferation and apoptosis such as tumor related diseases.

In one embodiment, the present invention provides RORα as target for the screening of compounds useful for the treatment of endocrine disorders, in particular disorders that are related to the synthesis of steroid hormones or the regulation of steriodogenesis. In all steroidogenic tissues, regardless of the hormones synthesized, the initial step in steroidogenic cells is the conversion of cholesterol to the first steroid, pregnenolone (Stocco, Ann Rev Physiol 63: 193-213; 2001).

In another embodiment, the present invention provides RORα as target for the screening of compounds useful for the treatment of disorders of the cholesterol homeostasis. Breakdown of cholesterol homeostasis causes disease states, the most common being atherosclerosis. Hypercholesterolemia is a well-known risk factor. Using statins the present inventions shows a direct link between the activity of RORα and a potent anti-atherosclerosis molecule (Table 5) demonstrating the usefulness of RORα as molecular target for the search of compounds to fight atherosclerosis and cardiovascular diseases.

In another embodiment, the present invention provides RORα as target for the screening of compounds useful for the treatment of metabolic disorders. It is known that a cascade of events initiates adipogenesis where C/EBP and PPARγ are important players. Furthermore, RORα is able to strongly induce PPARγ (Sundvold et al. Biochem. Biophys. Res. Com. 287: 383-390; 2001). SREBP promotes the adipogenic program and SREBP activity is sensitive to the level of intracellular cholesterol (Brown et al. Cell 89: 331-340,1997). Thus, in accordance with this invention, RORα is provided as a target for the screening of compounds useful for the treatment of disorders related to adipogenesis, development of obesity and insulin resistance, which can lead to type 2 diabetes. Furthermore, the mature adipocytes secrete factors that play a role in immunological responses, vascular disease and appetite regulation. Adipocytes derived factors include leptin, prostaglandin's and resistin. The present invention providing cholesterol as ligand of RORα thus provide RORα as target for screening for compounds useful for the treatment of diseases related to immune response, vascular disease and appetite regulation.

It has recently been shown that mesenchymal stem cells have the potential to differentiate into these three lineage (Pittenger et al., 1999 Science 284:143-147). Thus, an apparent reciprocal relationship is postulated to exist between the adipocyte and osteoblast phenotypes. This balance is switched toward adipocytes in osteoporotic patients. This invention provides RORα (as PPARγ or C/EBP) as important players in the adipogenesis pathway or in the differentiation of mesenchymal stem cells into adipocytic, chondrocytic or osteoblastic lineage. Thus, the present invention links RORα in this switch toward adipogenesis and therefore is a potential target for therapeutic intervention in osteoporosis.

In another embodiment, the present invention provides RORα as target for the screening of compounds useful for the treatment of inflammatory diseases. Molecular links have been established between cholesterol and cytokines showing the involvement of inflammation and immunity in atherogenesis. In addition, RORα is involved in inflammation (WO01/26737, Bourdji et al. J. Biol Chem.275: 12243-12250 2000, Delerive et al., EMBO reports 21: 4248; 2001).

In another embodiment, the present invention provides RORα as target for the screening of compounds useful for the treatment of skin disorders. RORα is highly expressed in skin (Becker-Andre, 1993; Biochem. Biophys. Res. Commun. 194:1371-1379). In addition, clinical observation of patients with genetic disorders of cholesterol biosynthesis report photosensitivity and patchy alopecia, as well as follicular atrophoderma.

In another embodiment, the present invention provides RORα as target for the screening of compounds useful for the treatment of Alzheimer disease. The lipoprotein allele ApoE4 is associated with an increased incidence of Alzheimer disease (Trittmatter et al. Proc. Natl. Acad. Sci.USA 90: 1977-1981; 1993); the depletion of plasma membrane cholesterol in hippocampal neurons inhibits the formation of Abeta (Simons et al. PNAS 95: 6460-6464;1998), the cleavage product of the amyloid precursor protein, that is a key factor in the pathogenesis of the disease. In addition the main characteristics of the RORα knock out mice is a severe ataxia and their cerebellum is markedly atrophied. This is implicated in rare inherited disease where people are subject to movement disorders.

### EXAMPLES

Cloning and Expression of (His)₆RORα-LBD304-556

A DNA fragment encoding part of polyhedrin promoter up to the ATG codon is amplified by PCR from the pBAKPac8 plasmid (Clontech) by using the oligonucleotide RS365 (5′-ACCATCTCGCAAATAAATAAG-3′) and MG384 (5′-ATGATGATGATGATGATGGCTGCTGCCCATGGTGGGAACTCGAGGCCTGCAGGG-3′). MG384 has a 5′extension not present on the template DNA but which is encoding for a Kozak sequence in front of the ATG codon and part of the His tag which will be present in the final engineered vector. The second PCR reaction is run with the oligonucleotides MG383 (5′-GCCATCATCATCATCATCATCTGGAAGTTCTGTTCCAGGGGCCCGCAGAATTAGAACACCTTGC-3′) and MG385 (5′-GTACCAGATCTTCTAGATTCGTTACCCATCAATTTGCATTG-3′) on a plasmid template encoding the ligand binding domain (aa304 to aa 556; numbering according to SWISS-PROT P35398-1) of the RORα protein. As for the first PCR fragment, the oligonucleotide MG383 has a 5′extension complementing the extension present on the first PCR fragment and which is added by the extension of the fragment by MG384. By mixing both new fragments and with an PCR amplification with MG365 and RS365 a new fragment encoding the Kozak sequence, the ATG, the (His)₆-tag and the cleavage site for the PreScission protease cleavage site (AmershamPharmacia) is introduced in front of the RORα ligand binding domain. This new fragment has at the both end two homology regions en common with the target plasmid pBAKPac8. The integration of the engineered gene into the cloning vector is done by using the method we described earlier (Geiser et al, BioTechniques 31 88-92 ,2001). DNA sequence analysis of the resulting clones confirms that the clone is as intended. The plasmid is called pXI338.

The plasmid pXI338 is co-transfected with linearised BacPAK6 (AcNPV) virus DNA into Sf-21 insect cells using lipofection. The viral supernatant harvested after five days is subjected to plaque purification to obtain homogenous virus populations, which are subsequently amplified on small scale and analyzed for production by Western blotting. A band of correct size is readily detectable using an anti-RORα antibody (Santa Cruz, Cat.No. sc-6062) in all six analyzed cell pellets. One viral isolate is chosen for further amplification; a master virus stock, followed by a working virus stock are generated by further amplification in Sf-9 cells; titers are determined by plaque assay. A kinetic experiment reveals optimal production conditions for RORα-sLBD using 1 MOI at 1.82×106 cells/ml (TOI) for 72 hours. Under these conditions a large fraction of the protein remained soluble in the insect cells. Two Wave Bioreactor runs are performed of approx. 10-13 liters each under the above described conditions. Cells are harvested by centrifugation for 10 minutes at 6000 g in a Heraeus Cryofuge M7000, and the pellets are stored at −80° C.

Purification and Characterization of (His)₆RORα-LBD304-556

(His)₆RORα-LBD₃₀₄₋₅₅₆ is purified by Ni—NTA chromatography followed by anion-exchange and size exclusion chromatography according to standard methods. From 20-g cell paste, around 15 mg of (His)₆RORα-LBD₃₀₄₋₅₅₆ is purified. The protein runs as a monomer on the size exclusion chromatography. N-terminal sequence analysis shows that the N-terminus is blocked. Mass spectrometry analysis shows a homogeneous molecular mass of 31′515.4 corresponding to Acetyl-desMet-(His)₆RORα-LBD₃₀₄₋₅₅₆ (Acet-GSSHHHHHHLEVLFQGPAELEH . . . MQIDG). Proteolytic cleavage of the N-terminal 6×His tag by the PreScission™ protease results in a homogeneous protein that however does not yield useful crystals. In contrast, uncleaved RORα-LBD leads to crystals suitable for X-ray diffraction analysis.

Crystallization

Recombinant human RORα-LBD in 50 mM Tris-HC; pH 7.5, 100 mM NaCl, 5 MM DTT is concentrated to 14 mg/ml. Crystallization is performed using a standard vapor diffusion hanging drop set-up, with VDX crystallization plates and siliconized microscope cover slips from Hampton Research. Crystallization droplets are made by mixing on the coverslips 2.0 μl of the protein stock solution with 2.0 μl of reservoir solution and equilibrated against 700 μl of reservoir solution at 20° C. Commercially available screening kits are used to find preliminary crystallization conditions. In the refined conditions, crystals grow within 2 weeks at 20° C. to a size of 0.15×0.15×0.3 mm with a reservoir of 100 mM Tris-HCl pH 8.4, 19% PEG 6000, 0.2M CaCl₂. The space group of the native crystals is P21, with unit cell parameters a=55.9 Å, b=49.9 Å, c=60.7 Å, β=98.7° and space group P2₁. There is one monomer per asymmetric unit. The crystals diffract at the synchrotron (SNBL at ESRF, Grenoble) to at least 1.88 Å.

X-Ray Data Collection

For the native data collection, a crystal grown as described above is transferred to 5 μl of solution containing 20% glycerol (in addition to the reservoir composition) for about 10 seconds. The crystal is then rapidly mounted in a nylon CryoLoop (Hampton Research) and directly frozen in a cold nitrogen stream for X-ray data collection at 105K. Diffraction data are collected with the mar345 image plate system of the Swiss-Norwegian beaniline of the European Synchrotron Radiation Facility (λ-0.8727 Å). A total of 230 images of 1.020 rotation each are collected in time mode (15 sec per frame) with a crystal-to-detector distance of 178 mm (using a readout plate-diameter of 180 mm). Raw diffraction data are processed and scaled with the HKL program suite version 1.96.6 (Otwinowski and Minor, 1996). Crystal data and data collection statistics for the native data are shown in Table 4. The space group of the native crystals is P2₁, with unit cell parameters a=55.9 Å, b=49.9 Å, c=60.7 Å, β=98.7°. There is one monomer per asymmetric unit. The estimated B-factor by Wilson plot is 30 Å². For the Hg-derivative data collection, a crystal is soaked previously for 1 hr in 5 μl of solution containing 4 mM methylmercuric acetate (in addition to the reservoir composition). Cryocooling is then done as for the native crystal. Diffraction data are collected with the mar345 image plate system of the Swiss-Norwegian beamline of the European Synchrotron Radiation Facility (λ=0.8727 Å). A total of 287 images of 1.0° rotation each are collected in time mode (15 sec per frame) with a crystal-to-detector distance of 178 mm (using a readout plate-dimeter of 180 mm). Raw diffraction data are processed and scaled with the HKL program suite version 1.96.6 (Otwinowski and Minor, 1996). Crystal data and data collection statistics for the Hg-derivative data are shown in Table 2. The space group of the Hg-derivative crystals is P2₁, with unit cell parameters a=55.6 Å, b=50.0 Å, c=60.1 Å, β=98.0°. There is one monomer per asymmetric unit. The estimated B-factor by Wilson plot is 29 Å².

Structure Solution

Attempts to solve the structure by molecular replacement with the programs AmoRe (Navaza, 1994) or MOLREP version 6.2.5. (Vagin & Teplyakov, J.Appl.Cryst. 30, 1022-1025, 1997) by using several different models based on the coordinates of the pdb-entries 21bd (hRARγ) or 1bsx (hTRβ) are not successful. Data from a single-wavelength experiment on the mercury-substituted crystal are thus used together with a native data set for the initial phasing by SIRAS. Anomalous as well as isomorphous difference Patterson maps reveal at least one common dominant peak. SnB version 2.1 (Weeks & Miller, J.Appl.Cryst. 32, 120-124, 1999) with DREAR normalization (Blessing & Smith, J.Appl.Cryst. 32, 664-670, 1999) using the observed anomalous differences is used to determine 4 Hg-sites. The heavy-atom parameters are subsequently refined using MLPHARE version 4.1 (CCP4, 1994). Subsequent density modification with DM (CCP4, 1994) result in an excellent experimental SIRAS-map. Skeletonization with mapman enables chain-tracing and model building with O version 7.0 (Jones et al., Acta Crystallogr. A47:110-19, 1991).

Refinement

After building the protein (residues His308-Phe544 had visible electron density) and insertion of 112 water molecules into the experimental SIRAS-map, several alternate cycles of refinement and manual rebuilding result in a model with R_{cryst}=28.1% (8 Å-1.88 Å), that give excellent 2Fo-Fc and Fo-Fc maps for a ligand in the LBP. The excellent quality of the electron density allows the unambiguous identification of the ligand as being cholest-5-en-3beta-ol (cholesterol). The cholesterol ligand is then built into the electron density and X-PLOR parameter- and structure-files can be generated with the program XPLO2D (Kleywegt G., CCP4/ESF-EACBM Newsletter on Protein Crystallography 31, 45-50, 1995) that can be used to generate the X-PLOR parameter- and structure-files. Further cycles of refinement and insertion of 119 more water molecules (leading to a total of 231 water molecules) yield the final R_{cryst}=24.8% and R_{free}=26.3% (no sigma cutoff, 8 Å-1.8 Å, working set of 25592 unique reflections, test set of 1279 reflections). In general, the electron density is of excellent quality, except for the loop 493-498 which has weak density (residues 308-544 are included in model). Refinement is done with X-PLOR 3.1 (A. Bruenger, X-PLOR Version 3.1: A system for X-ray Crystallography and NMR. Yale University Press, New Haven, Conn., USA, 1992) using the Engh and Huber force field for the protein (Engh & Huber, Acta Crystallogr. A47:392-400, 1991). The chain identifiers used are A for the protein (residues His308-Phe544, numbering according to SWISS-PROT P35398-1), L for the ligand (cholesterol: residue 1) and V for the water molecules (total of 231). The atom numbers used for the ligand cholesterol in the pdb-file are not the same as the atom numbers according to IUPAC-TUB.

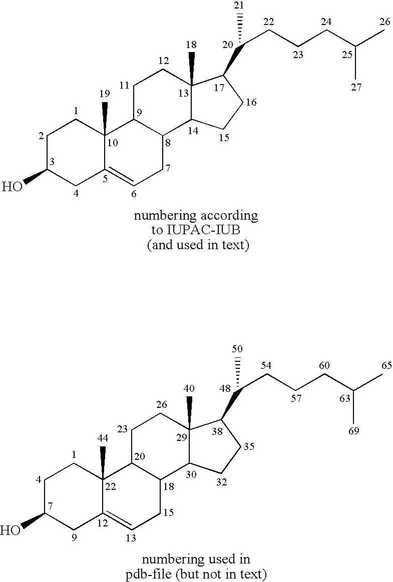

The quality of the model is assessed with X-PLOR 3.1 (A.Bruenger, id 1992) and PROCHECK v3.3 (Laskowski et al., J. Appl. Cryst. 1992; 26:283-91) (see Table 3). The final model of the complex RORα/cholesterol has good geometry (rms bond lengths=0.013 Å, nms bond angles=1.46°) and no residues are in disallowed regions of the Ramachandran plot, as determined by PROCHECK v3.3. Molecular graphics pictures are made with O version 7.0 (Jones et al., id 1991).

TABLE 1

| Number of crystals | 1 | |
|---|---|---|
| Space group | P2₁ | |
| Unit cell dimensions | 55.9 | Å, |
| | 49.9 | Å, |
| | 60.7 | Å |
| | β = 98.7° | |
| Number of monomers/a.u. | 1 | |
| Packing coefficient | 3.2 | Å³/Da |
| Resolution range | 15.0-1.88 | Å |
| Number of observations | 109,306 | |
| Number of rejected observations | 373 (0.34%) | |
| Number of unique reflections | 26,882 | |
| Wavelength | 0.8727 | Å |
| Overall | | |
| Data redundancy | 4.1 | |
| Data completeness | 99.2% | |
| <I/σ(I)> | 29.5 | |
| R_{sym}(I) | 0.056 | |
| Reflections with I ≧ 3σ(I) | 75.1% | |
| Highest resolution shell | | |
| Resolution range | 1.95-1.88 | Å |

| | |
|---|---|
| Completeness for shell | 93.2% |
| R_{sym}(I) for shell | 0.437 |
| Reflections with I > 3σ(I) | 30.5% |

TABLE 2

| Number of crystals | 1 | |
|---|---|---|
| Space group | P2₁ | |
| Unit cell dimensions | 55.6 | Å, |
| | 50.0 | Å, |
| | 60.1 | Å |
| | β = 98.0° | |
| Number of monomers/a.u. | 1 | |
| Packing coefficient | 3.2 | Å³/Da |
| Resolution range | 10.0-1.88 | Å |
| Number of observations | 121,716 | |
| Number of rejected observations | 4140 (3.4%) | |
| Number of unique reflections | 25,136 | |
| Wavelength | 0.8727 | Å |
| Overall | | |
| Data redundancy | 4.8 | |
| Data completeness | 93.6% | |
| < I/σ(I)> | 25.3 | |
| R_{sym}(I) | 0.057 | |
| Reflections with I ≧ 3σ(I) | 81.8% | |
| Highest resolution shell | | |
| Resolution range | 1.95-1.88 | Å |
| Completeness for shell | 76.2% | |
| R_{sym}(I) for shell | 0.354 | |
| Reflections with I ≧ 3σ(I) | 44.5% | |
| Resolution range used for phasing | 10.0-1.94 | Å |

| | |
|---|---|
| R_{merge}(F) between native and Hg | 23.8% |
| No. of common reflections | 23,396 |
| Phasing power for acentric data | 1.16 |
| Phasing power for centric data | 0.80 |
| Overall figure of merit | 0.314 |
| R_{cullis} on centric zone | 0.80 |
| Heavy atom site 1 (x, y, z, occ, Bfac) | −0.373, |
| | −0.546, |
| | −0.754, |
| | 0.387, |
| | 23.9 |
| Heavy atom site 2 (x, y, z, occ, Bfac) | −0.515, |
| | −0.611, |
| | −0.927, |
| | 0.429, |
| | 35.5 |
| Heavy atom site 3 (x, y, z, occ, Bfac) | −0.839, |
| | −0.478, |
| | −0.700, |
| | 0.265, |
| | 29.3 |
| Heavy atom site 4 (x, y, z, occ, Bfac) | −0.360, |
| | −0.797, |
| | −0.896, |
| | 0.270, |
| | 36.3 |

TABLE 3

| Data used in refinement | | |
|---|---|---|
| resolution range | 8.0-1.88 | Å |
| intensity cutoff (σ(F)) | 0.0 | |
| number of reflections (working set) | 25,592 | |
| number of reflections (test set) | 1,279 | |
| completeness (working + test set) | 99.0% | |
| Fit to data used in refinement | | |
| overall R_{cryst} | 0.248 | |
| overall R_{free} | 0.263 | |
| Number of non-hydrogen atoms | | |
| protein atoms | 1,953 | |
| ligand atoms | 28 | |
| water molecules | 231 | |
| Mean B values | | |
| mean B value for protein | 38.3 | Å² |
| mean B value for ligand | 20.1 | Å² |
| mean B value for water molecules | 51.8 | Å² |
| Rms deviations from ideal values | | |
| bond lengths | 0.013 | Å |

| | |
|---|---|
| bond angles | 1.46° |
| dihedral angles | 20.3° |
| improper angles | 1.3° |
| Residues in disallowed region of | 0 |
| Ramachandran plot | |
| PROCHECK G-factor | 0.28 |

Overall Structure of the RORα-LBD

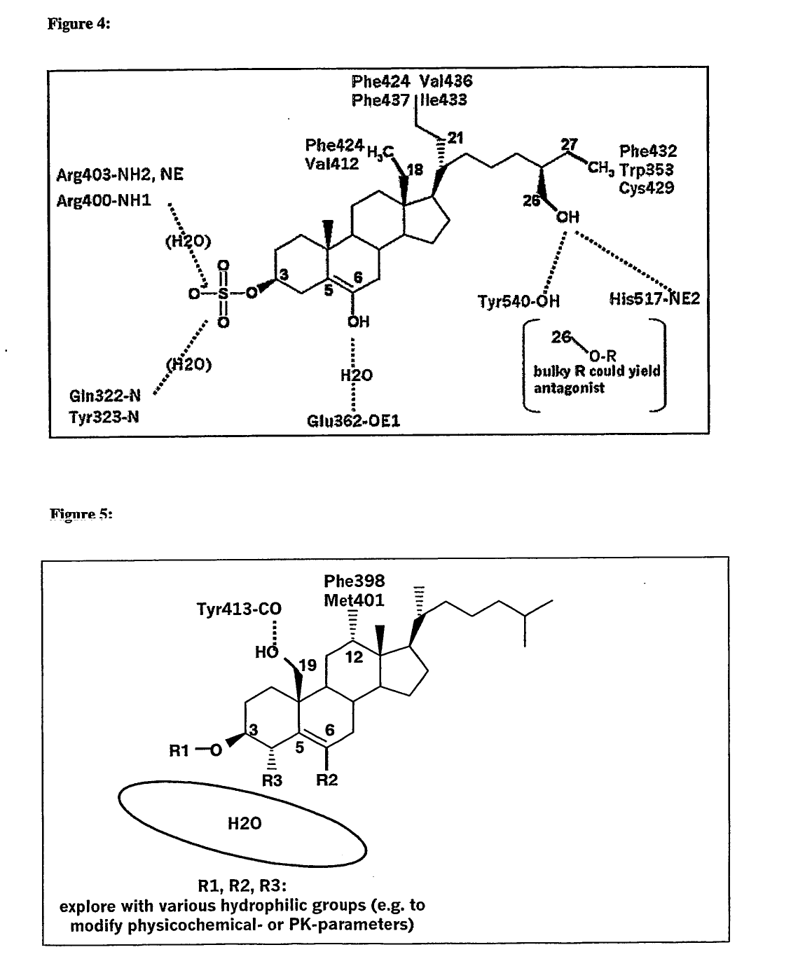

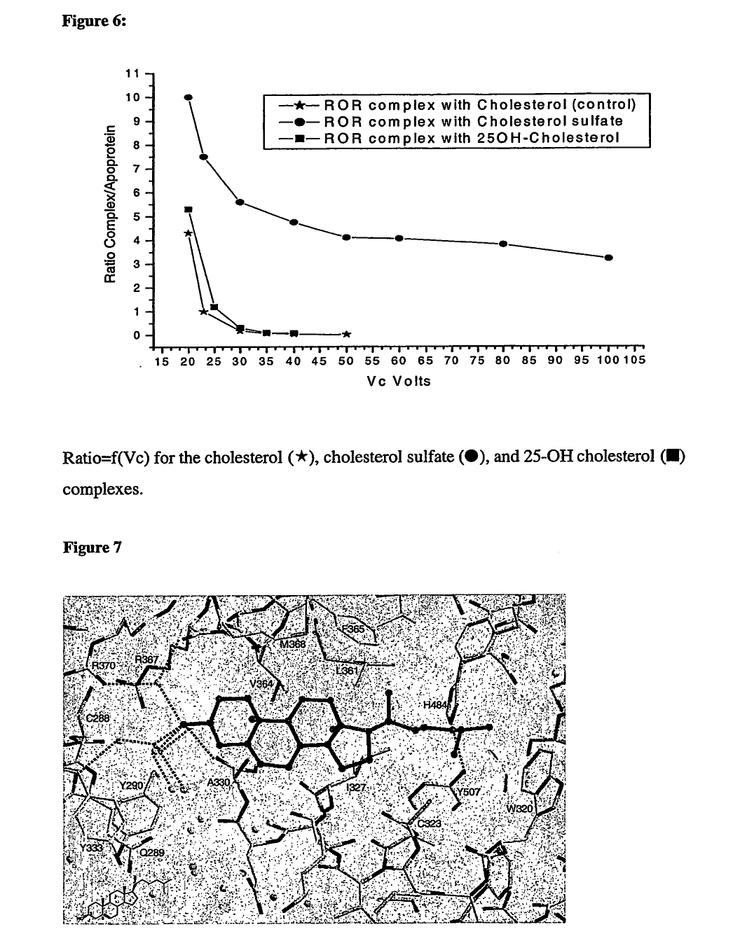

The RORα-LBD adopts the canonical fold for the NR-LBDs (Wurtz et al., Nat Struct Biol 3, 206 1996) and in addition has the two helices H2* and H11*. RORα-LBD is in an agonist-bound state, as judged by the position of H12 (see also FIGS. 2 and 3). H12 in this position, together with the H3-H4 region, forms the proper interaction surface, i.e. the complete AF-2, for the coactivator (reviewed in Renaud & Moras, Cell. Mol. Life Sci., 57, 1748-1769, 2000). No coactivator peptide is added in order to obtain this crystal structure. An additional H2* helix is also found between H2 and H3 for the peroxisome proliferator-activated receptors (PPARs; Nolte et al., Nature, 395, 137-143, 1998). HI 11* is unique to RORα-LBD (and RORβ-LBD, Stehlin et al., Embo J., 20, 5822-5832, 2001) among the known LBD structures; it roughly superposes with the middle part of loop 11-12 of RAR. The overall structure of RORα-LBD is similar to the one of RORβ-LBD (e.g. as judged by FIG. 4 in Stehlin et al., id 2001), but since the coordinates of RORβ-LBD are not available, no quantitative comparison with RORα-LBD can be made. For RORα-LBD, the putaive entrance site (as judged by the solvent accessible surface of the complex) for the ligand is located between H2 and H3, and not on the H12-side, as hypothesized e.g. for RAR-γ (Renaud et al, Nature, 378, 681-689,1995). In the crystal, the RORα-LBD molecule of the asymmetric unit does not form a dimer with a neighbouring molecule. This is consistent with the finding, that on native gels RORα-LBD behaves as a monomer. The following Cys-residues have reacted with methylmercuric acetate (c.f. table 2 for fractional coordinates of Hg-sites): Cys321 (site 3), Cys429 (site 1), Cys505 (site 4) and Cys514 (site 2). These reactive Cys-residues are thus candidates for mutations into Ser, in order to possibly obtain soluble expression in E. Coli. The protein species present in the crystallization setups correspond to the following sequences His₆-tag and PreScission™ cleavage site and residue 304-556 of RORα-LBD: Ac-GSSHHHHHHLEEVLFQGPAELHLA . . . ELFTSEFEPAMQIDG

In this crystal structure, well-defined electron density is found for the subsequence residue 308-544 (numbering according to Swissprot P35398-1).

Identification of the Ligand and Description of the Ligand Binding Pocket

A small-molecule X-ray structure of 26-OH-cholesterol from the CSD (entry FIZDUN) shows a perfect, unambigous fit (after removal of the 26-OH group and rotation of 1200 around the C24-C25 bond) into this unbiased electron density. The excellent quality of the high-resolution map thus allows the identification of the ligand as being cholest-5-en-3beta-ol (cholesterol). A closer look on Ligand binding pocket of RORα shows that C27 of the terminal isopropyl-group of cholesterol makes vdW-contacts with the sidechain of Trp353, while C26 makes vdW-contacts with the sidechain of Ile360. Substituents on C26 have the potential to influence the position of H12 (e.g. bulky substituents on C26 could displace H12 from its agonist-position, thus leading to an antagonistic derivative of cholesterol). H12 in this crystal structure adopts the agonist position. It is stabilized in the agonist position by the hydrogen bond (distance 2.8 Å) between OH-Tyr540 (on H12) and NE2-His517 (on H11). These two residues are conserved among the α-, β-, and γ-isotypes of ROR.

The LBP is essentially hydrophobic on the AF-2 side (H5 N-terminus, H6, H7, H10, H12) with the exception of Tyr540 and His517 which form an intermolecular hydrogen bond (distance between OH-TyrS40 and NE2-His517 is 2.8 Å). The LBP is more polar on the H3 side (loop 1-2, H3, H5 C-terminus). The main chain NHs of Gln322 and Tyr323 on loop 1-2 and the side chains of Arg400 and Arg403 on H5 contribute to the generation of a positive electrostatic potential. A negatively charged substituent (e.g. S04-) on the 3-ol group could thus yield a derivative with considerably increased affinity (FIG. 4). There are 12 well-ordered water molecules in the hydrophilic part. 5 of these water molecules are amongst the 7 water molecules (of the total of 231 water molecules) which have the lowest B-factors (14 Å²-24 Å²). The 3-ol group of cholesterol makes, via a network of well-ordered water molecules, water-mediated hydrogen bonds to NE-Arg403, NH2-Arg403, CO-Arg400, NH1-Arg400, NH-Tyr323, OE1-Gln322 and NH-Gln322.

The average B-value for the ligand (20.1 Å²) is lower than the average B-value for the protein (38.3 Å²), consistent with the fact that excellent electron density for all non-hydrogen atoms of cholesterol is visible. Cholesterol adopts thus a well defined, single conformation in the LBP. This is in contrast with the multiple low-energy conformations described for the non-natural ligand stearic acid present in the RORβ-LBD (Stehlin et al., id 2001). The following residues have a non-hydrogen atom closer than 4 Å to the ligand cholesterol: Trp353, Cys356, Lys359, Ile360, Ala363, Val397, Arg400, Met401, Val412, Tyr413, Phe414, Phe424, Leu427, Phe432, Val436 and His517.

Design of Cholesterol Derivative Binding to LBD of RORα

Overall, there is a very good fit of the ligand cholesterol to the LBP. Nevertheless, a comparison of the vdW-surface of the ligand with the vdW-surface of the LBP shows that there are still a few possibilities for derivatizations of cholesterol (FIGS. 4 and 5), which could increase the affinity. Additional hydrogen bonds could be gained with hydroxy-groups added to position 6 (hydrogen bond via water to OE1-Glu362), position 19 (hydrogen bond to CO-Tyr413) or position 26 (hydrogen bond to OH-Tyr540 and/or NE2-His517). Considerable electrostatic interaction energy could be gained with a charged group, e.g. SO₄⁻, added to position 3 (hydrogen bonds and electrostatic interactions via water molecules to NH1-Arg400, NH2-Arg403, NE-Arg403, NH-Gln322 and/or to NH-Tyr323). Additional vdW-interactions could be gained by additional methyl-groups added to position 12 (vdW-contacts to the sidechains of Phe398, Met401), position 18 (vdW-contacts to the sidechains of Val412, Phe398), position 27 (vdW-contacts to the sidechains of Trp353, Cys429, Phe432) or an additional ethyl-group added to position 21 (vdW-contacts to the sidechains of Phe424, Ile433, Val436, Phe437). Modifications in positions 4 and 6 could be utilized to modify, if necessary, the physicochemical or pharmacokinetic parameters, without considerably changing the affinity. Derivatives in position 26, with a bulky substituent, would have the potential to destabilize H12 in its agonist-position, thus conferring an antagonistic activity on the derivative.

Mechanism of Action for Cholesterol

The present X-ray structure promotes the following structural mechanism of action: Cholesterol (or possibly a cholesterol-derivative) enters the LBP from the H2,H3-side, possibly guided by the electrostatic field generated from Arg400 and Arg403. The isopropyl-end of cholesterol (or a derivative in this position) then influences the other end of the LBP, which is in contact with H12, thus regulating the binding of a coactivator to the LXXLL-binding site. A cholesterol-derivative with a bulky substituent on C26 could displace H12 from its agonist conformation, thus preventing coactivator binding, while a cholesterol derivative which further stabilizes the hydrogen bond between Tyr540 and His517 would further enforce the agonist conformation.

Selected Mutations of RORα-LBD

Using the coordinates from the RORα-LBD X-ray structure a series of point mutations in the LBP are designed which should prevent binding of cholesterol and in addition a mutation is proposed which should prevent/reduce H12-stabilization via loss of the hydrogen bond between Tyr540 (on H12) and His517 (Tyr 540→Phe 540 mutation). The details of the mutations are included below.

| | mutated | Mutated |
|---|---|---|
| clone name | amino acid | nucleic acids |
| SDM1-1 | Cys356 −> Leu356 | TGT −> TTA |
| SDM2-3 | Ala363 −> Leu363 | GCT −> CTT |
| SDM3-4 | Ala404 −> Gln404 | GCC −> CAA |
| SDM4-1 | Phe432 −> Trp432 | TTT −> TGG |
| SDM5-8 | His517 −> Trp517 | CAT −> TGG |
| SDM6-2 | Tyr540 −> Phe540 | TAC −> TTC |

In a transient transfection experiment, the transcriptional activity of the RORα mutants is compared to their wild type counterpart: U2OS cells are transfected with the expression vector for RORα (ROR) or its mutated form together with a luciferase reporter gene bearing a consensus response element for RORα (RORE-tk-luc). Luciferase activity is assayed in cells from 6 well plates and related to the activity in cells transfected with the wild type RORα expression plasmid. The results are normalized to the protein content. The figure shows the mean±SD and on the left panel the results are expressed as % of induction compared to the activity of the wild type RORα. As shown in Table 4 all mutations, in the LBP (except the mutation Phe 432→Trp 432) significantly/drastically reduce the transcriptional activity of RORα leading to the conclusion that indeed RORα in its active form is bound to cholesterol. The sidechain of the mutated Trp432 has the possibility to adopt a conformation for which no steric clash with cholesterol in the LBP occurs, if the sidechains of Arg516 and Lys 520 also accordingly change their conformations. Since the latter two residues are on the surface and their sidechain conformations are not stabilised by interactions, this provides an explanation for the only slight loss of transcriptional activity for the Phe432→Trp432 mutation, in contrast to the other mutations in the LBP, for which there is no alternate side-chain conformation possible which would prevent a steric clash with cholesterol. The mutation Tyr 540 ->Phe 540 leads to a ca. 40% loss in transcriptional activity, showing that the hydrogen bond between Tyr 540 and His 517 contributes in a significant amount to the stabilization of H12 in the agonist position.

TABLE 4

| a.a (Swisprot P35398 −1) | % Activity compared to WT |
|---|---|
| 356 | 33.3 |
| 363 | 18.18 |
| 404 | 8.33 |
| 432 | 90.9 |
| 517 | 10 |
| 540 | 54.54 |

Effects of Fluvastation, an Inhibitor of HMG CoA-Reductase, on RORα Transcriptional Activity

Mammalian cells receive cholesterol by uptake from lipoproteins (LDL-cholesterol) and are able to synthesize cholesterol through the mevalonate pathway. In a situation where cells are cultured under conditions essentially sterol free, a key transcription factor, SREBP will be proteolytically cleaved and this releases a transcription factor to the nucleus. This transcription factor is able to transcriptionally activate HMG-CoA reductase, which is a critical step in the cholesterol biosynthesis through the mevalonate pathway. Statins, which are know drugs for hypercholesterol state are specific inhibitors of the HMG-CoA reductase. When cells are cultivated in sterol free medium, their HMG-CoA reductase is strongly activated. In this experiment cells, cultivated in medium essentially sterol free, are treated with fluvastatin. A clear decrease in RORα activity is observed, leading to the conclusion that the lowering of the intracellular cholesterol level is translated by a decrease of RORα transcriptional activity (Table 5). U2OS cells are transfected with expression vector for RORα (ROR) together with a luciferase reporter gene bearing a consensus response element for RORα (RORE-tk-luc). Luciferase activity is assayed in cells from 6 well plates and related to the activity in cells transfected with or without treatment with fluvastatin. The results are normalized to the protein content.

TABLE 5

| Fluvastatin | Fold induction | ±SEM |
|---|---|---|
| Control | 76 | 14 |
| +5 μM | 48 | 7 |
| Control | 93 | 6 |
| +10 μM | 38 | 2 |

Cholesterol Sulfate Inhibition of RORα Binding to RORE

Various cholesterol derivatives including cholesterol sulfate (cpd No. 12 in Table 6): are screened in essentially cholesterol-free medium for binding of RORα to the RORE. The RORα protein is expressed in the baculovirus system. The other compounds are: No. 2: 5α-Cholestan-3-one (Steraloids C4550), 3: 4-Cholesten-3α-ol (C6090), 4: 5-Cholesten-3β, 6-diol (C6418), 5: 5-Cholesten-3β, 7α-diol 7-benzoate (C6425), 6: 5-Cholesten-3β, 7β-diol 7-benzoate (C6438), 7: 5-Cholesten-3β, 19-diol (C6470), 8: 5-, 25R-Cholesten-3β, 26-diol (C6570), 9: 5-Cholesten-24β-ethyl-3β-ol acetate(C6681), 10: 5-Cholesten-3α-ol (C6730), 11: 5-Cholesten-3β-ol (C6760), 12: 5-Cholesten-3β-ol sulfate, sodium salt (C6905), 13: 7, (5α-Cholesten-3β-ol (C7400), 14: 7-Dehydrocholesterol (Fluka 30800). This indicates that cholesterol sulfate, as predicted by the X-ray structure, is able to displace cholesterol.

Effect of Cholesterol and Cholesterol Derivative on ROR Alpha Transcriptional Activity

We next establish whether in eukaryotic cells partially depleted of cholesterol, RORα transcriptional activity can be reconstituted by addition of cholesterol. We therefore treat the cells with hydroxypropyl-β-cyclodextrin (HPCD), a cyclodextrin derivative known to function as a cholesterol shuttle. HPCD treatment is used in experiments aiming at the partial depletion of intracellular cholesterol. In order to prevent an increase of intracellular cholesterol through the activation of the mevalonate pathway, cells are also treated with lovastatin while they are fed with a medium containing LDL-free serum. Using a combination of HPCD and lovastatin we find that transcription of the RORE reporter is stimulated in response to cholesterol, epicholesterol and cholestanol and to an even greater extent by cholesterol sulfate and 7-dehydrocholesterol. In contrast all the hydroxycholesterols tested do not display significant activity and the cholesterol derivative 5-cholesten-24β-ethyl-3β-ol-acetate does not trigger any increase in RORα transcriptional activity as compared to vehicle (Table 6). These data correlate well with docking studies on cholesterol derivatives using our X-ray structure of RORα.

TABLE 6

| | Fold | |
|---|---|---|
| Compounds (10 μM) | induction | ±SEM |
| Control | 1 | 0.1 |
| Cholesterol | 3.3 | 0.1 |
| Epicholesterol | 2.8 | 0.44 |
| Cholestanol | 2.4 | 0.14 |
| 7-Dehydrochol | 4.6 | 0.33 |
| 22(R)-OH-Chol | 1.2 | 0.11 |
| 25-OH-Chol | 1.6 | 0.06 |
| 20(S)-OH-Chol | 1.2 | 0.11 |
| Chol. Sulfate | 5.4 | 0.31 |
| 27-OH-Chol | 1.5 | 0.14 |
| 5-Cholesten-24beta | 1 | 0.05 |

Ligand Exchange Screening by Mass Spectrometry

(His)₆RORα-LBD269-556 is produced in Sf9 cells and purified by Ni-NTA chromatography followed by size exclusion chromatography. The protein in Tris-HCl buffer at a concentration of 135 μM is incubated overnight at 4° C., with a 10-fold molar excess of 25-hydroxycholesterol (5-cholesten-3beta, 25-diol) or cholesterol sulfate (5-cholesten-3beta-ol-sulfate). Prior to mass spectrometry analysis, the protein is subjected to fast buffer exchange in 50 mM ammonium acetate pH 7.0 by size exclusion chromatography using disposable Centri∘Spin 20 columns (Princeton Separations, Adelphia, N.J.) according to manufacturer′s instructions. Mass spectrometry is carried out using a Q-Tof (Micromass, Manchester, UK) quadrupole time-of-flight hybrid tandem mass spectrometer equipped with a Micromass Z-type electrospray ionization source (ESI). The acquisition mass range is typically m/z 1500-4500 in 5 seconds. The mass spectrometer is tuned in order to allow detection of multiply-charged species of non-covalent complexes. The source block temperature and desolvation temperature are kept at 50° C. and 80° C., respectively. Sample cone voltage (Vc) is set to 23 volts for standard measurements. In-source induced fragmentation experiments are performed by increasing Vc up to 100 volts. The protein solution is infused at a flow rate of 10 μl/min. Data are recorded and processed using Masslynx software. Spectra are deconvoluted using MaxEnt analysis software (Micromass, Manchester, UK). The results show that both 25-OH cholesterol and cholesterol sulfate are able to fully displace cholesterol bound to the ROR-LBD. Moreover, the comparison at various cone-voltages (Vc) between the ligand/ROR-LBD-complex and the apo-ROR-LBD (without ligand) indicates that cholesterol and 25-OH cholesterol have a similar stability versus in-source collisions. In contrast, the cholesterol sulfate/ROR-LBD complex is more stable than cholesterol or 25-OH cholesterol complex.

Crystallization and X-Ray Structure of the Complex ROR(Alpha)/Cholesterol-Sulfate at 2.20 Å

Resolution: An Example of Structure Based Design

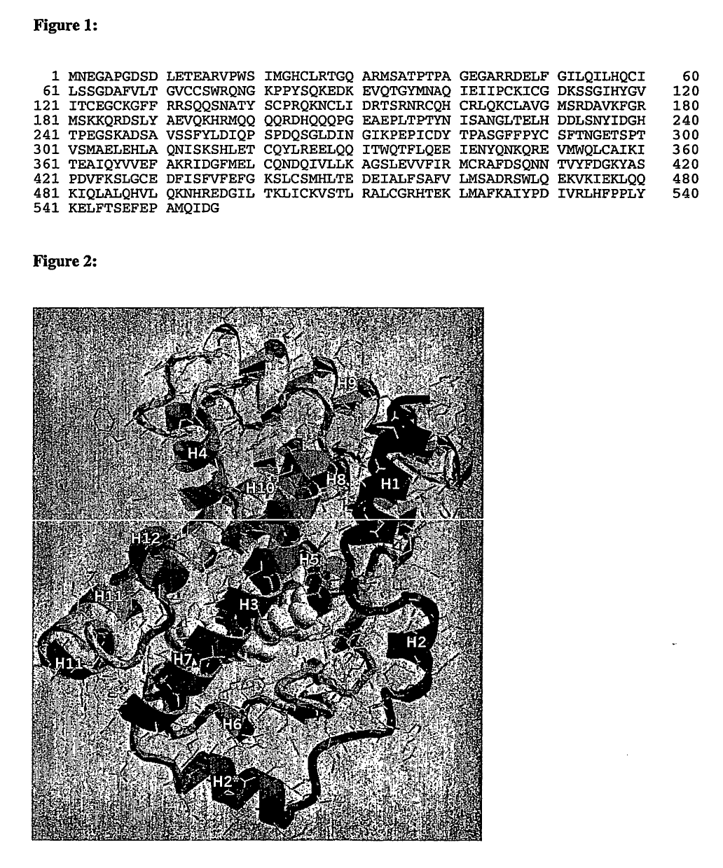

All amino acid residues relating to the complex ROR(alpha)/cholesterol-sulfate (e.g. the attached coordinates of the complex with cholesterol-sulfate, Table 9) are numbered according to splice variant Alpha-1 (i.e. P35398-2) Of SWISS-PROT entry P35398 (corresponding to the number of a given amino acid according to_SWISS-PROT P35398-1 as set out in FIG. 1 minus 33). All amino acid residues relating to the complex ROR(alpha)/cholesterol (e.g. the attached coordinates of the complex with cholesterol, Table 8) are numbered according to splice variant Alpha-2 (i.e. P35398-1) of SWISS-PROT entry P35398, except for FIGS. 7-11, where the numbering used is according to P35398-2, and except in the following discussion of the comparison with the cholesterol-sulfate complex. All amino acid residues specified in the claims are numbered according to splice variant Alpha-2 (i.e. P35398-1) of SWISS-PROT entry P35398, as set out in FIG. 1.

The proposal that cholesterol-sulfate is a ligand of ROR(alpha) is a result of structure based design, using the previously determined X-ray structure of ROR(alpha)/cholesterol at 1.63 Å resolution. In particular, the latter X-ray structure reveals that in the hydrophilic part of the LBP there is space for a substituent attached to the hydroxy-group of cholesterol, if water molecules are displaced. The presence of three arginines (Arg292, Arg370 and Arg367) and of two free backbone amide nitrogens (NH-Gln289 and NH-Tyr290) strongly suggests a negatively charged substituent with at least two hydrogen-bond acceptor functionalities (e.g. a sulfate-group). Docking studies lead to the prediction that cholesterol-sulfate should have higher affinity than cholesterol. Subsequently it is shown by MS-analysis that indeed cholesterol bound to ROR(alpha) LBD could be exchanged with cholesterol-sulfate.

The complex ROR(alpha)/cholesterol-sulfate could now be cocrystallized and the X-ray structure of the complex is solved at 2.20 Å resolution with an R_{cryst} of 19.4% and R_{free} of 21.9% for data from 20 Å to 2.20 Å. The observed binding mode shows the following features:
1). Cholesterol-sulfate and cholesterol have similar overall modes of binding, but cholesterol-sulfate is displaced slightly (e.g. corresponding C3-atoms by 0.85 Å) towards the hydrophilic, positively charged, part of the LBP. This can be explained by the optimization of electrostatic and hydrogen-bond interactions made by the sulfate-group.
2.) Seven well-ordered water molecules present for cholesterol in the hydrophilic part of the LBP have been displaced in the complex with cholesterol-sulfate. Only one conserved water molecule is still present which mediates interactions between the sulfate group and NH1-Arg367 and O-Ala330.
3.) The sulfate group makes direct hydrogen bond interactions with NH-Gln289, NH-Tyr290 and NH1-Arg370. This confirms the docking hypothesis, which led to the proposal of cholesterol-sulfate.
4.) The only significant changes in the protein parts of the complexes of ROR(alpha) with cholesterol and cholesterol-sulfate occur for the sidechain of Ile327 and the loop 1-2 (residues Gln289 and Tyr290).
   Molecular Biology, Fermentation, Purification and MS-Analysis

Generation of the construct (His)₆RORα-LBD₂₇₀₋₅₂₃, fermentation and purification are done as described above. The exchange of cholesterol by cholesterol-sulfate is done at 37° C. and confirmed by MS-analysis: Cholesterol sulfate is dissolved at 50 mM in DMSO and added at 1.0 mM final concentration to the (His)₆RORαLBD₂₇₀₋₅₂₃ solution at 73 μM. The resulting solution is incubated overnight at 37° C. and further purified by size exclusion chromatography on a SPX75 column, before concentrating to 17.6 mg/ml for crystallization trials. MS determination of the native complex is done as described previously (Kallen et al., Structure, Vol.10, 1697-1707, 2002). A control experiment is done by incubating the same amount of RORα LBD protein with 5% DMSO under identical conditions. The protein concentration is approximately 15 μM in 50 mM AcONH₄, pH 7.0. Both spectra are recorded under identical conditions with Vc=20 volts.

Crystallization

The protein used for crystallization is at 17.6 mg/ml, in 100 mM NaCl, 50 mM Tris-HCl pH7.5, 5 mM DTT. An ab inito search for crystallization conditions is undertaken. Trials are performed using a standard vapor diffusion hanging drop set-up, with VDX crystallization plates and siliconized microscope cover slips from Hampton Research. Crystallization droplets are set up at 4° C. by mixing on the coverslips 1.0 μl of the protein stock solution with 1.0 μl of a crystallization solution.

X-ray Data collection: A single crystal of approximate dimensions 60 μm×60 μm×200 μm is mounted with a nylon CryoLoop (Hampton Research) and flash-frozen in a cold nitrogen stream for X-ray data collection at 100K. Diffraction data are collected at the Swiss Light Source (operating at 300 mA), beamline X06SA, using a Marresearch CCD detector and an incident monochromatic X-ray beam with 0.9200 Å wavelength. In total, 226 images are collected with 1.0° rotation each, using an exposure time of 9 sec per frame and a crystal-to-detector distance of 150 mm. Raw diffraction data are processed and scaled with the HKL program suite version 1.96.1 (Otwinowski and Minor, 1996). The estimated B-factor by Wilson plot analysis is 32.9 Å². Crystal data and data collection statistics are shown in Table 7:

| Number of crystals | 1 | |
|---|---|---|
| Space group | P2₁ | |
| Unit cell dimensions | a = 54.4 | Å |
| | b = 49.9 | Å |
| | c = 60.7 | Å |
| No. of monomers/a.u. | 1 | |
| Packing coefficient | 3.0 | Å³/Da |
| Estimated solvent content | 58% | |
| Wavelength | 0.9200 | Å |
| Temperature | 100 | K |
| Resolution range | 20.0-2.2 | Å |
| No. of observations | 57,993 | |
| No. of unique reflections | 16,541 | |
| Overall | | |
| Resolution range | 20.0-2.2 | Å |
| Data redundancy | 3.5 | |
| Completeness | 99.7% | |
| <I/σ(I)> | 16.2 | |
| R_{merge} on intensities | 0.079 | |
| Reflections with I ≧ 3σ(I) | 66.4% | |
| Highest resolution shell | | |
| Resolution range | 2.28-2.20 | Å |

| | |
|---|---|
| Data redundancy | 2.8 |
| Completeness | 99.4% |
| <I/σ(I)> | 1.9 |
| R_{merge} on intensities | 0.362 |
| Reflections with I ≧ 3σ(I) | 25.9% |

Structure determination and refinement: The structure is determined using as starting model the coordinates of the complex ROR(alpha)/cholesterol refined to 1.63 Å resolution. The program REFMAC version 5.0 (CCP4, Acta Crystallogr. D50, 760-763, 1994) is used for refinement. Bulk solvent correction, an initial anisotropic B factor correction and restrained isotropic atomic B-factor refinement are applied. The refinement target is the maximum-likelihood target using amplitudes. No sigma cut-off is applied on the structure factor amplitudes. Cross-validation is used throughout refinement using a test set comprising 5.0% (829) of the unique reflections. Water molecules are identified with the program ARP/wARP and selected based on difference peak height (greater than 3.0σ) and distance criteria. Water molecules with temperature factors greater than 70 Å² are rejected. The program O version 7.0 (A.Jones et al., 1991) is used for model rebuilding. The refinement statistics for the final model are shown in Table 2. The final model of the complex ROR(alpha)/cholesterol-sulfate has good geometry (rms bond lengths=0.014 Å, rms bond angles=1.41°) and no residues are in a disallowed region of the Ramachandran plot.

Crystallization, data collection: The crystals used for data collection are obtained with a well solution composed of 0.2M MgCl₂, 16% w/v PEG4000, 0.1M Tris HCl, pH 8.5. The crystals reached maximal dimensions of up to 0.2 mm within 6 weeks. The complex of RORα LBD with cholesterol-sulfate is thus crystallized in a crystal form with a=54.4 Å, b=49.9 Å, c=60.7 Å, β=97.8°, P2₁ and 1 complex/asymmetric unit, which is similar to the crystal form previously obtained in the complex with cholesterol.

Conformation of cholesterol-sulfate bound to ROR(alpha) and its interactions: In general, the electron density is of excellent quality, except for amino acids 461-464 (L9-10), which has only weak density. The protein part of the refined model consists of the last two His-amino acids from the His-tag, followed by the PreScission™-site (LEVLFQG) and by amino acids 271-511 of the RORα-LBD. The refined model also contains 256 water molecules and 1 cholesterol-sulfate molecule.

The sulfate group makes direct hydrogen bond interactions with NH-Gln289 (3.0 Å), NH-Tyr290 (2.9 Å) and a bidentate interaction with NH1-Arg370 (3.0 Å, 3.1 Å). A water-mediated interaction is made with NH1-Arg367.

Comparison of the X-Ray Structures of Cholesterol-Sulfate and Cholesterol Bound to ROR(Alpha) LBD

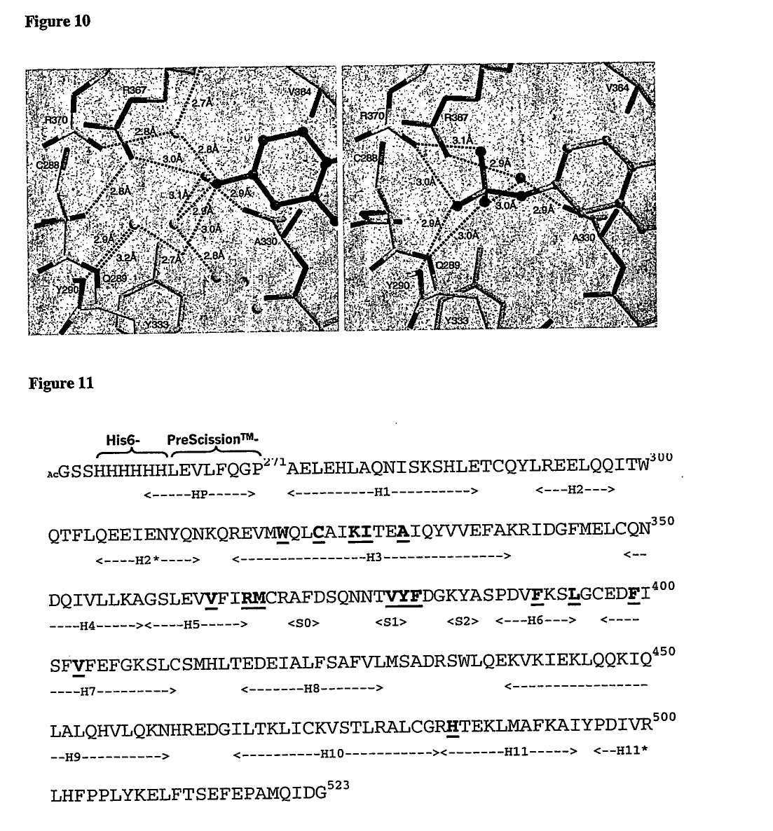

FIG. 10 shows a superposition (using Cα's of the respective LBD's) for the ROR(alpha) complexes with cholesterol and cholesterol-sulfate. The r.m.s.d for the Cα atoms of residues Pro270-Phe511 after superposition is 0.26 Å. The only significant changes in the protein parts occur for the sidechain of Ile327 and the loop 1-2 (residues Gln289 and Tyr290): The backbone NH-atoms for Gln289 and Tyr290 move by ca. 0.8 Å towards the sulfate-group (with a concomitant movement of the respective sidechains), in order to improve the interactions with the sulfate-group. The sidechain of Ile327 has to move slightly, in order to avoid a steric clash with the terminal isopropyl-group (FIG. 9). The comparison shows that cholesterol-sulfate and cholesterol have similar overall modes of binding, but cholesterol-sulfate is displaced slightly (e.g. corresponding C3-atoms by 0.85 Å and corresponding C2-atoms by 0.7 Å) towards the hydrophilic, positively charged, part of the LBP (FIG. 9). This can be explained by the optimization of electrostatic and hydrogen-bond interactions made by the sulfate-group, group. 7 well-ordered water molecules present for cholesterol in the hydrophilic part of the LBP have been displaced in the complex with cholesterol-sulfate (FIG. 10). Only one conserved water molecule is still present which mediates interactions between the sulfate group and NH1-Arg367 and O-Ala330. The sulfate group makes direct hydrogen bond interactions with NH-Gln289, NH-Tyr290 and NH1-Arg370. This confirms the docking hypothesis, which led to the proposal of cholesterol-sulfate.

TABLE 8

| | | | | | | | | | | |
|---|---|---|---|---|---|---|---|---|---|---|
| ATOM | 1 | CB | HIS | A | 308 | −3.470 | 26.612 | −1.587 | 1.00 | 57.47 |
| ATOM | 2 | CG | HIS | A | 308 | −4.960 | 26.750 | −1.571 | 1.00 | 68.25 |
| ATOM | 3 | CD2 | HIS | A | 308 | −5.766 | 27.800 | −1.862 | 1.00 | 72.02 |
| ATOM | 4 | ND1 | HIS | A | 308 | −5.794 | 25.720 | −1.190 | 1.00 | 71.55 |
| ATOM | 5 | CE1 | HIS | A | 308 | −7.049 | 26.131 | −1.245 | 1.00 | 75.57 |
| ATOM | 6 | NE2 | HIS | A | 308 | −7.061 | 27.389 | −1.652 | 1.00 | 73.93 |
| ATOM | 7 | C | HIS | A | 308 | −1.527 | 26.518 | −0.048 | 1.00 | 46.68 |
| ATOM | 8 | O | HIS | A | 308 | −1.408 | 25.970 | 1.039 | 1.00 | 46.34 |
| ATOM | 9 | N | HIS | A | 308 | −2.472 | 28.682 | −0.665 | 1.00 | 46.21 |
| ATOM | 10 | CA | HIS | A | 308 | −2.791 | 27.256 | −0.390 | 1.00 | 50.95 |
| ATOM | 11 | N | LEU | A | 309 | −0.598 | 26.489 | −0.995 | 1.00 | 43.13 |
| ATOM | 12 | CA | LEU | A | 309 | 0.692 | 25.856 | −0.780 | 1.00 | 43.49 |
| ATOM | 13 | CB | LEU | A | 309 | 1.517 | 25.900 | −2.069 | 1.00 | 41.03 |
| ATOM | 14 | CG | LEU | A | 309 | 2.967 | 25.402 | −2.033 | 1.00 | 39.69 |
| ATOM | 15 | CD1 | LEU | A | 309 | 2.988 | 23.898 | −1.765 | 1.00 | 39.26 |
| ATOM | 16 | CD2 | LEU | A | 309 | 3.668 | 25.742 | −3.348 | 1.00 | 33.46 |
| ATOM | 17 | C | LEU | A | 309 | 1.397 | 26.673 | 0.307 | 1.00 | 41.27 |
| ATOM | 18 | O | LEU | A | 309 | 1.987 | 26.121 | 1.217 | 1.00 | 39.43 |
| ATOM | 19 | N | ALA | A | 310 | 1.371 | 27.994 | 0.158 | 1.00 | 41.63 |
| ATOM | 20 | CA | ALA | A | 310 | 1.972 | 28.894 | 1.125 | 1.00 | 43.60 |
| ATOM | 21 | CB | ALA | A | 310 | 1.772 | 30.334 | 0.692 | 1.00 | 41.23 |
| ATOM | 22 | C | ALA | A | 310 | 1.324 | 28.638 | 2.494 | 1.00 | 44.99 |
| ATOM | 23 | O | ALA | A | 310 | 2.028 | 28.454 | 3.487 | 1.00 | 42.70 |
| ATOM | 24 | N | GLN | A | 311 | −0.011 | 28.589 | 2.531 | 1.00 | 46.22 |
| ATOM | 25 | CA | GLN | A | 311 | −0.765 | 28.330 | 3.767 | 1.00 | 48.70 |
| ATOM | 26 | CB | GLN | A | 311 | −2.266 | 28.239 | 3.472 | 1.00 | 55.01 |
| ATOM | 27 | CG | GLN | A | 311 | −3.081 | 29.513 | 3.686 | 1.00 | 69.31 |
| ATOM | 28 | CD | GLN | A | 311 | −4.596 | 29.289 | 3.479 | 1.00 | 78.89 |
| ATOM | 29 | OE1 | GLN | A | 311 | −5.137 | 28.224 | 3.832 | 1.00 | 83.81 |
| ATOM | 30 | NE2 | GLN | A | 311 | −5.275 | 30.278 | 2.876 | 1.00 | 82.00 |
| ATOM | 31 | C | GLN | A | 311 | −0.339 | 27.015 | 4.413 | 1.00 | 45.78 |
| ATOM | 32 | O | GLN | A | 311 | −0.043 | 26.949 | 5.599 | 1.00 | 43.47 |
| ATOM | 33 | N | ASN | A | 312 | −0.332 | 25.966 | 3.604 | 1.00 | 43.80 |
| ATOM | 34 | CA | ASN | A | 312 | 0.023 | 24.624 | 4.049 | 1.00 | 43.06 |
| ATOM | 35 | CB | ASN | A | 312 | −0.236 | 23.632 | 2.918 | 1.00 | 52.50 |
| ATOM | 36 | CG | ASN | A | 312 | 0.867 | 22.607 | 2.776 | 1.00 | 64.44 |
| ATOM | 37 | OD1 | ASN | A | 312 | 0.709 | 21.453 | 3.173 | 1.00 | 73.51 |
| ATOM | 38 | ND2 | ASN | A | 312 | 1.992 | 23.017 | 2.191 | 1.00 | 70.50 |
| ATOM | 39 | C | ASN | A | 312 | 1.437 | 24.436 | 4.606 | 1.00 | 40.68 |
| ATOM | 40 | O | ASN | A | 312 | 1.635 | 23.638 | 5.518 | 1.00 | 39.27 |
| ATOM | 41 | N | ILE | A | 313 | 2.424 | 25.072 | 3.974 | 1.00 | 39.40 |
| ATOM | 42 | CA | ILE | A | 313 | 3.824 | 24.979 | 4.407 | 1.00 | 38.82 |
| ATOM | 43 | CB | ILE | A | 313 | 4.802 | 25.421 | 3.253 | 1.00 | 34.58 |
| ATOM | 44 | CG2 | ILE | A | 313 | 6.169 | 25.806 | 3.799 | 1.00 | 36.01 |
| ATOM | 45 | CG1 | ILE | A | 313 | 4.956 | 24.284 | 2.240 | 1.00 | 37.16 |
| ATOM | 46 | CD1 | ILE | A | 313 | 6.005 | 24.537 | 1.154 | 1.00 | 35.61 |
| ATOM | 47 | C | ILE | A | 313 | 4.030 | 25.798 | 5.703 | 1.00 | 37.29 |
| ATOM | 48 | O | ILE | A | 313 | 4.786 | 25.399 | 6.585 | 1.00 | 39.16 |
| ATOM | 49 | N | SER | A | 314 | 3.298 | 26.906 | 5.823 | 1.00 | 35.32 |
| ATOM | 50 | CA | SER | A | 314 | 3.334 | 27.790 | 6.989 | 1.00 | 36.43 |
| ATOM | 51 | CB | SER | A | 314 | 2.457 | 29.014 | 6.728 | 1.00 | 37.35 |
| ATOM | 52 | OG | SER | A | 314 | 3.059 | 29.848 | 5.759 | 1.00 | 41.24 |
| ATOM | 53 | C | SER | A | 314 | 2.807 | 27.089 | 8.241 | 1.00 | 35.85 |
| ATOM | 54 | O | SER | A | 314 | 3.305 | 27.283 | 9.351 | 1.00 | 36.16 |
| ATOM | 55 | N | LYS | A | 315 | 1.777 | 26.288 | 8.033 | 1.00 | 35.61 |
| ATOM | 56 | CA | LYS | A | 315 | 1.131 | 25.547 | 9.094 | 1.00 | 36.35 |
| ATOM | 57 | CB | LYS | A | 315 | −0.183 | 24.969 | 8.564 | 1.00 | 34.96 |
| ATOM | 58 | CG | LYS | A | 315 | −1.051 | 24.316 | 9.597 | 1.00 | 38.82 |
| ATOM | 59 | CD | LYS | A | 315 | −2.470 | 24.232 | 9.084 | 1.00 | 49.73 |
| ATOM | 60 | CE | LYS | A | 315 | −3.386 | 23.556 | 10.082 | 1.00 | 56.14 |
| ATOM | 61 | NZ | LYS | A | 315 | −3.021 | 22.113 | 10.247 | 1.00 | 65.23 |
| ATOM | 62 | C | LYS | A | 315 | 2.056 | 24.438 | 9.571 | 1.00 | 36.74 |
| ATOM | 63 | O | LYS | A | 315 | 2.102 | 24.130 | 10.757 | 1.00 | 38.14 |
| ATOM | 64 | N | SER | A | 316 | 2.771 | 23.835 | 8.624 | 1.00 | 35.23 |
| ATOM | 65 | CA | SER | A | 316 | 3.708 | 22.757 | 8.904 | 1.00 | 31.03 |
| ATOM | 66 | CB | SER | A | 316 | 4.268 | 22.189 | 7.591 | 1.00 | 33.89 |
| ATOM | 67 | OG | SER | A | 316 | 3.275 | 21.504 | 6.838 | 1.00 | 36.00 |
| ATOM | 68 | C | SER | A | 316 | 4.842 | 23.293 | 9.769 | 1.00 | 31.80 |
| ATOM | 69 | O | SER | A | 316 | 5.223 | 22.667 | 10.760 | 1.00 | 30.02 |
| ATOM | 70 | N | HIS | A | 317 | 5.391 | 24.440 | 9.354 | 1.00 | 29.40 |
| ATOM | 71 | CA | HIS | A | 317 | 6.468 | 25.128 | 10.078 | 1.00 | 31.38 |
| ATOM | 72 | CB | HIS | A | 317 | 6.838 | 26.397 | 9.319 | 1.00 | 28.86 |
| ATOM | 73 | CG | HIS | A | 317 | 7.765 | 27.317 | 10.058 | 1.00 | 30.88 |
| ATOM | 74 | CD2 | HIS | A | 317 | 7.590 | 28.583 | 10.506 | 1.00 | 31.39 |
| ATOM | 75 | ND1 | HIS | A | 317 | 9.085 | 27.007 | 10.310 | 1.00 | 31.14 |
| ATOM | 76 | CE1 | HIS | A | 317 | 9.686 | 28.042 | 10.866 | 1.00 | 29.44 |
| ATOM | 77 | NE2 | HIS | A | 317 | 8.801 | 29.012 | 10.996 | 1.00 | 30.99 |
| ATOM | 78 | C | HIS | A | 317 | 5.964 | 25.489 | 11.486 | 1.00 | 32.96 |
| ATOM | 79 | O | HIS | A | 317 | 6.647 | 25.271 | 12.491 | 1.00 | 30.21 |
| ATOM | 80 | N | LEU | A | 318 | 4.766 | 26.066 | 11.519 | 1.00 | 35.80 |
| ATOM | 81 | CA | LEU | A | 318 | 4.099 | 26.456 | 12.754 | 1.00 | 39.30 |
| ATOM | 82 | CB | LEU | A | 318 | 2.651 | 26.888 | 12.454 | 1.00 | 42.10 |
| ATOM | 83 | CG | LEU | A | 318 | 1.664 | 27.026 | 13.629 | 1.00 | 45.53 |
| ATOM | 84 | CD1 | LEU | A | 318 | 1.898 | 28.331 | 14.354 | 1.00 | 42.84 |
| ATOM | 85 | CD2 | LEU | A | 318 | 0.233 | 26.963 | 13.127 | 1.00 | 46.58 |
| ATOM | 86 | C | LEU | A | 318 | 4.070 | 25.267 | 13.708 | 1.00 | 39.53 |
| ATOM | 87 | O | LEU | A | 318 | 4.581 | 25.337 | 14.829 | 1.00 | 45.11 |
| ATOM | 88 | N | GLU | A | 319 | 3.517 | 24.157 | 13.235 | 1.00 | 34.75 |
| ATOM | 89 | CA | GLU | A | 319 | 3.378 | 22.951 | 14.040 | 1.00 | 31.78 |
| ATOM | 90 | CB | GLU | A | 319 | 2.258 | 22.088 | 13.464 | 1.00 | 35.64 |
| ATOM | 91 | CG | GLU | A | 319 | 0.966 | 22.887 | 13.304 | 1.00 | 43.85 |
| ATOM | 92 | CD | GLU | A | 319 | −0.204 | 22.079 | 12.797 | 1.00 | 48.29 |
| ATOM | 93 | OE1 | GLU | A | 319 | −0.046 | 20.870 | 12.496 | 1.00 | 51.24 |
| ATOM | 94 | OE2 | GLU | A | 319 | −1.299 | 22.675 | 12.715 | 1.00 | 49.93 |
| ATOM | 95 | C | GLU | A | 319 | 4.605 | 22.092 | 14.335 | 1.00 | 31.28 |
| ATOM | 96 | O | GLU | A | 319 | 4.501 | 21.150 | 15.128 | 1.00 | 29.74 |
| ATOM | 97 | N | THR | A | 320 | 5.749 | 22.374 | 13.703 | 1.00 | 29.30 |
| ATOM | 98 | CA | THR | A | 320 | 6.948 | 21.589 | 13.957 | 1.00 | 25.31 |
| ATOM | 99 | CB | THR | A | 320 | 7.428 | 20.826 | 12.723 | 1.00 | 26.81 |
| ATOM | 100 | OG1 | THR | A | 320 | 7.760 | 21.760 | 11.697 | 1.00 | 31.74 |
| ATOM | 101 | CG2 | THR | A | 320 | 6.371 | 19.874 | 12.228 | 1.00 | 25.26 |
| ATOM | 102 | C | THR | A | 320 | 8.086 | 22.435 | 14.499 | 1.00 | 27.08 |
| ATOM | 103 | O | THR | A | 320 | 9.251 | 22.078 | 14.369 | 1.00 | 28.40 |
| ATOM | 104 | N | CYS | A | 321 | 7.754 | 23.591 | 15.058 | 1.00 | 27.84 |
| ATOM | 105 | CA | CYS | A | 321 | 8.758 | 24.440 | 15.690 | 1.00 | 32.58 |
| ATOM | 106 | CB | CYS | A | 321 | 8.575 | 25.897 | 15.291 | 1.00 | 35.52 |
| ATOM | 107 | SG | CYS | A | 321 | 9.587 | 26.379 | 13.907 | 1.00 | 31.41 |
| ATOM | 108 | C | CYS | A | 321 | 8.481 | 24.273 | 17.183 | 1.00 | 33.42 |
| ATOM | 109 | O | CYS | A | 321 | 7.315 | 24.272 | 17.584 | 1.00 | 33.09 |
| ATOM | 110 | N | GLN | A | 322 | 9.516 | 24.122 | 18.005 | 1.00 | 33.42 |
| ATOM | 111 | CA | GLN | A | 322 | 9.280 | 23.945 | 19.435 | 1.00 | 37.77 |
| ATOM | 112 | CB | GLN | A | 322 | 10.566 | 23.575 | 20.159 | 1.00 | 38.95 |
| ATOM | 113 | CG | GLN | A | 322 | 10.311 | 23.332 | 21.638 | 1.00 | 41.00 |
| ATOM | 114 | CD | GLN | A | 322 | 11.474 | 22.709 | 22.355 | 1.00 | 43.45 |
| ATOM | 115 | OE1 | GLN | A | 322 | 12.639 | 22.892 | 21.974 | 1.00 | 45.86 |
| ATOM | 116 | NE2 | GLN | A | 322 | 11.173 | 21.973 | 23.408 | 1.00 | 43.47 |
| ATOM | 117 | C | GLN | A | 322 | 8.595 | 25.133 | 20.143 | 1.00 | 38.21 |
| ATOM | 118 | O | GLN | A | 322 | 7.627 | 24.945 | 20.891 | 1.00 | 40.89 |
| ATOM | 119 | N | TYR | A | 323 | 9.087 | 26.348 | 19.893 | 1.00 | 37.01 |
| ATOM | 120 | CA | TYR | A | 323 | 8.518 | 27.545 | 20.513 | 1.00 | 39.53 |
| ATOM | 121 | CB | TYR | A | 323 | 9.576 | 28.318 | 21.306 | 1.00 | 37.75 |
| ATOM | 122 | CG | TYR | A | 323 | 10.245 | 27.509 | 22.370 | 1.00 | 36.25 |
| ATOM | 123 | CD1 | TYR | A | 323 | 11.551 | 27.058 | 22.191 | 1.00 | 40.18 |
| ATOM | 124 | CE1 | TYR | A | 323 | 12.185 | 26.279 | 23.139 | 1.00 | 43.63 |
| ATOM | 125 | CD2 | TYR | A | 323 | 9.576 | 27.164 | 23.537 | 1.00 | 38.29 |
| ATOM | 126 | CE2 | TYR | A | 323 | 10.196 | 26.386 | 24.500 | 1.00 | 43.87 |
| ATOM | 127 | CZ | TYR | A | 323 | 11.508 | 25.945 | 24.294 | 1.00 | 45.72 |
| ATOM | 128 | OH | TYR | A | 323 | 12.165 | 25.182 | 25.236 | 1.00 | 50.91 |
| ATOM | 129 | C | TYR | A | 323 | 7.916 | 28.496 | 19.502 | 1.00 | 41.39 |
| ATOM | 130 | O | TYR | A | 323 | 8.185 | 28.404 | 18.302 | 1.00 | 43.21 |
| ATOM | 131 | N | LEU | A | 324 | 7.149 | 29.451 | 20.011 | 1.00 | 38.45 |
| ATOM | 132 | CA | LEU | A | 324 | 6.556 | 30.442 | 19.158 | 1.00 | 36.36 |
| ATOM | 133 | CB | LEU | A | 324 | 5.260 | 30.960 | 19.761 | 1.00 | 40.65 |
| ATOM | 134 | CG | LEU | A | 324 | 4.135 | 29.917 | 19.804 | 1.00 | 47.37 |
| ATOM | 135 | CD1 | LEU | A | 324 | 3.021 | 30.333 | 20.790 | 1.00 | 48.02 |
| ATOM | 136 | CD2 | LEU | A | 324 | 3.569 | 29.698 | 18.390 | 1.00 | 48.49 |
| ATOM | 137 | C | LEU | A | 324 | 7.586 | 31.548 | 19.037 | 1.00 | 36.82 |
| ATOM | 138 | O | LEU | A | 324 | 8.369 | 31.791 | 19.955 | 1.00 | 35.71 |
| ATOM | 139 | N | ARG | A | 325 | 7.604 | 32.185 | 17.876 | 1.00 | 33.86 |
| ATOM | 140 | CA | ARG | A | 325 | 8.519 | 33.274 | 17.595 | 1.00 | 34.00 |
| ATOM | 141 | CB | ARG | A | 325 | 8.132 | 33.913 | 16.252 | 1.00 | 32.60 |
| ATOM | 142 | CG | ARG | A | 325 | 9.087 | 34.947 | 15.744 | 1.00 | 29.17 |
| ATOM | 143 | CD | ARG | A | 325 | 10.477 | 34.371 | 15.667 | 1.00 | 30.77 |
| ATOM | 144 | NE | ARG | A | 325 | 11.388 | 35.268 | 14.984 | 1.00 | 31.48 |
| ATOM | 145 | CZ | ARG | A | 325 | 11.340 | 35.518 | 13.681 | 1.00 | 41.33 |
| ATOM | 146 | NH1 | ARG | A | 325 | 10.421 | 34.931 | 12.912 | 1.00 | 39.89 |
| ATOM | 147 | NH2 | ARG | A | 325 | 12.195 | 36.383 | 13.147 | 1.00 | 42.61 |
| ATOM | 148 | C | ARG | A | 325 | 8.462 | 34.328 | 18.711 | 1.00 | 36.35 |
| ATOM | 149 | O | ARG | A | 325 | 9.503 | 34.826 | 19.145 | 1.00 | 37.18 |
| ATOM | 150 | N | GLU | A | 326 | 7.244 | 34.639 | 19.169 | 1.00 | 39.65 |
| ATOM | 151 | CA | GLU | A | 326 | 6.985 | 35.626 | 20.226 | 1.00 | 41.32 |
| ATOM | 152 | CB | GLU | A | 326 | 5.487 | 35.784 | 20.459 | 1.00 | 46.07 |
| ATOM | 153 | CG | GLU | A | 326 | 4.720 | 36.379 | 19.272 | 1.00 | 65.71 |
| ATOM | 154 | CD | GLU | A | 326 | 4.555 | 35.424 | 18.062 | 1.00 | 72.41 |
| ATOM | 155 | OE1 | GLU | A | 326 | 4.261 | 34.214 | 18.267 | 1.00 | 74.04 |
| ATOM | 156 | OE2 | GLU | A | 326 | 4.696 | 35.904 | 16.901 | 1.00 | 73.55 |
| ATOM | 157 | C | GLU | A | 326 | 7.659 | 35.211 | 21.520 | 1.00 | 38.16 |
| ATOM | 158 | O | GLU | A | 326 | 8.332 | 36.023 | 22.148 | 1.00 | 35.77 |
| ATOM | 159 | N | GLU | A | 327 | 7.487 | 33.938 | 21.880 | 1.00 | 35.48 |
| ATOM | 160 | CA | GLU | A | 327 | 8.092 | 33.353 | 23.077 | 1.00 | 38.23 |
| ATOM | 161 | CB | GLU | A | 327 | 7.911 | 31.833 | 23.082 | 1.00 | 46.45 |
| ATOM | 162 | CG | GLU | A | 327 | 6.486 | 31.293 | 23.134 | 1.00 | 57.52 |
| ATOM | 163 | CD | GLU | A | 327 | 6.452 | 29.752 | 23.125 | 1.00 | 63.49 |
| ATOM | 164 | OE1 | GLU | A | 327 | 7.441 | 29.113 | 23.557 | 1.00 | 68.14 |
| ATOM | 165 | OE2 | GLU | A | 327 | 5.445 | 29.172 | 22.667 | 1.00 | 68.00 |
| ATOM | 166 | C | GLU | A | 327 | 9.599 | 33.615 | 23.140 | 1.00 | 34.14 |
| ATOM | 167 | O | GLU | A | 327 | 10.098 | 34.218 | 24.076 | 1.00 | 34.19 |
| ATOM | 168 | N | LEU | A | 328 | 10.304 | 33.158 | 22.114 | 1.00 | 29.72 |
| ATOM | 169 | CA | LEU | A | 328 | 11.748 | 33.293 | 22.006 | 1.00 | 29.32 |
| ATOM | 170 | CB | LEU | A | 328 | 12.217 | 32.636 | 20.712 | 1.00 | 29.59 |
| ATOM | 171 | CG | LEU | A | 328 | 12.016 | 31.131 | 20.626 | 1.00 | 30.01 |
| ATOM | 172 | CD1 | LEU | A | 328 | 11.986 | 30.725 | 19.164 | 1.00 | 31.46 |
| ATOM | 173 | CD2 | LEU | A | 328 | 13.119 | 30.432 | 21.367 | 1.00 | 22.42 |
| ATOM | 174 | C | LEU | A | 328 | 12.267 | 34.715 | 22.041 | 1.00 | 28.85 |
| ATOM | 175 | O | LEU | A | 328 | 13.366 | 34.954 | 22.518 | 1.00 | 29.43 |
| ATOM | 176 | N | GLN | A | 329 | 11.486 | 35.654 | 21.520 | 1.00 | 33.80 |
| ATOM | 177 | CA | GLN | A | 329 | 11.901 | 37.056 | 21.481 | 1.00 | 37.54 |
| ATOM | 178 | CB | GLN | A | 329 | 11.093 | 37.808 | 20.439 | 1.00 | 43.64 |
| ATOM | 179 | CG | GLN | A | 329 | 11.132 | 37.198 | 19.050 | 1.00 | 53.22 |
| ATOM | 180 | CD | GLN | A | 329 | 10.283 | 37.983 | 18.065 | 1.00 | 59.04 |
| ATOM | 181 | OE1 | GLN | A | 329 | 9.035 | 37.966 | 18.127 | 1.00 | 59.51 |
| ATOM | 182 | NE2 | GLN | A | 329 | 10.953 | 38.720 | 17.174 | 1.00 | 59.14 |
| ATOM | 183 | C | GLN | A | 329 | 11.725 | 37.721 | 22.846 | 1.00 | 35.96 |
| ATOM | 184 | O | GLN | A | 329 | 12.525 | 38.562 | 23.241 | 1.00 | 30.61 |
| ATOM | 185 | N | GLN | A | 330 | 10.695 | 37.308 | 23.572 | 1.00 | 36.62 |
| ATOM | 186 | CA | GLN | A | 330 | 10.450 | 37.846 | 24.901 | 1.00 | 41.09 |
| ATOM | 187 | CB | GLN | A | 330 | 9.093 | 37.383 | 25.391 | 1.00 | 45.78 |
| ATOM | 188 | CG | GLN | A | 330 | 7.957 | 37.930 | 24.579 | 1.00 | 63.61 |
| ATOM | 189 | CD | GLN | A | 330 | 6.686 | 37.133 | 24.784 | 1.00 | 74.77 |
| ATOM | 190 | OE1 | GLN | A | 330 | 6.569 | 36.365 | 25.750 | 1.00 | 79.50 |
| ATOM | 191 | NE2 | GLN | A | 330 | 5.730 | 37.285 | 23.865 | 1.00 | 82.47 |
| ATOM | 192 | C | GLN | A | 330 | 11.515 | 37.477 | 25.951 | 1.00 | 38.55 |
| ATOM | 193 | O | GLN | A | 330 | 11.609 | 38.135 | 26.979 | 1.00 | 41.30 |
| ATOM | 194 | N | ILE | A | 331 | 12.305 | 36.429 | 25.715 | 1.00 | 32.00 |
| ATOM | 195 | CA | ILE | A | 331 | 13.313 | 36.015 | 26.686 | 1.00 | 23.76 |
| ATOM | 196 | CB | ILE | A | 331 | 13.078 | 34.596 | 27.169 | 1.00 | 22.66 |
| ATOM | 197 | CG2 | ILE | A | 331 | 11.656 | 34.435 | 27.630 | 1.00 | 21.34 |
| ATOM | 198 | CG1 | ILE | A | 331 | 13.355 | 33.619 | 26.053 | 1.00 | 24.33 |
| ATOM | 199 | CD1 | ILE | A | 331 | 13.364 | 32.235 | 26.555 | 1.00 | 23.84 |
| ATOM | 200 | C | ILE | A | 331 | 14.738 | 36.183 | 26.205 | 1.00 | 23.46 |
| ATOM | 201 | O | ILE | A | 331 | 15.657 | 35.455 | 26.592 | 1.00 | 27.34 |
| ATOM | 202 | N | THR | A | 332 | 14.918 | 37.180 | 25.354 | 1.00 | 23.06 |
| ATOM | 203 | CA | THR | A | 332 | 16.204 | 37.521 | 24.799 | 1.00 | 23.34 |
| ATOM | 204 | CB | THR | A | 332 | 15.962 | 38.552 | 23.680 | 1.00 | 31.64 |
| ATOM | 205 | OG1 | THR | A | 332 | 16.261 | 37.954 | 22.404 | 1.00 | 35.81 |
| ATOM | 206 | CG2 | THR | A | 332 | 16.735 | 39.863 | 23.912 | 1.00 | 37.99 |
| ATOM | 207 | C | THR | A | 332 | 17.180 | 38.051 | 25.857 | 1.00 | 20.21 |
| ATOM | 208 | O | THR | A | 332 | 18.401 | 37.887 | 25.749 | 1.00 | 21.02 |
| ATOM | 209 | N | TRP | A | 333 | 16.628 | 38.683 | 26.886 | 1.00 | 25.18 |
| ATOM | 210 | CA | TRP | A | 333 | 17.437 | 39.226 | 27.988 | 1.00 | 23.07 |
| ATOM | 211 | CB | TRP | A | 333 | 16.582 | 40.108 | 28.879 | 1.00 | 19.83 |
| ATOM | 212 | CG | TRP | A | 333 | 15.407 | 39.426 | 29.504 | 1.00 | 17.19 |
| ATOM | 213 | CD2 | TRP | A | 333 | 15.344 | 38.851 | 30.820 | 1.00 | 22.36 |
| ATOM | 214 | CE2 | TRP | A | 333 | 14.030 | 38.392 | 31.008 | 1.00 | 26.11 |
| ATOM | 215 | CE3 | TRP | A | 333 | 16.274 | 38.693 | 31.865 | 1.00 | 22.59 |
| ATOM | 216 | CD1 | TRP | A | 333 | 14.172 | 39.294 | 28.974 | 1.00 | 14.35 |
| ATOM | 217 | NE1 | TRP | A | 333 | 13.336 | 38.671 | 29.858 | 1.00 | 19.63 |
| ATOM | 218 | CZ2 | TRP | A | 333 | 13.607 | 37.778 | 32.213 | 1.00 | 25.43 |
| ATOM | 219 | CZ3 | TRP | A | 333 | 15.852 | 38.082 | 33.056 | 1.00 | 21.23 |
| ATOM | 220 | CH2 | TRP | A | 333 | 14.538 | 37.636 | 33.216 | 1.00 | 20.18 |
| ATOM | 221 | C | TRP | A | 333 | 18.028 | 38.116 | 28.826 | 1.00 | 23.38 |
| ATOM | 222 | O | TRP | A | 333 | 19.030 | 38.314 | 29.500 | 1.00 | 27.96 |
| ATOM | 223 | N | GLN | A | 334 | 17.436 | 36.925 | 28.730 | 1.00 | 24.06 |
| ATOM | 224 | CA | GLN | A | 334 | 17.893 | 35.767 | 29.492 | 1.00 | 22.85 |
| ATOM | 225 | CB | GLN | A | 334 | 16.804 | 34.712 | 29.586 | 1.00 | 22.07 |
| ATOM | 226 | CG | GLN | A | 334 | 15.575 | 35.251 | 30.240 | 1.00 | 25.58 |
| ATOM | 227 | CD | GLN | A | 334 | 14.492 | 34.228 | 30.427 | 1.00 | 29.55 |
| ATOM | 228 | OE1 | GLN | A | 334 | 14.621 | 33.066 | 30.029 | 1.00 | 32.54 |
| ATOM | 229 | NE2 | GLN | A | 334 | 13.388 | 34.664 | 31.006 | 1.00 | 31.52 |
| ATOM | 230 | C | GLN | A | 334 | 19.169 | 35.145 | 29.016 | 1.00 | 23.02 |
| ATOM | 231 | O | GLN | A | 334 | 19.180 | 34.020 | 28.519 | 1.00 | 27.49 |
| ATOM | 232 | N | THR | A | 335 | 20.257 | 35.878 | 29.179 | 1.00 | 21.73 |
| ATOM | 233 | CA | THR | A | 335 | 21.566 | 35.403 | 28.804 | 1.00 | 20.96 |
| ATOM | 234 | CB | THR | A | 335 | 22.436 | 36.595 | 28.396 | 1.00 | 26.82 |
| ATOM | 235 | OG1 | THR | A | 335 | 22.471 | 37.528 | 29.487 | 1.00 | 25.70 |
| ATOM | 236 | CG2 | THR | A | 335 | 21.881 | 37.286 | 27.109 | 1.00 | 18.02 |
| ATOM | 237 | C | THR | A | 335 | 22.237 | 34.647 | 29.978 | 1.00 | 24.27 |
| ATOM | 238 | O | THR | A | 335 | 21.794 | 34.762 | 31.133 | 1.00 | 25.14 |
| ATOM | 239 | N | PHE | A | 336 | 23.306 | 33.902 | 29.682 | 1.00 | 22.91 |
| ATOM | 240 | CA | PHE | A | 336 | 24.048 | 33.138 | 30.693 | 1.00 | 27.78 |
| ATOM | 241 | CB | PHE | A | 336 | 25.036 | 32.126 | 30.051 | 1.00 | 23.18 |
| ATOM | 242 | CG | PHE | A | 336 | 24.385 | 30.861 | 29.569 | 1.00 | 24.30 |
| ATOM | 243 | CD1 | PHE | A | 336 | 24.236 | 30.615 | 28.193 | 1.00 | 24.39 |
| ATOM | 244 | CD2 | PHE | A | 336 | 23.855 | 29.952 | 30.477 | 1.00 | 18.74 |
| ATOM | 245 | CE1 | PHE | A | 336 | 23.558 | 29.484 | 27.734 | 1.00 | 19.18 |
| ATOM | 246 | CE2 | PHE | A | 336 | 23.174 | 28.824 | 30.043 | 1.00 | 20.30 |
| ATOM | 247 | CZ | PHE | A | 336 | 23.018 | 28.582 | 28.667 | 1.00 | 24.72 |
| ATOM | 248 | C | PHE | A | 336 | 24.835 | 34.058 | 31.632 | 1.00 | 30.11 |
| ATOM | 249 | O | PHE | A | 336 | 25.560 | 34.953 | 31.182 | 1.00 | 27.94 |
| ATOM | 250 | N | LEU | A | 337 | 24.682 | 33.840 | 32.934 | 1.00 | 29.31 |
| ATOM | 251 | CA | LEU | A | 337 | 25.413 | 34.631 | 33.921 | 1.00 | 30.51 |
| ATOM | 252 | CB | LEU | A | 337 | 24.920 | 34.293 | 35.339 | 1.00 | 30.83 |
| ATOM | 253 | CG | LEU | A | 337 | 23.426 | 34.451 | 35.652 | 1.00 | 27.00 |
| ATOM | 254 | CD1 | LEU | A | 337 | 23.096 | 33.895 | 37.023 | 1.00 | 28.96 |
| ATOM | 255 | CD2 | LEU | A | 337 | 23.047 | 35.915 | 35.568 | 1.00 | 29.51 |
| ATOM | 256 | C | LEU | A | 337 | 26.902 | 34.311 | 33.755 | 1.00 | 30.04 |
| ATOM | 257 | O | LEU | A | 337 | 27.247 | 33.289 | 33.180 | 1.00 | 31.12 |
| ATOM | 258 | N | GLN | A | 338 | 27.779 | 35.200 | 34.226 | 1.00 | 32.35 |
| ATOM | 259 | CA | GLN | A | 338 | 29.231 | 35.036 | 34.098 | 1.00 | 32.95 |
| ATOM | 260 | CB | GLN | A | 338 | 29.954 | 36.189 | 34.782 | 1.00 | 36.81 |
| ATOM | 261 | CG | GLN | A | 338 | 31.330 | 36.423 | 34.214 | 1.00 | 45.74 |
| ATOM | 262 | CD | GLN | A | 338 | 31.292 | 36.625 | 32.691 | 1.00 | 60.39 |
| ATOM | 263 | OE1 | GLN | A | 338 | 30.414 | 37.330 | 32.158 | 1.00 | 62.23 |
| ATOM | 264 | NE2 | GLN | A | 338 | 32.237 | 35.995 | 31.985 | 1.00 | 63.44 |
| ATOM | 265 | C | GLN | A | 338 | 29.810 | 33.731 | 34.616 | 1.00 | 32.21 |
| ATOM | 266 | O | GLN | A | 338 | 30.818 | 33.230 | 34.122 | 1.00 | 31.25 |
| ATOM | 267 | N | GLU | A | 339 | 29.212 | 33.234 | 35.681 | 1.00 | 33.70 |
| ATOM | 268 | CA | GLU | A | 339 | 29.655 | 31.989 | 36.297 | 1.00 | 35.07 |
| ATOM | 269 | CB | GLU | A | 339 | 28.889 | 31.747 | 37.611 | 1.00 | 42.66 |
| ATOM | 270 | CG | GLU | A | 339 | 28.630 | 33.037 | 38.449 | 1.00 | 55.90 |
| ATOM | 271 | CD | GLU | A | 339 | 27.288 | 33.741 | 38.127 | 1.00 | 58.95 |
| ATOM | 272 | OE1 | GLU | A | 339 | 27.183 | 34.992 | 38.255 | 1.00 | 47.67 |
| ATOM | 273 | OE2 | GLU | A | 339 | 26.325 | 33.023 | 37.771 | 1.00 | 67.60 |
| ATOM | 274 | C | GLU | A | 339 | 29.340 | 30.878 | 35.319 | 1.00 | 27.81 |
| ATOM | 275 | O | GLU | A | 339 | 30.156 | 30.016 | 35.062 | 1.00 | 26.00 |
| ATOM | 276 | N | GLU | A | 340 | 28.125 | 30.906 | 34.795 | 1.00 | 26.98 |
| ATOM | 277 | CA | GLU | A | 340 | 27.678 | 29.911 | 33.831 | 1.00 | 28.10 |
| ATOM | 278 | CB | GLU | A | 340 | 26.223 | 30.163 | 33.457 | 1.00 | 29.51 |
| ATOM | 279 | CG | GLU | A | 340 | 25.282 | 30.053 | 34.636 | 1.00 | 26.99 |
| ATOM | 280 | CD | GLU | A | 340 | 23.849 | 30.355 | 34.284 | 1.00 | 30.46 |
| ATOM | 281 | OE1 | GLU | A | 340 | 22.970 | 29.488 | 34.510 | 1.00 | 34.56 |
| ATOM | 282 | OE2 | GLU | A | 340 | 23.580 | 31.471 | 33.802 | 1.00 | 31.24 |
| ATOM | 283 | C | GLU | A | 340 | 28.573 | 29.875 | 32.592 | 1.00 | 25.22 |
| ATOM | 284 | O | GLU | A | 340 | 28.910 | 28.798 | 32.096 | 1.00 | 24.52 |
| ATOM | 285 | N | ILE | A | 341 | 29.008 | 31.039 | 32.126 | 1.00 | 23.36 |
| ATOM | 286 | CA | ILE | A | 341 | 29.878 | 31.070 | 30.953 | 1.00 | 28.31 |
| ATOM | 287 | CB | ILE | A | 341 | 30.087 | 32.544 | 30.391 | 1.00 | 27.91 |
| ATOM | 288 | CG2 | ILE | A | 341 | 31.242 | 32.598 | 29.382 | 1.00 | 23.19 |
| ATOM | 289 | CG1 | ILE | A | 341 | 28.799 | 33.040 | 29.718 | 1.00 | 26.01 |
| ATOM | 290 | CD1 | ILE | A | 341 | 28.772 | 34.533 | 29.426 | 1.00 | 26.73 |
| ATOM | 291 | C | ILE | A | 341 | 31.210 | 30.382 | 31.208 | 1.00 | 29.36 |
| ATOM | 292 | O | ILE | A | 341 | 31.682 | 29.598 | 30.388 | 1.00 | 29.02 |
| ATOM | 293 | N | GLU | A | 342 | 31.800 | 30.642 | 32.372 | 1.00 | 36.68 |
| ATOM | 294 | CA | GLU | A | 342 | 33.101 | 30.052 | 32.700 | 1.00 | 37.76 |
| ATOM | 295 | CB | GLU | A | 342 | 33.692 | 30.615 | 34.011 | 1.00 | 50.01 |
| ATOM | 296 | CG | GLU | A | 342 | 32.840 | 30.441 | 35.298 | 1.00 | 68.37 |
| ATOM | 297 | CD | GLU | A | 342 | 32.978 | 29.073 | 36.006 | 1.00 | 77.44 |
| ATOM | 298 | OE1 | GLU | A | 342 | 34.115 | 28.691 | 36.384 | 1.00 | 81.72 |
| ATOM | 299 | OE2 | GLU | A | 342 | 31.938 | 28.398 | 36.218 | 1.00 | 79.74 |
| ATOM | 300 | C | GLU | A | 342 | 32.969 | 28.567 | 32.793 | 1.00 | 29.07 |
| ATOM | 301 | O | GLU | A | 342 | 33.873 | 27.835 | 32.421 | 1.00 | 25.34 |
| ATOM | 302 | N | ASN | A | 343 | 31.832 | 28.138 | 33.316 | 1.00 | 28.65 |
| ATOM | 303 | CA | ASN | A | 343 | 31.581 | 26.729 | 33.459 | 1.00 | 31.12 |
| ATOM | 304 | CB | ASN | A | 343 | 30.239 | 26.495 | 34.113 | 1.00 | 31.12 |
| ATOM | 305 | CG | ASN | A | 343 | 29.952 | 25.051 | 34.268 | 1.00 | 44.07 |
| ATOM | 306 | OD1 | ASN | A | 343 | 30.740 | 24.315 | 34.857 | 1.00 | 52.87 |
| ATOM | 307 | ND2 | ASN | A | 343 | 28.870 | 24.593 | 33.658 | 1.00 | 49.75 |
| ATOM | 308 | C | ASN | A | 343 | 31.693 | 26.050 | 32.091 | 1.00 | 31.48 |
| ATOM | 309 | O | ASN | A | 343 | 32.474 | 25.106 | 31.939 | 1.00 | 32.73 |
| ATOM | 310 | N | TYR | A | 344 | 30.980 | 26.578 | 31.089 | 1.00 | 31.34 |
| ATOM | 311 | CA | TYR | A | 344 | 31.056 | 26.028 | 29.720 | 1.00 | 27.38 |
| ATOM | 312 | CB | TYR | A | 344 | 30.133 | 26.778 | 28.747 | 1.00 | 19.95 |
| ATOM | 313 | CG | TYR | A | 344 | 28.678 | 26.486 | 28.906 | 1.00 | 15.00 |
| ATOM | 314 | CD1 | TYR | A | 344 | 27.802 | 27.473 | 29.313 | 1.00 | 12.78 |
| ATOM | 315 | CE1 | TYR | A | 344 | 26.453 | 27.216 | 29.464 | 1.00 | 15.93 |
| ATOM | 316 | CD2 | TYR | A | 344 | 28.169 | 25.217 | 28.649 | 1.00 | 16.37 |
| ATOM | 317 | CE2 | TYR | A | 344 | 26.805 | 24.939 | 28.802 | 1.00 | 17.65 |
| ATOM | 318 | CZ | TYR | A | 344 | 25.953 | 25.946 | 29.210 | 1.00 | 14.48 |
| ATOM | 319 | OH | TYR | A | 344 | 24.612 | 25.705 | 29.377 | 1.00 | 15.05 |
| ATOM | 320 | C | TYR | A | 344 | 32.467 | 26.132 | 29.189 | 1.00 | 27.55 |
| ATOM | 321 | O | TYR | A | 344 | 32.966 | 25.216 | 28.542 | 1.00 | 30.70 |
| ATOM | 322 | N | GLN | A | 345 | 33.125 | 27.253 | 29.451 | 1.00 | 28.25 |
| ATOM | 323 | CA | GLN | A | 345 | 34.474 | 27.392 | 28.940 | 1.00 | 31.21 |
| ATOM | 324 | CB | GLN | A | 345 | 34.982 | 28.819 | 29.063 | 1.00 | 29.90 |
| ATOM | 325 | CG | GLN | A | 345 | 34.201 | 29.825 | 28.254 | 1.00 | 35.96 |
| ATOM | 326 | CD | GLN | A | 345 | 34.801 | 31.209 | 28.343 | 1.00 | 40.32 |
| ATOM | 327 | OE1 | GLN | A | 345 | 35.654 | 31.469 | 29.187 | 1.00 | 43.48 |
| ATOM | 328 | NE2 | GLN | A | 345 | 34.375 | 32.101 | 27.469 | 1.00 | 40.57 |
| ATOM | 329 | C | GLN | A | 345 | 35.397 | 26.446 | 29.668 | 1.00 | 36.16 |
| ATOM | 330 | O | GLN | A | 345 | 36.485 | 26.128 | 29.179 | 1.00 | 36.49 |
| ATOM | 331 | N | ASN | A | 346 | 34.972 | 26.004 | 30.853 | 1.00 | 42.02 |
| ATOM | 332 | CA | ASN | A | 346 | 35.780 | 25.077 | 31.630 | 1.00 | 42.72 |
| ATOM | 333 | CB | ASN | A | 346 | 35.532 | 25.237 | 33.135 | 1.00 | 48.26 |
| ATOM | 334 | CG | ASN | A | 346 | 36.336 | 26.398 | 33.743 | 1.00 | 52.40 |
| ATOM | 335 | OD1 | ASN | A | 346 | 37.433 | 26.728 | 33.282 | 1.00 | 53.04 |
| ATOM | 336 | ND2 | ASN | A | 346 | 35.802 | 26.995 | 34.799 | 1.00 | 53.36 |
| ATOM | 337 | C | ASN | A | 346 | 35.633 | 23.619 | 31.189 | 1.00 | 40.45 |
| ATOM | 338 | O | ASN | A | 346 | 36.533 | 22.810 | 31.475 | 1.00 | 38.19 |
| ATOM | 339 | N | LYS | A | 347 | 34.559 | 23.293 | 30.449 | 1.00 | 32.64 |
| ATOM | 340 | CA | LYS | A | 347 | 34.351 | 21.912 | 29.980 | 1.00 | 26.11 |
| ATOM | 341 | CB | LYS | A | 347 | 32.985 | 21.742 | 29.338 | 1.00 | 22.43 |
| ATOM | 342 | CG | LYS | A | 347 | 31.860 | 22.141 | 30.218 | 1.00 | 21.17 |
| ATOM | 343 | CD | LYS | A | 347 | 30.533 | 21.903 | 29.569 | 1.00 | 25.53 |
| ATOM | 344 | CE | LYS | A | 347 | 29.436 | 22.235 | 30.550 | 1.00 | 31.64 |
| ATOM | 345 | NZ | LYS | A | 347 | 28.105 | 21.911 | 30.002 | 1.00 | 42.98 |
| ATOM | 346 | C | LYS | A | 347 | 35.417 | 21.514 | 28.979 | 1.00 | 25.51 |
| ATOM | 347 | O | LYS | A | 347 | 35.862 | 22.338 | 28.186 | 1.00 | 30.14 |
| ATOM | 348 | N | GLN | A | 348 | 35.873 | 20.269 | 29.058 | 1.00 | 23.11 |
| ATOM | 349 | CA | GLN | A | 348 | 36.891 | 19.771 | 28.128 | 1.00 | 29.12 |
| ATOM | 350 | CB | GLN | A | 348 | 37.252 | 18.338 | 28.502 | 1.00 | 37.39 |
| ATOM | 351 | CG | GLN | A | 348 | 37.330 | 18.100 | 30.007 | 1.00 | 50.83 |
| ATOM | 352 | CD | GLN | A | 348 | 38.742 | 17.956 | 30.494 | 1.00 | 55.85 |
| ATOM | 353 | OE1 | GLN | A | 348 | 39.428 | 17.011 | 30.122 | 1.00 | 61.65 |
| ATOM | 354 | NE2 | GLN | A | 348 | 39.190 | 18.883 | 31.342 | 1.00 | 65.17 |
| ATOM | 355 | C | GLN | A | 348 | 36.292 | 19.803 | 26.704 | 1.00 | 25.62 |
| ATOM | 356 | O | GLN | A | 348 | 35.067 | 19.729 | 26.570 | 1.00 | 25.40 |
| ATOM | 357 | N | ARG | A | 349 | 37.137 | 19.854 | 25.668 | 1.00 | 28.86 |
| ATOM | 358 | CA | ARG | A | 349 | 36.671 | 19.928 | 24.271 | 1.00 | 30.13 |
| ATOM | 359 | CB | ARG | A | 349 | 37.831 | 19.768 | 23.276 | 1.00 | 38.59 |
| ATOM | 360 | CG | ARG | A | 349 | 37.408 | 20.068 | 21.821 | 1.00 | 51.14 |
| ATOM | 361 | CD | ARG | A | 349 | 38.539 | 19.932 | 20.772 | 1.00 | 60.22 |
| ATOM | 362 | NE | ARG | A | 349 | 38.121 | 20.510 | 19.481 | 1.00 | 71.23 |
| ATOM | 363 | CZ | ARG | A | 349 | 38.525 | 20.126 | 18.263 | 1.00 | 72.91 |
| ATOM | 364 | NH1 | ARG | A | 349 | 39.392 | 19.130 | 18.090 | 1.00 | 72.73 |
| ATOM | 365 | NH2 | ARG | A | 349 | 38.041 | 20.753 | 17.198 | 1.00 | 71.22 |
| ATOM | 366 | C | ARG | A | 349 | 35.578 | 18.940 | 23.908 | 1.00 | 28.40 |
| ATOM | 367 | O | ARG | A | 349 | 34.527 | 19.322 | 23.387 | 1.00 | 26.63 |
| ATOM | 368 | N | GLU | A | 350 | 35.857 | 17.662 | 24.165 | 1.00 | 29.38 |
| ATOM | 369 | CA | GLU | A | 350 | 34.932 | 16.565 | 23.877 | 1.00 | 27.83 |
| ATOM | 370 | CB | GLU | A | 350 | 35.586 | 15.215 | 24.184 | 1.00 | 33.12 |
| ATOM | 371 | CG | GLU | A | 350 | 35.794 | 14.956 | 25.684 | 1.00 | 35.99 |
| ATOM | 372 | CD | GLU | A | 350 | 37.212 | 15.247 | 26.148 | 1.00 | 38.01 |
| ATOM | 373 | OE1 | GLU | A | 350 | 37.736 | 14.408 | 26.913 | 1.00 | 43.71 |
| ATOM | 374 | OE2 | GLU | A | 350 | 37.809 | 16.283 | 25.756 | 1.00 | 32.70 |
| ATOM | 375 | C | GLU | A | 350 | 33.640 | 16.687 | 24.654 | 1.00 | 24.52 |
| ATOM | 376 | O | GLU | A | 350 | 32.596 | 16.237 | 24.195 | 1.00 | 25.09 |
| ATOM | 377 | N | VAL | A | 351 | 33.709 | 17.268 | 25.847 | 1.00 | 22.09 |
| ATOM | 378 | CA | VAL | A | 351 | 32.513 | 17.457 | 26.645 | 1.00 | 20.23 |
| ATOM | 379 | CB | VAL | A | 351 | 32.854 | 17.885 | 28.107 | 1.00 | 20.75 |
| ATOM | 380 | CG1 | VAL | A | 351 | 31.583 | 18.062 | 28.911 | 1.00 | 19.11 |
| ATOM | 381 | CG2 | VAL | A | 351 | 33.778 | 16.851 | 28.759 | 1.00 | 24.36 |
| ATOM | 382 | C | VAL | A | 351 | 31.625 | 18.521 | 25.990 | 1.00 | 20.47 |
| ATOM | 383 | O | VAL | A | 351 | 30.405 | 18.343 | 25.878 | 1.00 | 21.64 |
| ATOM | 384 | N | MET | A | 352 | 32.211 | 19.646 | 25.592 | 1.00 | 23.84 |
| ATOM | 385 | CA | MET | A | 352 | 31.414 | 20.705 | 24.952 | 1.00 | 23.93 |
| ATOM | 386 | CB | MET | A | 352 | 32.235 | 22.008 | 24.824 | 1.00 | 28.98 |
| ATOM | 387 | CG | MET | A | 352 | 31.437 | 23.318 | 24.552 | 1.00 | 26.03 |
| ATOM | 388 | SD | MET | A | 352 | 30.054 | 23.586 | 25.661 | 1.00 | 27.59 |
| ATOM | 389 | CE | MET | A | 352 | 29.087 | 24.826 | 24.802 | 1.00 | 28.37 |
| ATOM | 390 | C | MET | A | 352 | 30.897 | 20.191 | 23.580 | 1.00 | 21.58 |
| ATOM | 391 | O | MET | A | 352 | 29.743 | 20.392 | 23.241 | 1.00 | 23.25 |
| ATOM | 392 | N | TRP | A | 353 | 31.714 | 19.450 | 22.847 | 1.00 | 20.93 |
| ATOM | 393 | CA | TRP | A | 353 | 31.249 | 18.904 | 21.567 | 1.00 | 22.11 |
| ATOM | 394 | CB | TRP | A | 353 | 32.375 | 18.137 | 20.884 | 1.00 | 23.30 |
| ATOM | 395 | CG | TRP | A | 353 | 33.205 | 18.935 | 19.922 | 1.00 | 29.85 |
| ATOM | 396 | CD2 | TRP | A | 353 | 33.052 | 18.969 | 18.489 | 1.00 | 29.97 |
| ATOM | 397 | CE2 | TRP | A | 353 | 34.093 | 19.776 | 17.974 | 1.00 | 30.39 |
| ATOM | 398 | CE3 | TRP | A | 353 | 32.135 | 18.385 | 17.592 | 1.00 | 30.52 |
| ATOM | 399 | CD1 | TRP | A | 353 | 34.298 | 19.711 | 20.209 | 1.00 | 30.84 |
| ATOM | 400 | NE1 | TRP | A | 353 | 34.838 | 20.216 | 19.038 | 1.00 | 32.97 |
| ATOM | 401 | CZ2 | TRP | A | 353 | 34.248 | 20.015 | 16.599 | 1.00 | 32.23 |
| ATOM | 402 | CZ3 | TRP | A | 353 | 32.291 | 18.623 | 16.228 | 1.00 | 29.92 |
| ATOM | 403 | CH2 | TRP | A | 353 | 33.344 | 19.432 | 15.748 | 1.00 | 30.15 |
| ATOM | 404 | C | TRP | A | 353 | 30.022 | 17.992 | 21.733 | 1.00 | 19.54 |
| ATOM | 405 | O | TRP | A | 353 | 29.027 | 18.132 | 21.014 | 1.00 | 21.17 |
| ATOM | 406 | N | GLN | A | 354 | 30.059 | 17.088 | 22.714 | 1.00 | 20.89 |
| ATOM | 407 | CA | GLN | A | 354 | 28.938 | 16.158 | 22.939 | 1.00 | 19.30 |
| ATOM | 408 | CB | GLN | A | 354 | 29.263 | 15.167 | 24.077 | 1.00 | 23.50 |
| ATOM | 409 | CG | GLN | A | 354 | 28.393 | 13.888 | 24.093 | 1.00 | 24.75 |
| ATOM | 410 | CD | GLN | A | 354 | 27.436 | 13.788 | 25.282 | 1.00 | 23.74 |
| ATOM | 411 | OE1 | GLN | A | 354 | 27.297 | 14.737 | 26.049 | 1.00 | 29.52 |
| ATOM | 412 | NE2 | GLN | A | 354 | 26.767 | 12.629 | 25.427 | 1.00 | 20.53 |
| ATOM | 413 | C | GLN | A | 354 | 27.647 | 16.875 | 23.248 | 1.00 | 16.93 |
| ATOM | 414 | O | GLN | A | 354 | 26.566 | 16.516 | 22.759 | 1.00 | 16.41 |
| ATOM | 415 | N | LEU | A | 355 | 27.747 | 17.885 | 24.098 | 1.00 | 19.29 |
| ATOM | 416 | CA | LEU | A | 355 | 26.574 | 18.645 | 24.468 | 1.00 | 15.90 |
| ATOM | 417 | CB | LEU | A | 355 | 26.913 | 19.657 | 25.579 | 1.00 | 15.22 |
| ATOM | 418 | CG | LEU | A | 355 | 25.802 | 20.622 | 25.993 | 1.00 | 17.93 |
| ATOM | 419 | CD1 | LEU | A | 355 | 24.582 | 19.962 | 26.610 | 1.00 | 14.68 |
| ATOM | 420 | CD2 | LEU | A | 355 | 26.418 | 21.667 | 26.918 | 1.00 | 21.31 |
| ATOM | 421 | C | LEU | A | 355 | 25.993 | 19.357 | 23.240 | 1.00 | 11.91 |
| ATOM | 422 | O | LEU | A | 355 | 24.790 | 19.348 | 23.054 | 1.00 | 14.23 |
| ATOM | 423 | N | CYS | A | 356 | 26.827 | 20.005 | 22.441 | 1.00 | 16.77 |
| ATOM | 424 | CA | CYS | A | 356 | 26.307 | 20.691 | 21.244 | 1.00 | 22.19 |
| ATOM | 425 | CB | CYS | A | 356 | 27.436 | 21.450 | 20.561 | 1.00 | 21.05 |
| ATOM | 426 | SG | CYS | A | 356 | 28.067 | 22.797 | 21.523 | 1.00 | 23.18 |
| ATOM | 427 | C | CYS | A | 356 | 25.640 | 19.676 | 20.263 | 1.00 | 21.08 |
| ATOM | 428 | O | CYS | A | 356 | 24.584 | 19.943 | 19.679 | 1.00 | 22.08 |
| ATOM | 429 | N | ALA | A | 357 | 26.228 | 18.483 | 20.160 | 1.00 | 22.51 |
| ATOM | 430 | CA | ALA | A | 357 | 25.678 | 17.449 | 19.304 | 1.00 | 19.05 |
| ATOM | 431 | CB | ALA | A | 357 | 26.606 | 16.267 | 19.264 | 1.00 | 25.06 |
| ATOM | 432 | C | ALA | A | 357 | 24.296 | 17.048 | 19.778 | 1.00 | 20.99 |
| ATOM | 433 | O | ALA | A | 357 | 23.387 | 16.919 | 18.975 | 1.00 | 21.56 |
| ATOM | 434 | N | ILE | A | 358 | 24.104 | 16.887 | 21.092 | 1.00 | 21.52 |
| ATOM | 435 | CA | ILE | A | 358 | 22.782 | 16.522 | 21.627 | 1.00 | 19.00 |
| ATOM | 436 | CB | ILE | A | 358 | 22.812 | 16.382 | 23.202 | 1.00 | 19.36 |
| ATOM | 437 | CG2 | ILE | A | 358 | 21.374 | 16.383 | 23.779 | 1.00 | 15.59 |
| ATOM | 438 | CG1 | ILE | A | 358 | 23.623 | 15.153 | 23.614 | 1.00 | 22.16 |
| ATOM | 439 | CD1 | ILE | A | 358 | 24.073 | 15.174 | 25.079 | 1.00 | 23.75 |
| ATOM | 440 | C | ILE | A | 358 | 21.747 | 17.597 | 21.291 | 1.00 | 18.17 |
| ATOM | 441 | O | ILE | A | 358 | 20.590 | 17.322 | 20.947 | 1.00 | 17.46 |
| ATOM | 442 | N | LYS | A | 359 | 22.171 | 18.843 | 21.443 | 1.00 | 22.77 |
| ATOM | 443 | CA | LYS | A | 359 | 21.291 | 19.983 | 21.206 | 1.00 | 21.40 |
| ATOM | 444 | CB | LYS | A | 359 | 21.931 | 21.234 | 21.809 | 1.00 | 23.15 |
| ATOM | 445 | CG | LYS | A | 359 | 22.175 | 21.133 | 23.334 | 1.00 | 24.28 |
| ATOM | 446 | CD | LYS | A | 359 | 20.879 | 20.855 | 24.063 | 1.00 | 24.08 |
| ATOM | 447 | CE | LYS | A | 359 | 21.061 | 20.872 | 25.562 | 1.00 | 31.76 |
| ATOM | 448 | NZ | LYS | A | 359 | 19.730 | 20.695 | 26.175 | 1.00 | 29.13 |
| ATOM | 449 | C | LYS | A | 359 | 20.925 | 20.166 | 19.721 | 1.00 | 16.10 |
| ATOM | 450 | O | LYS | A | 359 | 19.764 | 20.377 | 19.403 | 1.00 | 20.28 |
| ATOM | 451 | N | ILE | A | 360 | 21.914 | 20.094 | 18.841 | 1.00 | 15.62 |
| ATOM | 452 | CA | ILE | A | 360 | 21.666 | 20.199 | 17.402 | 1.00 | 18.38 |
| ATOM | 453 | CB | ILE | A | 360 | 22.959 | 19.988 | 16.595 | 1.00 | 24.95 |
| ATOM | 454 | CG2 | ILE | A | 360 | 22.659 | 19.672 | 15.136 | 1.00 | 28.87 |
| ATOM | 455 | CG1 | ILE | A | 360 | 23.846 | 21.221 | 16.659 | 1.00 | 25.71 |
| ATOM | 456 | CD1 | ILE | A | 360 | 25.260 | 20.940 | 16.214 | 1.00 | 28.76 |
| ATOM | 457 | C | ILE | A | 360 | 20.707 | 19.085 | 17.026 | 1.00 | 16.86 |
| ATOM | 458 | O | ILE | A | 360 | 19.678 | 19.341 | 16.433 | 1.00 | 23.45 |
| ATOM | 459 | N | THR | A | 361 | 20.986 | 17.861 | 17.467 | 1.00 | 17.32 |
| ATOM | 460 | CA | THR | A | 361 | 20.131 | 16.724 | 17.118 | 1.00 | 17.15 |
| ATOM | 461 | CB | THR | A | 361 | 20.647 | 15.411 | 17.703 | 1.00 | 21.09 |
| ATOM | 462 | OG1 | THR | A | 361 | 22.030 | 15.263 | 17.385 | 1.00 | 21.21 |
| ATOM | 463 | CG2 | THR | A | 361 | 19.842 | 14.207 | 17.146 | 1.00 | 23.13 |
| ATOM | 464 | C | THR | A | 361 | 18.687 | 16.887 | 17.514 | 1.00 | 22.12 |
| ATOM | 465 | O | THR | A | 361 | 17.786 | 16.414 | 16.813 | 1.00 | 22.98 |
| ATOM | 466 | N | GLU | A | 362 | 18.455 | 17.530 | 18.660 | 1.00 | 22.63 |
| ATOM | 467 | CA | GLU | A | 362 | 17.092 | 17.761 | 19.121 | 1.00 | 21.83 |
| ATOM | 468 | CB | GLU | A | 362 | 17.076 | 18.335 | 20.541 | 1.00 | 25.44 |
| ATOM | 469 | CG | GLU | A | 362 | 15.729 | 18.156 | 21.228 | 1.00 | 45.77 |
| ATOM | 470 | CD | GLU | A | 362 | 15.382 | 19.260 | 22.253 | 1.00 | 61.04 |
| ATOM | 471 | OE1 | GLU | A | 362 | 16.281 | 19.648 | 23.059 | 1.00 | 67.71 |
| ATOM | 472 | OE2 | GLU | A | 362 | 14.199 | 19.723 | 22.249 | 1.00 | 58.48 |
| ATOM | 473 | C | GLU | A | 362 | 16.407 | 18.717 | 18.137 | 1.00 | 17.52 |
| ATOM | 474 | O | GLU | A | 362 | 15.226 | 18.567 | 17.836 | 1.00 | 18.31 |
| ATOM | 475 | N | ALA | A | 363 | 17.148 | 19.711 | 17.656 | 1.00 | 17.89 |
| ATOM | 476 | CA | ALA | A | 363 | 16.613 | 20.652 | 16.656 | 1.00 | 22.83 |
| ATOM | 477 | CB | ALA | A | 363 | 17.624 | 21.753 | 16.358 | 1.00 | 21.95 |
| ATOM | 478 | C | ALA | A | 363 | 16.304 | 19.896 | 15.348 | 1.00 | 21.89 |
| ATOM | 479 | O | ALA | A | 363 | 15.286 | 20.140 | 14.706 | 1.00 | 24.25 |
| ATOM | 480 | N | ILE | A | 364 | 17.196 | 18.994 | 14.952 | 1.00 | 23.12 |
| ATOM | 481 | CA | ILE | A | 364 | 16.995 | 18.214 | 13.715 | 1.00 | 23.74 |
| ATOM | 482 | CB | ILE | A | 364 | 18.255 | 17.379 | 13.357 | 1.00 | 20.60 |
| ATOM | 483 | CG2 | ILE | A | 364 | 17.943 | 16.320 | 12.288 | 1.00 | 20.36 |
| ATOM | 484 | CG1 | ILE | A | 364 | 19.349 | 18.329 | 12.855 | 1.00 | 16.57 |
| ATOM | 485 | CD1 | ILE | A | 364 | 20.748 | 17.766 | 12.820 | 1.00 | 17.77 |
| ATOM | 486 | C | ILE | A | 364 | 15.709 | 17.382 | 13.705 | 1.00 | 28.19 |
| ATOM | 487 | O | ILE | A | 364 | 14.969 | 17.380 | 12.715 | 1.00 | 29.54 |
| ATOM | 488 | N | GLN | A | 365 | 15.370 | 16.784 | 14.842 | 1.00 | 28.50 |
| ATOM | 489 | CA | GLN | A | 365 | 14.159 | 15.976 | 14.937 | 1.00 | 28.61 |
| ATOM | 490 | CB | GLN | A | 365 | 14.049 | 15.352 | 16.309 | 1.00 | 34.56 |
| ATOM | 491 | CG | GLN | A | 365 | 15.324 | 14.645 | 16.719 | 1.00 | 46.55 |
| ATOM | 492 | CD | GLN | A | 365 | 15.071 | 13.640 | 17.800 | 1.00 | 53.78 |
| ATOM | 493 | OE1 | GLN | A | 365 | 13.968 | 13.106 | 17.901 | 1.00 | 64.74 |
| ATOM | 494 | NE2 | GLN | A | 365 | 16.077 | 13.366 | 18.617 | 1.00 | 55.00 |
| ATOM | 495 | C | GLN | A | 365 | 12.893 | 16.737 | 14.620 | 1.00 | 26.94 |
| ATOM | 496 | O | GLN | A | 365 | 11.919 | 16.145 | 14.172 | 1.00 | 30.81 |
| ATOM | 497 | N | TYR | A | 366 | 12.884 | 18.036 | 14.903 | 1.00 | 26.99 |
| ATOM | 498 | CA | TYR | A | 366 | 11.733 | 18.889 | 14.598 | 1.00 | 23.20 |
| ATOM | 499 | CB | TYR | A | 366 | 11.822 | 20.212 | 15.376 | 1.00 | 25.77 |
| ATOM | 500 | CG | TYR | A | 366 | 11.345 | 20.112 | 16.816 | 1.00 | 27.13 |
| ATOM | 501 | CD1 | TYR | A | 366 | 12.246 | 20.050 | 17.880 | 1.00 | 27.24 |
| ATOM | 502 | CE1 | TYR | A | 366 | 11.787 | 19.956 | 19.198 | 1.00 | 32.73 |
| ATOM | 503 | CD2 | TYR | A | 366 | 9.984 | 20.075 | 17.103 | 1.00 | 29.26 |
| ATOM | 504 | CE2 | TYR | A | 366 | 9.521 | 19.976 | 18.402 | 1.00 | 33.96 |
| ATOM | 505 | CZ | TYR | A | 366 | 10.420 | 19.917 | 19.445 | 1.00 | 36.94 |
| ATOM | 506 | OH | TYR | A | 366 | 9.925 | 19.808 | 20.729 | 1.00 | 45.77 |
| ATOM | 507 | C | TYR | A | 366 | 11.743 | 19.165 | 13.084 | 1.00 | 22.71 |
| ATOM | 508 | O | TYR | A | 366 | 10.688 | 19.258 | 12.450 | 1.00 | 21.29 |
| ATOM | 509 | N | VAL | A | 367 | 12.948 | 19.314 | 12.527 | 1.00 | 23.15 |
| ATOM | 510 | CA | VAL | A | 367 | 13.130 | 19.536 | 11.085 | 1.00 | 25.23 |
| ATOM | 511 | CB | VAL | A | 367 | 14.586 | 19.907 | 10.742 | 1.00 | 22.53 |
| ATOM | 512 | CG1 | VAL | A | 367 | 14.798 | 19.914 | 9.224 | 1.00 | 20.69 |
| ATOM | 513 | CG2 | VAL | A | 367 | 14.878 | 21.280 | 11.292 | 1.00 | 17.32 |
| ATOM | 514 | C | VAL | A | 367 | 12.650 | 18.303 | 10.281 | 1.00 | 28.13 |
| ATOM | 515 | O | VAL | A | 367 | 12.027 | 18.449 | 9.236 | 1.00 | 28.63 |
| ATOM | 516 | N | VAL | A | 368 | 12.929 | 17.098 | 10.771 | 1.00 | 27.16 |
| ATOM | 517 | CA | VAL | A | 368 | 12.450 | 15.890 | 10.116 | 1.00 | 27.59 |
| ATOM | 518 | CB | VAL | A | 368 | 13.048 | 14.606 | 10.759 | 1.00 | 23.81 |
| ATOM | 519 | CG1 | VAL | A | 368 | 12.340 | 13.380 | 10.226 | 1.00 | 29.32 |
| ATOM | 520 | CG2 | VAL | A | 368 | 14.550 | 14.505 | 10.469 | 1.00 | 18.99 |
| ATOM | 521 | C | VAL | A | 368 | 10.894 | 15.861 | 10.144 | 1.00 | 32.37 |
| ATOM | 522 | O | VAL | A | 368 | 10.268 | 15.429 | 9.159 | 1.00 | 33.74 |
| ATOM | 523 | N | GLU | A | 369 | 10.268 | 16.324 | 11.242 | 1.00 | 26.37 |
| ATOM | 524 | CA | GLU | A | 369 | 8.801 | 16.364 | 11.318 | 1.00 | 26.23 |
| ATOM | 525 | CB | GLU | A | 369 | 8.281 | 16.756 | 12.712 | 1.00 | 29.58 |
| ATOM | 526 | CG | GLU | A | 369 | 8.304 | 15.647 | 13.763 | 1.00 | 33.78 |
| ATOM | 527 | CD | GLU | A | 369 | 7.736 | 14.303 | 13.284 | 1.00 | 34.63 |
| ATOM | 528 | OE1 | GLU | A | 369 | 6.610 | 14.253 | 12.741 | 1.00 | 36.64 |
| ATOM | 529 | OE2 | GLU | A | 369 | 8.430 | 13.280 | 13.476 | 1.00 | 40.81 |
| ATOM | 530 | C | GLU | A | 369 | 8.269 | 17.358 | 10.291 | 1.00 | 26.20 |
| ATOM | 531 | O | GLU | A | 369 | 7.213 | 17.143 | 9.713 | 1.00 | 29.54 |
| ATOM | 532 | N | PHE | A | 370 | 8.970 | 18.476 | 10.118 | 1.00 | 24.75 |
| ATOM | 533 | CA | PHE | A | 370 | 8.607 | 19.475 | 9.114 | 1.00 | 24.70 |
| ATOM | 534 | CB | PHE | A | 370 | 9.615 | 20.615 | 9.162 | 1.00 | 23.84 |
| ATOM | 535 | CG | PHE | A | 370 | 9.415 | 21.663 | 8.093 | 1.00 | 24.23 |
| ATOM | 536 | CD1 | PHE | A | 370 | 8.265 | 22.444 | 8.071 | 1.00 | 24.20 |
| ATOM | 537 | CD2 | PHE | A | 370 | 10.415 | 21.918 | 7.161 | 1.00 | 24.12 |
| ATOM | 538 | CE1 | PHE | A | 370 | 8.120 | 23.468 | 7.150 | 1.00 | 27.38 |
| ATOM | 539 | CE2 | PHE | A | 370 | 10.276 | 22.943 | 6.234 | 1.00 | 26.73 |
| ATOM | 540 | CZ | PHE | A | 370 | 9.125 | 23.722 | 6.231 | 1.00 | 24.57 |
| ATOM | 541 | C | PHE | A | 370 | 8.648 | 18.805 | 7.712 | 1.00 | 26.16 |
| ATOM | 542 | O | PHE | A | 370 | 7.666 | 18.841 | 6.966 | 1.00 | 24.95 |
| ATOM | 543 | N | ALA | A | 371 | 9.780 | 18.172 | 7.395 | 1.00 | 25.24 |
| ATOM | 544 | CA | ALA | A | 371 | 9.988 | 17.459 | 6.130 | 1.00 | 31.38 |
| ATOM | 545 | CB | ALA | A | 371 | 11.281 | 16.681 | 6.171 | 1.00 | 27.13 |
| ATOM | 546 | C | ALA | A | 371 | 8.829 | 16.525 | 5.794 | 1.00 | 33.49 |
| ATOM | 547 | O | ALA | A | 371 | 8.328 | 16.550 | 4.674 | 1.00 | 35.75 |
| ATOM | 548 | N | LYS | A | 372 | 8.380 | 15.730 | 6.766 | 1.00 | 32.11 |
| ATOM | 549 | CA | LYS | A | 372 | 7.260 | 14.817 | 6.547 | 1.00 | 32.07 |
| ATOM | 550 | CB | LYS | A | 372 | 7.025 | 13.969 | 7.780 | 1.00 | 31.18 |
| ATOM | 551 | CG | LYS | A | 372 | 8.142 | 13.030 | 8.132 | 1.00 | 34.27 |
| ATOM | 552 | CD | LYS | A | 372 | 7.714 | 12.198 | 9.322 | 1.00 | 41.43 |
| ATOM | 553 | CE | LYS | A | 372 | 8.898 | 11.535 | 9.996 | 1.00 | 47.18 |
| ATOM | 554 | NZ | LYS | A | 372 | 8.490 | 10.731 | 11.203 | 1.00 | 52.21 |
| ATOM | 555 | C | LYS | A | 372 | 5.920 | 15.464 | 6.175 | 1.00 | 36.43 |
| ATOM | 556 | O | LYS | A | 372 | 5.047 | 14.804 | 5.612 | 1.00 | 39.27 |
| ATOM | 557 | N | ARG | A | 373 | 5.742 | 16.735 | 6.511 | 1.00 | 38.37 |
| ATOM | 558 | CA | ARG | A | 373 | 4.484 | 17.435 | 6.242 | 1.00 | 40.66 |
| ATOM | 559 | CB | ARG | A | 373 | 4.201 | 18.447 | 7.355 | 1.00 | 45.55 |
| ATOM | 560 | CG | ARG | A | 373 | 4.681 | 18.002 | 8.714 | 1.00 | 54.93 |
| ATOM | 561 | CD | ARG | A | 373 | 3.682 | 17.131 | 9.441 | 1.00 | 59.22 |
| ATOM | 562 | NE | ARG | A | 373 | 2.817 | 17.960 | 10.278 | 1.00 | 65.72 |
| ATOM | 563 | CZ | ARG | A | 373 | 2.860 | 17.988 | 11.607 | 1.00 | 63.53 |
| ATOM | 564 | NH1 | ARG | A | 373 | 3.723 | 17.222 | 12.261 | 1.00 | 61.86 |
| ATOM | 565 | NH2 | ARG | A | 373 | 2.057 | 18.802 | 12.281 | 1.00 | 66.65 |
| ATOM | 566 | C | ARG | A | 373 | 4.503 | 18.179 | 4.916 | 1.00 | 41.35 |
| ATOM | 567 | O | ARG | A | 373 | 3.496 | 18.773 | 4.516 | 1.00 | 40.31 |
| ATOM | 568 | N | ILE | A | 374 | 5.669 | 18.203 | 4.271 | 1.00 | 41.14 |
| ATOM | 569 | CA | ILE | A | 374 | 5.806 | 18.883 | 2.996 | 1.00 | 40.72 |
| ATOM | 570 | CB | ILE | A | 374 | 7.237 | 19.329 | 2.697 | 1.00 | 36.14 |
| ATOM | 571 | CG2 | ILE | A | 374 | 7.298 | 19.939 | 1.299 | 1.00 | 32.36 |
| ATOM | 572 | CG1 | ILE | A | 374 | 7.675 | 20.387 | 3.702 | 1.00 | 34.12 |
| ATOM | 573 | CD1 | ILE | A | 374 | 9.159 | 20.603 | 3.712 | 1.00 | 36.75 |
| ATOM | 574 | C | ILE | A | 374 | 5.302 | 18.054 | 1.831 | 1.00 | 45.90 |
| ATOM | 575 | O | ILE | A | 374 | 5.733 | 16.929 | 1.583 | 1.00 | 42.19 |
| ATOM | 576 | N | ASP | A | 375 | 4.301 | 18.625 | 1.183 | 1.00 | 50.81 |
| ATOM | 577 | CA | ASP | A | 375 | 3.654 | 18.084 | −0.002 | 1.00 | 52.36 |
| ATOM | 578 | CB | ASP | A | 375 | 2.859 | 19.229 | −0.649 | 1.00 | 60.93 |
| ATOM | 579 | CG | ASP | A | 375 | 3.543 | 20.613 | −0.449 | 1.00 | 68.87 |
| ATOM | 580 | OD1 | ASP | A | 375 | 4.421 | 20.987 | −1.277 | 1.00 | 67.94 |
| ATOM | 581 | OD2 | ASP | A | 375 | 3.225 | 21.301 | 0.561 | 1.00 | 63.03 |
| ATOM | 582 | C | ASP | A | 375 | 4.690 | 17.547 | −0.998 | 1.00 | 48.88 |
| ATOM | 583 | O | ASP | A | 375 | 5.454 | 18.309 | −1.595 | 1.00 | 48.00 |
| ATOM | 584 | N | GLY | A | 376 | 4.743 | 16.234 | −1.152 | 1.00 | 45.67 |
| ATOM | 585 | CA | GLY | A | 376 | 5.683 | 15.669 | −2.103 | 1.00 | 43.48 |
| ATOM | 586 | C | GLY | A | 376 | 6.816 | 14.872 | −1.514 | 1.00 | 40.36 |
| ATOM | 587 | O | GLY | A | 376 | 7.200 | 13.837 | −2.052 | 1.00 | 38.28 |
| ATOM | 588 | N | PHE | A | 377 | 7.339 | 15.346 | −0.394 | 1.00 | 37.97 |
| ATOM | 589 | CA | PHE | A | 377 | 8.448 | 14.677 | 0.270 | 1.00 | 34.67 |
| ATOM | 590 | CB | PHE | A | 377 | 8.826 | 15.430 | 1.567 | 1.00 | 34.08 |
| ATOM | 591 | CG | PHE | A | 377 | 10.054 | 14.887 | 2.245 | 1.00 | 29.16 |
| ATOM | 592 | CD1 | PHE | A | 377 | 11.305 | 15.422 | 1.962 | 1.00 | 27.28 |
| ATOM | 593 | CD2 | PHE | A | 377 | 9.964 | 13.778 | 3.096 | 1.00 | 30.07 |
| ATOM | 594 | CE1 | PHE | A | 377 | 12.453 | 14.859 | 2.496 | 1.00 | 28.02 |
| ATOM | 595 | CE2 | PHE | A | 377 | 11.095 | 13.205 | 3.637 | 1.00 | 29.05 |
| ATOM | 596 | CZ | PHE | A | 377 | 12.353 | 13.746 | 3.333 | 1.00 | 28.74 |
| ATOM | 597 | C | PHE | A | 377 | 8.203 | 13.197 | 0.572 | 1.00 | 34.90 |
| ATOM | 598 | O | PHE | A | 377 | 9.076 | 12.361 | 0.305 | 1.00 | 35.66 |
| ATOM | 599 | N | MET | A | 378 | 7.060 | 12.871 | 1.169 | 1.00 | 34.38 |
| ATOM | 600 | CA | MET | A | 378 | 6.766 | 11.491 | 1.512 | 1.00 | 38.67 |
| ATOM | 601 | CB | MET | A | 378 | 5.565 | 11.393 | 2.457 | 1.00 | 41.83 |
| ATOM | 602 | CG | MET | A | 378 | 5.882 | 11.754 | 3.909 | 1.00 | 48.90 |
| ATOM | 603 | SD | MET | A | 378 | 7.422 | 10.982 | 4.496 | 1.00 | 56.77 |
| ATOM | 604 | CE | MET | A | 378 | 6.884 | 9.291 | 4.787 | 1.00 | 57.67 |
| ATOM | 605 | C | MET | A | 378 | 6.574 | 10.586 | 0.301 | 1.00 | 42.95 |
| ATOM | 606 | O | MET | A | 378 | 6.564 | 9.363 | 0.428 | 1.00 | 43.36 |
| ATOM | 607 | N | GLU | A | 379 | 6.427 | 11.184 | −0.877 | 1.00 | 44.95 |
| ATOM | 608 | CA | GLU | A | 379 | 6.248 | 10.399 | −2.086 | 1.00 | 45.80 |
| ATOM | 609 | CB | GLU | A | 379 | 5.359 | 11.144 | −3.071 | 1.00 | 52.39 |
| ATOM | 610 | CG | GLU | A | 379 | 3.943 | 11.354 | −2.587 | 1.00 | 61.32 |
| ATOM | 611 | CD | GLU | A | 379 | 3.127 | 12.219 | −3.537 | 1.00 | 71.44 |
| ATOM | 612 | OE1 | GLU | A | 379 | 3.681 | 13.182 | −4.126 | 1.00 | 73.32 |
| ATOM | 613 | OE2 | GLU | A | 379 | 1.920 | 11.933 | −3.693 | 1.00 | 77.96 |
| ATOM | 614 | C | GLU | A | 379 | 7.581 | 10.057 | −2.741 | 1.00 | 43.64 |
| ATOM | 615 | O | GLU | A | 379 | 7.655 | 9.144 | −3.553 | 1.00 | 43.83 |
| ATOM | 616 | N | LEU | A | 380 | 8.633 | 10.794 | −2.409 | 1.00 | 40.33 |
| ATOM | 617 | CA | LEU | A | 380 | 9.939 | 10.521 | −2.986 | 1.00 | 40.49 |
| ATOM | 618 | CB | LEU | A | 380 | 10.949 | 11.562 | −2.536 | 1.00 | 39.57 |
| ATOM | 619 | CG | LEU | A | 380 | 10.996 | 12.909 | −3.242 | 1.00 | 43.35 |
| ATOM | 620 | CD1 | LEU | A | 380 | 9.660 | 13.313 | −3.786 | 1.00 | 48.51 |
| ATOM | 621 | CD2 | LEU | A | 380 | 11.496 | 13.947 | −2.266 | 1.00 | 43.53 |
| ATOM | 622 | C | LEU | A | 380 | 10.374 | 9.151 | −2.509 | 1.00 | 43.17 |
| ATOM | 623 | O | LEU | A | 380 | 9.702 | 8.546 | −1.675 | 1.00 | 41.79 |
| ATOM | 624 | N | CYS | A | 381 | 11.480 | 8.650 | −3.053 | 1.00 | 46.89 |
| ATOM | 625 | CA | CYS | A | 381 | 11.988 | 7.339 | −2.651 | 1.00 | 51.11 |
| ATOM | 626 | CB | CYS | A | 351 | 12.676 | 6.632 | −3.838 | 1.00 | 48.63 |
| ATOM | 627 | SG | CYS | A | 381 | 14.146 | 7.457 | −4.487 | 1.00 | 48.71 |
| ATOM | 628 | C | CYS | A | 381 | 12.938 | 7.479 | −1.437 | 1.00 | 53.70 |
| ATOM | 629 | O | CYS | A | 381 | 13.731 | 8.427 | −1.357 | 1.00 | 54.89 |
| ATOM | 630 | N | GLN | A | 382 | 12.813 | 6.559 | −0.478 | 1.00 | 56.40 |
| ATOM | 631 | CA | GLN | A | 382 | 13.625 | 6.552 | 0.747 | 1.00 | 57.37 |
| ATOM | 632 | CB | GLN | A | 382 | 13.717 | 5.125 | 1.299 | 1.00 | 66.42 |
| ATOM | 633 | CG | GLN | A | 382 | 14.527 | 4.981 | 2.596 | 1.00 | 80.23 |
| ATOM | 634 | CD | GLN | A | 382 | 14.924 | 3.529 | 2.908 | 1.00 | 87.56 |
| ATOM | 635 | OE1 | GLN | A | 382 | 14.364 | 2.579 | 2.351 | 1.00 | 93.99 |
| ATOM | 636 | NE2 | GLN | A | 382 | 15.916 | 3.361 | 3.781 | 1.00 | 90.30 |
| ATOM | 637 | C | GLN | A | 382 | 15.030 | 7.108 | 0.529 | 1.00 | 52.28 |
| ATOM | 638 | O | GLN | A | 382 | 15.534 | 7.891 | 1.315 | 1.00 | 52.44 |
| ATOM | 639 | N | ASN | A | 383 | 15.644 | 6.716 | −0.571 | 1.00 | 48.24 |
| ATOM | 640 | CA | ASN | A | 383 | 16.975 | 7.166 | −0.891 | 1.00 | 46.79 |
| ATOM | 641 | CB | ASN | A | 383 | 17.393 | 6.604 | −2.241 | 1.00 | 55.14 |
| ATOM | 642 | CG | ASN | A | 383 | 17.496 | 5.100 | −2.232 | 1.00 | 63.92 |
| ATOM | 643 | OD1 | ASN | A | 383 | 18.603 | 4.559 | −2.272 | 1.00 | 68.19 |
| ATOM | 644 | ND2 | ASN | A | 383 | 16.350 | 4.405 | −2.198 | 1.00 | 65.25 |
| ATOM | 645 | C | ASN | A | 383 | 17.000 | 8.669 | −0.986 | 1.00 | 42.13 |
| ATOM | 646 | O | ASN | A | 383 | 17.853 | 9.331 | −0.408 | 1.00 | 38.96 |
| ATOM | 647 | N | ASP | A | 384 | 16.064 | 9.203 | −1.749 | 1.00 | 35.42 |
| ATOM | 648 | CA | ASP | A | 384 | 16.015 | 10.633 | −1.945 | 1.00 | 35.36 |
| ATOM | 649 | CB | ASP | A | 384 | 15.137 | 10.984 | −3.146 | 1.00 | 35.18 |
| ATOM | 650 | CG | ASP | A | 384 | 15.790 | 10.618 | −4.491 | 1.00 | 36.49 |
| ATOM | 651 | OD1 | ASP | A | 384 | 16.835 | 9.919 | −4.525 | 1.00 | 35.12 |
| ATOM | 652 | OD2 | ASP | A | 384 | 15.245 | 11.049 | −5.519 | 1.00 | 34.59 |
| ATOM | 653 | C | ASP | A | 384 | 15.578 | 11.376 | −0.701 | 1.00 | 30.33 |
| ATOM | 654 | O | ASP | A | 384 | 15.999 | 12.501 | −0.484 | 1.00 | 27.59 |
| ATOM | 655 | N | GLN | A | 385 | 14.713 | 10.760 | 0.098 | 1.00 | 31.50 |
| ATOM | 656 | CA | GLN | A | 385 | 14.280 | 11.403 | 1.348 | 1.00 | 33.11 |
| ATOM | 657 | CB | GLN | A | 385 | 13.215 | 10.577 | 2.033 | 1.00 | 28.85 |
| ATOM | 658 | CG | GLN | A | 385 | 11.932 | 10.516 | 1.281 | 1.00 | 29.25 |
| ATOM | 659 | CD | GLN | A | 385 | 10.981 | 9.578 | 1.942 | 1.00 | 35.13 |
| ATOM | 660 | OE1 | GLN | A | 385 | 11.392 | 8.704 | 2.711 | 1.00 | 39.80 |
| ATOM | 661 | NE2 | GLN | A | 385 | 9.703 | 9.737 | 1.663 | 1.00 | 34.78 |
| ATOM | 662 | C | GLN | A | 385 | 15.488 | 11.552 | 2.277 | 1.00 | 31.53 |
| ATOM | 663 | O | GLN | A | 385 | 15.751 | 12.632 | 2.816 | 1.00 | 29.68 |
| ATOM | 664 | N | ILE | A | 386 | 16.251 | 10.466 | 2.394 | 1.00 | 29.20 |
| ATOM | 665 | CA | ILE | A | 386 | 17.452 | 10.430 | 3.208 | 1.00 | 31.16 |
| ATOM | 666 | CB | ILE | A | 386 | 17.992 | 8.987 | 3.310 | 1.00 | 30.97 |
| ATOM | 667 | CG2 | ILE | A | 386 | 19.411 | 8.959 | 3.925 | 1.00 | 29.51 |
| ATOM | 668 | CG1 | ILE | A | 386 | 16.982 | 8.148 | 4.103 | 1.00 | 31.82 |
| ATOM | 669 | CD1 | ILE | A | 386 | 17.452 | 6.761 | 4.458 | 1.00 | 39.13 |
| ATOM | 670 | C | ILE | A | 386 | 18.518 | 11.411 | 2.696 | 1.00 | 30.95 |
| ATOM | 671 | O | ILE | A | 386 | 19.068 | 12.189 | 3.473 | 1.00 | 31.12 |
| ATOM | 672 | N | VAL | A | 387 | 18.788 | 11.400 | 1.392 | 1.00 | 30.52 |
| ATOM | 673 | CA | VAL | A | 387 | 19.771 | 12.314 | 0.800 | 1.00 | 26.90 |
| ATOM | 674 | CB | VAL | A | 387 | 19.877 | 12.122 | −0.763 | 1.00 | 25.37 |
| ATOM | 675 | CG1 | VAL | A | 387 | 20.555 | 13.328 | −1.412 | 1.00 | 20.02 |
| ATOM | 676 | CG2 | VAL | A | 387 | 20.658 | 10.864 | −1.088 | 1.00 | 22.89 |
| ATOM | 677 | C | VAL | A | 387 | 19.394 | 13.786 | 1.104 | 1.00 | 28.03 |
| ATOM | 678 | O | VAL | A | 387 | 20.265 | 14.602 | 1.446 | 1.00 | 27.40 |
| ATOM | 679 | N | LEU | A | 388 | 18.099 | 14.114 | 0.975 | 1.00 | 27.01 |
| ATOM | 680 | CA | LEU | A | 388 | 17.606 | 15.482 | 1.212 | 1.00 | 25.83 |
| ATOM | 681 | CB | LEU | A | 388 | 16.149 | 15.631 | 0.776 | 1.00 | 25.27 |
| ATOM | 682 | CG | LEU | A | 388 | 15.822 | 15.597 | −0.723 | 1.00 | 23.66 |
| ATOM | 683 | CD1 | LEU | A | 388 | 14.344 | 15.786 | −0.898 | 1.00 | 24.29 |
| ATOM | 684 | CD2 | LEU | A | 388 | 16.587 | 16.686 | −1.461 | 1.00 | 27.16 |
| ATOM | 685 | C | LEU | A | 388 | 17.738 | 15.871 | 2.683 | 1.00 | 26.40 |
| ATOM | 686 | O | LEU | A | 388 | 18.094 | 16.998 | 2.998 | 1.00 | 25.91 |
| ATOM | 687 | N | LEU | A | 389 | 17.461 | 14.928 | 3.572 | 1.00 | 25.45 |
| ATOM | 688 | CA | LEU | A | 389 | 17.578 | 15.192 | 4.994 | 1.00 | 23.81 |
| ATOM | 689 | CB | LEU | A | 389 | 16.915 | 14.096 | 5.803 | 1.00 | 23.38 |
| ATOM | 690 | CG | LEU | A | 389 | 15.402 | 14.206 | 5.789 | 1.00 | 22.32 |
| ATOM | 691 | CD1 | LEU | A | 389 | 14.785 | 13.003 | 6.476 | 1.00 | 25.13 |
| ATOM | 692 | CD2 | LEU | A | 389 | 14.984 | 15.501 | 6.427 | 1.00 | 19.09 |
| ATOM | 693 | C | LEU | A | 389 | 19.020 | 15.299 | 5.376 | 1.00 | 21.15 |
| ATOM | 694 | O | LEU | A | 389 | 19.410 | 16.257 | 6.028 | 1.00 | 26.81 |
| ATOM | 695 | N | LYS | A | 390 | 19.849 | 14.373 | 4.916 | 1.00 | 18.69 |
| ATOM | 696 | CA | LYS | A | 390 | 21.250 | 14.429 | 5.273 | 1.00 | 21.34 |
| ATOM | 697 | CB | LYS | A | 390 | 22.050 | 13.326 | 4.592 | 1.00 | 24.41 |
| ATOM | 698 | CG | LYS | A | 390 | 21.938 | 11.960 | 5.200 | 1.00 | 29.06 |
| ATOM | 699 | CD | LYS | A | 390 | 23.067 | 11.081 | 4.673 | 1.00 | 31.45 |
| ATOM | 700 | CE | LYS | A | 390 | 23.062 | 9.718 | 5.342 | 1.00 | 40.63 |
| ATOM | 701 | NZ | LYS | A | 390 | 24.240 | 8.890 | 4.933 | 1.00 | 46.61 |
| ATOM | 702 | C | LYS | A | 390 | 21.884 | 15.751 | 4.907 | 1.00 | 25.73 |
| ATOM | 703 | O | LYS | A | 390 | 22.706 | 16.287 | 5.644 | 1.00 | 26.32 |
| ATOM | 704 | N | ALA | A | 391 | 21.478 | 16.295 | 3.770 | 1.00 | 25.48 |
| ATOM | 705 | CA | ALA | A | 391 | 22.065 | 17.527 | 3.281 | 1.00 | 23.46 |
| ATOM | 706 | CB | ALA | A | 391 | 22.076 | 17.505 | 1.766 | 1.00 | 26.48 |
| ATOM | 707 | C | ALA | A | 391 | 21.401 | 18.795 | 3.750 | 1.00 | 20.95 |
| ATOM | 708 | O | ALA | A | 391 | 22.074 | 19.789 | 4.005 | 1.00 | 27.25 |
| ATOM | 709 | N | GLY | A | 392 | 20.082 | 18.773 | 3.838 | 1.00 | 21.07 |
| ATOM | 710 | CA | GLY | A | 392 | 19.349 | 19.965 | 4.202 | 1.00 | 22.76 |
| ATOM | 711 | C | GLY | A | 392 | 18.923 | 20.133 | 5.638 | 1.00 | 23.17 |
| ATOM | 712 | O | GLY | A | 392 | 18.420 | 21.184 | 5.972 | 1.00 | 20.90 |
| ATOM | 713 | N | SER | A | 393 | 19.087 | 19.105 | 6.471 | 1.00 | 23.99 |
| ATOM | 714 | CA | SER | A | 393 | 18.706 | 19.178 | 7.889 | 1.00 | 23.62 |
| ATOM | 715 | CB | SER | A | 393 | 19.056 | 17.874 | 8.593 | 1.00 | 20.71 |
| ATOM | 716 | OG | SER | A | 393 | 17.926 | 17.045 | 8.510 | 1.00 | 31.96 |
| ATOM | 717 | C | SER | A | 393 | 19.343 | 20.322 | 8.656 | 1.00 | 17.30 |
| ATOM | 718 | O | SER | A | 393 | 18.645 | 21.149 | 9.223 | 1.00 | 19.88 |
| ATOM | 719 | N | LEU | A | 394 | 20.670 | 20.332 | 8.677 | 1.00 | 20.09 |
| ATOM | 720 | CA | LEU | A | 394 | 21.442 | 21.348 | 9.365 | 1.00 | 21.08 |
| ATOM | 721 | CB | LEU | A | 394 | 22.940 | 21.032 | 9.274 | 1.00 | 19.93 |
| ATOM | 722 | CG | LEU | A | 394 | 23.831 | 20.918 | 10.516 | 1.00 | 26.08 |
| ATOM | 723 | CD1 | LEU | A | 394 | 25.207 | 21.380 | 10.158 | 1.00 | 21.54 |
| ATOM | 724 | CD2 | LEU | A | 394 | 23.285 | 21.730 | 11.705 | 1.00 | 22.96 |
| ATOM | 725 | C | LEU | A | 394 | 21.161 | 22.713 | 8.753 | 1.00 | 23.43 |
| ATOM | 726 | O | LEU | A | 394 | 21.132 | 23.717 | 9.462 | 1.00 | 21.36 |
| ATOM | 727 | N | GLU | A | 395 | 20.942 | 22.759 | 7.436 | 1.00 | 22.13 |
| ATOM | 728 | CA | GLU | A | 395 | 20.642 | 24.037 | 6.787 | 1.00 | 20.91 |
| ATOM | 729 | CB | GLU | A | 395 | 20.574 | 23.890 | 5.260 | 1.00 | 22.76 |
| ATOM | 730 | CG | GLU | A | 395 | 21.881 | 23.459 | 4.611 | 1.00 | 23.62 |
| ATOM | 731 | CD | GLU | A | 395 | 21.809 | 23.461 | 3.084 | 1.00 | 26.69 |
| ATOM | 732 | OE1 | GLU | A | 395 | 22.852 | 23.482 | 2.434 | 1.00 | 27.60 |
| ATOM | 733 | OE2 | GLU | A | 395 | 20.709 | 23.428 | 2.526 | 1.00 | 22.32 |
| ATOM | 734 | C | GLU | A | 395 | 19.342 | 24.632 | 7.318 | 1.00 | 18.31 |
| ATOM | 735 | O | GLU | A | 395 | 19.270 | 25.832 | 7.574 | 1.00 | 20.03 |
| ATOM | 736 | N | VAL | A | 396 | 18.311 | 23.810 | 7.498 | 1.00 | 18.03 |
| ATOM | 737 | CA | VAL | A | 396 | 17.043 | 24.331 | 8.031 | 1.00 | 24.17 |
| ATOM | 738 | CB | VAL | A | 396 | 15.891 | 23.342 | 7.818 | 1.00 | 20.05 |
| ATOM | 739 | CG1 | VAL | A | 396 | 14.587 | 23.854 | 8.517 | 1.00 | 18.41 |
| ATOM | 740 | CG2 | VAL | A | 396 | 15.663 | 23.177 | 6.288 | 1.00 | 22.83 |
| ATOM | 741 | C | VAL | A | 396 | 17.174 | 24.744 | 9.525 | 1.00 | 25.12 |
| ATOM | 742 | O | VAL | A | 396 | 16.476 | 25.639 | 10.006 | 1.00 | 26.40 |
| ATOM | 743 | N | VAL | A | 397 | 18.077 | 24.073 | 10.232 | 1.00 | 23.34 |
| ATOM | 744 | CA | VAL | A | 397 | 18.358 | 24.401 | 11.617 | 1.00 | 18.37 |
| ATOM | 745 | CB | VAL | A | 397 | 19.296 | 23.338 | 12.232 | 1.00 | 19.79 |
| ATOM | 746 | CG1 | VAL | A | 397 | 19.915 | 23.829 | 13.534 | 1.00 | 20.93 |
| ATOM | 747 | CG2 | VAL | A | 397 | 18.517 | 22.069 | 12.472 | 1.00 | 16.14 |
| ATOM | 748 | C | VAL | A | 397 | 19.006 | 25.802 | 11.635 | 1.00 | 17.79 |
| ATOM | 749 | O | VAL | A | 397 | 18.547 | 26.676 | 12.359 | 1.00 | 21.81 |
| ATOM | 750 | N | PHE | A | 398 | 19.981 | 26.058 | 10.760 | 1.00 | 16.57 |
| ATOM | 751 | CA | PHE | A | 398 | 20.625 | 27.355 | 10.735 | 1.00 | 12.50 |
| ATOM | 752 | CB | PHE | A | 398 | 21.910 | 27.288 | 9.955 | 1.00 | 16.85 |
| ATOM | 753 | CG | PHE | A | 398 | 23.017 | 26.588 | 10.669 | 1.00 | 22.50 |
| ATOM | 754 | CD1 | PHE | A | 398 | 23.316 | 26.899 | 11.986 | 1.00 | 21.63 |
| ATOM | 755 | CD2 | PHE | A | 398 | 23.796 | 25.645 | 10.014 | 1.00 | 21.83 |
| ATOM | 756 | CE1 | PHE | A | 398 | 24.375 | 26.279 | 12.623 | 1.00 | 22.54 |
| ATOM | 757 | CE2 | PHE | A | 398 | 24.859 | 25.021 | 10.652 | 1.00 | 23.29 |
| ATOM | 758 | CZ | PHE | A | 398 | 25.151 | 25.334 | 11.953 | 1.00 | 22.58 |
| ATOM | 759 | C | PHE | A | 398 | 19.718 | 28.486 | 10.249 | 1.00 | 19.24 |
| ATOM | 760 | O | PHE | A | 398 | 19.944 | 29.669 | 10.570 | 1.00 | 19.14 |
| ATOM | 761 | N | ILE | A | 399 | 18.663 | 28.138 | 9.513 | 1.00 | 20.43 |
| ATOM | 762 | CA | ILE | A | 399 | 17.685 | 29.145 | 9.077 | 1.00 | 21.75 |
| ATOM | 763 | CB | ILE | A | 399 | 16.790 | 28.652 | 7.848 | 1.00 | 23.69 |
| ATOM | 764 | CG2 | ILE | A | 399 | 15.574 | 29.562 | 7.656 | 1.00 | 17.51 |
| ATOM | 765 | CG1 | ILE | A | 399 | 17.608 | 28.583 | 6.538 | 1.00 | 26.64 |
| ATOM | 766 | CD1 | ILE | A | 399 | 16.942 | 27.745 | 5.397 | 1.00 | 17.97 |
| ATOM | 767 | C | ILE | A | 399 | 16.771 | 29.382 | 10.296 | 1.00 | 18.17 |
| ATOM | 768 | O | ILE | A | 399 | 16.484 | 30.507 | 10.662 | 1.00 | 20.59 |
| ATOM | 769 | N | ARG | A | 400 | 16.317 | 28.307 | 10.921 | 1.00 | 19.22 |
| ATOM | 770 | CA | ARG | A | 400 | 15.451 | 28.415 | 12.087 | 1.00 | 20.62 |
| ATOM | 771 | CB | ARG | A | 400 | 15.000 | 27.029 | 12.553 | 1.00 | 17.24 |
| ATOM | 772 | CG | ARG | A | 400 | 13.783 | 26.537 | 11.852 | 1.00 | 15.74 |
| ATOM | 773 | CD | ARG | A | 400 | 13.420 | 25.143 | 12.246 | 1.00 | 17.01 |
| ATOM | 774 | NE | ARG | A | 400 | 12.189 | 24.760 | 11.553 | 1.00 | 21.84 |
| ATOM | 775 | CZ | ARG | A | 400 | 11.371 | 23.775 | 11.909 | 1.00 | 21.38 |
| ATOM | 776 | NH1 | ARG | A | 400 | 11.643 | 23.021 | 12.976 | 1.00 | 27.51 |
| ATOM | 777 | NH2 | ARG | A | 400 | 10.221 | 23.610 | 11.261 | 1.00 | 19.58 |
| ATOM | 778 | C | ARG | A | 400 | 16.132 | 29.177 | 13.235 | 1.00 | 22.12 |
| ATOM | 779 | O | ARG | A | 400 | 15.456 | 29.834 | 14.016 | 1.00 | 22.87 |
| ATOM | 780 | N | MET | A | 401 | 17.462 | 29.140 | 13.283 | 1.00 | 20.39 |
| ATOM | 781 | CA | MET | A | 401 | 18.251 | 29.813 | 14.320 | 1.00 | 22.78 |
| ATOM | 782 | CB | MET | A | 401 | 19.740 | 29.612 | 14.026 | 1.00 | 21.38 |
| ATOM | 783 | CG | MET | A | 401 | 20.681 | 30.082 | 15.096 | 1.00 | 17.22 |
| ATOM | 784 | SD | MET | A | 401 | 22.356 | 30.190 | 14.524 | 1.00 | 24.87 |
| ATOM | 785 | CE | MET | A | 401 | 22.858 | 28.674 | 14.579 | 1.00 | 30.52 |
| ATOM | 786 | C | MET | A | 401 | 17.942 | 31.308 | 14.448 | 1.00 | 27.16 |
| ATOM | 787 | O | MET | A | 401 | 18.177 | 31.913 | 15.495 | 1.00 | 24.54 |
| ATOM | 788 | N | CYS | A | 402 | 17.451 | 31.901 | 13.362 | 1.00 | 28.58 |
| ATOM | 789 | CA | CYS | A | 402 | 17.102 | 33.327 | 13.340 | 1.00 | 33.94 |
| ATOM | 790 | CB | CYS | A | 402 | 16.827 | 33.801 | 11.911 | 1.00 | 34.97 |
| ATOM | 791 | SG | CYS | A | 402 | 18.382 | 33.864 | 11.142 | 1.00 | 54.57 |
| ATOM | 792 | C | CYS | A | 402 | 15.947 | 33.699 | 14.236 | 1.00 | 33.06 |
| ATOM | 793 | O | CYS | A | 402 | 15.871 | 34.838 | 14.722 | 1.00 | 36.03 |
| ATOM | 794 | N | ARG | A | 403 | 15.033 | 32.759 | 14.438 | 1.00 | 30.48 |
| ATOM | 795 | CA | ARG | A | 403 | 13.903 | 33.013 | 15.316 | 1.00 | 30.17 |
| ATOM | 796 | CB | ARG | A | 403 | 12.846 | 31.936 | 15.165 | 1.00 | 27.27 |
| ATOM | 797 | CG | ARG | A | 403 | 12.529 | 31.529 | 13.760 | 1.00 | 32.18 |
| ATOM | 798 | CD | ARG | A | 403 | 11.217 | 30.784 | 13.777 | 1.00 | 32.00 |
| ATOM | 799 | NE | ARG | A | 403 | 11.202 | 29.740 | 14.794 | 1.00 | 37.42 |
| ATOM | 800 | CZ | ARG | A | 403 | 10.188 | 29.482 | 15.616 | 1.00 | 33.92 |
| ATOM | 801 | NH1 | ARG | A | 403 | 9.078 | 30.195 | 15.575 | 1.00 | 34.55 |
| ATOM | 802 | NH2 | ARG | A | 403 | 10.257 | 28.436 | 16.428 | 1.00 | 42.16 |
| ATOM | 803 | C | ARG | A | 403 | 14.368 | 32.993 | 16.780 | 1.00 | 29.68 |
| ATOM | 804 | O | ARG | A | 403 | 13.653 | 33.472 | 17.649 | 1.00 | 30.31 |
| ATOM | 805 | N | ALA | A | 404 | 15.556 | 32.431 | 17.016 | 1.00 | 26.70 |
| ATOM | 806 | CA | ALA | A | 404 | 16.145 | 32.267 | 18.336 | 1.00 | 21.83 |
| ATOM | 807 | CB | ALA | A | 404 | 16.328 | 30.768 | 18.612 | 1.00 | 17.43 |
| ATOM | 808 | C | ALA | A | 404 | 17.490 | 32.962 | 18.394 | 1.00 | 23.99 |
| ATOM | 809 | O | ALA | A | 404 | 18.371 | 32.543 | 19.134 | 1.00 | 26.66 |
| ATOM | 810 | N | PHE | A | 405 | 17.694 | 33.990 | 17.573 | 1.00 | 26.59 |
| ATOM | 811 | CA | PHE | A | 405 | 18.977 | 34.705 | 17.557 | 1.00 | 24.98 |
| ATOM | 812 | CB | PHE | A | 405 | 19.713 | 34.470 | 16.227 | 1.00 | 18.47 |
| ATOM | 813 | CG | PHE | A | 405 | 21.110 | 35.018 | 16.187 | 1.00 | 17.64 |
| ATOM | 814 | CD1 | PHE | A | 405 | 22.202 | 34.177 | 16.313 | 1.00 | 16.69 |
| ATOM | 815 | CD2 | PHE | A | 405 | 21.345 | 36.370 | 15.956 | 1.00 | 19.09 |
| ATOM | 816 | CE1 | PHE | A | 405 | 23.514 | 34.669 | 16.199 | 1.00 | 21.32 |
| ATOM | 817 | CE2 | PHE | A | 405 | 22.651 | 36.871 | 15.841 | 1.00 | 17.33 |
| ATOM | 818 | CZ | PHE | A | 405 | 23.734 | 36.032 | 15.957 | 1.00 | 19.99 |
| ATOM | 819 | C | PHE | A | 405 | 18.765 | 36.197 | 17.805 | 1.00 | 28.98 |
| ATOM | 820 | O | PHE | A | 405 | 17.845 | 36.789 | 17.256 | 1.00 | 31.78 |
| ATOM | 821 | N | ASP | A | 406 | 19.581 | 36.763 | 18.703 | 1.00 | 31.70 |
| ATOM | 822 | CA | ASP | A | 406 | 19.556 | 38.180 | 19.084 | 1.00 | 30.27 |
| ATOM | 823 | CB | ASP | A | 406 | 19.786 | 38.339 | 20.598 | 1.00 | 30.55 |
| ATOM | 824 | CG | ASP | A | 406 | 19.689 | 39.798 | 21.081 | 1.00 | 34.22 |
| ATOM | 825 | OD1 | ASP | A | 406 | 19.722 | 40.747 | 20.278 | 1.00 | 31.76 |
| ATOM | 826 | OD2 | ASP | A | 406 | 19.575 | 40.004 | 22.299 | 1.00 | 33.44 |
| ATOM | 827 | C | ASP | A | 406 | 20.671 | 38.897 | 18.330 | 1.00 | 29.96 |
| ATOM | 828 | O | ASP | A | 406 | 21.821 | 38.953 | 18.804 | 1.00 | 29.82 |
| ATOM | 829 | N | SER | A | 407 | 20.302 | 39.510 | 17.203 | 1.00 | 28.41 |
| ATOM | 830 | CA | SER | A | 407 | 21.263 | 40.224 | 16.374 | 1.00 | 34.69 |
| ATOM | 831 | CB | SER | A | 407 | 20.597 | 40.719 | 15.093 | 1.00 | 37.05 |
| ATOM | 832 | OG | SER | A | 407 | 21.531 | 40.766 | 14.018 | 1.00 | 51.86 |
| ATOM | 833 | C | SER | A | 407 | 21.922 | 41.381 | 17.115 | 1.00 | 34.14 |
| ATOM | 834 | O | SER | A | 407 | 23.147 | 41.501 | 17.139 | 1.00 | 35.01 |
| ATOM | 835 | N | GLN | A | 408 | 21.113 | 42.206 | 17.764 | 1.00 | 38.76 |
| ATOM | 836 | CA | GLN | A | 408 | 21.632 | 43.345 | 18.519 | 1.00 | 40.20 |
| ATOM | 837 | CB | GLN | A | 408 | 20.516 | 43.981 | 19.353 | 1.00 | 48.77 |
| ATOM | 838 | CG | GLN | A | 408 | 19.250 | 44.338 | 18.577 | 1.00 | 63.31 |
| ATOM | 839 | CD | GLN | A | 408 | 18.001 | 44.400 | 19.467 | 1.00 | 70.33 |
| ATOM | 840 | OE1 | GLN | A | 408 | 17.761 | 45.387 | 20.175 | 1.00 | 73.70 |
| ATOM | 841 | NE2 | GLN | A | 408 | 17.185 | 43.347 | 19.405 | 1.00 | 72.43 |
| ATOM | 842 | C | GLN | A | 408 | 22.744 | 42.908 | 19.463 | 1.00 | 36.52 |
| ATOM | 843 | O | GLN | A | 408 | 23.827 | 43.493 | 19.484 | 1.00 | 36.77 |
| ATOM | 844 | N | ASN | A | 409 | 22.514 | 41.803 | 20.156 | 1.00 | 32.86 |
| ATOM | 845 | CA | ASN | A | 409 | 23.466 | 41.338 | 21.132 | 1.00 | 31.16 |
| ATOM | 846 | CB | ASN | A | 409 | 22.722 | 41.083 | 22.438 | 1.00 | 34.90 |
| ATOM | 847 | CG | ASN | A | 409 | 22.115 | 42.371 | 23.024 | 1.00 | 35.74 |
| ATOM | 848 | OD1 | ASN | A | 409 | 22.850 | 43.282 | 23.401 | 1.00 | 38.31 |
| ATOM | 849 | ND2 | ASN | A | 409 | 20.784 | 42.457 | 23.076 | 1.00 | 31.20 |
| ATOM | 850 | C | ASN | A | 409 | 24.369 | 40.175 | 20.752 | 1.00 | 32.11 |
| ATOM | 851 | O | ASN | A | 409 | 25.179 | 39.729 | 21.574 | 1.00 | 34.18 |
| ATOM | 852 | N | ASN | A | 410 | 24.291 | 39.743 | 19.494 | 1.00 | 29.63 |
| ATOM | 853 | CA | ASN | A | 410 | 25.128 | 38.646 | 18.979 | 1.00 | 27.78 |
| ATOM | 854 | CB | ASN | A | 410 | 26.597 | 39.063 | 18.877 | 1.00 | 24.50 |
| ATOM | 855 | CG | ASN | A | 410 | 27.360 | 38.235 | 17.866 | 1.00 | 25.12 |
| ATOM | 856 | OD1 | ASN | A | 410 | 26.851 | 37.955 | 16.780 | 1.00 | 29.04 |
| ATOM | 857 | ND2 | ASN | A | 410 | 28.568 | 37.813 | 18.224 | 1.00 | 27.47 |
| ATOM | 858 | C | ASN | A | 410 | 25.016 | 37.476 | 19.928 | 1.00 | 28.72 |
| ATOM | 859 | O | ASN | A | 410 | 26.016 | 36.970 | 20.457 | 1.00 | 27.57 |
| ATOM | 860 | N | THR | A | 411 | 23.813 | 36.948 | 20.020 | 1.00 | 27.68 |
| ATOM | 861 | CA | THR | A | 411 | 23.602 | 35.905 | 20.977 | 1.00 | 26.24 |
| ATOM | 862 | CB | THR | A | 411 | 23.208 | 36.681 | 22.253 | 1.00 | 27.76 |
| ATOM | 863 | OG1 | THR | A | 411 | 24.162 | 36.446 | 23.305 | 1.00 | 31.38 |
| ATOM | 864 | CG2 | THR | A | 411 | 21.784 | 36.514 | 22.604 | 1.00 | 16.40 |
| ATOM | 865 | C | THR | A | 411 | 22.577 | 34.896 | 20.464 | 1.00 | 22.40 |
| ATOM | 866 | O | THR | A | 411 | 21.649 | 35.282 | 19.779 | 1.00 | 24.61 |
| ATOM | 867 | N | VAL | A | 412 | 22.768 | 33.612 | 20.771 | 1.00 | 20.76 |
| ATOM | 868 | CA | VAL | A | 412 | 21.857 | 32.528 | 20.343 | 1.00 | 19.57 |
| ATOM | 869 | CB | VAL | A | 412 | 22.607 | 31.487 | 19.475 | 1.00 | 19.83 |
| ATOM | 870 | CG1 | VAL | A | 412 | 21.655 | 30.780 | 18.524 | 1.00 | 18.01 |
| ATOM | 871 | CG2 | VAL | A | 412 | 23.691 | 32.130 | 18.740 | 1.00 | 33.67 |
| ATOM | 872 | C | VAL | A | 412 | 21.278 | 31.708 | 21.508 | 1.00 | 18.12 |
| ATOM | 873 | O | VAL | A | 412 | 22.009 | 31.325 | 22.411 | 1.00 | 23.18 |
| ATOM | 874 | N | TYR | A | 413 | 19.990 | 31.386 | 21.453 | 1.00 | 16.25 |
| ATOM | 875 | CA | TYR | A | 413 | 19.320 | 30.575 | 22.465 | 1.00 | 17.45 |
| ATOM | 876 | CB | TYR | A | 413 | 17.855 | 30.512 | 22.122 | 1.00 | 17.78 |
| ATOM | 877 | CG | TYR | A | 413 | 16.935 | 29.951 | 23.181 | 1.00 | 25.60 |
| ATOM | 878 | CD1 | TYR | A | 413 | 17.039 | 30.319 | 24.529 | 1.00 | 24.61 |
| ATOM | 879 | CE1 | TYR | A | 413 | 16.122 | 29.840 | 25.472 | 1.00 | 21.19 |
| ATOM | 880 | CD2 | TYR | A | 413 | 15.906 | 29.103 | 22.819 | 1.00 | 24.64 |
| ATOM | 881 | CE2 | TYR | A | 413 | 14.991 | 28.629 | 23.739 | 1.00 | 26.94 |
| ATOM | 882 | CZ | TYR | A | 413 | 15.097 | 28.993 | 25.065 | 1.00 | 28.21 |
| ATOM | 883 | OH | TYR | A | 413 | 14.145 | 28.487 | 25.945 | 1.00 | 27.94 |
| ATOM | 884 | C | TYR | A | 413 | 19.906 | 29.164 | 22.453 | 1.00 | 23.48 |
| ATOM | 885 | O | TYR | A | 413 | 19.656 | 28.401 | 21.518 | 1.00 | 24.97 |
| ATOM | 886 | N | PHE | A | 414 | 20.684 | 28.828 | 23.488 | 1.00 | 22.58 |
| ATOM | 887 | CA | PHE | A | 414 | 21.360 | 27.534 | 23.625 | 1.00 | 21.94 |
| ATOM | 888 | CB | PHE | A | 414 | 22.835 | 27.685 | 23.195 | 1.00 | 18.84 |
| ATOM | 889 | CG | PHE | A | 414 | 23.734 | 26.507 | 23.555 | 1.00 | 23.28 |
| ATOM | 890 | CD1 | PHE | A | 414 | 23.637 | 25.283 | 22.871 | 1.00 | 23.77 |
| ATOM | 891 | CD2 | PHE | A | 414 | 24.718 | 26.634 | 24.556 | 1.00 | 20.51 |
| ATOM | 892 | CE1 | PHE | A | 414 | 24.510 | 24.201 | 23.178 | 1.00 | 25.81 |
| ATOM | 893 | CE2 | PHE | A | 414 | 25.586 | 25.569 | 24.865 | 1.00 | 17.09 |
| ATOM | 894 | CZ | PHE | A | 414 | 25.483 | 24.350 | 24.178 | 1.00 | 23.54 |
| ATOM | 895 | C | PHE | A | 414 | 21.303 | 26.977 | 25.057 | 1.00 | 25.66 |
| ATOM | 896 | O | PHE | A | 414 | 21.612 | 27.677 | 26.024 | 1.00 | 22.04 |
| ATOM | 897 | N | ASP | A | 415 | 20.866 | 25.731 | 25.196 | 1.00 | 25.08 |
| ATOM | 898 | CA | ASP | A | 415 | 20.849 | 25.114 | 26.513 | 1.00 | 22.21 |
| ATOM | 899 | CB | ASP | A | 415 | 22.303 | 24.853 | 26.921 | 1.00 | 17.07 |
| ATOM | 900 | CG | ASP | A | 415 | 22.441 | 23.735 | 27.894 | 1.00 | 18.13 |
| ATOM | 901 | OD1 | ASP | A | 415 | 21.457 | 23.014 | 28.110 | 1.00 | 18.33 |
| ATOM | 902 | OD2 | ASP | A | 415 | 23.551 | 23.579 | 28.420 | 1.00 | 20.42 |
| ATOM | 903 | C | ASP | A | 415 | 20.130 | 25.970 | 27.579 | 1.00 | 25.14 |
| ATOM | 904 | O | ASP | A | 415 | 20.700 | 26.259 | 28.633 | 1.00 | 29.15 |
| ATOM | 905 | N | GLY | A | 416 | 18.912 | 26.408 | 27.269 | 1.00 | 20.65 |
| ATOM | 906 | CA | GLY | A | 416 | 18.118 | 27.176 | 28.201 | 1.00 | 18.89 |
| ATOM | 907 | C | GLY | A | 416 | 18.258 | 28.682 | 28.257 | 1.00 | 13.58 |
| ATOM | 908 | O | GLY | A | 416 | 17.349 | 29.327 | 28.744 | 1.00 | 19.33 |
| ATOM | 909 | N | LYS | A | 417 | 19.364 | 29.254 | 27.817 | 1.00 | 14.55 |
| ATOM | 910 | CA | LYS | A | 417 | 19.498 | 30.716 | 27.861 | 1.00 | 18.31 |
| ATOM | 911 | CB | LYS | A | 417 | 20.345 | 31.134 | 29.074 | 1.00 | 20.71 |
| ATOM | 912 | CG | LYS | A | 417 | 19.682 | 30.877 | 30.428 | 1.00 | 22.06 |
| ATOM | 913 | CD | LYS | A | 417 | 20.679 | 31.167 | 31.538 | 1.00 | 23.89 |
| ATOM | 914 | CE | LYS | A | 417 | 19.970 | 31.232 | 32.891 | 1.00 | 26.28 |
| ATOM | 915 | NZ | LYS | A | 417 | 20.937 | 31.696 | 33.916 | 1.00 | 27.48 |
| ATOM | 916 | C | LYS | A | 417 | 20.183 | 31.202 | 26.592 | 1.00 | 23.82 |
| ATOM | 917 | O | LYS | A | 417 | 20.635 | 30.382 | 25.781 | 1.00 | 21.77 |
| ATOM | 918 | N | TYR | A | 418 | 20.315 | 32.526 | 26.443 | 1.00 | 21.57 |
| ATOM | 919 | CA | TYR | A | 418 | 20.972 | 33.115 | 25.270 | 1.00 | 19.31 |
| ATOM | 920 | CB | TYR | A | 418 | 20.369 | 34.492 | 24.921 | 1.00 | 18.21 |
| ATOM | 921 | CG | TYR | A | 418 | 19.066 | 34.407 | 24.142 | 1.00 | 21.12 |
| ATOM | 922 | CD1 | TYR | A | 418 | 17.856 | 34.087 | 24.777 | 1.00 | 18.03 |
| ATOM | 923 | CE1 | TYR | A | 418 | 16.682 | 33.930 | 24.048 | 1.00 | 22.16 |
| ATOM | 924 | CD2 | TYR | A | 418 | 19.060 | 34.576 | 22.743 | 1.00 | 19.41 |
| ATOM | 925 | CE2 | TYR | A | 418 | 17.903 | 34.419 | 22.007 | 1.00 | 16.83 |
| ATOM | 926 | CZ | TYR | A | 418 | 16.728 | 34.099 | 22.648 | 1.00 | 24.12 |
| ATOM | 927 | OH | TYR | A | 418 | 15.601 | 33.944 | 21.890 | 1.00 | 22.47 |
| ATOM | 928 | C | TYR | A | 418 | 22.471 | 33.183 | 25.451 | 1.00 | 19.85 |
| ATOM | 929 | O | TYR | A | 418 | 22.974 | 33.820 | 26.385 | 1.00 | 25.51 |
| ATOM | 930 | N | ALA | A | 419 | 23.180 | 32.482 | 24.570 | 1.00 | 16.24 |
| ATOM | 931 | CA | ALA | A | 419 | 24.630 | 32.365 | 24.577 | 1.00 | 15.65 |
| ATOM | 932 | CB | ALA | A | 419 | 24.986 | 30.903 | 24.326 | 1.00 | 17.04 |
| ATOM | 933 | C | ALA | A | 419 | 25.463 | 33.245 | 23.629 | 1.00 | 25.93 |
| ATOM | 934 | O | ALA | A | 419 | 25.168 | 33.359 | 22.432 | 1.00 | 27.89 |
| ATOM | 935 | N | SER | A | 420 | 26.540 | 33.817 | 24.157 | 1.00 | 23.17 |
| ATOM | 936 | CA | SER | A | 420 | 27.455 | 34.619 | 23.360 | 1.00 | 24.99 |
| ATOM | 937 | CB | SER | A | 420 | 28.202 | 35.577 | 24.280 | 1.00 | 27.55 |
| ATOM | 938 | OG | SER | A | 420 | 29.050 | 34.861 | 25.170 | 1.00 | 31.44 |
| ATOM | 939 | C | SER | A | 420 | 28.442 | 33.622 | 22.735 | 1.00 | 25.61 |
| ATOM | 940 | O | SER | A | 420 | 28.462 | 32.461 | 23.132 | 1.00 | 26.24 |
| ATOM | 941 | N | PRO | A | 421 | 29.267 | 34.040 | 21.748 | 1.00 | 27.91 |
| ATOM | 942 | CD | PRO | A | 421 | 29.281 | 35.313 | 20.998 | 1.00 | 28.16 |
| ATOM | 943 | CA | PRO | A | 421 | 30.209 | 33.071 | 21.160 | 1.00 | 27.21 |
| ATOM | 944 | CB | PRO | A | 421 | 31.014 | 33.928 | 20.178 | 1.00 | 25.80 |
| ATOM | 945 | CG | PRO | A | 421 | 30.031 | 34.959 | 19.743 | 1.00 | 24.69 |
| ATOM | 946 | C | PRO | A | 421 | 31.158 | 32.380 | 22.163 | 1.00 | 34.03 |
| ATOM | 947 | O | PRO | A | 421 | 31.509 | 31.199 | 21.988 | 1.00 | 31.00 |
| ATOM | 948 | N | ASP | A | 422 | 31.627 | 33.142 | 23.158 | 1.00 | 33.32 |
| ATOM | 949 | CA | ASP | A | 422 | 32.570 | 32.623 | 24.154 | 1.00 | 35.81 |
| ATOM | 950 | CB | ASP | A | 422 | 33.136 | 33.742 | 25.030 | 1.00 | 44.04 |
| ATOM | 951 | CG | ASP | A | 422 | 32.246 | 34.946 | 25.069 | 1.00 | 54.68 |
| ATOM | 952 | OD1 | ASP | A | 422 | 32.454 | 35.892 | 24.251 | 1.00 | 54.42 |
| ATOM | 953 | OD2 | ASP | A | 422 | 31.327 | 34.920 | 25.913 | 1.00 | 58.40 |
| ATOM | 954 | C | ASP | A | 422 | 32.104 | 31.415 | 24.972 | 1.00 | 29.08 |
| ATOM | 955 | O | ASP | A | 422 | 32.923 | 30.698 | 25.550 | 1.00 | 29.48 |
| ATOM | 956 | N | VAL | A | 423 | 30.796 | 31.180 | 24.968 | 1.00 | 24.96 |
| ATOM | 957 | CA | VAL | A | 423 | 30.197 | 30.031 | 25.620 | 1.00 | 22.62 |
| ATOM | 958 | CB | VAL | A | 423 | 28.671 | 30.120 | 25.495 | 1.00 | 19.09 |
| ATOM | 959 | CG1 | VAL | A | 423 | 28.025 | 28.771 | 25.617 | 1.00 | 20.56 |
| ATOM | 960 | CG2 | VAL | A | 423 | 28.115 | 31.068 | 26.536 | 1.00 | 21.12 |
| ATOM | 961 | C | VAL | A | 423 | 30.724 | 28.783 | 24.894 | 1.00 | 26.71 |
| ATOM | 962 | O | VAL | A | 423 | 30.975 | 27.746 | 25.499 | 1.00 | 24.85 |
| ATOM | 963 | N | PHE | A | 424 | 31.040 | 28.951 | 23.611 | 1.00 | 26.18 |
| ATOM | 964 | CA | PHE | A | 424 | 31.507 | 27.854 | 22.774 | 1.00 | 22.48 |
| ATOM | 965 | CB | PHE | A | 424 | 30.860 | 27.963 | 21.371 | 1.00 | 23.86 |
| ATOM | 966 | CG | PHE | A | 424 | 29.341 | 28.011 | 21.394 | 1.00 | 20.24 |
| ATOM | 967 | CD1 | PHE | A | 424 | 28.668 | 29.227 | 21.384 | 1.00 | 15.91 |
| ATOM | 968 | CD2 | PHE | A | 424 | 28.598 | 26.843 | 21.509 | 1.00 | 15.76 |
| ATOM | 969 | CE1 | PHE | A | 424 | 27.290 | 29.270 | 21.500 | 1.00 | 16.18 |
| ATOM | 970 | CE2 | PHE | A | 424 | 27.238 | 26.880 | 21.623 | 1.00 | 13.58 |
| ATOM | 971 | CZ | PHE | A | 424 | 26.573 | 28.099 | 21.624 | 1.00 | 16.90 |
| ATOM | 972 | C | PHE | A | 424 | 33.014 | 27.757 | 22.663 | 1.00 | 23.66 |
| ATOM | 973 | O | PHE | A | 424 | 33.539 | 26.929 | 21.911 | 1.00 | 24.93 |
| ATOM | 974 | N | LYS | A | 425 | 33.727 | 28.552 | 23.451 | 1.00 | 26.33 |
| ATOM | 975 | CA | LYS | A | 425 | 35.186 | 28.527 | 23.392 | 1.00 | 29.12 |
| ATOM | 976 | CB | LYS | A | 425 | 35.776 | 29.467 | 24.452 | 1.00 | 32.35 |
| ATOM | 977 | CG | LYS | A | 425 | 37.306 | 29.529 | 24.457 | 1.00 | 31.98 |
| ATOM | 978 | CD | LYS | A | 425 | 37.762 | 30.593 | 25.418 | 1.00 | 39.70 |
| ATOM | 979 | CE | LYS | A | 425 | 39.265 | 30.718 | 25.450 | 1.00 | 48.00 |
| ATOM | 980 | NZ | LYS | A | 425 | 39.725 | 31.844 | 26.336 | 1.00 | 53.67 |
| ATOM | 981 | C | LYS | A | 425 | 35.889 | 27.157 | 23.476 | 1.00 | 31.57 |
| ATOM | 982 | O | LYS | A | 425 | 36.792 | 26.863 | 22.673 | 1.00 | 27.27 |
| ATOM | 983 | N | SER | A | 426 | 35.521 | 26.339 | 24.465 | 1.00 | 30.78 |
| ATOM | 984 | CA | SER | A | 426 | 36.182 | 25.050 | 24.607 | 1.00 | 33.82 |
| ATOM | 985 | CB | SER | A | 426 | 35.862 | 24.390 | 25.959 | 1.00 | 30.28 |
| ATOM | 986 | OG | SER | A | 426 | 34.486 | 24.161 | 26.133 | 1.00 | 37.92 |
| ATOM | 987 | C | SER | A | 426 | 35.992 | 24.104 | 23.417 | 1.00 | 35.48 |
| ATOM | 988 | O | SER | A | 426 | 36.713 | 23.122 | 23.286 | 1.00 | 37.65 |
| ATOM | 989 | N | LEU | A | 427 | 35.109 | 24.463 | 22.488 | 1.00 | 35.14 |
| ATOM | 990 | CA | LEU | A | 427 | 34.879 | 23.644 | 21.300 | 1.00 | 33.29 |
| ATOM | 991 | CB | LEU | A | 427 | 33.771 | 24.270 | 20.461 | 1.00 | 30.08 |
| ATOM | 992 | CG | LEU | A | 427 | 32.682 | 23.462 | 19.781 | 1.00 | 28.00 |
| ATOM | 993 | CD1 | LEU | A | 427 | 32.151 | 22.380 | 20.671 | 1.00 | 25.88 |
| ATOM | 994 | CD2 | LEU | A | 427 | 31.574 | 24.418 | 19.397 | 1.00 | 25.10 |
| ATOM | 995 | C | LEU | A | 427 | 36.166 | 23.655 | 20.494 | 1.00 | 33.91 |
| ATOM | 996 | O | LEU | A | 427 | 36.533 | 22.656 | 19.901 | 1.00 | 36.72 |
| ATOM | 997 | N | GLY | A | 428 | 36.862 | 24.790 | 20.503 | 1.00 | 39.65 |
| ATOM | 998 | CA | GLY | A | 428 | 38.105 | 24.932 | 19.750 | 1.00 | 40.93 |
| ATOM | 999 | C | GLY | A | 428 | 37.884 | 25.241 | 18.269 | 1.00 | 43.39 |
| ATOM | 1000 | O | GLY | A | 428 | 38.806 | 25.108 | 17.455 | 1.00 | 42.81 |
| ATOM | 1001 | N | CYS | A | 429 | 36.670 | 25.685 | 17.927 | 1.00 | 43.35 |
| ATOM | 1002 | CA | CYS | A | 429 | 36.291 | 25.995 | 16.544 | 1.00 | 43.84 |
| ATOM | 1003 | CB | CYS | A | 429 | 35.025 | 25.224 | 16.176 | 1.00 | 43.95 |
| ATOM | 1004 | SG | CYS | A | 429 | 35.244 | 23.452 | 16.181 | 1.00 | 50.28 |
| ATOM | 1005 | C | CYS | A | 429 | 36.012 | 27.475 | 16.409 | 1.00 | 42.57 |
| ATOM | 1006 | O | CYS | A | 429 | 35.003 | 27.873 | 15.838 | 1.00 | 39.45 |
| ATOM | 1007 | N | GLU | A | 430 | 36.934 | 28.292 | 16.901 | 1.00 | 43.43 |
| ATOM | 1008 | CA | GLU | A | 430 | 36.761 | 29.740 | 16.874 | 1.00 | 45.32 |
| ATOM | 1009 | CB | GLU | A | 430 | 38.051 | 30.447 | 17.301 | 1.00 | 53.81 |
| ATOM | 1010 | CG | GLU | A | 430 | 38.849 | 29.738 | 18.439 | 1.00 | 70.87 |
| ATOM | 1011 | CD | GLU | A | 430 | 38.051 | 29.456 | 19.737 | 1.00 | 77.73 |
| ATOM | 1012 | OE1 | GLU | A | 430 | 37.138 | 30.245 | 20.098 | 1.00 | 80.51 |
| ATOM | 1013 | OE2 | GLU | A | 430 | 38.365 | 28.437 | 20.408 | 1.00 | 78.46 |
| ATOM | 1014 | C | GLU | A | 430 | 36.236 | 30.320 | 15.552 | 1.00 | 40.15 |
| ATOM | 1015 | O | GLU | A | 430 | 35.304 | 31.127 | 15.563 | 1.00 | 35.32 |
| ATOM | 1016 | N | ASP | A | 431 | 36.782 | 29.870 | 14.420 | 1.00 | 36.97 |
| ATOM | 1017 | CA | ASP | A | 431 | 36.355 | 30.383 | 13.101 | 1.00 | 37.50 |
| ATOM | 1018 | CB | ASP | A | 431 | 37.352 | 30.007 | 12.009 | 1.00 | 45.12 |
| ATOM | 1019 | CG | ASP | A | 431 | 38.743 | 30.507 | 12.303 | 1.00 | 50.38 |
| ATOM | 1020 | OD1 | ASP | A | 431 | 39.005 | 31.715 | 12.088 | 1.00 | 52.92 |
| ATOM | 1021 | OD2 | ASP | A | 431 | 39.564 | 29.686 | 12.767 | 1.00 | 58.71 |
| ATOM | 1022 | C | ASP | A | 431 | 34.953 | 29.983 | 12.668 | 1.00 | 31.17 |
| ATOM | 1023 | O | ASP | A | 431 | 34.169 | 30.832 | 12.249 | 1.00 | 29.39 |
| ATOM | 1024 | N | PHE | A | 432 | 34.642 | 28.697 | 12.805 | 1.00 | 25.82 |
| ATOM | 1025 | CA | PHE | A | 432 | 33.334 | 28.162 | 12.467 | 1.00 | 24.06 |
| ATOM | 1026 | CB | PHE | A | 432 | 33.343 | 26.640 | 12.666 | 1.00 | 23.24 |
| ATOM | 1027 | CG | PHE | A | 432 | 31.981 | 26.008 | 12.634 | 1.00 | 26.00 |
| ATOM | 1028 | CD1 | PHE | A | 432 | 31.359 | 25.728 | 11.428 | 1.00 | 24.09 |
| ATOM | 1029 | CD2 | PHE | A | 432 | 31.301 | 25.727 | 13.826 | 1.00 | 24.00 |
| ATOM | 1030 | CE1 | PHE | A | 432 | 30.077 | 25.182 | 11.407 | 1.00 | 25.60 |
| ATOM | 1031 | CE2 | PHE | A | 432 | 30.017 | 25.182 | 13.819 | 1.00 | 23.27 |
| ATOM | 1032 | CZ | PHE | A | 432 | 29.399 | 24.907 | 12.608 | 1.00 | 27.15 |
| ATOM | 1033 | C | PHE | A | 432 | 32.290 | 28.823 | 13.368 | 1.00 | 24.49 |
| ATOM | 1034 | O | PHE | A | 432 | 31.166 | 29.081 | 12.939 | 1.00 | 23.40 |
| ATOM | 1035 | N | ILE | A | 433 | 32.667 | 29.123 | 14.614 | 1.00 | 23.33 |
| ATOM | 1036 | CA | ILE | A | 433 | 31.729 | 29.746 | 15.545 | 1.00 | 21.41 |
| ATOM | 1037 | CB | ILE | A | 433 | 32.226 | 29.697 | 17.038 | 1.00 | 24.82 |
| ATOM | 1038 | CG2 | ILE | A | 433 | 31.307 | 30.551 | 17.946 | 1.00 | 24.79 |
| ATOM | 1039 | CG1 | ILE | A | 433 | 32.233 | 28.244 | 17.554 | 1.00 | 22.78 |
| ATOM | 1040 | CD1 | ILE | A | 433 | 30.846 | 27.630 | 17.696 | 1.00 | 21.04 |
| ATOM | 1041 | C | ILE | A | 433 | 31.453 | 31.179 | 15.113 | 1.00 | 17.19 |
| ATOM | 1042 | O | ILE | A | 433 | 30.293 | 31.589 | 15.046 | 1.00 | 21.19 |
| ATOM | 1043 | N | SER | A | 434 | 32.491 | 31.937 | 14.770 | 1.00 | 23.08 |
| ATOM | 1044 | CA | SER | A | 434 | 32.257 | 33.319 | 14.327 | 1.00 | 25.60 |
| ATOM | 1045 | CB | SER | A | 434 | 33.561 | 34.097 | 14.162 | 1.00 | 28.06 |
| ATOM | 1046 | OG | SER | A | 434 | 34.547 | 33.294 | 13.558 | 1.00 | 37.81 |
| ATOM | 1047 | C | SER | A | 434 | 31.465 | 33.276 | 13.028 | 1.00 | 23.82 |
| ATOM | 1048 | O | SER | A | 434 | 30.564 | 34.072 | 12.821 | 1.00 | 23.76 |
| ATOM | 1049 | N | PHE | A | 435 | 31.752 | 32.279 | 12.199 | 1.00 | 24.82 |
| ATOM | 1050 | CA | PHE | A | 435 | 31.034 | 32.096 | 10.947 | 1.00 | 24.08 |
| ATOM | 1051 | CB | PHE | A | 435 | 31.646 | 30.923 | 10.161 | 1.00 | 26.80 |
| ATOM | 1052 | CG | PHE | A | 435 | 31.106 | 30.767 | 8.748 | 1.00 | 30.19 |
| ATOM | 1053 | CD1 | PHE | A | 435 | 30.205 | 31.686 | 8.209 | 1.00 | 35.50 |
| ATOM | 1054 | CD2 | PHE | A | 435 | 31.487 | 29.678 | 7.961 | 1.00 | 33.97 |
| ATOM | 1055 | CE1 | PHE | A | 435 | 29.684 | 31.523 | 6.906 | 1.00 | 34.39 |
| ATOM | 1056 | CE2 | PHE | A | 435 | 30.977 | 29.505 | 6.657 | 1.00 | 34.83 |
| ATOM | 1057 | CZ | PHE | A | 435 | 30.074 | 30.430 | 6.136 | 1.00 | 31.71 |
| ATOM | 1058 | C | PHE | A | 435 | 29.548 | 31.844 | 11.264 | 1.00 | 26.45 |
| ATOM | 1059 | O | PHE | A | 435 | 28.665 | 32.480 | 10.688 | 1.00 | 28.86 |
| ATOM | 1060 | N | VAL | A | 436 | 29.255 | 30.967 | 12.223 | 1.00 | 22.48 |
| ATOM | 1061 | CA | VAL | A | 436 | 27.865 | 30.691 | 12.559 | 1.00 | 17.41 |
| ATOM | 1062 | CB | VAL | A | 436 | 27.757 | 29.624 | 13.695 | 1.00 | 17.64 |
| ATOM | 1063 | CG1 | VAL | A | 436 | 26.377 | 29.570 | 14.228 | 1.00 | 22.49 |
| ATOM | 1064 | CG2 | VAL | A | 436 | 28.122 | 28.278 | 13.195 | 1.00 | 17.15 |
| ATOM | 1065 | C | VAL | A | 436 | 27.164 | 31.978 | 12.991 | 1.00 | 17.44 |
| ATOM | 1066 | O | VAL | A | 436 | 26.053 | 32.278 | 12.534 | 1.00 | 18.61 |
| ATOM | 1067 | N | PHE | A | 437 | 27.803 | 32.741 | 13.874 | 1.00 | 19.96 |
| ATOM | 1068 | CA | PHE | A | 437 | 27.184 | 33.971 | 14.362 | 1.00 | 22.62 |
| ATOM | 1069 | CB | PHE | A | 437 | 27.946 | 34.539 | 15.573 | 1.00 | 24.58 |
| ATOM | 1070 | CG | PHE | A | 437 | 27.559 | 33.905 | 16.891 | 1.00 | 23.31 |
| ATOM | 1071 | CD1 | PHE | A | 437 | 27.912 | 32.593 | 17.180 | 1.00 | 24.58 |
| ATOM | 1072 | CD2 | PHE | A | 437 | 26.855 | 34.627 | 17.841 | 1.00 | 23.23 |
| ATOM | 1073 | CE1 | PHE | A | 437 | 27.572 | 32.012 | 18.402 | 1.00 | 22.25 |
| ATOM | 1074 | CE2 | PHE | A | 437 | 26.514 | 34.055 | 19.059 | 1.00 | 23.56 |
| ATOM | 1075 | CZ | PHE | A | 437 | 26.874 | 32.746 | 19.334 | 1.00 | 18.83 |
| ATOM | 1076 | C | PHE | A | 437 | 27.010 | 35.034 | 13.274 | 1.00 | 22.54 |
| ATOM | 1077 | O | PHE | A | 437 | 25.985 | 35.716 | 13.232 | 1.00 | 27.26 |
| ATOM | 1078 | N | GLU | A | 438 | 28.001 | 35.176 | 12.400 | 1.00 | 26.36 |
| ATOM | 1079 | CA | GLU | A | 438 | 27.898 | 36.157 | 11.302 | 1.00 | 27.43 |
| ATOM | 1080 | CB | GLU | A | 438 | 29.164 | 36.179 | 10.440 | 1.00 | 30.05 |
| ATOM | 1081 | CG | GLU | A | 438 | 29.073 | 37.131 | 9.227 | 1.00 | 31.17 |
| ATOM | 1082 | CD | GLU | A | 438 | 30.417 | 37.447 | 8.605 | 1.00 | 31.35 |
| ATOM | 1083 | OE1 | GLU | A | 438 | 31.384 | 36.685 | 8.801 | 1.00 | 27.62 |
| ATOM | 1084 | OE2 | GLU | A | 438 | 30.509 | 38.491 | 7.932 | 1.00 | 40.19 |
| ATOM | 1085 | C | GLU | A | 438 | 26.674 | 35.839 | 10.450 | 1.00 | 25.73 |
| ATOM | 1086 | O | GLU | A | 438 | 25.918 | 36.730 | 10.093 | 1.00 | 28.14 |
| ATOM | 1087 | N | PHE | A | 439 | 26.449 | 34.555 | 10.188 | 1.00 | 26.08 |
| ATOM | 1088 | CA | PHE | A | 439 | 25.292 | 34.118 | 9.433 | 1.00 | 24.60 |
| ATOM | 1089 | CB | PHE | A | 439 | 25.398 | 32.619 | 9.135 | 1.00 | 26.02 |
| ATOM | 1090 | CG | PHE | A | 439 | 24.280 | 32.098 | 8.283 | 1.00 | 27.70 |
| ATOM | 1091 | CD1 | PHE | A | 439 | 24.304 | 32.270 | 6.904 | 1.00 | 29.06 |
| ATOM | 1092 | CD2 | PHE | A | 439 | 23.177 | 31.483 | 8.855 | 1.00 | 31.10 |
| ATOM | 1093 | CE1 | PHE | A | 439 | 23.251 | 31.842 | 6.123 | 1.00 | 24.89 |
| ATOM | 1094 | CE2 | PHE | A | 439 | 22.111 | 31.050 | 8.069 | 1.00 | 31.13 |
| ATOM | 1095 | CZ | PHE | A | 439 | 22.153 | 31.234 | 6.701 | 1.00 | 28.01 |
| ATOM | 1096 | C | PHE | A | 439 | 23.964 | 34.426 | 10.162 | 1.00 | 28.45 |
| ATOM | 1097 | O | PHE | A | 439 | 22.958 | 34.770 | 9.518 | 1.00 | 28.20 |
| ATOM | 1098 | N | GLY | A | 440 | 23.926 | 34.257 | 11.491 | 1.00 | 26.29 |
| ATOM | 1099 | CA | GLY | A | 440 | 22.699 | 34.550 | 12.217 | 1.00 | 20.95 |
| ATOM | 1100 | C | GLY | A | 440 | 22.380 | 36.035 | 12.079 | 1.00 | 24.76 |
| ATOM | 1101 | O | GLY | A | 440 | 21.247 | 36.459 | 11.831 | 1.00 | 24.56 |
| ATOM | 1102 | N | LYS | A | 441 | 23.409 | 36.842 | 12.249 | 1.00 | 25.11 |
| ATOM | 1103 | CA | LYS | A | 441 | 23.283 | 38.290 | 12.135 | 1.00 | 31.81 |
| ATOM | 1104 | CB | LYS | A | 441 | 24.674 | 38.871 | 12.293 | 1.00 | 34.53 |
| ATOM | 1105 | CG | LYS | A | 441 | 24.720 | 40.343 | 12.482 | 1.00 | 48.07 |
| ATOM | 1106 | CD | LYS | A | 441 | 25.618 | 40.643 | 13.668 | 1.00 | 58.50 |
| ATOM | 1107 | CE | LYS | A | 441 | 25.088 | 39.955 | 14.934 | 1.00 | 61.35 |
| ATOM | 1108 | NZ | LYS | A | 441 | 25.698 | 40.550 | 16.152 | 1.00 | 67.81 |
| ATOM | 1109 | C | LYS | A | 441 | 22.741 | 38.659 | 10.733 | 1.00 | 34.70 |
| ATOM | 1110 | O | LYS | A | 441 | 21.767 | 39.411 | 10.579 | 1.00 | 32.85 |
| ATOM | 1111 | N | SER | A | 442 | 23.408 | 38.091 | 9.729 | 1.00 | 33.69 |
| ATOM | 1112 | CA | SER | A | 442 | 23.113 | 38.278 | 8.312 | 1.00 | 32.10 |
| ATOM | 1113 | CB | SER | A | 442 | 24.060 | 37.413 | 7.493 | 1.00 | 34.00 |
| ATOM | 1114 | OG | SER | A | 442 | 23.706 | 37.415 | 6.142 | 1.00 | 42.63 |
| ATOM | 1115 | C | SER | A | 442 | 21.699 | 37.939 | 7.953 | 1.00 | 29.45 |
| ATOM | 1116 | O | SER | A | 442 | 21.022 | 38.710 | 7.283 | 1.00 | 28.07 |
| ATOM | 1117 | N | LEU | A | 443 | 21.252 | 36.769 | 8.383 | 1.00 | 30.75 |
| ATOM | 1118 | CA | LEU | A | 443 | 19.903 | 36.341 | 8.091 | 1.00 | 28.97 |
| ATOM | 1119 | CB | LEU | A | 443 | 19.754 | 34.844 | 8.362 | 1.00 | 32.66 |
| ATOM | 1120 | CG | LEU | A | 443 | 19.232 | 33.966 | 7.225 | 1.00 | 34.63 |
| ATOM | 1121 | CD1 | LEU | A | 443 | 19.001 | 32.579 | 7.758 | 1.00 | 34.61 |
| ATOM | 1122 | CD2 | LEU | A | 443 | 17.933 | 34.502 | 6.669 | 1.00 | 35.80 |
| ATOM | 1123 | C | LEU | A | 443 | 18.900 | 37.163 | 8.897 | 1.00 | 33.40 |
| ATOM | 1124 | O | LEU | A | 443 | 17.761 | 37.366 | 8.461 | 1.00 | 31.79 |
| ATOM | 1125 | N | CYS | A | 444 | 19.330 | 37.656 | 10.063 | 1.00 | 39.70 |
| ATOM | 1126 | CA | CYS | A | 444 | 18.474 | 38.478 | 10.937 | 1.00 | 44.50 |
| ATOM | 1127 | CB | CYS | A | 444 | 19.117 | 38.687 | 12.312 | 1.00 | 50.28 |
| ATOM | 1128 | SG | CYS | A | 444 | 18.752 | 37.411 | 13.539 | 1.00 | 54.66 |
| ATOM | 1129 | C | CYS | A | 444 | 18.177 | 39.845 | 10.346 | 1.00 | 44.03 |
| ATOM | 1130 | O | CYS | A | 444 | 17.053 | 40.343 | 10.468 | 1.00 | 42.14 |
| ATOM | 1131 | N | SER | A | 445 | 19.190 | 40.456 | 9.733 | 1.00 | 43.30 |
| ATOM | 1132 | CA | SER | A | 445 | 19.020 | 41.765 | 9.118 | 1.00 | 47.44 |
| ATOM | 1133 | CB | SER | A | 445 | 20.343 | 42.266 | 8.518 | 1.00 | 48.13 |
| ATOM | 1134 | OG | SER | A | 445 | 20.752 | 41.518 | 7.383 | 1.00 | 50.15 |
| ATOM | 1135 | C | SER | A | 445 | 17.885 | 41.815 | 8.071 | 1.00 | 48.45 |
| ATOM | 1136 | O | SER | A | 445 | 17.459 | 42.901 | 7.663 | 1.00 | 53.44 |
| ATOM | 1137 | N | MET | A | 446 | 17.393 | 40.656 | 7.636 | 1.00 | 44.69 |
| ATOM | 1138 | CA | MET | A | 446 | 16.306 | 40.631 | 6.665 | 1.00 | 43.05 |
| ATOM | 1139 | CB | MET | A | 446 | 16.389 | 39.386 | 5.789 | 1.00 | 40.50 |
| ATOM | 1140 | CG | MET | A | 446 | 17.577 | 39.419 | 4.842 | 1.00 | 41.60 |
| ATOM | 1141 | SD | MET | A | 446 | 17.833 | 37.906 | 3.917 | 1.00 | 47.28 |
| ATOM | 1142 | CE | MET | A | 446 | 19.506 | 37.667 | 4.191 | 1.00 | 42.50 |
| ATOM | 1143 | C | MET | A | 446 | 14.953 | 40.725 | 7.355 | 1.00 | 46.65 |
| ATOM | 1144 | O | MET | A | 446 | 13.971 | 41.150 | 6.746 | 1.00 | 50.18 |
| ATOM | 1145 | N | HIS | A | 447 | 14.921 | 40.382 | 8.643 | 1.00 | 46.34 |
| ATOM | 1146 | CA | HIS | A | 447 | 13.702 | 40.426 | 9.420 | 1.00 | 48.76 |
| ATOM | 1147 | CB | HIS | A | 447 | 13.259 | 41.882 | 9.589 | 1.00 | 60.69 |
| ATOM | 1148 | CG | HIS | A | 447 | 12.149 | 42.066 | 10.578 | 1.00 | 78.35 |
| ATOM | 1149 | CD2 | HIS | A | 447 | 11.722 | 41.273 | 11.592 | 1.00 | 83.48 |
| ATOM | 1150 | ND1 | HIS | A | 447 | 11.308 | 43.163 | 10.569 | 1.00 | 85.45 |
| ATOM | 1151 | CE1 | HIS | A | 447 | 10.405 | 43.032 | 11.529 | 1.00 | 86.49 |
| ATOM | 1152 | NE2 | HIS | A | 447 | 10.633 | 41.893 | 12.161 | 1.00 | 87.93 |
| ATOM | 1153 | C | HIS | A | 447 | 12.618 | 39.583 | 8.729 | 1.00 | 47.17 |
| ATOM | 1154 | O | HIS | A | 447 | 11.618 | 40.114 | 8.233 | 1.00 | 48.76 |
| ATOM | 1155 | N | LEU | A | 448 | 12.853 | 38.272 | 8.654 | 1.00 | 43.85 |
| ATOM | 1156 | CA | LEU | A | 448 | 11.922 | 37.320 | 8.021 | 1.00 | 37.21 |
| ATOM | 1157 | CB | LEU | A | 448 | 12.667 | 36.021 | 7.633 | 1.00 | 36.92 |
| ATOM | 1158 | CG | LEU | A | 448 | 14.004 | 36.045 | 6.867 | 1.00 | 36.45 |
| ATOM | 1159 | CD1 | LEU | A | 448 | 14.486 | 34.629 | 6.601 | 1.00 | 35.34 |
| ATOM | 1160 | CD2 | LEU | A | 448 | 13.867 | 36.798 | 5.553 | 1.00 | 41.17 |
| ATOM | 1161 | C | LEU | A | 448 | 10.703 | 36.961 | 8.887 | 1.00 | 34.64 |
| ATOM | 1162 | O | LEU | A | 448 | 10.847 | 36.731 | 10.083 | 1.00 | 35.66 |
| ATOM | 1163 | N | THR | A | 449 | 9.512 | 36.911 | 8.288 | 1.00 | 31.11 |
| ATOM | 1164 | CA | THR | A | 449 | 8.305 | 36.549 | 9.033 | 1.00 | 28.99 |
| ATOM | 1165 | CB | THR | A | 449 | 7.006 | 36.973 | 8.313 | 1.00 | 30.22 |
| ATOM | 1166 | OG1 | THR | A | 449 | 6.777 | 36.098 | 7.201 | 1.00 | 29.38 |
| ATOM | 1167 | CG2 | THR | A | 449 | 7.081 | 38.431 | 7.839 | 1.00 | 25.55 |
| ATOM | 1168 | C | THR | A | 449 | 8.271 | 35.030 | 9.134 | 1.00 | 29.64 |
| ATOM | 1169 | O | THR | A | 449 | 8.953 | 34.351 | 8.371 | 1.00 | 31.91 |
| ATOM | 1170 | N | GLU | A | 450 | 7.479 | 34.491 | 10.051 | 1.00 | 26.47 |
| ATOM | 1171 | CA | GLU | A | 450 | 7.392 | 33.047 | 10.182 | 1.00 | 27.53 |
| ATOM | 1172 | CB | GLU | A | 450 | 6.328 | 32.679 | 11.204 | 1.00 | 25.91 |
| ATOM | 1173 | CG | GLU | A | 450 | 6.730 | 33.026 | 12.617 | 1.00 | 27.73 |
| ATOM | 1174 | CD | GLU | A | 450 | 7.853 | 32.146 | 13.117 | 1.00 | 24.37 |
| ATOM | 1175 | OE1 | GLU | A | 450 | 7.554 | 31.004 | 13.513 | 1.00 | 29.52 |
| ATOM | 1176 | OE2 | GLU | A | 450 | 9.021 | 32.586 | 13.108 | 1.00 | 28.01 |
| ATOM | 1177 | C | GLU | A | 450 | 7.104 | 32.350 | 8.844 | 1.00 | 29.72 |
| ATOM | 1178 | O | GLU | A | 450 | 7.748 | 31.372 | 8.492 | 1.00 | 29.06 |
| ATOM | 1179 | N | ASP | A | 451 | 6.181 | 32.899 | 8.070 | 1.00 | 32.63 |
| ATOM | 1180 | CA | ASP | A | 451 | 5.830 | 32.306 | 6.780 | 1.00 | 30.31 |
| ATOM | 1181 | CB | ASP | A | 451 | 4.615 | 33.023 | 6.182 | 1.00 | 34.34 |
| ATOM | 1182 | CG | ASP | A | 451 | 3.314 | 32.660 | 6.891 | 1.00 | 39.42 |
| ATOM | 1183 | OD1 | ASP | A | 451 | 3.330 | 31.766 | 7.755 | 1.00 | 42.60 |
| ATOM | 1184 | OD2 | ASP | A | 451 | 2.261 | 33.254 | 6.573 | 1.00 | 46.92 |
| ATOM | 1185 | C | ASP | A | 451 | 7.005 | 32.321 | 5.813 | 1.00 | 27.29 |
| ATOM | 1186 | O | ASP | A | 451 | 7.238 | 31.364 | 5.090 | 1.00 | 27.40 |
| ATOM | 1187 | N | GLU | A | 452 | 7.765 | 33.404 | 5.821 | 1.00 | 29.17 |
| ATOM | 1188 | CA | GLU | A | 452 | 8.928 | 33.510 | 4.946 | 1.00 | 33.64 |
| ATOM | 1189 | CB | GLU | A | 452 | 9.521 | 34.927 | 5.022 | 1.00 | 34.38 |
| ATOM | 1190 | CG | GLU | A | 452 | 8.592 | 35.990 | 4.410 | 1.00 | 38.12 |
| ATOM | 1191 | CD | GLU | A | 452 | 9.061 | 37.426 | 4.609 | 1.00 | 40.47 |
| ATOM | 1192 | OE1 | GLU | A | 452 | 10.047 | 37.667 | 5.330 | 1.00 | 42.73 |
| ATOM | 1193 | OE2 | GLU | A | 452 | 8.424 | 38.339 | 4.047 | 1.00 | 45.30 |
| ATOM | 1194 | C | GLU | A | 452 | 9.976 | 32.432 | 5.286 | 1.00 | 35.02 |
| ATOM | 1195 | O | GLU | A | 452 | 10.558 | 31.814 | 4.378 | 1.00 | 32.26 |
| ATOM | 1196 | N | ILE | A | 453 | 10.200 | 32.209 | 6.589 | 1.00 | 31.95 |
| ATOM | 1197 | CA | ILE | A | 453 | 11.159 | 31.194 | 7.061 | 1.00 | 28.64 |
| ATOM | 1198 | CB | ILE | A | 453 | 11.397 | 31.290 | 8.615 | 1.00 | 31.75 |
| ATOM | 1199 | CG2 | ILE | A | 453 | 12.135 | 30.045 | 9.139 | 1.00 | 28.09 |
| ATOM | 1200 | CG1 | ILE | A | 453 | 12.218 | 32.538 | 8.952 | 1.00 | 29.57 |
| ATOM | 1201 | CD1 | ILE | A | 453 | 12.224 | 32.863 | 10.422 | 1.00 | 27.64 |
| ATOM | 1202 | C | ILE | A | 453 | 10.640 | 29.793 | 6.707 | 1.00 | 25.34 |
| ATOM | 1203 | O | ILE | A | 453 | 11.408 | 28.904 | 6.331 | 1.00 | 27.62 |
| ATOM | 1204 | N | ALA | A | 454 | 9.331 | 29.604 | 6.834 | 1.00 | 25.41 |
| ATOM | 1205 | CA | ALA | A | 454 | 8.698 | 28.317 | 6.524 | 1.00 | 28.05 |
| ATOM | 1206 | CB | ALA | A | 454 | 7.194 | 28.381 | 6.798 | 1.00 | 23.33 |
| ATOM | 1207 | C | ALA | A | 454 | 8.941 | 27.940 | 5.064 | 1.00 | 30.98 |
| ATOM | 1208 | O | ALA | A | 454 | 9.408 | 26.839 | 4.763 | 1.00 | 27.28 |
| ATOM | 1209 | N | LEU | A | 455 | 8.696 | 28.899 | 4.173 | 1.00 | 34.22 |
| ATOM | 1210 | CA | LEU | A | 455 | 8.850 | 28.677 | 2.744 | 1.00 | 33.15 |
| ATOM | 1211 | CB | LEU | A | 455 | 8.023 | 29.706 | 1.945 | 1.00 | 35.88 |
| ATOM | 1212 | CG | LEU | A | 455 | 6.504 | 29.399 | 1.973 | 1.00 | 35.93 |
| ATOM | 1213 | CD1 | LEU | A | 455 | 5.706 | 30.626 | 1.672 | 1.00 | 39.84 |
| ATOM | 1214 | CD2 | LEU | A | 455 | 6.159 | 28.277 | 0.989 | 1.00 | 32.00 |
| ATOM | 1215 | C | LEU | A | 455 | 10.307 | 28.601 | 2.328 | 1.00 | 30.16 |
| ATOM | 1216 | O | LEU | A | 455 | 10.676 | 27.724 | 1.540 | 1.00 | 32.09 |
| ATOM | 1217 | N | PHE | A | 456 | 11.150 | 29.460 | 2.894 | 1.00 | 26.03 |
| ATOM | 1218 | CA | PHE | A | 456 | 12.564 | 29.400 | 2.561 | 1.00 | 25.28 |
| ATOM | 1219 | CB | PHE | A | 456 | 13.313 | 30.595 | 3.142 | 1.00 | 27.10 |
| ATOM | 1220 | CG | PHE | A | 456 | 14.766 | 30.654 | 2.734 | 1.00 | 32.50 |
| ATOM | 1221 | CD1 | PHE | A | 456 | 15.151 | 30.366 | 1.421 | 1.00 | 31.72 |
| ATOM | 1222 | CD2 | PHE | A | 456 | 15.754 | 30.994 | 3.660 | 1.00 | 32.12 |
| ATOM | 1223 | CE1 | PHE | A | 456 | 16.484 | 30.414 | 1.040 | 1.00 | 31.33 |
| ATOM | 1224 | CE2 | PHE | A | 456 | 17.097 | 31.046 | 3.285 | 1.00 | 34.09 |
| ATOM | 1225 | CZ | PHE | A | 456 | 17.465 | 30.755 | 1.971 | 1.00 | 33.73 |
| ATOM | 1226 | C | PHE | A | 456 | 13.165 | 28.073 | 3.061 | 1.00 | 27.08 |
| ATOM | 1227 | O | PHE | A | 456 | 14.077 | 27.522 | 2.452 | 1.00 | 24.18 |
| ATOM | 1228 | N | SER | A | 457 | 12.626 | 27.547 | 4.162 | 1.00 | 26.66 |
| ATOM | 1229 | CA | SER | A | 457 | 13.084 | 26.278 | 4.719 | 1.00 | 24.70 |
| ATOM | 1230 | CB | SER | A | 457 | 12.366 | 25.986 | 6.034 | 1.00 | 22.68 |
| ATOM | 1231 | OG | SER | A | 457 | 12.761 | 26.899 | 7.025 | 1.00 | 28.15 |
| ATOM | 1232 | C | SER | A | 457 | 12.734 | 25.169 | 3.748 | 1.00 | 23.81 |
| ATOM | 1233 | O | SER | A | 457 | 13.561 | 24.315 | 3.425 | 1.00 | 21.55 |
| ATOM | 1234 | N | ALA | A | 458 | 11.470 | 25.154 | 3.337 | 1.00 | 24.49 |
| ATOM | 1235 | CA | ALA | A | 458 | 10.992 | 24.142 | 2.397 | 1.00 | 27.62 |
| ATOM | 1236 | CB | ALA | A | 458 | 9.526 | 24.345 | 2.126 | 1.00 | 26.08 |
| ATOM | 1237 | C | ALA | A | 458 | 11.811 | 24.190 | 1.095 | 1.00 | 25.57 |
| ATOM | 1238 | O | ALA | A | 458 | 12.205 | 23.161 | 0.571 | 1.00 | 28.96 |
| ATOM | 1239 | N | PHE | A | 459 | 12.153 | 25.399 | 0.660 | 1.00 | 27.37 |
| ATOM | 1240 | CA | PHE | A | 459 | 12.945 | 25.642 | −0.553 | 1.00 | 28.58 |
| ATOM | 1241 | CB | PHE | A | 459 | 13.083 | 27.162 | −0.758 | 1.00 | 28.53 |
| ATOM | 1242 | CG | PHE | A | 459 | 13.907 | 27.558 | −1.956 | 1.00 | 33.04 |
| ATOM | 1243 | CD1 | PHE | A | 459 | 13.402 | 27.404 | −3.255 | 1.00 | 37.29 |
| ATOM | 1244 | CD2 | PHE | A | 459 | 15.168 | 28.122 | −1.789 | 1.00 | 32.57 |
| ATOM | 1245 | CE1 | PHE | A | 459 | 14.142 | 27.809 | −4.360 | 1.00 | 33.63 |
| ATOM | 1246 | CE2 | PHE | A | 459 | 15.920 | 28.533 | −2.886 | 1.00 | 36.16 |
| ATOM | 1247 | CZ | PHE | A | 459 | 15.407 | 28.378 | −4.175 | 1.00 | 37.49 |
| ATOM | 1248 | C | PHE | A | 459 | 14.331 | 25.008 | −0.495 | 1.00 | 28.01 |
| ATOM | 1249 | O | PHE | A | 459 | 14.743 | 24.252 | −1.372 | 1.00 | 29.21 |
| ATOM | 1250 | N | VAL | A | 460 | 15.067 | 25.334 | 0.553 | 1.00 | 29.05 |
| ATOM | 1251 | CA | VAL | A | 460 | 16.407 | 24.798 | 0.701 | 1.00 | 26.40 |
| ATOM | 1252 | CB | VAL | A | 460 | 17.143 | 25.531 | 1.839 | 1.00 | 28.85 |
| ATOM | 1253 | CG1 | VAL | A | 460 | 18.470 | 24.967 | 2.044 | 1.00 | 34.54 |
| ATOM | 1254 | CG2 | VAL | A | 460 | 17.319 | 26.974 | 1.484 | 1.00 | 30.19 |
| ATOM | 1255 | C | VAL | A | 460 | 16.385 | 23.268 | 0.889 | 1.00 | 24.40 |
| ATOM | 1256 | O | VAL | A | 460 | 17.295 | 22.563 | 0.447 | 1.00 | 25.65 |
| ATOM | 1257 | N | LEU | A | 461 | 15.307 | 22.743 | 1.469 | 1.00 | 24.80 |
| ATOM | 1258 | CA | LEU | A | 461 | 15.194 | 21.297 | 1.700 | 1.00 | 26.95 |
| ATOM | 1259 | CB | LEU | A | 461 | 14.034 | 21.015 | 2.653 | 1.00 | 28.61 |
| ATOM | 1260 | CG | LEU | A | 461 | 14.024 | 19.600 | 3.228 | 1.00 | 30.23 |
| ATOM | 1261 | CD1 | LEU | A | 461 | 15.049 | 19.523 | 4.335 | 1.00 | 30.35 |
| ATOM | 1262 | CD2 | LEU | A | 461 | 12.637 | 19.257 | 3.751 | 1.00 | 37.76 |
| ATOM | 1263 | C | LEU | A | 461 | 14.966 | 20.496 | 0.416 | 1.00 | 27.27 |
| ATOM | 1264 | O | LEU | A | 461 | 15.566 | 19.430 | 0.198 | 1.00 | 26.82 |
| ATOM | 1265 | N | MET | A | 462 | 14.023 | 20.991 | −0.380 | 1.00 | 27.27 |
| ATOM | 1266 | CA | MET | A | 462 | 13.650 | 20.378 | −1.640 | 1.00 | 30.35 |
| ATOM | 1267 | CB | MET | A | 462 | 12.196 | 20.707 | −1.964 | 1.00 | 30.76 |
| ATOM | 1268 | CG | MET | A | 462 | 11.213 | 20.255 | −0.874 | 1.00 | 41.65 |
| ATOM | 1269 | SD | MET | A | 462 | 11.153 | 18.462 | −0.489 | 1.00 | 43.91 |
| ATOM | 1270 | CE | MET | A | 462 | 10.018 | 17.928 | −1.767 | 1.00 | 47.33 |
| ATOM | 1271 | C | MET | A | 462 | 14.574 | 20.870 | −2.730 | 1.00 | 29.93 |
| ATOM | 1272 | O | MET | A | 462 | 14.135 | 21.418 | −3.729 | 1.00 | 33.71 |
| ATOM | 1273 | N | SER | A | 463 | 15.864 | 20.657 | −2.524 | 1.00 | 30.81 |
| ATOM | 1274 | CA | SER | A | 463 | 16.881 | 21.064 | −3.470 | 1.00 | 35.27 |
| ATOM | 1275 | CB | SER | A | 463 | 18.143 | 21.489 | −2.730 | 1.00 | 36.89 |
| ATOM | 1276 | OG | SER | A | 463 | 18.963 | 22.273 | −3.578 | 1.00 | 50.70 |
| ATOM | 1277 | C | SER | A | 463 | 17.174 | 19.889 | −4.411 | 1.00 | 36.64 |
| ATOM | 1278 | O | SER | A | 463 | 17.599 | 18.819 | −3.976 | 1.00 | 32.89 |
| ATOM | 1279 | N | ALA | A | 464 | 16.925 | 20.096 | −5.702 | 1.00 | 39.89 |
| ATOM | 1280 | CA | ALA | A | 464 | 17.127 | 19.053 | −6.698 | 1.00 | 40.73 |
| ATOM | 1281 | CB | ALA | A | 464 | 16.425 | 19.425 | −7.980 | 1.00 | 39.42 |
| ATOM | 1282 | C | ALA | A | 464 | 18.585 | 18.728 | −6.969 | 1.00 | 42.63 |
| ATOM | 1283 | O | ALA | A | 464 | 18.897 | 17.616 | −7.401 | 1.00 | 48.91 |
| ATOM | 1284 | N | ASP | A | 465 | 19.481 | 19.663 | −6.656 | 1.00 | 43.62 |
| ATOM | 1285 | CA | ASP | A | 465 | 20.905 | 19.461 | −6.908 | 1.00 | 43.08 |
| ATOM | 1286 | CB | ASP | A | 465 | 21.546 | 20.754 | −7.398 | 1.00 | 49.21 |
| ATOM | 1287 | CG | ASP | A | 465 | 21.620 | 21.805 | −6.324 | 1.00 | 54.48 |
| ATOM | 1288 | OD1 | ASP | A | 465 | 22.753 | 22.243 | −6.029 | 1.00 | 56.04 |
| ATOM | 1289 | OD2 | ASP | A | 465 | 20.555 | 22.184 | −5.783 | 1.00 | 57.56 |
| ATOM | 1290 | C | ASP | A | 465 | 21.766 | 18.855 | −5.803 | 1.00 | 41.94 |
| ATOM | 1291 | O | ASP | A | 465 | 22.946 | 19.191 | −5.677 | 1.00 | 46.76 |
| ATOM | 1292 | N | ARG | A | 466 | 21.190 | 17.995 | −4.976 | 1.00 | 37.97 |
| ATOM | 1293 | CA | ARG | A | 466 | 21.987 | 17.342 | −3.953 | 1.00 | 34.33 |
| ATOM | 1294 | CB | ARG | A | 466 | 21.112 | 16.818 | −2.804 | 1.00 | 31.22 |
| ATOM | 1295 | CG | ARG | A | 466 | 20.380 | 17.869 | −2.006 | 1.00 | 27.68 |
| ATOM | 1296 | CD | ARG | A | 466 | 21.340 | 18.803 | −1.302 | 1.00 | 27.64 |
| ATOM | 1297 | NE | ARG | A | 466 | 20.588 | 19.665 | −0.400 | 1.00 | 26.47 |
| ATOM | 1298 | CZ | ARG | A | 466 | 21.076 | 20.728 | 0.234 | 1.00 | 24.01 |
| ATOM | 1299 | NH1 | ARG | A | 466 | 22.341 | 21.082 | 0.092 | 1.00 | 20.35 |
| ATOM | 1300 | NH2 | ARG | A | 466 | 20.266 | 21.477 | 0.969 | 1.00 | 25.10 |
| ATOM | 1301 | C | ARG | A | 466 | 22.613 | 16.155 | −4.681 | 1.00 | 32.20 |
| ATOM | 1302 | O | ARG | A | 466 | 21.981 | 15.542 | −5.543 | 1.00 | 34.80 |
| ATOM | 1303 | N | SER | A | 467 | 23.852 | 15.835 | −4.343 | 1.00 | 31.72 |
| ATOM | 1304 | CA | SER | A | 467 | 24.512 | 14.700 | −4.961 | 1.00 | 29.23 |
| ATOM | 1305 | CB | SER | A | 467 | 25.915 | 14.497 | −4.373 | 1.00 | 30.36 |
| ATOM | 1306 | OG | SER | A | 467 | 26.750 | 15.613 | −4.579 | 1.00 | 34.62 |
| ATOM | 1307 | C | SER | A | 467 | 23.705 | 13.442 | −4.680 | 1.00 | 29.79 |
| ATOM | 1308 | O | SER | A | 467 | 23.050 | 13.327 | −3.653 | 1.00 | 27.96 |
| ATOM | 1309 | N | TRP | A | 468 | 23.760 | 12.504 | −5.610 | 1.00 | 27.51 |
| ATOM | 1310 | CA | TRP | A | 468 | 23.114 | 11.203 | −5.476 | 1.00 | 27.13 |
| ATOM | 1311 | CB | TRP | A | 468 | 23.703 | 10.431 | −4.286 | 1.00 | 30.38 |
| ATOM | 1312 | CG | TRP | A | 468 | 25.196 | 10.650 | −4.109 | 1.00 | 33.34 |
| ATOM | 1313 | CD2 | TRP | A | 468 | 26.241 | 10.375 | −5.068 | 1.00 | 34.52 |
| ATOM | 1314 | CE2 | TRP | A | 468 | 27.453 | 10.853 | −4.510 | 1.00 | 34.97 |
| ATOM | 1315 | CE3 | TRP | A | 468 | 26.271 | 9.778 | −6.345 | 1.00 | 33.99 |
| ATOM | 1316 | CD1 | TRP | A | 468 | 25.807 | 11.246 | −3.043 | 1.00 | 35.05 |
| ATOM | 1317 | NE1 | TRP | A | 468 | 27.152 | 11.376 | −3.278 | 1.00 | 35.95 |
| ATOM | 1318 | CZ2 | TRP | A | 468 | 28.688 | 10.755 | −5.182 | 1.00 | 32.97 |
| ATOM | 1319 | CZ3 | TRP | A | 468 | 27.494 | 9.677 | −7.012 | 1.00 | 29.70 |
| ATOM | 1320 | CH2 | TRP | A | 468 | 28.686 | 10.167 | −6.425 | 1.00 | 34.34 |
| ATOM | 1321 | C | TRP | A | 468 | 21.602 | 11.121 | −5.472 | 1.00 | 27.07 |
| ATOM | 1322 | O | TRP | A | 468 | 21.042 | 10.084 | −5.105 | 1.00 | 29.52 |
| ATOM | 1323 | N | LEU | A | 469 | 20.921 | 12.190 | −5.874 | 1.00 | 27.42 |
| ATOM | 1324 | CA | LEU | A | 469 | 19.456 | 12.139 | −5.945 | 1.00 | 30.07 |
| ATOM | 1325 | CB | LEU | A | 469 | 18.910 | 13.539 | −6.099 | 1.00 | 28.28 |
| ATOM | 1326 | CG | LEU | A | 469 | 18.898 | 14.353 | −4.824 | 1.00 | 24.65 |
| ATOM | 1327 | CD1 | LEU | A | 469 | 18.463 | 15.758 | −5.160 | 1.00 | 26.09 |
| ATOM | 1328 | CD2 | LEU | A | 469 | 17.929 | 13.704 | −3.867 | 1.00 | 20.08 |
| ATOM | 1329 | C | LEU | A | 469 | 19.028 | 11.294 | −7.155 | 1.00 | 33.83 |
| ATOM | 1330 | O | LEU | A | 469 | 19.735 | 11.285 | −8.146 | 1.00 | 39.39 |
| ATOM | 1331 | N | GLN | A | 470 | 17.916 | 10.564 | −7.077 | 1.00 | 34.69 |
| ATOM | 1332 | CA | GLN | A | 470 | 17.463 | 9.757 | −8.224 | 1.00 | 39.38 |
| ATOM | 1333 | CB | GLN | A | 470 | 16.832 | 8.443 | −7.779 | 1.00 | 40.10 |
| ATOM | 1334 | CG | GLN | A | 470 | 17.796 | 7.485 | −7.120 | 1.00 | 50.51 |
| ATOM | 1335 | CD | GLN | A | 470 | 17.111 | 6.245 | −6.571 | 1.00 | 55.73 |
| ATOM | 1336 | OE1 | GLN | A | 470 | 15.993 | 5.911 | −6.964 | 1.00 | 57.73 |
| ATOM | 1337 | NE2 | GLN | A | 470 | 17.776 | 5.565 | −5.643 | 1.00 | 58.82 |
| ATOM | 1338 | C | GLN | A | 470 | 16.444 | 10.524 | −9.050 | 1.00 | 40.91 |
| ATOM | 1339 | O | GLN | A | 470 | 16.619 | 10.727 | −10.250 | 1.00 | 42.98 |
| ATOM | 1340 | N | GLU | A | 471 | 15.383 | 10.960 | −8.386 | 1.00 | 40.83 |
| ATOM | 1341 | CA | GLU | A | 471 | 14.308 | 11.720 | −9.013 | 1.00 | 40.88 |
| ATOM | 1342 | CB | GLU | A | 471 | 13.001 | 11.447 | −8.269 | 1.00 | 40.93 |
| ATOM | 1343 | CG | GLU | A | 471 | 12.699 | 9.972 | −8.077 | 1.00 | 48.61 |
| ATOM | 1344 | CD | GLU | A | 471 | 11.560 | 9.737 | −7.098 | 1.00 | 53.74 |
| ATOM | 1345 | OE1 | GLU | A | 471 | 10.392 | 10.051 | −7.438 | 1.00 | 52.66 |
| ATOM | 1346 | OE2 | GLU | A | 471 | 11.841 | 9.243 | −5.981 | 1.00 | 59.20 |
| ATOM | 1347 | C | GLU | A | 471 | 14.588 | 13.235 | −9.046 | 1.00 | 41.53 |
| ATOM | 1348 | O | GLU | A | 471 | 13.780 | 14.027 | −8.551 | 1.00 | 42.72 |
| ATOM | 1349 | N | LYS | A | 472 | 15.707 | 13.634 | −9.657 | 1.00 | 41.50 |
| ATOM | 1350 | CA | LYS | A | 472 | 16.086 | 15.044 | −9.750 | 1.00 | 41.09 |
| ATOM | 1351 | CB | LYS | A | 472 | 17.332 | 15.198 | −10.614 | 1.00 | 38.72 |
| ATOM | 1352 | CG | LYS | A | 472 | 18.592 | 14.669 | −9.971 | 1.00 | 43.30 |
| ATOM | 1353 | CD | LYS | A | 472 | 19.731 | 15.695 | −10.033 | 1.00 | 46.29 |
| ATOM | 1354 | CE | LYS | A | 472 | 20.679 | 15.517 | −8.831 | 1.00 | 50.44 |
| ATOM | 1355 | NZ | LYS | A | 472 | 21.892 | 16.398 | −8.804 | 1.00 | 46.13 |
| ATOM | 1356 | C | LYS | A | 472 | 14.978 | 15.941 | −10.287 | 1.00 | 43.56 |
| ATOM | 1357 | O | LYS | A | 472 | 14.628 | 16.963 | −9.689 | 1.00 | 43.75 |
| ATOM | 1358 | N | VAL | A | 473 | 14.399 | 15.511 | −11.402 | 1.00 | 46.51 |
| ATOM | 1359 | CA | VAL | A | 473 | 13.326 | 16.236 | −12.084 | 1.00 | 43.88 |
| ATOM | 1360 | CB | VAL | A | 473 | 12.963 | 15.516 | −13.416 | 1.00 | 49.53 |
| ATOM | 1361 | CG1 | VAL | A | 473 | 11.696 | 16.128 | −14.040 | 1.00 | 51.00 |
| ATOM | 1362 | CG2 | VAL | A | 473 | 14.155 | 15.611 | −14.395 | 1.00 | 49.56 |
| ATOM | 1363 | C | VAL | A | 473 | 12.076 | 16.466 | −11.235 | 1.00 | 37.01 |
| ATOM | 1364 | O | VAL | A | 473 | 11.536 | 17.572 | −11.212 | 1.00 | 33.80 |
| ATOM | 1365 | N | LYS | A | 474 | 11.609 | 15.415 | −10.570 | 1.00 | 36.39 |
| ATOM | 1366 | CA | LYS | A | 474 | 10.440 | 15.508 | −9.695 | 1.00 | 36.93 |
| ATOM | 1367 | CB | LYS | A | 474 | 10.084 | 14.126 | −9.147 | 1.00 | 35.35 |
| ATOM | 1368 | CG | LYS | A | 474 | 8.886 | 14.077 | −8.218 | 1.00 | 38.41 |
| ATOM | 1369 | CD | LYS | A | 474 | 8.579 | 12.626 | −7.848 | 1.00 | 46.48 |
| ATOM | 1370 | CE | LYS | A | 474 | 7.746 | 12.480 | −6.569 | 1.00 | 53.40 |
| ATOM | 1371 | NZ | LYS | A | 474 | 6.298 | 12.790 | −6.709 | 1.00 | 57.86 |
| ATOM | 1372 | C | LYS | A | 474 | 10.713 | 16.495 | −8.537 | 1.00 | 37.36 |
| ATOM | 1373 | O | LYS | A | 474 | 9.887 | 17.359 | −8.250 | 1.00 | 36.85 |
| ATOM | 1374 | N | ILE | A | 475 | 11.897 | 16.422 | −7.927 | 1.00 | 37.31 |
| ATOM | 1375 | CA | ILE | A | 475 | 12.231 | 17.328 | −6.820 | 1.00 | 37.61 |
| ATOM | 1376 | CB | ILE | A | 475 | 13.535 | 16.887 | −6.067 | 1.00 | 35.04 |
| ATOM | 1377 | CG2 | ILE | A | 475 | 13.834 | 17.877 | −4.931 | 1.00 | 34.59 |
| ATOM | 1378 | CG1 | ILE | A | 475 | 13.368 | 15.464 | −5.488 | 1.00 | 27.42 |
| ATOM | 1379 | CD1 | ILE | A | 475 | 14.680 | 14.711 | −5.243 | 1.00 | 18.44 |
| ATOM | 1380 | C | ILE | A | 475 | 12.349 | 18.766 | −7.352 | 1.00 | 40.05 |
| ATOM | 1381 | O | ILE | A | 475 | 11.882 | 19.727 | −6.721 | 1.00 | 38.84 |
| ATOM | 1382 | N | GLU | A | 476 | 12.913 | 18.896 | −8.550 | 1.00 | 41.69 |
| ATOM | 1383 | CA | GLU | A | 476 | 13.066 | 20.184 | −9.207 | 1.00 | 43.30 |
| ATOM | 1384 | CB | GLU | A | 476 | 13.755 | 19.984 | −10.552 | 1.00 | 48.95 |
| ATOM | 1385 | CG | GLU | A | 476 | 13.732 | 21.196 | −11.464 | 1.00 | 62.73 |
| ATOM | 1386 | CD | GLU | A | 476 | 14.355 | 22.442 | −10.846 | 1.00 | 70.08 |
| ATOM | 1387 | OE1 | GLU | A | 476 | 15.464 | 22.338 | −10.274 | 1.00 | 76.26 |
| ATOM | 1388 | OE2 | GLU | A | 476 | 13.741 | 23.532 | −10.947 | 1.00 | 75.60 |
| ATOM | 1389 | C | GLU | A | 476 | 11.705 | 20.850 | −9.413 | 1.00 | 42.70 |
| ATOM | 1390 | O | GLU | A | 476 | 11.547 | 22.050 | −9.183 | 1.00 | 38.37 |
| ATOM | 1391 | N | LYS | A | 477 | 10.724 | 20.066 | −9.854 | 1.00 | 41.95 |
| ATOM | 1392 | CA | LYS | A | 477 | 9.379 | 20.580 | −10.072 | 1.00 | 45.24 |
| ATOM | 1393 | CB | LYS | A | 477 | 8.461 | 19.496 | −10.643 | 1.00 | 49.85 |
| ATOM | 1394 | CG | LYS | A | 477 | 8.971 | 18.848 | −11.905 | 1.00 | 59.55 |
| ATOM | 1395 | CD | LYS | A | 477 | 9.158 | 19.879 | −12.984 | 1.00 | 69.14 |
| ATOM | 1396 | CE | LYS | A | 477 | 9.721 | 19.258 | −14.243 | 1.00 | 75.50 |
| ATOM | 1397 | NZ | LYS | A | 477 | 9.631 | 20.220 | −15.382 | 1.00 | 82.60 |
| ATOM | 1398 | C | LYS | A | 477 | 8.798 | 21.075 | −8.753 | 1.00 | 45.20 |
| ATOM | 1399 | O | LYS | A | 477 | 8.303 | 22.207 | −8.668 | 1.00 | 44.88 |
| ATOM | 1400 | N | LEU | A | 478 | 8.850 | 20.216 | −7.728 | 1.00 | 42.02 |
| ATOM | 1401 | CA | LEU | A | 478 | 8.339 | 20.575 | −6.403 | 1.00 | 39.03 |
| ATOM | 1402 | CB | LEU | A | 478 | 8.506 | 19.411 | −5.419 | 1.00 | 38.50 |
| ATOM | 1403 | CG | LEU | A | 478 | 7.562 | 18.213 | −5.609 | 1.00 | 38.77 |
| ATOM | 1404 | CD1 | LEU | A | 478 | 7.919 | 17.062 | −4.658 | 1.00 | 40.42 |
| ATOM | 1405 | CD2 | LEU | A | 478 | 6.126 | 18.653 | −5.389 | 1.00 | 36.80 |
| ATOM | 1406 | C | LEU | A | 478 | 9.020 | 21.856 | −5.875 | 1.00 | 37.92 |
| ATOM | 1407 | O | LEU | A | 478 | 8.332 | 22.767 | −5.399 | 1.00 | 37.49 |
| ATOM | 1408 | N | GLN | A | 479 | 10.344 | 21.963 | −6.057 | 1.00 | 37.80 |
| ATOM | 1409 | CA | GLN | A | 479 | 11.083 | 23.144 | −5.607 | 1.00 | 38.11 |
| ATOM | 1410 | CB | GLN | A | 479 | 12.610 | 22.987 | −5.772 | 1.00 | 36.91 |
| ATOM | 1411 | CG | GLN | A | 479 | 13.425 | 24.177 | −5.189 | 1.00 | 40.17 |
| ATOM | 1412 | CD | GLN | A | 479 | 14.938 | 24.122 | −5.452 | 1.00 | 42.02 |
| ATOM | 1413 | OE1 | GLN | A | 479 | 15.411 | 23.469 | −6.392 | 1.00 | 48.01 |
| ATOM | 1414 | NE2 | GLN | A | 479 | 15.702 | 24.820 | −4.616 | 1.00 | 46.19 |
| ATOM | 1415 | C | GLN | A | 479 | 10.589 | 24.379 | −6.343 | 1.00 | 39.25 |
| ATOM | 1416 | O | GLN | A | 479 | 10.392 | 25.426 | −5.718 | 1.00 | 42.08 |
| ATOM | 1417 | N | GLN | A | 480 | 10.348 | 24.251 | −7.650 | 1.00 | 39.34 |
| ATOM | 1418 | CA | GLN | A | 480 | 9.855 | 25.371 | −8.460 | 1.00 | 43.07 |
| ATOM | 1419 | CB | GLN | A | 480 | 9.547 | 24.917 | −9.894 | 1.00 | 51.07 |
| ATOM | 1420 | CG | GLN | A | 480 | 10.733 | 24.407 | −10.707 | 1.00 | 64.44 |
| ATOM | 1421 | CD | GLN | A | 480 | 10.291 | 23.533 | −11.889 | 1.00 | 73.00 |
| ATOM | 1422 | OE1 | GLN | A | 480 | 9.132 | 23.582 | −12.310 | 1.00 | 75.34 |
| ATOM | 1423 | NE2 | GLN | A | 480 | 11.211 | 22.732 | −12.425 | 1.00 | 72.93 |
| ATOM | 1424 | C | GLN | A | 480 | 8.591 | 25.987 | −7.853 | 1.00 | 37.24 |
| ATOM | 1425 | O | GLN | A | 480 | 8.499 | 27.198 | −7.658 | 1.00 | 35.48 |
| ATOM | 1426 | N | LYS | A | 481 | 7.620 | 25.139 | −7.545 | 1.00 | 35.17 |
| ATOM | 1427 | CA | LYS | A | 481 | 6.381 | 25.614 | −6.957 | 1.00 | 37.36 |
| ATOM | 1428 | CB | LYS | A | 481 | 5.397 | 24.470 | −6.801 | 1.00 | 41.26 |
| ATOM | 1429 | CG | LYS | A | 481 | 4.877 | 23.975 | −8.112 | 1.00 | 49.18 |
| ATOM | 1430 | CD | LYS | A | 481 | 3.683 | 23.105 | −7.907 | 1.00 | 59.24 |
| ATOM | 1431 | CE | LYS | A | 481 | 2.989 | 22.839 | −9.229 | 1.00 | 68.40 |
| ATOM | 1432 | NZ | LYS | A | 481 | 1.798 | 21.950 | −9.050 | 1.00 | 76.15 |
| ATOM | 1433 | C | LYS | A | 481 | 6.599 | 26.317 | −5.628 | 1.00 | 37.73 |
| ATOM | 1434 | O | LYS | A | 481 | 5.976 | 27.353 | −5.360 | 1.00 | 35.72 |
| ATOM | 1435 | N | ILE | A | 482 | 7.486 | 25.748 | −4.806 | 1.00 | 38.76 |
| ATOM | 1436 | CA | ILE | A | 482 | 7.831 | 26.299 | −3.494 | 1.00 | 36.16 |
| ATOM | 1437 | CB | ILE | A | 482 | 8.817 | 25.381 | −2.705 | 1.00 | 32.89 |
| ATOM | 1438 | CG2 | ILE | A | 482 | 9.404 | 26.130 | −1.514 | 1.00 | 34.55 |
| ATOM | 1439 | CG1 | ILE | A | 482 | 8.112 | 24.093 | −2.234 | 1.00 | 28.40 |
| ATOM | 1440 | CD1 | ILE | A | 482 | 9.070 | 23.005 | −1.769 | 1.00 | 24.86 |
| ATOM | 1441 | C | ILE | A | 482 | 8.447 | 27.681 | −3.639 | 1.00 | 35.37 |
| ATOM | 1442 | O | ILE | A | 482 | 8.152 | 28.579 | −2.864 | 1.00 | 36.18 |
| ATOM | 1443 | N | GLN | A | 483 | 9.256 | 27.862 | −4.668 | 1.00 | 38.47 |
| ATOM | 1444 | CA | GLN | A | 483 | 9.867 | 29.164 | −4.892 | 1.00 | 39.76 |
| ATOM | 1445 | CB | GLN | A | 483 | 10.932 | 29.071 | −5.950 | 1.00 | 40.59 |
| ATOM | 1446 | CG | GLN | A | 483 | 11.712 | 30.342 | −6.053 | 1.00 | 49.20 |
| ATOM | 1447 | CD | GLN | A | 483 | 12.711 | 30.296 | −7.192 | 1.00 | 58.50 |
| ATOM | 1448 | OE1 | GLN | A | 483 | 12.820 | 29.303 | −7.861 | 1.00 | 65.68 |
| ATOM | 1449 | NE2 | GLN | A | 483 | 13.440 | 31.368 | −7.409 | 1.00 | 65.23 |
| ATOM | 1450 | C | GLN | A | 483 | 8.843 | 30.239 | −5.284 | 1.00 | 40.84 |
| ATOM | 1451 | O | GLN | A | 483 | 8.969 | 31.422 | −4.923 | 1.00 | 38.66 |
| ATOM | 1452 | N | LEU | A | 484 | 7.802 | 29.820 | −6.002 | 1.00 | 39.88 |
| ATOM | 1453 | CA | LEU | A | 484 | 6.754 | 30.730 | −6.439 | 1.00 | 42.14 |
| ATOM | 1454 | CB | LEU | A | 484 | 5.812 | 30.033 | −7.416 | 1.00 | 45.74 |
| ATOM | 1455 | CG | LEU | A | 484 | 6.403 | 29.624 | −8.765 | 1.00 | 48.71 |
| ATOM | 1456 | CD1 | LEU | A | 484 | 5.337 | 28.943 | −9.615 | 1.00 | 48.20 |
| ATOM | 1457 | CD2 | LEU | A | 484 | 6.966 | 30.856 | −9.472 | 1.00 | 50.22 |
| ATOM | 1458 | C | LEU | A | 484 | 5.957 | 31.241 | −5.252 | 1.00 | 42.55 |
| ATOM | 1459 | O | LEU | A | 484 | 5.622 | 32.424 | −5.181 | 1.00 | 42.64 |
| ATOM | 1460 | N | ALA | A | 485 | 5.626 | 30.329 | −4.338 | 1.00 | 43.56 |
| ATOM | 1461 | CA | ALA | A | 485 | 4.872 | 30.672 | −3.128 | 1.00 | 40.84 |
| ATOM | 1462 | CB | ALA | A | 485 | 4.350 | 29.403 | −2.449 | 1.00 | 41.40 |
| ATOM | 1463 | C | ALA | A | 485 | 5.729 | 31.497 | −2.162 | 1.00 | 37.33 |
| ATOM | 1464 | O | ALA | A | 485 | 5.204 | 32.298 | −1.388 | 1.00 | 37.52 |
| ATOM | 1465 | N | LEU | A | 486 | 7.047 | 31.293 | −2.215 | 1.00 | 35.33 |
| ATOM | 1466 | CA | LEU | A | 486 | 7.979 | 32.036 | −1.380 | 1.00 | 38.02 |
| ATOM | 1467 | CB | LEU | A | 486 | 9.371 | 31.401 | −1.395 | 1.00 | 32.53 |
| ATOM | 1468 | CG | LEU | A | 486 | 10.451 | 32.287 | −0.758 | 1.00 | 33.21 |
| ATOM | 1469 | CD1 | LEU | A | 486 | 10.176 | 32.478 | 0.723 | 1.00 | 32.27 |
| ATOM | 1470 | CD2 | LEU | A | 486 | 11.818 | 31.693 | −0.965 | 1.00 | 31.01 |
| ATOM | 1471 | C | LEU | A | 486 | 8.049 | 33.457 | −1.942 | 1.00 | 42.86 |
| ATOM | 1472 | O | LEU | A | 486 | 8.077 | 34.446 | −1.190 | 1.00 | 44.35 |
| ATOM | 1473 | N | GLN | A | 487 | 8.070 | 33.545 | −3.271 | 1.00 | 44.64 |
| ATOM | 1474 | CA | GLN | A | 487 | 8.112 | 34.813 | −3.998 | 1.00 | 47.16 |
| ATOM | 1475 | CB | GLN | A | 487 | 8.145 | 34.502 | −5.486 | 1.00 | 51.86 |
| ATOM | 1476 | CG | GLN | A | 487 | 9.000 | 35.408 | −6.310 | 1.00 | 61.53 |
| ATOM | 1477 | CD | GLN | A | 487 | 9.486 | 34.714 | −7.571 | 1.00 | 65.93 |
| ATOM | 1478 | OE1 | GLN | A | 487 | 9.083 | 33.587 | −7.865 | 1.00 | 66.76 |
| ATOM | 1479 | NE2 | GLN | A | 487 | 10.369 | 35.373 | −8.311 | 1.00 | 70.60 |
| ATOM | 1480 | C | GLN | A | 487 | 6.823 | 35.579 | −3.655 | 1.00 | 47.05 |
| ATOM | 1481 | O | GLN | A | 487 | 6.844 | 36.754 | −3.307 | 1.00 | 45.17 |
| ATOM | 1482 | N | HIS | A | 488 | 5.702 | 34.874 | −3.741 | 1.00 | 47.07 |
| ATOM | 1483 | CA | HIS | A | 488 | 4.387 | 35.405 | −3.423 | 1.00 | 48.67 |
| ATOM | 1484 | CB | HIS | A | 488 | 3.374 | 34.280 | −3.608 | 1.00 | 51.60 |
| ATOM | 1485 | CG | HIS | A | 488 | 2.095 | 34.484 | −2.868 | 1.00 | 59.15 |
| ATOM | 1486 | CD2 | HIS | A | 488 | 1.638 | 33.939 | −1.715 | 1.00 | 62.57 |
| ATOM | 1487 | ND1 | HIS | A | 488 | 1.097 | 35.317 | −3.322 | 1.00 | 63.45 |
| ATOM | 1488 | CE1 | HIS | A | 488 | 0.074 | 35.272 | −2.485 | 1.00 | 64.94 |
| ATOM | 1489 | NE2 | HIS | A | 488 | 0.378 | 34.444 | −1.500 | 1.00 | 65.61 |
| ATOM | 1490 | C | HIS | A | 488 | 4.334 | 35.962 | −1.986 | 1.00 | 49.05 |
| ATOM | 1491 | O | HIS | A | 488 | 3.925 | 37.097 | −1.757 | 1.00 | 48.47 |
| ATOM | 1492 | N | VAL | A | 489 | 4.755 | 35.154 | −1.021 | 1.00 | 48.07 |
| ATOM | 1493 | CA | VAL | A | 489 | 4.763 | 35.571 | 0.381 | 1.00 | 45.44 |
| ATOM | 1494 | CB | VAL | A | 489 | 5.220 | 34.401 | 1.302 | 1.00 | 46.07 |
| ATOM | 1495 | CG1 | VAL | A | 489 | 5.592 | 34.911 | 2.692 | 1.00 | 44.85 |
| ATOM | 1496 | CG2 | VAL | A | 489 | 4.115 | 33.372 | 1.409 | 1.00 | 41.76 |
| ATOM | 1497 | C | VAL | A | 489 | 5.682 | 36.773 | 0.580 | 1.00 | 43.28 |
| ATOM | 1498 | O | VAL | A | 489 | 5.319 | 37.741 | 1.229 | 1.00 | 41.87 |
| ATOM | 1499 | N | LEU | A | 490 | 6.866 | 36.706 | −0.006 | 1.00 | 44.44 |
| ATOM | 1500 | CA | LEU | A | 490 | 7.865 | 37.763 | 0.103 | 1.00 | 49.79 |
| ATOM | 1501 | CB | LEU | A | 490 | 9.075 | 37.410 | −0.766 | 1.00 | 50.68 |
| ATOM | 1502 | CG | LEU | A | 490 | 10.389 | 36.884 | −0.181 | 1.00 | 54.00 |
| ATOM | 1503 | CD1 | LEU | A | 490 | 10.223 | 36.100 | 1.115 | 1.00 | 54.10 |
| ATOM | 1504 | CD2 | LEU | A | 490 | 11.034 | 36.035 | −1.241 | 1.00 | 54.17 |
| ATOM | 1505 | C | LEU | A | 490 | 7.338 | 39.122 | −0.310 | 1.00 | 52.92 |
| ATOM | 1506 | O | LEU | A | 490 | 7.224 | 40.037 | 0.498 | 1.00 | 50.59 |
| ATOM | 1507 | N | GLN | A | 491 | 7.014 | 39.243 | −1.587 | 1.00 | 60.06 |
| ATOM | 1508 | CA | GLN | A | 491 | 6.513 | 40.494 | −2.107 | 1.00 | 66.69 |
| ATOM | 1509 | CB | GLN | A | 491 | 6.560 | 40.523 | −3.638 | 1.00 | 72.00 |
| ATOM | 1510 | CG | GLN | A | 491 | 6.078 | 39.292 | −4.373 | 1.00 | 75.89 |
| ATOM | 1511 | CD | GLN | A | 491 | 6.340 | 39.394 | −5.862 | 1.00 | 79.38 |
| ATOM | 1512 | OE1 | GLN | A | 491 | 7.133 | 40.231 | −6.302 | 1.00 | 82.32 |
| ATOM | 1513 | NE2 | GLN | A | 491 | 5.672 | 38.554 | −6.647 | 1.00 | 81.82 |
| ATOM | 1514 | C | GLN | A | 491 | 5.144 | 40.863 | −1.555 | 1.00 | 68.27 |
| ATOM | 1515 | O | GLN | A | 491 | 4.820 | 42.045 | −1.433 | 1.00 | 69.36 |
| ATOM | 1516 | N | LYS | A | 492 | 4.360 | 39.863 | −1.168 | 1.00 | 69.33 |
| ATOM | 1517 | CA | LYS | A | 492 | 3.055 | 40.123 | −0.581 | 1.00 | 70.57 |
| ATOM | 1518 | CB | LYS | A | 492 | 2.464 | 38.825 | −0.036 | 1.00 | 63.87 |
| ATOM | 1519 | CG | LYS | A | 492 | 1.419 | 39.011 | 1.021 | 1.00 | 55.18 |
| ATOM | 1520 | CD | LYS | A | 492 | 0.936 | 37.684 | 1.518 | 1.00 | 50.15 |
| ATOM | 1521 | CE | LYS | A | 492 | 0.170 | 36.960 | 0.445 | 1.00 | 47.92 |
| ATOM | 1522 | NZ | LYS | A | 492 | −0.429 | 35.700 | 0.955 | 1.00 | 49.79 |
| ATOM | 1523 | C | LYS | A | 492 | 3.258 | 41.101 | 0.561 | 1.00 | 76.10 |
| ATOM | 1524 | O | LYS | A | 492 | 2.447 | 41.991 | 0.778 | 1.00 | 78.57 |
| ATOM | 1525 | N | ASN | A | 493 | 4.387 | 40.955 | 1.245 | 1.00 | 83.09 |
| ATOM | 1526 | CA | ASN | A | 493 | 4.724 | 41.795 | 2.380 | 1.00 | 90.65 |
| ATOM | 1527 | CB | ASN | A | 493 | 5.326 | 40.936 | 3.510 | 1.00 | 91.76 |
| ATOM | 1528 | CG | ASN | A | 493 | 4.413 | 39.791 | 3.960 | 1.00 | 91.33 |
| ATOM | 1529 | OD1 | ASN | A | 493 | 3.483 | 39.989 | 4.743 | 1.00 | 90.45 |
| ATOM | 1530 | ND2 | ASN | A | 493 | 4.727 | 38.576 | 3.525 | 1.00 | 90.60 |
| ATOM | 1531 | C | ASN | A | 493 | 5.703 | 42.929 | 2.053 | 1.00 | 96.54 |
| ATOM | 1532 | O | ASN | A | 493 | 5.445 | 44.097 | 2.361 | 1.00 | 97.40 |
| ATOM | 1533 | N | HIS | A | 494 | 6.815 | 42.593 | 1.401 | 1.00 | 104.63 |
| ATOM | 1534 | CA | HIS | A | 494 | 7.847 | 43.592 | 1.111 | 1.00 | 112.99 |
| ATOM | 1535 | CB | HIS | A | 494 | 9.092 | 43.302 | 1.972 | 1.00 | 118.20 |
| ATOM | 1536 | CG | HIS | A | 494 | 8.776 | 42.865 | 3.375 | 1.00 | 123.22 |
| ATOM | 1537 | CD2 | HIS | A | 494 | 8.606 | 41.630 | 3.906 | 1.00 | 124.65 |
| ATOM | 1538 | ND1 | HIS | A | 494 | 8.580 | 43.754 | 4.409 | 1.00 | 125.31 |
| ATOM | 1539 | CE1 | HIS | A | 494 | 8.306 | 43.087 | 5.517 | 1.00 | 125.71 |
| ATOM | 1540 | NE2 | HIS | A | 494 | 8.314 | 41.796 | 5.238 | 1.00 | 124.45 |
| ATOM | 1541 | C | HIS | A | 494 | 8.283 | 43.810 | −0.343 | 1.00 | 115.21 |
| ATOM | 1542 | O | HIS | A | 494 | 9.414 | 43.476 | −0.719 | 1.00 | 113.88 |
| ATOM | 1543 | N | ARG | A | 495 | 7.411 | 44.411 | −1.150 | 1.00 | 118.74 |
| ATOM | 1544 | CA | ARG | A | 495 | 7.771 | 44.693 | −2.539 | 1.00 | 123.08 |
| ATOM | 1545 | CB | ARG | A | 495 | 6.532 | 44.895 | −3.411 | 1.00 | 124.19 |
| ATOM | 1546 | CG | ARG | A | 495 | 5.922 | 43.611 | −3.923 | 1.00 | 125.95 |
| ATOM | 1547 | CD | ARG | A | 495 | 4.905 | 43.869 | −5.022 | 1.00 | 128.86 |
| ATOM | 1548 | NE | ARG | A | 495 | 4.097 | 42.688 | −5.336 | 1.00 | 130.65 |
| ATOM | 1549 | CZ | ARG | A | 495 | 2.771 | 42.700 | −5.469 | 1.00 | 131.44 |
| ATOM | 1550 | NH1 | ARG | A | 495 | 2.089 | 43.829 | −5.316 | 1.00 | 132.26 |
| ATOM | 1551 | NH2 | ARG | A | 495 | 2.122 | 41.580 | −5.755 | 1.00 | 132.05 |
| ATOM | 1552 | C | ARG | A | 495 | 8.677 | 45.927 | −2.632 | 1.00 | 124.82 |
| ATOM | 1553 | O | ARG | A | 495 | 9.086 | 46.332 | −3.723 | 1.00 | 125.80 |
| ATOM | 1554 | N | GLU | A | 496 | 8.979 | 46.520 | −1.479 | 1.00 | 125.27 |
| ATOM | 1555 | CA | GLU | A | 496 | 9.840 | 47.694 | −1.403 | 1.00 | 125.62 |
| ATOM | 1556 | CB | GLU | A | 496 | 10.086 | 48.053 | 0.060 | 1.00 | 125.24 |
| ATOM | 1557 | CG | GLU | A | 496 | 10.364 | 46.856 | 0.953 | 1.00 | 125.43 |
| ATOM | 1558 | CD | GLU | A | 496 | 10.161 | 47.175 | 2.414 | 1.00 | 127.70 |
| ATOM | 1559 | OE1 | GLU | A | 496 | 11.014 | 47.880 | 2.992 | 1.00 | 129.15 |
| ATOM | 1560 | OE2 | GLU | A | 496 | 9.137 | 46.733 | 2.981 | 1.00 | 127.94 |
| ATOM | 1561 | C | GLU | A | 496 | 11.162 | 47.439 | −2.117 | 1.00 | 126.43 |
| ATOM | 1562 | O | GLU | A | 496 | 11.731 | 48.346 | −2.723 | 1.00 | 127.96 |
| ATOM | 1563 | N | ASP | A | 497 | 11.635 | 46.195 | −2.047 | 1.00 | 126.16 |
| ATOM | 1564 | CA | ASP | A | 497 | 12.886 | 45.790 | −2.693 | 1.00 | 124.22 |
| ATOM | 1565 | CB | ASP | A | 497 | 14.036 | 45.643 | −1.669 | 1.00 | 124.95 |
| ATOM | 1566 | CG | ASP | A | 497 | 13.719 | 46.260 | −0.317 | 1.00 | 125.55 |
| ATOM | 1567 | OD1 | ASP | A | 497 | 13.970 | 47.468 | −0.132 | 1.00 | 125.55 |
| ATOM | 1568 | OD2 | ASP | A | 497 | 13.221 | 45.530 | 0.565 | 1.00 | 126.80 |
| ATOM | 1569 | C | ASP | A | 497 | 12.646 | 44.446 | −3.392 | 1.00 | 121.48 |
| ATOM | 1570 | O | ASP | A | 497 | 11.524 | 44.142 | −3.807 | 1.00 | 121.28 |
| ATOM | 1571 | N | GLY | A | 498 | 13.711 | 43.655 | −3.527 | 1.00 | 118.36 |
| ATOM | 1572 | CA | GLY | A | 498 | 13.637 | 42.337 | −4.142 | 1.00 | 112.17 |
| ATOM | 1573 | C | GLY | A | 498 | 14.617 | 41.465 | −3.374 | 1.00 | 106.96 |
| ATOM | 1574 | O | GLY | A | 498 | 15.677 | 41.104 | −3.889 | 1.00 | 107.39 |
| ATOM | 1575 | N | ILE | A | 499 | 14.253 | 41.158 | −2.128 | 1.00 | 100.38 |
| ATOM | 1576 | CA | ILE | A | 499 | 15.058 | 40.378 | −1.187 | 1.00 | 90.88 |
| ATOM | 1577 | CB | ILE | A | 499 | 14.545 | 40.644 | 0.259 | 1.00 | 89.37 |
| ATOM | 1578 | CG2 | ILE | A | 499 | 14.172 | 39.362 | 0.988 | 1.00 | 88.94 |
| ATOM | 1579 | CG1 | ILE | A | 499 | 15.573 | 41.483 | 1.013 | 1.00 | 88.59 |
| ATOM | 1580 | CD1 | ILE | A | 499 | 16.015 | 42.736 | 0.254 | 1.00 | 88.83 |
| ATOM | 1581 | C | ILE | A | 499 | 15.254 | 38.885 | −1.469 | 1.00 | 85.97 |
| ATOM | 1582 | O | ILE | A | 499 | 16.120 | 38.243 | −0.872 | 1.00 | 81.39 |
| ATOM | 1583 | N | LEU | A | 500 | 14.482 | 38.353 | −2.412 | 1.00 | 82.32 |
| ATOM | 1584 | CA | LEU | A | 500 | 14.572 | 36.945 | −2.784 | 1.00 | 79.33 |
| ATOM | 1585 | CB | LEU | A | 500 | 13.593 | 36.610 | −3.926 | 1.00 | 75.45 |
| ATOM | 1586 | CG | LEU | A | 500 | 13.480 | 35.160 | −4.427 | 1.00 | 72.35 |
| ATOM | 1587 | CD1 | LEU | A | 500 | 13.020 | 34.209 | −3.345 | 1.00 | 70.46 |
| ATOM | 1588 | CD2 | LEU | A | 500 | 12.509 | 35.104 | −5.569 | 1.00 | 74.01 |
| ATOM | 1589 | C | LEU | A | 500 | 15.994 | 36.594 | −3.191 | 1.00 | 78.86 |
| ATOM | 1590 | O | LEU | A | 500 | 16.443 | 35.483 | −2.943 | 1.00 | 80.08 |
| ATOM | 1591 | N | THR | A | 501 | 16.709 | 37.548 | −3.786 | 1.00 | 78.15 |
| ATOM | 1592 | CA | THR | A | 501 | 18.085 | 37.316 | −4.224 | 1.00 | 79.02 |
| ATOM | 1593 | CB | THR | A | 501 | 18.654 | 38.487 | −5.065 | 1.00 | 82.06 |
| ATOM | 1594 | OG1 | THR | A | 501 | 18.870 | 39.635 | −4.225 | 1.00 | 84.30 |
| ATOM | 1595 | CG2 | THR | A | 501 | 17.708 | 38.834 | −6.214 | 1.00 | 83.64 |
| ATOM | 1596 | C | THR | A | 501 | 18.935 | 37.193 | −2.988 | 1.00 | 77.23 |
| ATOM | 1597 | O | THR | A | 501 | 19.694 | 36.236 | −2.831 | 1.00 | 77.82 |
| ATOM | 1598 | N | LYS | A | 502 | 18.776 | 38.185 | −2.117 | 1.00 | 74.83 |
| ATOM | 1599 | CA | LYS | A | 502 | 19.478 | 38.276 | −0.847 | 1.00 | 72.92 |
| ATOM | 1600 | CB | LYS | A | 502 | 18.834 | 39.402 | −0.025 | 1.00 | 79.85 |
| ATOM | 1601 | CG | LYS | A | 502 | 19.726 | 40.108 | 0.995 | 1.00 | 85.84 |
| ATOM | 1602 | CD | LYS | A | 502 | 18.937 | 41.231 | 1.690 | 1.00 | 89.85 |
| ATOM | 1603 | CE | LYS | A | 502 | 19.744 | 41.963 | 2.764 | 1.00 | 94.60 |
| ATOM | 1604 | NZ | LYS | A | 502 | 18.918 | 42.984 | 3.487 | 1.00 | 95.90 |
| ATOM | 1605 | C | LYS | A | 502 | 19.282 | 36.922 | −0.158 | 1.00 | 66.91 |
| ATOM | 1606 | O | LYS | A | 502 | 20.227 | 36.332 | 0.360 | 1.00 | 65.72 |
| ATOM | 1607 | N | LEU | A | 503 | 18.060 | 36.409 | −0.267 | 1.00 | 59.20 |
| ATOM | 1608 | CA | LEU | A | 503 | 17.662 | 35.134 | 0.306 | 1.00 | 52.88 |
| ATOM | 1609 | CB | LEU | A | 503 | 16.158 | 34.971 | 0.156 | 1.00 | 47.50 |
| ATOM | 1610 | CG | LEU | A | 503 | 15.466 | 34.377 | 1.365 | 1.00 | 49.58 |
| ATOM | 1611 | CD1 | LEU | A | 503 | 16.066 | 34.928 | 2.664 | 1.00 | 46.78 |
| ATOM | 1612 | CD2 | LEU | A | 503 | 13.990 | 34.688 | 1.270 | 1.00 | 51.22 |
| ATOM | 1613 | C | LEU | A | 503 | 18.374 | 33.938 | −0.314 | 1.00 | 51.55 |
| ATOM | 1614 | O | LEU | A | 503 | 19.200 | 33.301 | 0.334 | 1.00 | 49.38 |
| ATOM | 1615 | N | ILE | A | 504 | 18.087 | 33.645 | −1.579 | 1.00 | 50.92 |
| ATOM | 1616 | CA | ILE | A | 504 | 18.715 | 32.499 | −2.231 | 1.00 | 51.60 |
| ATOM | 1617 | CB | ILE | A | 504 | 18.054 | 32.153 | −3.588 | 1.00 | 55.42 |
| ATOM | 1618 | CG2 | ILE | A | 504 | 16.718 | 31.464 | −3.360 | 1.00 | 52.28 |
| ATOM | 1619 | CG1 | ILE | A | 504 | 17.860 | 33.415 | −4.418 | 1.00 | 59.46 |
| ATOM | 1620 | CD1 | ILE | A | 504 | 16.623 | 33.368 | −5.320 | 1.00 | 67.68 |
| ATOM | 1621 | C | ILE | A | 504 | 20.233 | 32.598 | −2.336 | 1.00 | 49.75 |
| ATOM | 1622 | O | ILE | A | 504 | 20.917 | 31.593 | −2.485 | 1.00 | 50.38 |
| ATOM | 1623 | N | CYS | A | 505 | 20.773 | 33.798 | −2.192 | 1.00 | 48.02 |
| ATOM | 1624 | CA | CYS | A | 505 | 22.221 | 33.974 | −2.220 | 1.00 | 49.31 |
| ATOM | 1625 | CB | CYS | A | 505 | 22.557 | 35.465 | −2.457 | 1.00 | 58.96 |
| ATOM | 1626 | SG | CYS | A | 505 | 23.762 | 36.304 | −1.354 | 1.00 | 77.11 |
| ATOM | 1627 | C | CYS | A | 505 | 22.791 | 33.433 | −0.891 | 1.00 | 46.24 |
| ATOM | 1628 | O | CYS | A | 505 | 23.956 | 33.014 | −0.803 | 1.00 | 41.30 |
| ATOM | 1629 | N | LYS | A | 506 | 21.939 | 33.398 | 0.131 | 1.00 | 40.06 |
| ATOM | 1630 | CA | LYS | A | 506 | 22.336 | 32.890 | 1.425 | 1.00 | 37.35 |
| ATOM | 1631 | CB | LYS | A | 506 | 21.286 | 33.221 | 2.481 | 1.00 | 40.11 |
| ATOM | 1632 | CG | LYS | A | 506 | 20.957 | 34.686 | 2.596 | 1.00 | 43.49 |
| ATOM | 1633 | CD | LYS | A | 506 | 22.217 | 35.514 | 2.611 | 1.00 | 51.34 |
| ATOM | 1634 | CE | LYS | A | 506 | 22.343 | 36.269 | 3.891 | 1.00 | 55.65 |
| ATOM | 1635 | NZ | LYS | A | 506 | 22.139 | 35.376 | 5.071 | 1.00 | 63.77 |
| ATOM | 1636 | C | LYS | A | 506 | 22.466 | 31.389 | 1.309 | 1.00 | 36.27 |
| ATOM | 1637 | O | LYS | A | 506 | 23.267 | 30.786 | 2.005 | 1.00 | 40.04 |
| ATOM | 1638 | N | VAL | A | 507 | 21.692 | 30.784 | 0.417 | 1.00 | 32.90 |
| ATOM | 1639 | CA | VAL | A | 507 | 21.735 | 29.341 | 0.227 | 1.00 | 35.32 |
| ATOM | 1640 | CB | VAL | A | 507 | 20.809 | 28.932 | −0.926 | 1.00 | 39.14 |
| ATOM | 1641 | CG1 | VAL | A | 507 | 20.872 | 27.438 | −1.155 | 1.00 | 41.89 |
| ATOM | 1642 | CG2 | VAL | A | 507 | 19.382 | 29.364 | −0.630 | 1.00 | 35.73 |
| ATOM | 1643 | C | VAL | A | 507 | 23.146 | 28.782 | −0.016 | 1.00 | 35.01 |
| ATOM | 1644 | O | VAL | A | 507 | 23.447 | 27.642 | 0.318 | 1.00 | 36.47 |
| ATOM | 1645 | N | SER | A | 508 | 24.009 | 29.584 | −0.616 | 1.00 | 36.29 |
| ATOM | 1646 | CA | SER | A | 508 | 25.372 | 29.159 | −0.886 | 1.00 | 37.66 |
| ATOM | 1647 | CB | SER | A | 508 | 25.996 | 30.084 | −1.941 | 1.00 | 38.93 |
| ATOM | 1648 | OG | SER | A | 508 | 27.309 | 29.693 | −2.294 | 1.00 | 45.66 |
| ATOM | 1649 | C | SER | A | 508 | 26.192 | 29.172 | 0.420 | 1.00 | 37.79 |
| ATOM | 1650 | O | SER | A | 508 | 27.026 | 28.280 | 0.647 | 1.00 | 37.44 |
| ATOM | 1651 | N | THR | A | 509 | 25.949 | 30.178 | 1.267 | 1.00 | 35.49 |
| ATOM | 1652 | CA | THR | A | 509 | 26.627 | 30.326 | 2.553 | 1.00 | 32.31 |
| ATOM | 1653 | CB | THR | A | 509 | 26.250 | 31.644 | 3.236 | 1.00 | 35.78 |
| ATOM | 1654 | OG1 | THR | A | 509 | 26.405 | 32.720 | 2.305 | 1.00 | 42.43 |
| ATOM | 1655 | CG2 | THR | A | 509 | 27.157 | 31.895 | 4.437 | 1.00 | 35.82 |
| ATOM | 1656 | C | THR | A | 509 | 26.231 | 29.187 | 3.469 | 1.00 | 28.28 |
| ATOM | 1657 | O | THR | A | 509 | 27.065 | 28.665 | 4.178 | 1.00 | 32.41 |
| ATOM | 1658 | N | LEU | A | 510 | 24.958 | 28.812 | 3.452 | 1.00 | 28.16 |
| ATOM | 1659 | CA | LEU | A | 510 | 24.452 | 27.696 | 4.249 | 1.00 | 29.00 |
| ATOM | 1660 | CB | LEU | A | 510 | 22.994 | 27.394 | 3.885 | 1.00 | 26.48 |
| ATOM | 1661 | CG | LEU | A | 510 | 21.819 | 28.079 | 4.571 | 1.00 | 29.80 |
| ATOM | 1662 | CD1 | LEU | A | 510 | 20.544 | 27.623 | 3.907 | 1.00 | 28.25 |
| ATOM | 1663 | CD2 | LEU | A | 510 | 21.793 | 27.717 | 6.063 | 1.00 | 30.36 |
| ATOM | 1664 | C | LEU | A | 510 | 25.267 | 26.437 | 3.969 | 1.00 | 32.43 |
| ATOM | 1665 | O | LEU | A | 510 | 25.639 | 25.698 | 4.885 | 1.00 | 32.33 |
| ATOM | 1666 | N | ARG | A | 511 | 25.524 | 26.194 | 2.685 | 1.00 | 31.40 |
| ATOM | 1667 | CA | ARG | A | 511 | 26.272 | 25.027 | 2.233 | 1.00 | 28.94 |
| ATOM | 1668 | CB | ARG | A | 511 | 26.170 | 24.909 | 0.706 | 1.00 | 34.59 |
| ATOM | 1669 | CG | ARG | A | 511 | 24.716 | 24.741 | 0.261 | 1.00 | 34.26 |
| ATOM | 1670 | CD | ARG | A | 511 | 24.547 | 24.611 | −1.225 | 1.00 | 36.35 |
| ATOM | 1671 | NE | ARG | A | 511 | 23.192 | 24.185 | −1.556 | 1.00 | 31.13 |
| ATOM | 1672 | CZ | ARG | A | 511 | 22.887 | 23.356 | −2.545 | 1.00 | 31.68 |
| ATOM | 1673 | NH1 | ARG | A | 511 | 23.839 | 22.856 | −3.324 | 1.00 | 32.54 |
| ATOM | 1674 | NH2 | ARG | A | 511 | 21.628 | 23.004 | −2.730 | 1.00 | 27.68 |
| ATOM | 1675 | C | ARG | A | 511 | 27.710 | 25.049 | 2.695 | 1.00 | 26.34 |
| ATOM | 1676 | O | ARG | A | 511 | 28.270 | 24.011 | 3.015 | 1.00 | 28.74 |
| ATOM | 1677 | N | ALA | A | 512 | 28.314 | 26.235 | 2.693 | 1.00 | 25.39 |
| ATOM | 1678 | CA | ALA | A | 512 | 29.687 | 26.409 | 3.161 | 1.00 | 24.68 |
| ATOM | 1679 | CB | ALA | A | 512 | 30.157 | 27.796 | 2.830 | 1.00 | 22.96 |
| ATOM | 1680 | C | ALA | A | 512 | 29.732 | 26.195 | 4.691 | 1.00 | 27.69 |
| ATOM | 1681 | O | ALA | A | 512 | 30.622 | 25.518 | 5.231 | 1.00 | 27.18 |
| ATOM | 1652 | N | LEU | A | 513 | 28.773 | 26.814 | 5.373 | 1 00 | 27 .15 |
| ATOM | 1683 | CA | LEU | A | 513 | 28.638 | 26.708 | 6.815 | 1.00 | 26.64 |
| ATOM | 1684 | CB | LEU | A | 513 | 27.427 | 27.528 | 7.256 | 1.00 | 24.45 |
| ATOM | 1685 | CG | LEU | A | 513 | 27.228 | 27.696 | 8.758 | 1.00 | 28.40 |
| ATOM | 1686 | CD1 | LEU | A | 513 | 28.492 | 28.230 | 9.403 | 1.00 | 25.85 |
| ATOM | 1687 | CD2 | LEU | A | 513 | 26.053 | 28.607 | 9.013 | 1.00 | 30.72 |
| ATOM | 1688 | C | LEU | A | 513 | 28.490 | 25.209 | 7.192 | 1.00 | 29.10 |
| ATOM | 1689 | O | LEU | A | 513 | 29.259 | 24.683 | 8.008 | 1.00 | 31.91 |
| ATOM | 1690 | N | CYS | A | 514 | 27.543 | 24.512 | 6.566 | 1.00 | 26.57 |
| ATOM | 1691 | CA | CYS | A | 514 | 27.351 | 23.104 | 6.851 | 1.00 | 26.98 |
| ATOM | 1692 | CB | CYS | A | 514 | 26.025 | 22.614 | 6.269 | 1.00 | 25.74 |
| ATOM | 1693 | SG | CYS | A | 514 | 24.579 | 23.438 | 7.009 | 1.00 | 30.96 |
| ATOM | 1694 | C | CYS | A | 514 | 28.538 | 22.250 | 6.404 | 1.00 | 28.07 |
| ATOM | 1695 | O | CYS | A | 514 | 28.764 | 21.161 | 6.931 | 1.00 | 27.30 |
| ATOM | 1696 | N | GLY | A | 515 | 29.298 | 22.741 | 5.431 | 1.00 | 29.78 |
| ATOM | 1697 | CA | GLY | A | 515 | 30.477 | 22.027 | 4.980 | 1.00 | 29.40 |
| ATOM | 1698 | C | GLY | A | 515 | 31.570 | 22.004 | 6.040 | 1.00 | 30.10 |
| ATOM | 1699 | O | GLY | A | 515 | 32.190 | 20.965 | 6.266 | 1.00 | 30.71 |
| ATOM | 1700 | N | ARG | A | 516 | 31.810 | 23.142 | 6.693 | 1.00 | 33.97 |
| ATOM | 1701 | CA | ARG | A | 516 | 32.811 | 23.217 | 7.748 | 1.00 | 34.03 |
| ATOM | 1702 | CB | ARG | A | 516 | 33.065 | 24.652 | 8.178 | 1.00 | 37.07 |
| ATOM | 1703 | CG | ARG | A | 516 | 33.921 | 25.429 | 7.198 | 1.00 | 52.01 |
| ATOM | 1704 | CD | ARG | A | 516 | 34.465 | 26.715 | 7.807 | 1.00 | 56.98 |
| ATOM | 1705 | NE | ARG | A | 516 | 35.467 | 26.438 | 8.836 | 1.00 | 61.64 |
| ATOM | 1706 | CZ | ARG | A | 516 | 35.927 | 27.342 | 9.699 | 1.00 | 62.65 |
| ATOM | 1707 | NH1 | ARG | A | 516 | 35.466 | 28.590 | 9.665 | 1.00 | 61.40 |
| ATOM | 1708 | NH2 | ARG | A | 516 | 36.879 | 27.005 | 10.566 | 1.00 | 60.38 |
| ATOM | 1709 | C | ARG | A | 516 | 32.380 | 22.395 | 8.947 | 1.00 | 33.89 |
| ATOM | 1710 | O | ARG | A | 516 | 33.227 | 21.847 | 9.634 | 1.00 | 33.31 |
| ATOM | 1711 | N | HIS | A | 517 | 31.075 | 22.322 | 9.215 | 1.00 | 33.19 |
| ATOM | 1712 | CA | HIS | A | 517 | 30.616 | 21.517 | 10.337 | 1.00 | 34.62 |
| ATOM | 1713 | CB | HIS | A | 517 | 29.085 | 21.466 | 10.440 | 1.00 | 34.29 |
| ATOM | 1714 | CG | HIS | A | 517 | 28.576 | 20.462 | 11.440 | 1.00 | 33.44 |
| ATOM | 1715 | CD2 | HIS | A | 517 | 28.646 | 20.433 | 12.793 | 1.00 | 31.33 |
| ATOM | 1716 | ND1 | HIS | A | 517 | 27.909 | 19.311 | 11.072 | 1.00 | 28.67 |
| ATOM | 1717 | CE1 | HIS | A | 517 | 27.589 | 18.619 | 12.151 | 1.00 | 27.08 |
| ATOM | 1718 | NE2 | HIS | A | 517 | 28.027 | 19.279 | 13.208 | 1.00 | 28.43 |
| ATOM | 1719 | C | HIS | A | 517 | 31.147 | 20.107 | 10.127 | 1.00 | 36.73 |
| ATOM | 1720 | O | HIS | A | 517 | 31.825 | 19.567 | 10.994 | 1.00 | 34.74 |
| ATOM | 1721 | N | THR | A | 518 | 30.884 | 19.541 | 8.950 | 1.00 | 36.75 |
| ATOM | 1722 | CA | THR | A | 518 | 31.343 | 18.192 | 8.662 | 1.00 | 36.81 |
| ATOM | 1723 | CB | THR | A | 518 | 30.692 | 17.620 | 7.406 | 1.00 | 35.05 |
| ATOM | 1724 | OG1 | THR | A | 518 | 29.327 | 17.303 | 7.688 | 1.00 | 37.15 |
| ATOM | 1725 | CG2 | THR | A | 518 | 31.382 | 16.346 | 6.980 | 1.00 | 38.73 |
| ATOM | 1726 | C | THR | A | 518 | 32.862 | 18.100 | 8.608 | 1.00 | 37.39 |
| ATOM | 1727 | O | THR | A | 518 | 33.430 | 17.077 | 8.962 | 1.00 | 42.45 |
| ATOM | 1728 | N | GLU | A | 519 | 33.531 | 19.175 | 8.220 | 1.00 | 38.67 |
| ATOM | 1729 | CA | GLU | A | 519 | 34.990 | 19.166 | 8.196 | 1.00 | 43.58 |
| ATOM | 1730 | CB | GLU | A | 519 | 35.516 | 20.438 | 7.500 | 1.00 | 54.27 |
| ATOM | 1731 | CG | GLU | A | 519 | 35.261 | 20.537 | 5.964 | 1.00 | 63.20 |
| ATOM | 1732 | CD | GLU | A | 519 | 35.380 | 21.975 | 5.406 | 1.00 | 65.12 |
| ATOM | 1733 | OE1 | GLU | A | 519 | 34.782 | 22.258 | 4.342 | 1.00 | 66.49 |
| ATOM | 1734 | OE2 | GLU | A | 519 | 36.053 | 22.826 | 6.035 | 1.00 | 67.73 |
| ATOM | 1735 | C | GLU | A | 519 | 35.516 | 19.099 | 9.649 | 1.00 | 42.46 |
| ATOM | 1736 | O | GLU | A | 519 | 36.470 | 18.382 | 9.959 | 1.00 | 39.74 |
| ATOM | 1737 | N | LYS | A | 520 | 34.857 | 19.843 | 10.535 | 1.00 | 41.75 |
| ATOM | 1738 | CA | LYS | A | 520 | 35.222 | 19.895 | 11.945 | 1.00 | 37.07 |
| ATOM | 1739 | CB | LYS | A | 520 | 34.481 | 21.049 | 12.651 | 1.00 | 41.10 |
| ATOM | 1740 | CG | LYS | A | 520 | 34.939 | 22.467 | 12.235 | 1.00 | 44.66 |
| ATOM | 1741 | CD | LYS | A | 520 | 36.383 | 22.748 | 12.670 | 1.00 | 53.92 |
| ATOM | 1742 | CE | LYS | A | 520 | 37.078 | 23.819 | 11.819 | 1.00 | 56.95 |
| ATOM | 1743 | NZ | LYS | A | 520 | 37.313 | 23.411 | 10.388 | 1.00 | 63.31 |
| ATOM | 1744 | C | LYS | A | 520 | 34.911 | 18.560 | 12.618 | 1.00 | 34.80 |
| ATOM | 1745 | O | LYS | A | 520 | 35.770 | 17.983 | 13.278 | 1.00 | 34.89 |
| ATOM | 1746 | N | LEU | A | 521 | 33.703 | 18.051 | 12.394 | 1.00 | 33.45 |
| ATOM | 1747 | CA | LEU | A | 521 | 33.270 | 16.782 | 12.956 | 1.00 | 33.31 |
| ATOM | 1748 | CB | LEU | A | 521 | 31.839 | 16.460 | 12.526 | 1.00 | 26.37 |
| ATOM | 1749 | CG | LEU | A | 521 | 31.268 | 15.095 | 12.905 | 1.00 | 24.78 |
| ATOM | 1750 | CD1 | LEU | A | 521 | 31.380 | 14.865 | 14.394 | 1.00 | 29.22 |
| ATOM | 1751 | CD2 | LEU | A | 521 | 29.832 | 15.021 | 12.520 | 1.00 | 24.85 |
| ATOM | 1752 | C | LEU | A | 521 | 34.214 | 15.633 | 12.601 | 1.00 | 38.43 |
| ATOM | 1753 | O | LEU | A | 521 | 34.564 | 14.843 | 13.477 | 1.00 | 38.10 |
| ATOM | 1754 | N | MET | A | 522 | 34.694 | 15.581 | 11.356 | 1.00 | 39.97 |
| ATOM | 1755 | CA | MET | A | 522 | 35.601 | 14.507 | 10.947 | 1.00 | 41.97 |
| ATOM | 1756 | CB | MET | A | 522 | 35.725 | 14.431 | 9.426 | 1.00 | 48.01 |
| ATOM | 1757 | CG | MET | A | 522 | 34.430 | 14.035 | 8.707 | 1.00 | 58.55 |
| ATOM | 1758 | SD | MET | A | 522 | 33.355 | 12.804 | 9.548 | 1.00 | 67.62 |
| ATOM | 1759 | CE | MET | A | 522 | 34.455 | 11.351 | 9.732 | 1.00 | 65.55 |
| ATOM | 1760 | C | MET | A | 522 | 36.982 | 14.574 | 11.596 | 1.00 | 40.03 |
| ATOM | 1761 | O | MET | A | 522 | 37.578 | 13.539 | 11.900 | 1.00 | 37.83 |
| ATOM | 1762 | N | ALA | A | 523 | 37.497 | 15.782 | 11.798 | 1.00 | 37.94 |
| ATOM | 1763 | CA | ALA | A | 523 | 38.790 | 15.936 | 12.452 | 1.00 | 39.72 |
| ATOM | 1764 | CB | ALA | A | 523 | 39.272 | 17.353 | 12.304 | 1.00 | 45.50 |
| ATOM | 1765 | C | ALA | A | 523 | 38.640 | 15.587 | 13.938 | 1.00 | 41.81 |
| ATOM | 1766 | O | ALA | A | 523 | 39.523 | 14.997 | 14.547 | 1.00 | 44.06 |
| ATOM | 1767 | N | PHE | A | 524 | 37.509 | 15.971 | 14.519 | 1.00 | 41.49 |
| ATOM | 1768 | CA | PHE | A | 524 | 37.238 | 15.674 | 15.915 | 1.00 | 39.19 |
| ATOM | 1769 | CB | PHE | A | 524 | 35.923 | 16.334 | 16.360 | 1.00 | 35.12 |
| ATOM | 1770 | CG | PHE | A | 524 | 35.511 | 15.998 | 17.781 | 1.00 | 30.17 |
| ATOM | 1771 | CD1 | PHE | A | 524 | 35.968 | 16.762 | 18.852 | 1.00 | 28.22 |
| ATOM | 1772 | CD2 | PHE | A | 524 | 34.644 | 14.924 | 18.040 | 1.00 | 26.69 |
| ATOM | 1773 | CE1 | PHE | A | 524 | 35.569 | 16.465 | 20.166 | 1.00 | 25.14 |
| ATOM | 1774 | CE2 | PHE | A | 524 | 34.240 | 14.620 | 19.341 | 1.00 | 27.45 |
| ATOM | 1775 | CZ | PHE | A | 524 | 34.709 | 15.398 | 20.408 | 1.00 | 25.33 |
| ATOM | 1776 | C | PHE | A | 524 | 37.151 | 14.157 | 16.093 | 1.00 | 38.05 |
| ATOM | 1777 | O | PHE | A | 524 | 37.788 | 13.602 | 16.989 | 1.00 | 39.38 |
| ATOM | 1778 | N | LYS | A | 525 | 36.370 | 13.494 | 15.240 | 1.00 | 33.74 |
| ATOM | 1779 | CA | LYS | A | 525 | 36.188 | 12.053 | 15.336 | 1.00 | 32.28 |
| ATOM | 1780 | CB | LYS | A | 525 | 35.150 | 11.566 | 14.341 | 1.00 | 32.43 |
| ATOM | 1781 | CG | LYS | A | 525 | 35.058 | 10.061 | 14.265 | 1.00 | 32.27 |
| ATOM | 1782 | CD | LYS | A | 525 | 34.049 | 9.588 | 13.234 | 1.00 | 39.70 |
| ATOM | 1783 | CE | LYS | A | 525 | 34.182 | 8.085 | 13.027 | 1.00 | 43.60 |
| ATOM | 1784 | NZ | LYS | A | 525 | 33.052 | 7.475 | 12.274 | 1.00 | 52.36 |
| ATOM | 1785 | C | LYS | A | 525 | 37.486 | 11.292 | 15.141 | 1.00 | 37.02 |
| ATOM | 1786 | O | LYS | A | 525 | 37.572 | 10.113 | 15.458 | 1.00 | 38.38 |
| ATOM | 1787 | N | ALA | A | 526 | 38.514 | 11.972 | 14.651 | 1.00 | 38.57 |
| ATOM | 1788 | CA | ALA | A | 526 | 39.796 | 11.312 | 14.442 | 1.00 | 42.31 |
| ATOM | 1789 | CB | ALA | A | 526 | 40.639 | 12.098 | 13.429 | 1.00 | 42.02 |
| ATOM | 1790 | C | ALA | A | 526 | 40.523 | 11.230 | 15.774 | 1.00 | 43.27 |
| ATOM | 1791 | O | ALA | A | 526 | 41.174 | 10.226 | 16.091 | 1.00 | 43.91 |
| ATOM | 1792 | N | ILE | A | 527 | 40.348 | 12.293 | 16.555 | 1.00 | 41.76 |
| ATOM | 1793 | CA | ILE | A | 527 | 40.961 | 12.455 | 17.866 | 1.00 | 40.21 |
| ATOM | 1794 | CB | ILE | A | 527 | 41.127 | 13.946 | 18.166 | 1.00 | 40.67 |
| ATOM | 1795 | CG2 | ILE | A | 527 | 41.824 | 14.145 | 19.503 | 1.00 | 42.82 |
| ATOM | 1796 | CG1 | ILE | A | 527 | 41.897 | 14.612 | 17.026 | 1.00 | 39.18 |
| ATOM | 1797 | CD1 | ILE | A | 527 | 41.864 | 16.128 | 17.066 | 1.00 | 37.42 |
| ATOM | 1798 | C | ILE | A | 527 | 40.195 | 11.786 | 19.015 | 1.00 | 38.56 |
| ATOM | 1799 | O | ILE | A | 527 | 40.800 | 11.309 | 19.974 | 1.00 | 38.98 |
| ATOM | 1800 | N | TYR | A | 528 | 38.871 | 11.727 | 18.900 | 1.00 | 34.57 |
| ATOM | 1801 | CA | TYR | A | 528 | 38.023 | 11.135 | 19.930 | 1.00 | 31.38 |
| ATOM | 1802 | CB | TYR | A | 528 | 37.209 | 12.229 | 20.645 | 1.00 | 31.31 |
| ATOM | 1803 | CG | TYR | A | 528 | 38.046 | 13.379 | 21.159 | 1.00 | 34.92 |
| ATOM | 1804 | CD1 | TYR | A | 528 | 38.337 | 14.471 | 20.345 | 1.00 | 34.80 |
| ATOM | 1805 | CE1 | TYR | A | 528 | 39.157 | 15.513 | 20.789 | 1.00 | 39.08 |
| ATOM | 1806 | CD2 | TYR | A | 528 | 38.592 | 13.355 | 22.442 | 1.00 | 34.45 |
| ATOM | 1807 | CE2 | TYR | A | 528 | 39.417 | 14.394 | 22.895 | 1.00 | 37.74 |
| ATOM | 1808 | CZ | TYR | A | 528 | 39.695 | 15.468 | 22.062 | 1.00 | 39.56 |
| ATOM | 1809 | OH | TYR | A | 528 | 40.520 | 16.489 | 22.489 | 1.00 | 45.36 |
| ATOM | 1810 | C | TYR | A | 528 | 37.066 | 10.113 | 19.333 | 1.00 | 32.21 |
| ATOM | 1811 | O | TYR | A | 528 | 35.843 | 10.289 | 19.388 | 1.00 | 35.37 |
| ATOM | 1812 | N | PRO | A | 529 | 37.601 | 9.010 | 18.777 | 1.00 | 32.25 |
| ATOM | 1813 | CD | PRO | A | 529 | 39.034 | 8.705 | 18.673 | 1.00 | 29.41 |
| ATOM | 1814 | CA | PRO | A | 529 | 36.809 | 7.939 | 18.160 | 1.00 | 30.81 |
| ATOM | 1815 | CB | PRO | A | 529 | 37.875 | 6.895 | 17.822 | 1.00 | 30.02 |
| ATOM | 1816 | CG | PRO | A | 529 | 39.061 | 7.703 | 17.564 | 1.00 | 29.92 |
| ATOM | 1817 | C | PRO | A | 529 | 35.706 | 7.331 | 19.040 | 1.00 | 33.28 |
| ATOM | 1818 | O | PRO | A | 529 | 34.553 | 7.201 | 18.606 | 1.00 | 32.51 |
| ATOM | 1819 | N | ASP | A | 530 | 36.066 | 6.953 | 20.272 | 1.00 | 35.37 |
| ATOM | 1820 | CA | ASP | A | 530 | 35.110 | 6.338 | 21.195 | 1.00 | 35.88 |
| ATOM | 1821 | CB | ASP | A | 530 | 35.831 | 5.563 | 22.293 | 1.00 | 46.50 |
| ATOM | 1822 | CG | ASP | A | 530 | 36.564 | 4.341 | 21.758 | 1.00 | 58.80 |
| ATOM | 1823 | OD1 | ASP | A | 530 | 36.010 | 3.219 | 21.852 | 1.00 | 64.43 |
| ATOM | 1824 | OD2 | ASP | A | 530 | 37.697 | 4.503 | 21.240 | 1.00 | 63.77 |
| ATOM | 1825 | C | ASP | A | 530 | 34.127 | 7.309 | 21.799 | 1.00 | 29.27 |
| ATOM | 1826 | O | ASP | A | 530 | 33.037 | 6.909 | 22.221 | 1.00 | 29.58 |
| ATOM | 1827 | N | ILE | A | 531 | 34.525 | 8.577 | 21.892 | 1.00 | 30.58 |
| ATOM | 1828 | CA | ILE | A | 531 | 33.629 | 9.593 | 22.426 | 1.00 | 29.77 |
| ATOM | 1829 | CB | ILE | A | 531 | 34.302 | 10.998 | 22.576 | 1.00 | 35.01 |
| ATOM | 1830 | CG2 | ILE | A | 531 | 33.230 | 12.090 | 22.817 | 1.00 | 33.87 |
| ATOM | 1831 | CG1 | ILE | A | 531 | 35.247 | 10.998 | 23.781 | 1.00 | 36.43 |
| ATOM | 1832 | CD1 | ILE | A | 531 | 34.549 | 10.604 | 25.107 | 1.00 | 36.43 |
| ATOM | 1833 | C | ILE | A | 531 | 32.500 | 9.673 | 21.440 | 1.00 | 29.02 |
| ATOM | 1834 | O | ILE | A | 531 | 31.344 | 9.560 | 21.819 | 1.00 | 33.33 |
| ATOM | 1835 | N | VAL | A | 532 | 32.841 | 9.787 | 20.158 | 1.00 | 29.79 |
| ATOM | 1836 | CA | VAL | A | 532 | 31.814 | 9.857 | 19.129 | 1.00 | 25.98 |
| ATOM | 1837 | CB | VAL | A | 532 | 32.446 | 10.141 | 17.746 | 1.00 | 27.17 |
| ATOM | 1838 | CG1 | VAL | A | 532 | 31.376 | 10.244 | 16.699 | 1.00 | 26.57 |
| ATOM | 1839 | CG2 | VAL | A | 532 | 33.246 | 11.437 | 17.788 | 1.00 | 23.54 |
| ATOM | 1840 | C | VAL | A | 532 | 30.984 | 8.567 | 19.111 | 1.00 | 22.88 |
| ATOM | 1841 | O | VAL | A | 532 | 29.765 | 8.596 | 19.258 | 1.00 | 25.44 |
| ATOM | 1842 | N | ARG | A | 533 | 31.662 | 7.429 | 19.048 | 1.00 | 24.52 |
| ATOM | 1843 | CA | ARG | A | 533 | 30.980 | 6.136 | 18.997 | 1.00 | 23.99 |
| ATOM | 1844 | CB | ARG | A | 533 | 32.028 | 5.030 | 18.941 | 1.00 | 28.41 |
| ATOM | 1845 | CG | ARG | A | 533 | 31.444 | 3.657 | 18.777 | 1.00 | 37.43 |
| ATOM | 1846 | CD | ARG | A | 533 | 32.551 | 2.627 | 18.668 | 1.00 | 45.29 |
| ATOM | 1847 | WE | ARG | A | 533 | 32.255 | 1.490 | 19.528 | 1.00 | 51.73 |
| ATOM | 1848 | CZ | ARG | A | 533 | 32.897 | 1.228 | 20.658 | 1.00 | 55.49 |
| ATOM | 1849 | NH1 | ARG | A | 533 | 33.899 | 2.010 | 21.052 | 1.00 | 51.92 |
| ATOM | 1850 | NH2 | ARG | A | 533 | 32.459 | 0.254 | 21.445 | 1.00 | 59.77 |
| ATOM | 1851 | C | ARG | A | 533 | 30.024 | 5.861 | 20.136 | 1.00 | 24.93 |
| ATOM | 1852 | O | ARG | A | 533 | 28.866 | 5.504 | 19.935 | 1.00 | 24.06 |
| ATOM | 1853 | N | LEU | A | 534 | 30.519 | 6.093 | 21.348 | 1.00 | 30.94 |
| ATOM | 1854 | CA | LEU | A | 534 | 29.778 | 5.846 | 22.582 | 1.00 | 26.49 |
| ATOM | 1855 | CB | LEU | A | 534 | 30.773 | 5.408 | 23.658 | 1.00 | 28.50 |
| ATOM | 1856 | CG | LEU | A | 534 | 31.461 | 4.072 | 23.339 | 1.00 | 28.71 |
| ATOM | 1857 | CD1 | LEU | A | 534 | 32.692 | 3.806 | 24.193 | 1.00 | 29.88 |
| ATOM | 1858 | CD2 | LEU | A | 534 | 30.436 | 2.985 | 23.497 | 1.00 | 29.34 |
| ATOM | 1859 | C | LEU | A | 534 | 28.877 | 6.956 | 23.118 | 1.00 | 24.89 |
| ATOM | 1860 | O | LEU | A | 534 | 27.803 | 6.669 | 23.649 | 1.00 | 24.01 |
| ATOM | 1861 | N | HIS | A | 535 | 29.234 | 8.214 | 22.875 | 1.00 | 25.63 |
| ATOM | 1862 | CA | HIS | A | 535 | 28.458 | 9.317 | 23.439 | 1.00 | 27.16 |
| ATOM | 1863 | CB | HIS | A | 535 | 29.358 | 10.074 | 24.423 | 1.00 | 26.58 |
| ATOM | 1864 | CG | HIS | A | 535 | 30.001 | 9.174 | 25.430 | 1.00 | 25.50 |
| ATOM | 1865 | CD2 | HIS | A | 535 | 31.245 | 8.641 | 25.487 | 1.00 | 25.11 |
| ATOM | 1866 | ND1 | HIS | A | 535 | 29.302 | 8.631 | 26.487 | 1.00 | 26.41 |
| ATOM | 1867 | CE1 | HIS | A | 535 | 30.086 | 7.802 | 27.151 | 1.00 | 24.19 |
| ATOM | 1868 | NE2 | HIS | A | 535 | 31.270 | 7.790 | 26.564 | 1.00 | 26.77 |
| ATOM | 1869 | C | HIS | A | 535 | 27.669 | 10.284 | 22.553 | 1.00 | 28.58 |
| ATOM | 1870 | O | HIS | A | 535 | 26.881 | 11.073 | 23.082 | 1.00 | 27.81 |
| ATOM | 1871 | N | PHE | A | 536 | 27.851 | 10.228 | 21.226 | 1.00 | 33.00 |
| ATOM | 1872 | CA | PHE | A | 536 | 27.092 | 11.108 | 20.313 | 1.00 | 31.11 |
| ATOM | 1873 | CB | PHE | A | 536 | 27.895 | 11.401 | 19.043 | 1.00 | 29.90 |
| ATOM | 1874 | CG | PHE | A | 536 | 28.915 | 12.512 | 19.192 | 1.00 | 29.99 |
| ATOM | 1875 | CD1 | PHE | A | 536 | 29.678 | 12.643 | 20.337 | 1.00 | 24.61 |
| ATOM | 1876 | CD2 | PHE | A | 536 | 29.132 | 13.406 | 18.153 | 1.00 | 25.96 |
| ATOM | 1877 | CE1 | PHE | A | 536 | 30.644 | 13.642 | 20.439 | 1.00 | 23.54 |
| ATOM | 1878 | CE2 | PHE | A | 536 | 30.095 | 14.406 | 18.256 | 1.00 | 24.50 |
| ATOM | 1879 | CZ | PHE | A | 536 | 30.849 | 14.523 | 19.394 | 1.00 | 24.21 |
| ATOM | 1880 | C | PHE | A | 536 | 25.713 | 10.487 | 19.970 | 1.00 | 31.08 |
| ATOM | 1881 | O | PHE | A | 536 | 25.581 | 9.259 | 19.956 | 1.00 | 34.36 |
| ATOM | 1882 | N | PRO | A | 537 | 24.664 | 11.321 | 19.756 | 1.00 | 27.00 |
| ATOM | 1883 | CD | PRO | A | 537 | 24.632 | 12.793 | 19.845 | 1.00 | 25.60 |
| ATOM | 1884 | CA | PRO | A | 537 | 23.335 | 10.795 | 19.432 | 1.00 | 25.30 |
| ATOM | 1885 | CB | PRO | A | 537 | 22.501 | 12.068 | 19.256 | 1.00 | 23.10 |
| ATOM | 1886 | CG | PRO | A | 537 | 23.189 | 13.062 | 20.157 | 1.00 | 22.43 |
| ATOM | 1887 | C | PRO | A | 537 | 23.392 | 9.975 | 18.128 | 1.00 | 30.48 |
| ATOM | 1888 | O | PRO | A | 537 | 24.122 | 10.336 | 17.185 | 1.00 | 28.64 |
| ATOM | 1889 | N | PRO | A | 538 | 22.690 | 8.823 | 18.091 | 1.00 | 31.20 |
| ATOM | 1890 | CD | PRO | A | 538 | 22.017 | 8.173 | 19.227 | 1.00 | 28.91 |
| ATOM | 1891 | CA | PRO | A | 538 | 22.654 | 7.946 | 16.908 | 1.00 | 32.16 |
| ATOM | 1892 | CB | PRO | A | 538 | 21.592 | 6.931 | 17.293 | 1.00 | 29.35 |
| ATOM | 1893 | CG | PRO | A | 538 | 21.911 | 6.731 | 18.759 | 1.00 | 28.32 |
| ATOM | 1894 | C | PRO | A | 538 | 22.311 | 8.708 | 15.623 | 1.00 | 32.64 |
| ATOM | 1895 | O | PRO | A | 538 | 23.086 | 8.680 | 14.669 | 1.00 | 30.42 |
| ATOM | 1896 | N | LEU | A | 539 | 21.207 | 9.460 | 15.652 | 1.00 | 34.18 |
| ATOM | 1897 | CA | LEU | A | 539 | 20.773 | 10.273 | 14.510 | 1.00 | 30.02 |
| ATOM | 1898 | CB | LEU | A | 539 | 19.512 | 11.069 | 14.895 | 1.00 | 29.47 |
| ATOM | 1899 | CG | LEU | A | 539 | 18.858 | 11.924 | 13.802 | 1.00 | 29.66 |
| ATOM | 1900 | CD1 | LEU | A | 539 | 18.592 | 11.029 | 12.608 | 1.00 | 31.73 |
| ATOM | 1901 | CD2 | LEU | A | 539 | 17.583 | 12.604 | 14.267 | 1.00 | 25.29 |
| ATOM | 1902 | C | LEU | A | 539 | 21.887 | 11.215 | 14.009 | 1.00 | 29.19 |
| ATOM | 1903 | O | LEU | A | 539 | 22.131 | 11.326 | 12.816 | 1.00 | 30.25 |
| ATOM | 1904 | N | TYR | A | 540 | 22.581 | 11.877 | 14.925 | 1.00 | 25.32 |
| ATOM | 1905 | CA | TYR | A | 540 | 23.643 | 12.778 | 14.546 | 1.00 | 22.42 |
| ATOM | 1906 | CB | TYR | A | 540 | 24.280 | 13.355 | 15.791 | 1.00 | 21.19 |
| ATOM | 1907 | CG | TYR | A | 540 | 25.343 | 14.387 | 15.535 | 1.00 | 17.26 |
| ATOM | 1908 | CD1 | TYR | A | 540 | 25.016 | 15.733 | 15.400 | 1.00 | 17.30 |
| ATOM | 1909 | CE1 | TYR | A | 540 | 25.982 | 16.676 | 15.216 | 1.00 | 12.79 |
| ATOM | 1910 | CD2 | TYR | A | 540 | 26.680 | 14.028 | 15.464 | 1.00 | 19.93 |
| ATOM | 1911 | CE2 | TYR | A | 540 | 27.657 | 14.976 | 15.268 | 1.00 | 20.13 |
| ATOM | 1912 | CZ | TYR | A | 540 | 27.294 | 16.299 | 15.150 | 1.00 | 13.88 |
| ATOM | 1913 | OH | TYR | A | 540 | 28.276 | 17.244 | 14.997 | 1.00 | 21.75 |
| ATOM | 1914 | C | TYR | A | 540 | 24.708 | 12.009 | 13.780 | 1.00 | 32.70 |
| ATOM | 1915 | O | TYR | A | 540 | 25.296 | 12.508 | 12.811 | 1.00 | 34.57 |
| ATOM | 1916 | N | LYS | A | 541 | 25.008 | 10.802 | 14.244 | 1.00 | 35.66 |
| ATOM | 1917 | CA | LYS | A | 541 | 26.029 | 9.999 | 13.574 | 1.00 | 36.65 |
| ATOM | 1918 | CB | LYS | A | 541 | 26.482 | 8.874 | 14.497 | 1.00 | 32.71 |
| ATOM | 1919 | CG | LYS | A | 541 | 27.219 | 9.383 | 15.714 | 1.00 | 31.67 |
| ATOM | 1920 | CD | LYS | A | 541 | 27.712 | 8.227 | 16.528 | 1.00 | 26.84 |
| ATOM | 1921 | CE | LYS | A | 541 | 26.561 | 7.418 | 17.035 | 1.00 | 22.56 |
| ATOM | 1922 | NZ | LYS | A | 541 | 27.091 | 6.148 | 17.574 | 1.00 | 31.19 |
| ATOM | 1923 | C | LYS | A | 541 | 25.563 | 9.467 | 12.204 | 1.00 | 32.35 |
| ATOM | 1924 | O | LYS | A | 541 | 26.324 | 9.441 | 11.250 | 1.00 | 34.75 |
| ATOM | 1925 | N | GLU | A | 542 | 24.298 | 9.069 | 12.126 | 1.00 | 32.04 |
| ATOM | 1926 | CA | GLU | A | 542 | 23.726 | 8.570 | 10.888 | 1.00 | 35.69 |
| ATOM | 1927 | CB | GLU | A | 542 | 22.316 | 8.074 | 11.129 | 1.00 | 38.46 |
| ATOM | 1928 | CG | GLU | A | 542 | 22.269 | 6.772 | 11.888 | 1.00 | 52.51 |
| ATOM | 1929 | CD | GLU | A | 542 | 20.882 | 6.443 | 12.403 | 1.00 | 59.88 |
| ATOM | 1930 | OE1 | GLU | A | 542 | 20.795 | 5.892 | 13.531 | 1.00 | 65.14 |
| ATOM | 1931 | OE2 | GLU | A | 542 | 19.889 | 6.732 | 11.684 | 1.00 | 59.01 |
| ATOM | 1932 | C | GLU | A | 542 | 23.661 | 9.668 | 9.855 | 1.00 | 36.75 |
| ATOM | 1933 | O | GLU | A | 542 | 23.668 | 9.393 | 8.666 | 1.00 | 39.22 |
| ATOM | 1934 | N | LEU | A | 543 | 23.557 | 10.913 | 10.312 | 1.00 | 35.21 |
| ATOM | 1935 | CA | LEU | A | 543 | 23.449 | 12.047 | 9.407 | 1.00 | 33.98 |
| ATOM | 1936 | CB | LEU | A | 543 | 22.549 | 13.145 | 9.990 | 1.00 | 31.70 |
| ATOM | 1937 | CG | LEU | A | 543 | 21.045 | 12.927 | 10.118 | 1.00 | 34.06 |
| ATOM | 1938 | CD1 | LEU | A | 543 | 20.457 | 14.088 | 10.891 | 1.00 | 35.24 |
| ATOM | 1939 | CD2 | LEU | A | 543 | 20.388 | 12.826 | 8.761 | 1.00 | 35.42 |
| ATOM | 1940 | C | LEU | A | 543 | 24.731 | 12.702 | 8.959 | 1.00 | 32.29 |
| ATOM | 1941 | O | LEU | A | 543 | 24.776 | 13.231 | 7.859 | 1.00 | 39.03 |
| ATOM | 1942 | N | PHE | A | 544 | 25.781 | 12.651 | 9.762 | 1.00 | 29.28 |
| ATOM | 1943 | CA | PHE | A | 544 | 26.997 | 13.354 | 9.389 | 1.00 | 29.14 |
| ATOM | 1944 | CB | PHE | A | 544 | 27.203 | 14.561 | 10.330 | 1.00 | 32.64 |
| ATOM | 1945 | CG | PHE | A | 544 | 25.969 | 15.425 | 10.528 | 1.00 | 32.31 |
| ATOM | 1946 | CD1 | PHE | A | 544 | 25.295 | 15.431 | 11.746 | 1.00 | 27.53 |
| ATOM | 1947 | CD2 | PHE | A | 544 | 25.491 | 16.247 | 9.501 | 1.00 | 32.38 |
| ATOM | 1948 | CE1 | PHE | A | 544 | 24.165 | 16.239 | 11.943 | 1.00 | 26.56 |
| ATOM | 1949 | CE2 | PHE | A | 544 | 24.354 | 17.065 | 9.690 | 1.00 | 29.30 |
| ATOM | 1950 | CZ | PHE | A | 544 | 23.694 | 17.057 | 10.914 | 1.00 | 30.03 |
| ATOM | 1951 | C | PHE | A | 544 | 28.269 | 12.505 | 9.356 | 1.00 | 33.29 |
| ATOM | 1952 | O | PHE | A | 544 | 28.193 | 11.266 | 9.571 | 1.00 | 36.40 |
| ATOM | 1953 | OXT | PHE | A | 544 | 29.349 | 13.102 | 9.110 | 1.00 | 34.60 |
| ATOM | 1954 | O1 | HOH | V | 1 | 19.571 | 24.015 | 22.830 | 1.00 | 11.92 |
| ATOM | 1955 | O1 | HOH | V | 2 | 12.600 | 24.091 | 16.912 | 1.00 | 16.18 |
| ATOM | 1956 | O1 | HOH | V | 3 | 14.052 | 22.894 | 14.638 | 1.00 | 22.41 |
| ATOM | 1957 | O1 | HOH | V | 4 | 28.663 | 16.841 | 27.507 | 1.00 | 23.15 |
| ATOM | 1958 | O1 | HOH | V | 5 | 26.725 | 9.526 | 26.728 | 1.00 | 24.50 |
| ATOM | 1959 | O1 | HOH | V | 6 | 18.179 | 21.587 | 21.082 | 1.00 | 24.52 |
| ATOM | 1960 | O1 | HOH | V | 7 | 34.584 | 18.654 | 31.591 | 1.00 | 24.62 |
| ATOM | 1961 | O1 | HOH | V | 8 | 38.207 | 8.705 | 22.227 | 1.00 | 25.07 |
| ATOM | 1962 | O1 | HOH | V | 9 | 18.077 | 19.002 | 0.819 | 1.00 | 25.07 |
| ATOM | 1963 | O1 | HOH | V | 10 | 17.420 | 26.679 | 24.799 | 1.00 | 25.52 |
| ATOM | 1964 | O1 | HOH | V | 11 | 11.110 | 25.828 | 9.180 | 1.00 | 25.56 |
| ATOM | 1965 | O1 | HOH | V | 12 | 25.371 | 34.354 | 26.992 | 1.00 | 25.89 |
| ATOM | 1966 | O1 | HOH | V | 13 | 35.321 | 27.213 | 19.620 | 1.00 | 25.99 |
| ATOM | 1967 | O1 | HOH | V | 14 | 18.045 | 26.166 | 21.645 | 1.00 | 26.14 |
| ATOM | 1968 | O1 | HOH | V | 15 | 19.454 | 10.080 | 17.919 | 1.00 | 26.31 |
| ATOM | 1969 | O1 | HOH | V | 16 | 37.357 | 26.490 | 13.415 | 1.00 | 26.89 |
| ATOM | 1970 | O1 | HOH | V | 17 | 11.508 | 26.772 | 18.302 | 1.00 | 27.31 |
| ATOM | 1971 | O1 | HOH | V | 18 | 15.147 | 25.780 | 21.426 | 1.00 | 27.73 |
| ATOM | 1972 | O1 | HOH | V | 19 | 26.400 | 37.545 | 37.765 | 1.00 | 27.84 |
| ATOM | 1973 | O1 | HOH | V | 20 | 24.927 | 38.184 | 32.702 | 1.00 | 28.88 |
| ATOM | 1974 | O1 | HOH | V | 21 | 22.535 | 18.724 | 7.093 | 1.00 | 29.31 |
| ATOM | 1975 | O1 | HOH | V | 22 | 19.050 | 8.455 | −3.987 | 1.00 | 29.80 |
| ATOM | 1976 | O1 | HOH | V | 23 | 20.732 | 38.540 | 24.291 | 1.00 | 30.07 |
| ATOM | 1977 | O1 | HOH | V | 24 | 14.054 | 28.783 | 15.745 | 1.00 | 31.20 |
| ATOM | 1978 | O1 | HOH | V | 25 | 25.356 | 15.005 | 28.051 | 1.00 | 31.83 |
| ATOM | 1979 | O1 | HOH | V | 26 | 33.279 | 26.348 | 25.938 | 1.00 | 32.16 |
| ATOM | 1980 | O1 | HOH | V | 27 | 14.590 | 28.615 | 28.744 | 1.00 | 32.70 |
| ATOM | 1981 | O1 | HOH | V | 28 | 4.102 | 35.198 | 9.164 | 1.00 | 32.70 |
| ATOM | 1982 | O1 | HOH | V | 29 | 13.577 | 30.615 | 29.768 | 1.00 | 32.70 |
| ATOM | 1983 | O1 | HOH | V | 30 | 28.564 | 37.851 | 14.236 | 1.00 | 32.76 |
| ATOM | 1984 | O1 | HOH | V | 31 | 22.927 | 14.143 | 0.962 | 1.00 | 33.39 |
| ATOM | 1985 | O1 | HOH | V | 32 | 27.550 | 38.081 | 22.254 | 1.00 | 33.51 |
| ATOM | 1986 | O1 | HOH | V | 33 | 4.343 | 30.099 | 9.792 | 1.00 | 33.68 |
| ATOM | 1987 | O1 | HOH | V | 34 | 13.758 | 27.478 | 19.451 | 1.00 | 33.73 |
| ATOM | 1988 | O1 | HOH | V | 35 | 31.045 | 36.851 | 16.729 | 1.00 | 33.87 |
| ATOM | 1989 | O1 | HOH | V | 36 | 19.213 | 14.762 | 21.135 | 1.00 | 33.94 |
| ATOM | 1990 | O1 | HOH | V | 37 | 30.260 | 38.889 | 20.375 | 1.00 | 34.05 |
| ATOM | 1991 | O1 | HOH | V | 38 | 21.211 | 20.354 | 29.122 | 1.00 | 34.10 |
| ATOM | 1992 | O1 | HOH | V | 39 | 32.966 | 5.622 | 27.344 | 1.00 | 34.62 |
| ATOM | 1993 | O1 | HOH | V | 40 | 26.116 | 6.668 | 20.629 | 1.00 | 35.56 |
| ATOM | 1994 | O1 | HOH | V | 41 | −1.516 | 28.517 | 7.655 | 1.00 | 35.63 |
| ATOM | 1995 | O1 | HOH | V | 42 | 34.189 | 32.470 | 17.850 | 1.00 | 35.68 |
| ATOM | 1996 | O1 | HOH | V | 43 | 24.220 | 21.292 | 3.001 | 1.00 | 35.74 |
| ATOM | 1997 | O1 | HOH | V | 44 | 5.910 | 27.836 | 16.119 | 1.00 | 36.07 |
| ATOM | 1998 | O1 | HOH | V | 45 | 26.026 | 15.360 | 5.513 | 1.00 | 36.54 |
| ATOM | 1999 | O1 | HOH | V | 46 | 24.021 | 14.774 | −1.204 | 1.00 | 36.65 |
| ATOM | 2000 | O1 | HOH | V | 47 | 20.363 | 26.930 | 31.179 | 1.00 | 36.70 |
| ATOM | 2001 | O1 | HOH | V | 48 | 35.665 | 32.840 | 10.425 | 1.00 | 36.70 |
| ATOM | 2002 | O1 | HOH | V | 49 | 26.360 | 37.660 | 34.946 | 1.00 | 36.83 |
| ATOM | 2003 | O1 | HOH | V | 50 | 25.128 | 17.207 | −1.881 | 1.00 | 36.96 |
| ATOM | 2004 | O1 | HOH | V | 51 | 24.114 | 21.504 | 30.329 | 1.00 | 37.03 |
| ATOM | 2005 | O1 | HOH | V | 52 | 15.366 | 43.743 | 10.778 | 1.00 | 37.39 |
| ATOM | 2006 | O1 | HOH | V | 53 | 30.933 | 6.183 | 15.530 | 1.00 | 37.82 |
| ATOM | 2007 | O1 | HOH | V | 54 | 4.304 | 36.868 | 5.949 | 1.00 | 38.21 |
| ATOM | 2008 | O1 | HOH | V | 55 | 14.763 | 35.710 | 19.412 | 1.00 | 39.01 |
| ATOM | 2009 | O1 | HOH | V | 56 | 1.357 | 20.195 | 9.921 | 1.00 | 39.03 |
| ATOM | 2010 | O1 | HOH | V | 57 | 13.913 | 23.892 | 19.724 | 1.00 | 39.09 |
| ATOM | 2011 | O1 | HOH | V | 58 | 12.354 | 12.577 | −11.744 | 1.00 | 39.57 |
| ATOM | 2012 | O1 | HOH | V | 59 | 19.367 | 4.873 | 15.945 | 1.00 | 39.60 |
| ATOM | 2013 | O1 | HOH | V | 60 | 28.823 | 27.044 | −1.138 | 1.00 | 39.87 |
| ATOM | 2014 | O1 | HOH | V | 61 | 24.086 | 5.629 | 14.333 | 1.00 | 39.92 |
| ATOM | 2015 | O1 | HOH | V | 62 | 6.227 | 36.542 | 12.153 | 1.00 | 39.94 |
| ATOM | 2016 | O1 | HOH | V | 63 | 25.257 | 19.031 | 30.271 | 1.00 | 40.01 |
| ATOM | 2017 | O1 | HOH | V | 64 | 33.091 | 35.051 | 17.676 | 1.00 | 40.07 |
| ATOM | 2018 | O1 | HOH | V | 65 | 33.832 | 31.154 | 20.549 | 1.00 | 40.37 |
| ATOM | 2019 | O1 | HOH | V | 66 | 40.477 | 15.296 | 9.510 | 1.00 | 41.20 |
| ATOM | 2020 | O1 | HOH | V | 67 | 23.525 | 9.325 | −8.918 | 1.00 | 41.87 |
| ATOM | 2021 | O1 | HOH | V | 68 | 18.624 | 25.089 | −4.128 | 1.00 | 42.15 |
| ATOM | 2022 | O1 | HOH | V | 69 | 24.673 | 39.002 | −1.542 | 1.00 | 42.21 |
| ATOM | 2023 | O1 | HOH | V | 70 | 25.134 | 15.085 | 2.723 | 1.00 | 42.21 |
| ATOM | 2024 | O1 | HOH | V | 71 | 10.336 | 29.797 | 26.075 | 1.00 | 42.37 |
| ATOM | 2025 | O1 | HOH | V | 72 | 16.798 | 18.655 | −11.711 | 1.00 | 42.43 |
| ATOM | 2026 | O1 | HOH | V | 73 | −2.391 | 33.028 | 0.604 | 1.00 | 42.69 |
| ATOM | 2027 | O1 | HOH | V | 74 | 7.033 | 20.764 | 20.270 | 1.00 | 43.01 |
| ATOM | 2028 | O1 | HOH | V | 75 | 27.375 | 26.586 | 32.414 | 1.00 | 43.08 |
| ATOM | 2029 | O1 | HOH | V | 76 | 24.651 | 12.458 | 27.335 | 1.00 | 43.14 |
| ATOM | 2030 | O1 | HOH | V | 77 | 21.223 | 24.850 | 0.260 | 1.00 | 43.31 |
| ATOM | 2031 | O1 | HOH | V | 78 | 13.059 | 10.272 | 13.532 | 1.00 | 43.63 |
| ATOM | 2032 | O1 | HOH | V | 79 | 27.284 | 19.103 | 8.210 | 1.00 | 44.00 |
| ATOM | 2033 | O1 | HOH | V | 80 | 34.897 | 34.595 | 21.757 | 1.00 | 44.35 |
| ATOM | 2034 | O1 | HOH | V | 81 | 19.496 | 24.289 | −1.468 | 1.00 | 44.41 |
| ATOM | 2035 | O1 | HOH | V | 82 | 26.589 | 22.429 | 32.257 | 1.00 | 44.58 |
| ATOM | 2036 | O1 | HOH | V | 83 | 41.875 | 11.753 | 22.776 | 1.00 | 44.72 |
| ATOM | 2037 | O1 | HOH | V | 84 | 24.041 | 16.824 | 29.300 | 1.00 | 44.91 |
| ATOM | 2038 | O1 | HOH | V | 85 | 39.182 | 23.600 | 24.591 | 1.00 | 45.03 |
| ATOM | 2039 | O1 | HOH | V | 86 | 16.711 | 29.367 | 31.469 | 1.00 | 45.22 |
| ATOM | 2040 | O1 | HOH | V | 87 | 26.474 | 37.247 | 27.330 | 1.00 | 45.42 |
| ATOM | 2041 | O1 | HOH | V | 88 | 10.580 | 10.952 | 7.001 | 1.00 | 45.46 |
| ATOM | 2042 | O1 | HOH | V | 89 | 17.919 | 17.134 | 23.482 | 1.00 | 45.53 |
| ATOM | 2043 | O1 | HOH | V | 90 | 22.700 | 27.169 | 33.013 | 1.00 | 45.86 |
| ATOM | 2044 | O1 | HOH | V | 91 | 20.218 | 40.609 | 29.025 | 1.00 | 46.30 |
| ATOM | 2045 | O1 | HOH | V | 92 | 21.955 | 40.569 | 26.103 | 1.00 | 46.31 |
| ATOM | 2046 | O1 | HOH | V | 93 | 5.333 | 26.234 | 18.852 | 1.00 | 46.91 |
| ATOM | 2047 | O1 | HOH | V | 94 | 6.403 | 18.038 | 15.920 | 1.00 | 47.12 |
| ATOM | 2048 | O1 | HOH | V | 95 | 37.307 | 11.015 | 10.807 | 1.00 | 47.31 |
| ATOM | 2049 | O1 | HOH | V | 96 | 11.338 | 13.464 | 13.985 | 1.00 | 47.96 |
| ATOM | 2050 | O1 | HOH | V | 97 | 10.441 | 37.707 | 30.346 | 1.00 | 48.02 |
| ATOM | 2051 | O1 | HOH | V | 98 | 30.888 | 36.428 | 14.084 | 1.00 | 48.48 |
| ATOM | 2052 | O1 | HOH | V | 99 | 27.882 | 17.980 | 29.841 | 1.00 | 48.50 |
| ATOM | 2053 | O1 | HOH | V | 100 | 33.749 | 37.917 | 8.734 | 1.00 | 48.51 |
| ATOM | 2054 | O1 | HOH | V | 101 | 18.379 | 27.870 | 33.046 | 1.00 | 48.64 |
| ATOM | 2055 | O1 | HOH | V | 102 | 35.449 | 31.668 | 8.141 | 1.00 | 48.94 |
| ATOM | 2056 | O1 | HOH | V | 103 | 29.164 | 17.601 | 3.576 | 1.00 | 49.29 |
| ATOM | 2057 | O1 | HOH | V | 104 | 33.653 | 32.899 | 6.888 | 1.00 | 49.36 |
| ATOM | 2058 | O1 | HOH | V | 105 | 42.507 | 15.827 | 13.475 | 1.00 | 49.69 |
| ATOM | 2059 | O1 | HOH | V | 106 | 37.222 | 20.712 | 33.061 | 1.00 | 49.71 |
| ATOM | 2060 | O1 | HOH | V | 107 | 19.173 | 42.140 | 26.556 | 1.00 | 49.87 |
| ATOM | 2061 | O1 | HOH | V | 108 | −1.128 | 28.133 | 10.338 | 1.00 | 50.07 |
| ATOM | 2062 | O1 | HOH | V | 109 | 13.605 | 40.750 | 25.634 | 1.00 | 50.16 |
| ATOM | 2063 | O1 | HOH | V | 110 | −1.457 | 28.059 | −4.001 | 1.00 | 50.19 |
| ATOM | 2064 | O1 | HOH | V | 111 | −0.092 | 31.118 | 6.416 | 1.00 | 50.25 |
| ATOM | 2065 | O1 | HOH | V | 112 | 3.374 | 39.612 | −3.935 | 1.00 | 50.31 |
| ATOM | 2066 | O1 | HOH | V | 113 | 32.127 | 18.267 | 32.763 | 1.00 | 50.37 |
| ATOM | 2067 | O1 | HOH | V | 114 | 18.258 | 23.101 | 26.041 | 1.00 | 50.51 |
| ATOM | 2068 | O1 | HOH | V | 115 | 26.516 | 26.089 | −3.694 | 1.00 | 50.63 |
| ATOM | 2069 | O1 | HOH | V | 116 | 13.352 | 17.048 | 19.784 | 1.00 | 50.83 |
| ATOM | 2070 | O1 | HOH | V | 117 | 10.647 | 6.108 | 12.167 | 1.00 | 50.93 |
| ATOM | 2071 | O1 | HOH | V | 118 | 26.146 | 17.547 | 1.891 | 1.00 | 50.94 |
| ATOM | 2072 | O1 | HOH | V | 119 | 15.203 | 21.870 | 20.133 | 1.00 | 50.98 |
| ATOM | 2073 | O1 | HOH | V | 120 | 32.029 | 18.786 | 4.116 | 1.00 | 51.10 |
| ATOM | 2074 | O1 | HOH | V | 121 | 22.114 | 18.269 | 27.109 | 1.00 | 51.46 |
| ATOM | 2075 | O1 | HOH | V | 122 | 25.668 | 18.396 | 5.657 | 1.00 | 51.52 |
| ATOM | 2076 | O1 | HOH | V | 123 | 41.989 | 18.102 | 20.145 | 1.00 | 51.77 |
| ATOM | 2077 | O1 | HOH | V | 124 | 36.078 | 5.753 | 14.297 | 1.00 | 52.16 |
| ATOM | 2078 | O1 | HOH | V | 125 | −4.191 | 23.385 | −0.546 | 1.00 | 52.19 |
| ATOM | 2079 | O1 | HOH | V | 126 | 38.840 | 23.465 | 28.263 | 1.00 | 52.44 |
| ATOM | 2080 | O1 | HOH | V | 127 | 17.889 | 40.107 | 16.024 | 1.00 | 52.46 |
| ATOM | 2081 | O1 | HOH | V | 128 | 10.480 | 31.213 | 29.646 | 1.00 | 52.47 |
| ATOM | 2082 | O1 | HOH | V | 129 | 11.041 | 40.539 | 4.064 | 1.00 | 52.63 |
| ATOM | 2083 | O1 | HOH | V | 130 | 25.662 | 37.407 | 30.124 | 1.00 | 52.65 |
| ATOM | 2084 | O1 | HOH | V | 131 | 37.583 | 19.513 | 14.379 | 1.00 | 52.79 |
| ATOM | 2085 | O1 | HOH | V | 132 | 31.355 | 36.654 | 28.009 | 1.00 | 52.82 |
| ATOM | 2086 | O1 | HOH | V | 133 | 24.495 | 25.685 | 32.162 | 1.00 | 52.92 |
| ATOM | 2087 | O1 | HOH | V | 134 | 29.710 | 0.923 | 19.113 | 1.00 | 52.92 |
| ATOM | 2088 | O1 | HOH | V | 135 | 17.608 | 9.016 | 9.185 | 1.00 | 52.96 |
| ATOM | 2089 | O1 | HOH | V | 136 | 24.883 | 4.742 | 16.973 | 1.00 | 53.32 |
| ATOM | 2090 | O1 | HOH | V | 137 | 29.325 | 41.144 | 15.563 | 1.00 | 54.00 |
| ATOM | 2091 | O1 | HOH | V | 138 | 8.148 | 32.691 | 27.089 | 1.00 | 54.14 |
| ATOM | 2092 | O1 | HOH | V | 139 | 25.869 | 44.302 | 17.088 | 1.00 | 54.60 |
| ATOM | 2093 | O1 | HOH | V | 140 | 31.180 | 24.098 | 0.471 | 1.00 | 54.96 |
| ATOM | 2094 | O1 | HOH | V | 141 | 32.092 | 39.604 | 16.380 | 1.00 | 55.48 |
| ATOM | 2095 | O1 | HOH | V | 142 | 20.031 | 28.982 | 35.641 | 1.00 | 55.95 |
| ATOM | 2096 | O1 | HOH | V | 143 | 19.537 | 17.716 | 26.209 | 1.00 | 56.58 |
| ATOM | 2097 | O1 | HOH | V | 144 | 3.004 | 26.615 | 21.765 | 1.00 | 56.65 |
| ATOM | 2098 | O1 | HOH | V | 145 | 3.566 | 13.601 | 10.033 | 1.00 | 56.98 |
| ATOM | 2099 | O1 | HOH | V | 146 | 16.090 | 48.803 | −0.839 | 1.00 | 57.02 |
| ATOM | 2100 | O1 | HOH | V | 147 | 41.521 | 30.957 | 16.321 | 1.00 | 57.24 |
| ATOM | 2101 | O1 | HOH | V | 148 | 21.322 | 6.331 | 6.002 | 1.00 | 57.58 |
| ATOM | 2102 | O1 | HOH | V | 149 | 9.375 | 39.538 | 12.218 | 1.00 | 57.59 |
| ATOM | 2103 | O1 | HOH | V | 150 | 15.176 | 39.661 | 18.686 | 1.00 | 58.07 |
| ATOM | 2104 | O1 | HOH | V | 151 | 20.363 | 24.179 | 31.362 | 1.00 | 58.21 |
| ATOM | 2105 | O1 | HOH | V | 152 | 14.157 | 40.583 | 21.313 | 1.00 | 58.26 |
| ATOM | 2106 | O1 | HOH | V | 153 | 13.420 | 36.512 | 16.363 | 1.00 | 58.51 |
| ATOM | 2107 | O1 | HOH | V | 154 | 1.911 | 36.126 | 4.549 | 1.00 | 58.55 |
| ATOM | 2108 | O1 | HOH | V | 155 | 16.108 | 6.884 | 12.075 | 1.00 | 58.70 |
| ATOM | 2109 | O1 | HOH | V | 156 | −4.815 | 29.631 | −4.704 | 1.00 | 58.74 |
| ATOM | 2110 | O1 | HOH | V | 157 | 17.728 | 4.118 | 0.882 | 1.00 | 59.17 |
| ATOM | 2111 | O1 | HOH | V | 158 | 11.034 | 33.423 | 31.437 | 1.00 | 59.38 |
| ATOM | 2112 | O1 | HOH | V | 159 | 39.277 | 19.752 | 9.565 | 1.00 | 59.80 |
| ATOM | 2113 | O1 | HOH | V | 160 | 20.830 | 23.224 | −9.945 | 1.00 | 59.89 |
| ATOM | 2114 | O1 | HOH | V | 161 | 29.709 | 18.612 | 31.510 | 1.00 | 60.09 |
| ATOM | 2115 | O1 | HOH | V | 162 | 27.074 | 2.621 | 20.642 | 1.00 | 60.12 |
| ATOM | 2116 | O1 | HOH | V | 163 | 5.858 | 22.161 | −4.489 | 1.00 | 60.19 |
| ATOM | 2117 | O1 | HOH | V | 164 | 15.034 | 44.034 | 5.590 | 1.00 | 60.39 |
| ATOM | 2118 | O1 | HOH | V | 165 | 33.009 | 22.978 | 33.387 | 1.00 | 60.43 |
| ATOM | 2119 | O1 | HOH | V | 166 | 2.030 | 21.719 | −4.397 | 1.00 | 60.59 |
| ATOM | 2120 | O1 | HOH | V | 167 | 3.774 | 21.532 | 3.731 | 1.00 | 60.68 |
| ATOM | 2121 | O1 | HOH | V | 168 | 28.412 | 13.665 | −7.177 | 1.00 | 60.83 |
| ATOM | 2122 | O1 | HOH | V | 169 | 39.061 | 22.162 | 31.337 | 1.00 | 61.22 |
| ATOM | 2123 | O1 | HOH | V | 170 | 30.385 | 11.086 | 11.347 | 1.00 | 61.45 |
| ATOM | 2124 | O1 | HOH | V | 171 | 38.929 | 11.728 | 26.423 | 1.00 | 61.62 |
| ATOM | 2125 | O1 | HOH | V | 172 | 9.596 | 6.343 | −6.409 | 1.00 | 61.85 |
| ATOM | 2126 | O1 | HOH | V | 173 | 27.960 | 21.516 | 2.108 | 1.00 | 61.90 |
| ATOM | 2127 | O1 | HOH | V | 174 | 4.313 | 13.515 | −0.097 | 1.00 | 62.15 |
| ATOM | 2128 | O1 | HOH | V | 175 | −4.186 | 27.811 | 7.260 | 1.00 | 62.60 |
| ATOM | 2129 | O1 | HOH | V | 176 | 10.940 | 41.489 | 27.508 | 1.00 | 63.29 |
| ATOM | 2130 | O1 | HOH | V | 177 | 24.701 | 19.822 | −1.623 | 1.00 | 63.64 |
| ATOM | 2131 | O1 | HOH | V | 178 | 42.644 | 18.535 | 10.330 | 1.00 | 63.68 |
| ATOM | 2132 | O1 | HOH | V | 179 | 1.986 | 36.706 | 26.540 | 1.00 | 63.68 |
| ATOM | 2133 | O1 | HOH | V | 180 | 22.345 | 47.189 | 18.548 | 1.00 | 64.72 |
| ATOM | 2134 | O1 | HOH | V | 181 | 7.492 | 6.994 | 1.249 | 1.00 | 64.77 |
| ATOM | 2135 | O1 | HOH | V | 182 | 29.348 | 37.819 | 26.783 | 1.00 | 64.90 |
| ATOM | 2136 | O1 | HOH | V | 183 | 39.883 | 20.258 | 25.832 | 1.00 | 65.05 |
| ATOM | 2137 | O1 | HOH | V | 184 | 33.197 | 24.977 | 3.656 | 1.00 | 65.28 |
| ATOM | 2138 | O1 | HOH | V | 185 | 1.167 | 34.045 | 3.205 | 1.00 | 65.41 |
| ATOM | 2139 | O1 | HOH | V | 186 | 36.275 | 32.735 | 23.649 | 1.00 | 65.48 |
| ATOM | 2140 | O1 | HOH | V | 187 | −2.787 | 30.904 | −0.828 | 1.00 | 65.58 |
| ATOM | 2141 | O1 | HOH | V | 188 | 6.538 | 23.682 | −10.695 | 1.00 | 66.34 |
| ATOM | 2142 | O1 | HOH | V | 189 | 10.682 | 8.724 | 11.380 | 1.00 | 66.87 |
| ATOM | 2143 | O1 | HOH | V | 190 | 14.198 | 8.869 | −12.442 | 1.00 | 67.21 |
| ATOM | 2144 | O1 | HOH | V | 191 | −2.267 | 38.672 | −2.479 | 1.00 | 67.22 |
| ATOM | 2145 | O1 | HOH | V | 192 | 29.224 | 8.950 | 12.107 | 1.00 | 67.30 |
| ATOM | 2146 | O1 | HOH | V | 193 | 11.819 | 8.883 | 6.281 | 1.00 | 67.62 |
| ATOM | 2147 | O1 | HOH | V | 194 | 38.489 | 16.915 | 8.462 | 1.00 | 68.36 |
| ATOM | 2148 | O1 | HOH | V | 195 | 33.987 | 7.482 | 15.967 | 1.00 | 68.84 |
| ATOM | 2149 | O1 | HOH | V | 196 | 4.892 | 34.328 | −7.351 | 1.00 | 68.88 |
| ATOM | 2150 | O1 | HOH | V | 197 | 39.056 | 27.510 | 8.823 | 1.00 | 68.92 |
| ATOM | 2151 | O1 | HOH | V | 198 | 9.884 | 6.802 | 3.712 | 1.00 | 69.08 |
| ATOM | 2152 | O1 | HOH | V | 199 | 37.843 | 34.495 | 12.256 | 1.00 | 69.20 |
| ATOM | 2153 | O1 | HOH | V | 200 | 34.349 | 36.343 | 19.667 | 1.00 | 69.76 |
| ATOM | 2154 | O1 | HOH | V | 201 | 38.474 | 1.028 | 20.411 | 1.00 | 70.03 |
| ATOM | 2155 | O1 | HOH | V | 202 | 27.053 | 38.768 | 25.134 | 1.00 | 70.09 |
| ATOM | 2156 | O1 | HOH | V | 203 | 28.267 | 37.799 | 29.494 | 1.00 | 70.65 |
| ATOM | 2157 | O1 | HOH | V | 204 | 25.427 | 35.915 | 1.694 | 1.00 | 71.85 |
| ATOM | 2158 | O1 | HOH | V | 205 | 18.375 | 3.341 | 9.734 | 1.00 | 72.08 |
| ATOM | 2159 | O1 | HOH | V | 206 | 29.055 | 24.527 | −1.260 | 1.00 | 72.11 |
| ATOM | 2160 | O1 | HOH | V | 207 | 15.436 | 3.667 | −5.477 | 1.00 | 72.50 |
| ATOM | 2161 | O1 | HOH | V | 208 | 2.845 | 25.343 | 17.594 | 1.00 | 72.71 |
| ATOM | 2162 | O1 | HOH | V | 209 | 31.127 | 39.615 | 33.793 | 1.00 | 74.83 |
| ATOM | 2163 | O1 | HOH | V | 210 | 15.559 | 12.402 | −12.936 | 1.00 | 75.70 |
| ATOM | 2164 | O1 | HOH | V | 211 | 40.158 | 26.133 | 22.103 | 1.00 | 76.15 |
| ATOM | 2165 | O1 | HOH | V | 212 | 3.811 | 32.891 | 15.563 | 1.00 | 77.51 |
| ATOM | 2166 | O1 | HOH | V | 213 | 21.251 | 45.356 | 7.011 | 1.00 | 78.54 |
| ATOM | 2167 | O1 | HOH | V | 214 | 31.582 | 39.863 | 24.269 | 1.00 | 79.14 |
| ATOM | 2168 | O1 | HOH | V | 215 | −0.088 | 20.677 | 7.373 | 1.00 | 79.61 |
| ATOM | 2169 | O1 | HOH | V | 216 | 34.466 | 33.712 | 35.188 | 1.00 | 80.04 |
| ATOM | 2170 | O1 | HOH | V | 217 | 5.299 | 14.605 | 2.187 | 1.00 | 82.20 |
| ATOM | 2171 | O1 | HOH | V | 218 | −0.119 | 43.183 | −1.190 | 1.00 | 82.43 |
| ATOM | 2172 | O1 | HOH | V | 219 | 21.612 | 10.359 | −10.786 | 1.00 | 83.22 |
| ATOM | 2173 | O1 | HOH | V | 220 | 18.145 | 42.688 | 23.379 | 1.00 | 83.87 |
| ATOM | 2174 | O1 | HOH | V | 221 | 38.141 | 26.882 | 26.796 | 1.00 | 84.27 |
| ATOM | 2175 | O1 | HOH | V | 222 | 15.645 | 37.869 | 15.459 | 1.00 | 86.80 |
| ATOM | 2176 | O1 | HOH | V | 223 | 33.347 | 0.052 | 24.088 | 1.00 | 88.51 |
| ATOM | 2177 | O1 | HOH | V | 224 | 26.717 | 9.812 | 6.181 | 1.00 | 89.08 |
| ATOM | 2178 | O1 | HOH | V | 225 | 13.496 | 7.603 | 15.584 | 1.00 | 89.47 |
| ATOM | 2179 | O1 | HOH | V | 226 | 0.897 | 31.611 | 9.140 | 1.00 | 89.58 |
| ATOM | 2180 | O1 | HOH | V | 227 | 13.018 | 26.525 | −7.628 | 1.00 | 90.92 |
| ATOM | 2181 | O1 | HOH | V | 228 | 6.286 | 19.849 | 17.755 | 1.00 | 91.50 |
| ATOM | 2182 | O1 | HOH | V | 229 | 19.825 | 7.712 | 0.669 | 1.00 | 92.59 |
| ATOM | 2183 | O1 | HOH | V | 230 | 16.141 | 11.181 | 20.218 | 1.00 | 93.48 |
| ATOM | 2184 | O1 | HOH | V | 231 | 30.341 | 41.381 | 18.146 | 1.00 | 94.31 |
| ATOM | 2185 | C1 | CHO | L | 1 | 18.565 | 26.648 | 16.097 | 1.00 | 17.77 |
| ATOM | 2186 | C4 | CHO | L | 1 | 17.062 | 26.995 | 16.208 | 1.00 | 14.30 |
| ATOM | 2187 | C7 | CHO | L | 1 | 16.300 | 25.791 | 16.806 | 1.00 | 16.71 |
| ATOM | 2188 | C9 | CHO | L | 1 | 16.836 | 25.493 | 18.221 | 1.00 | 17.53 |
| ATOM | 2189 | C12 | CHO | L | 1 | 18.329 | 25.274 | 18.190 | 1.00 | 21.09 |
| ATOM | 2190 | C13 | CHO | L | 1 | 18.814 | 24.179 | 18.791 | 1.00 | 17.61 |
| ATOM | 2191 | C15 | CHO | L | 1 | 20.283 | 23.851 | 18.848 | 1.00 | 16.99 |
| ATOM | 2192 | C18 | CHO | L | 1 | 21.159 | 25.087 | 18.576 | 1.00 | 18.07 |
| ATOM | 2193 | C20 | CHO | L | 1 | 20.643 | 25.806 | 17.292 | 1.00 | 18.92 |
| ATOM | 2194 | C22 | CHO | L | 1 | 19.182 | 26.326 | 17.485 | 1.00 | 20.91 |
| ATOM | 2195 | C23 | CHO | L | 1 | 21.613 | 26.937 | 16.851 | 1.00 | 19.33 |
| ATOM | 2196 | C26 | CHO | L | 1 | 23.077 | 26.440 | 16.727 | 1.00 | 23.26 |
| ATOM | 2197 | C29 | CHO | L | 1 | 23.552 | 25.816 | 18.063 | 1.00 | 24.98 |
| ATOM | 2198 | C30 | CHO | L | 1 | 22.616 | 24.628 | 18.351 | 1.00 | 19.40 |
| ATOM | 2199 | C32 | CHO | L | 1 | 23.316 | 23.866 | 19.486 | 1.00 | 18.81 |
| ATOM | 2200 | C35 | CHO | L | 1 | 24.818 | 24.074 | 19.179 | 1.00 | 18.34 |
| ATOM | 2201 | C38 | CHO | L | 1 | 24.905 | 25.059 | 17.980 | 1.00 | 20.78 |
| ATOM | 2202 | C40 | CHO | L | 1 | 23.518 | 26.864 | 19.214 | 1.00 | 25.58 |
| ATOM | 2203 | C44 | CHO | L | 1 | 19.179 | 27.650 | 18.304 | 1.00 | 21.74 |
| ATOM | 2204 | C48 | CHO | L | 1 | 26.193 | 25.932 | 18.008 | 1.00 | 18.44 |
| ATOM | 2205 | C50 | CHO | L | 1 | 26.277 | 26.915 | 16.822 | 1.00 | 20.10 |
| ATOM | 2206 | C54 | CHO | L | 1 | 27.502 | 25.106 | 18.059 | 1.00 | 18.74 |
| ATOM | 2207 | C57 | CHO | L | 1 | 27.623 | 23.980 | 17.005 | 1.00 | 19.99 |
| ATOM | 2208 | C60 | CHO | L | 1 | 29.075 | 23.453 | 17.059 | 1.00 | 22.47 |
| ATOM | 2209 | C63 | CHO | L | 1 | 29.308 | 22.160 | 16.245 | 1.00 | 22.68 |
| ATOM | 2210 | C65 | CHO | L | 1 | 30.827 | 21.916 | 16.132 | 1.00 | 21.49 |
| ATOM | 2211 | C69 | CHO | L | 1 | 28.658 | 20.944 | 16.934 | 1.00 | 23.08 |
| ATOM | 2212 | O73 | CHO | L | 1 | 14.905 | 26.073 | 16.943 | 1.00 | 22.77 |
| END | | | | | | | | | | |

TABLE 9

| | | | | | | | | | | | |
|---|---|---|---|---|---|---|---|---|---|---|---|
| ATOM | 1 | N | HIS | A | 261 | −18.369 | 28.759 | −10.025 | 1.00 | 60.80 | N |
| ATOM | 2 | CA | HIS | A | 261 | −17.481 | 28.056 | −11.002 | 1.00 | 61.14 | C |
| ATOM | 3 | CB | HIS | A | 261 | −18.304 | 27.204 | −11.961 | 1.00 | 61.45 | C |
| ATOM | 4 | CG | HIS | A | 261 | −17.529 | 26.743 | −13.153 | 1.00 | 63.15 | C |
| ATOM | 5 | ND1 | HIS | A | 261 | −17.467 | 27.465 | −14.327 | 1.00 | 65.59 | N |
| ATOM | 6 | CE1 | HIS | A | 261 | −16.705 | 26.824 | −15.195 | 1.00 | 66.06 | C |
| ATOM | 7 | NE2 | HIS | A | 261 | −16.255 | 25.723 | −14.620 | 1.00 | 66.44 | N |
| ATOM | 8 | CD2 | HIS | A | 261 | −16.754 | 25.650 | −13.342 | 1.00 | 65.64 | C |
| ATOM | 9 | C | HIS | A | 261 | −16.640 | 29.033 | −11.812 | 1.00 | 60.44 | C |
| ATOM | 10 | O | HIS | A | 261 | −15.409 | 29.012 | −11.759 | 1.00 | 60.45 | O |
| ATOM | 11 | N | HIS | A | 262 | −17.309 | 29.814 | −12.647 | 1.00 | 59.75 | N |
| ATOM | 12 | CA | HIS | A | 262 | −16.661 | 30.904 | −13.361 | 1.00 | 59.30 | C |
| ATOM | 13 | CB | HIS | A | 262 | −17.695 | 31.652 | −14.199 | 1.00 | 58.98 | C |
| ATOM | 14 | CG | HIS | A | 262 | −18.358 | 30.763 | −15.209 | 1.00 | 59.57 | C |
| ATOM | 15 | ND1 | HIS | A | 262 | −19.391 | 29.908 | −14.883 | 1.00 | 59.43 | N |
| ATOM | 16 | CE1 | HIS | A | 262 | −19.744 | 29.220 | −15.954 | 1.00 | 60.29 | C |
| ATOM | 17 | NE2 | HIS | A | 262 | −18.967 | 29.586 | −16.959 | 1.00 | 59.72 | N |
| ATOM | 18 | CD2 | HIS | A | 262 | −18.082 | 30.538 | −16.516 | 1.00 | 59.13 | C |
| ATOM | 19 | C | HIS | A | 262 | −15.815 | 31.792 | −12.437 | 1.00 | 59.09 | C |
| ATOM | 20 | O | HIS | A | 262 | −14.698 | 32.162 | −12.810 | 1.00 | 58.61 | O |
| ATOM | 21 | N | LEU | A | 263 | −16.300 | 32.109 | −11.235 | 1.00 | 58.77 | N |
| ATOM | 22 | CA | LEU | A | 263 | −15.449 | 32.829 | −10.282 | 1.00 | 58.96 | C |
| ATOM | 23 | CB | LEU | A | 263 | −16.221 | 33.286 | −9.044 | 1.00 | 58.85 | C |
| ATOM | 24 | CG | LEU | A | 263 | −16.866 | 34.666 | −9.101 | 1.00 | 58.28 | C |
| ATOM | 25 | CD1 | LEU | A | 263 | −17.642 | 34.934 | −7.824 | 1.00 | 57.90 | C |
| ATOM | 26 | CD2 | LEU | A | 263 | −15.841 | 35.758 | −9.332 | 1.00 | 57.75 | C |
| ATOM | 27 | C | LEU | A | 263 | −14.261 | 31.965 | −9.840 | 1.00 | 59.44 | C |
| ATOM | 28 | O | LEU | A | 263 | −13.154 | 32.463 | −9.682 | 1.00 | 59.40 | O |
| ATOM | 29 | N | GLU | A | 264 | −14.497 | 30.672 | −9.638 | 1.00 | 60.29 | N |
| ATOM | 30 | CA | GLU | A | 264 | −13.450 | 29.748 | −9.204 | 1.00 | 60.91 | C |
| ATOM | 31 | CB | GLU | A | 264 | −14.038 | 28.349 | −8.952 | 1.00 | 61.72 | C |
| ATOM | 32 | CG | GLU | A | 264 | −13.227 | 27.462 | −8.006 | 1.00 | 64.54 | C |
| ATOM | 33 | CD | GLU | A | 264 | −13.691 | 25.995 | −8.002 | 1.00 | 68.63 | C |
| ATOM | 34 | OE1 | GLU | A | 264 | −14.800 | 25.693 | −7.476 | 1.00 | 69.87 | O |
| ATOM | 35 | OE2 | GLU | A | 264 | −12.934 | 25.135 | −8.534 | 1.00 | 70.91 | O |
| ATOM | 36 | C | GLU | A | 264 | −12.339 | 29.694 | −10.251 | 1.00 | 60.26 | C |
| ATOM | 37 | O | GLU | A | 264 | −11.161 | 29.596 | −9.920 | 1.00 | 60.38 | O |
| ATOM | 38 | N | VAL | A | 265 | −12.709 | 29.797 | −11.516 | 1.00 | 59.34 | N |
| ATOM | 39 | CA | VAL | A | 265 | −11.722 | 29.766 | −12.574 | 1.00 | 58.79 | C |
| ATOM | 40 | CB | VAL | A | 265 | −12.393 | 29.559 | −13.923 | 1.00 | 58.56 | C |
| ATOM | 41 | CG1 | VAL | A | 265 | −11.436 | 29.892 | −15.064 | 1.00 | 58.68 | C |
| ATOM | 42 | CG2 | VAL | A | 265 | −12.905 | 28.140 | −14.025 | 1.00 | 57.58 | C |
| ATOM | 43 | C | VAL | A | 265 | −10.878 | 31.034 | −12.599 | 1.00 | 58.95 | C |
| ATOM | 44 | O | VAL | A | 265 | −9.689 | 30.984 | −12.896 | 1.00 | 58.69 | O |
| ATOM | 45 | N | LEU | A | 266 | −11.488 | 32.164 | −12.282 | 1.00 | 59.10 | N |
| ATOM | 46 | CA | LEU | A | 266 | −10.780 | 33.433 | −12.254 | 1.00 | 59.95 | C |
| ATOM | 47 | CB | LEU | A | 266 | −11.780 | 34.586 | −12.145 | 1.00 | 59.85 | C |
| ATOM | 48 | CG | LEU | A | 266 | −12.590 | 34.829 | −13.408 | 1.00 | 59.43 | C |
| ATOM | 49 | CD1 | LEU | A | 266 | −13.498 | 36.026 | −13.226 | 1.00 | 58.82 | C |
| ATOM | 50 | CD2 | LEU | A | 266 | −11.655 | 35.012 | −14.604 | 1.00 | 58.96 | C |
| ATOM | 51 | C | LEU | A | 266 | −9.777 | 33.556 | −11.108 | 1.00 | 60.75 | C |
| ATOM | 52 | O | LEU | A | 266 | −8.907 | 34.423 | −11.140 | 1.00 | 60.71 | O |
| ATOM | 53 | N | PHE | A | 267 | −9.914 | 32.713 | −10.094 | 1.00 | 62.00 | N |
| ATOM | 54 | CA | PHE | A | 267 | −9.035 | 32.758 | −8.931 | 1.00 | 63.40 | C |
| ATOM | 55 | CB | PHE | A | 267 | −9.881 | 32.790 | −7.656 | 1.00 | 63.54 | C |
| ATOM | 56 | CG | PHE | A | 267 | −10.498 | 34.119 | −7.397 | 1.00 | 64.83 | C |
| ATOM | 57 | CD1 | PHE | A | 267 | −11.812 | 34.368 | −7.719 | 1.00 | 66.41 | C |
| ATOM | 58 | CE1 | PHE | A | 267 | −12.367 | 35.615 | −7.500 | 1.00 | 67.05 | C |
| ATOM | 59 | CZ | PHE | A | 267 | −11.605 | 36.617 | −6.969 | 1.00 | 67.09 | C |
| ATOM | 60 | CE2 | PHE | A | 267 | −10.298 | 36.382 | −6.649 | 1.00 | 67.35 | C |
| ATOM | 61 | CD2 | PHE | A | 267 | −9.745 | 35.138 | −6.864 | 1.00 | 66.42 | C |
| ATOM | 62 | C | PHE | A | 267 | −8.042 | 31.595 | −8.858 | 1.00 | 64.32 | C |
| ATOM | 63 | O | PHE | A | 267 | −7.031 | 31.680 | −8.158 | 1.00 | 65.16 | O |
| ATOM | 64 | N | GLN | A | 268 | −8.325 | 30.519 | −9.587 | 1.00 | 64.99 | N |
| ATOM | 65 | CA | GLN | A | 268 | −7.514 | 29.299 | −9.542 | 1.00 | 65.36 | C |
| ATOM | 66 | CB | GLN | A | 268 | −7.800 | 28.433 | −10.770 | 1.00 | 65.80 | C |
| ATOM | 67 | CG | GLN | A | 268 | −7.456 | 26.956 | −10.580 | 1.00 | 67.88 | C |
| ATOM | 68 | CD | GLN | A | 268 | −7.793 | 26.122 | −11.809 | 1.00 | 70.56 | C |
| ATOM | 69 | OE1 | GLN | A | 268 | −8.555 | 26.568 | −12.681 | 1.00 | 72.72 | O |
| ATOM | 70 | NE2 | GLN | A | 268 | −7.223 | 24.919 | −11.891 | 1.00 | 71.16 | N |
| ATOM | 71 | C | GLN | A | 268 | −6.001 | 29.518 | −9.415 | 1.00 | 64.81 | C |
| ATOM | 72 | O | GLN | A | 268 | −5.350 | 28.832 | −8.630 | 1.00 | 64.77 | O |
| ATOM | 73 | N | GLY | A | 269 | −5.441 | 30.451 | −10.180 | 1.00 | 64.05 | N |
| ATOM | 74 | CA | GLY | A | 269 | −4.005 | 30.686 | −10.129 | 1.00 | 63.44 | C |
| ATOM | 75 | C | GLY | A | 269 | −3.523 | 31.020 | −8.723 | 1.00 | 62.74 | C |
| ATOM | 76 | O | GLY | A | 269 | −3.081 | 30.147 | −7.971 | 1.00 | 62.53 | O |
| ATOM | 77 | N | PRO | A | 270 | −3.623 | 32.295 | −8.369 | 1.00 | 61.62 | N |
| ATOM | 78 | CA | PRO | A | 270 | −3.226 | 32.784 | −7.046 | 1.00 | 60.71 | C |
| ATOM | 79 | CB | PRO | A | 270 | −3.788 | 34.203 | −7.019 | 1.00 | 61.06 | C |
| ATOM | 80 | CG | PRO | A | 270 | −3.818 | 34.625 | −8.460 | 1.00 | 61.54 | C |
| ATOM | 81 | CD | PRO | A | 270 | −4.122 | 33.374 | −9.238 | 1.00 | 61.80 | C |
| ATOM | 82 | C | PRO | A | 270 | −3.805 | 31.980 | −5.894 | 1.00 | 59.59 | C |
| ATOM | 83 | O | PRO | A | 270 | −3.131 | 31.832 | −4.878 | 1.00 | 59.46 | O |
| ATOM | 84 | N | ALA | A | 271 | −5.019 | 31.464 | −6.034 | 1.00 | 57.85 | N |
| ATOM | 85 | CA | ALA | A | 271 | −5.583 | 30.679 | −4.955 | 1.00 | 56.85 | C |
| ATOM | 86 | CB | ALA | A | 271 | −6.933 | 30.115 | −5.330 | 1.00 | 56.89 | C |
| ATOM | 87 | C | ALA | A | 271 | −4.642 | 29.553 | −4.624 | 1.00 | 55.87 | C |
| ATOM | 88 | O | ALA | A | 271 | −4.414 | 29.277 | −3.461 | 1.00 | 54.85 | O |
| ATOM | 89 | N | GLU | A | 272 | −4.123 | 28.906 | −5.669 | 1.00 | 55.03 | N |
| ATOM | 90 | CA | GLU | A | 272 | −3.219 | 27.766 | −5.542 | 1.00 | 54.75 | C |
| ATOM | 91 | CB | GLU | A | 272 | −2.898 | 27.199 | −6.926 | 1.00 | 55.29 | C |
| ATOM | 92 | CG | GLU | A | 272 | −2.191 | 25.854 | −6.930 | 1.00 | 58.02 | C |
| ATOM | 93 | CD | GLU | A | 272 | −3.117 | 24.691 | −6.580 | 1.00 | 62.00 | C |
| ATOM | 94 | OE1 | GLU | A | 272 | −4.000 | 24.868 | −5.694 | 1.00 | 62.47 | O |
| ATOM | 95 | OE2 | GLU | A | 272 | −2.966 | 23.598 | −7.202 | 1.00 | 63.34 | O |
| ATOM | 96 | C | GLU | A | 272 | −1.923 | 28.139 | −4.819 | 1.00 | 53.60 | C |
| ATOM | 97 | O | GLU | A | 272 | −1.420 | 27.370 | −4.019 | 1.00 | 52.90 | O |
| ATOM | 98 | N | LEU | A | 273 | −1.392 | 29.323 | −5.100 | 1.00 | 53.00 | N |
| ATOM | 99 | CA | LEU | A | 273 | −0.187 | 29.775 | −4.440 | 1.00 | 52.74 | C |
| ATOM | 100 | CB | LEU | A | 273 | 0.295 | 31.091 | −5.029 | 1.00 | 53.24 | C |
| ATOM | 101 | CG | LEU | A | 273 | 0.578 | 31.167 | −6.522 | 1.00 | 54.92 | C |
| ATOM | 102 | CD1 | LEU | A | 273 | 1.291 | 32.486 | −6.819 | 1.00 | 55.95 | C |
| ATOM | 103 | CD2 | LEU | A | 273 | 1.400 | 29.983 | −7.007 | 1.00 | 56.45 | C |
| ATOM | 104 | C | LEU | A | 273 | −0.489 | 30.012 | −2.984 | 1.00 | 51.81 | C |
| ATOM | 105 | O | LEU | A | 273 | 0.302 | 29.668 | −2.103 | 1.00 | 51.57 | O |
| ATOM | 106 | N | GLU | A | 274 | −1.638 | 30.629 | −2.729 | 1.00 | 50.55 | N |
| ATOM | 107 | CA | GLU | A | 274 | −2.025 | 30.937 | −1.362 | 1.00 | 49.74 | C |
| ATOM | 108 | CB | GLU | A | 274 | −3.276 | 31.827 | −1.321 | 1.00 | 49.87 | C |
| ATOM | 109 | CG | GLU | A | 274 | −3.526 | 32.505 | 0.016 | 1.00 | 50.96 | C |
| ATOM | 110 | CD | GLU | A | 274 | −2.357 | 33.359 | 0.481 | 1.00 | 52.00 | C |
| ATOM | 111 | OE1 | GLU | A | 274 | −1.827 | 34.142 | −0.341 | 1.00 | 52.15 | O |
| ATOM | 112 | OE2 | GLU | A | 274 | −1.974 | 33.236 | 1.665 | 1.00 | 56.29 | O |
| ATOM | 113 | C | GLU | A | 274 | −2.248 | 29.627 | −0.629 | 1.00 | 48.42 | C |
| ATOM | 114 | O | GLU | A | 274 | −1.817 | 29.475 | 0.505 | 1.00 | 48.21 | O |
| ATOM | 115 | N | HIS | A | 275 | −2.879 | 28.668 | −1.285 | 1.00 | 47.05 | N |
| ATOM | 116 | CA | HIS | A | 275 | −3.148 | 27.394 | −0.623 | 1.00 | 46.52 | C |
| ATOM | 117 | CB | HIS | A | 275 | −3.928 | 26.445 | −1.530 | 1.00 | 46.60 | C |
| ATOM | 118 | CG | HIS | A | 275 | −4.241 | 25.113 | −0.905 | 1.00 | 48.13 | C |
| ATOM | 119 | ND1 | HIS | A | 275 | −5.321 | 24.914 | −0.070 | 1.00 | 49.08 | N |
| ATOM | 120 | CE1 | HIS | A | 275 | −5.360 | 23.647 | 0.312 | 1.00 | 51.04 | C |
| ATOM | 121 | NE2 | HIS | A | 275 | −4.345 | 23.009 | −0.248 | 1.00 | 50.51 | N |
| ATOM | 122 | CD2 | HIS | A | 275 | −3.631 | 23.903 | −1.019 | 1.00 | 50.94 | C |
| ATOM | 123 | C | HIS | A | 275 | −1.857 | 26.721 | −0.193 | 1.00 | 45.34 | C |
| ATOM | 124 | O | HIS | A | 275 | −1.766 | 26.218 | 0.917 | 1.00 | 45.39 | O |
| ATOM | 125 | N | LEU | A | 276 | −0.866 | 26.692 | −1.078 | 1.00 | 44.20 | N |
| ATOM | 126 | CA | LEU | A | 276 | 0.405 | 26.056 | −0.762 | 1.00 | 43.43 | C |
| ATOM | 127 | CB | LEU | A | 276 | 1.243 | 25.889 | −2.034 | 1.00 | 43.73 | C |
| ATOM | 128 | CG | LEU | A | 276 | 2.652 | 25.291 | −1.877 | 1.00 | 43.56 | C |
| ATOM | 129 | CD1 | LEU | A | 276 | 2.568 | 23.818 | −1.557 | 1.00 | 43.50 | C |
| ATOM | 130 | CD2 | LEU | A | 276 | 3.486 | 25.536 | −3.127 | 1.00 | 43.26 | C |
| ATOM | 131 | C | LEU | A | 276 | 1.172 | 26.881 | 0.310 | 1.00 | 42.47 | C |
| ATOM | 132 | O | LEU | A | 276 | 1.879 | 26.346 | 1.153 | 1.00 | 40.97 | O |
| ATOM | 133 | N | ALA | A | 277 | 1.035 | 28.190 | 0.302 | 1.00 | 41.76 | N |
| ATOM | 134 | CA | ALA | A | 277 | 1.737 | 28.961 | 1.324 | 1.00 | 41.55 | C |
| ATOM | 135 | CB | ALA | A | 277 | 1.716 | 30.419 | 1.004 | 1.00 | 41.80 | C |
| ATOM | 136 | C | ALA | A | 277 | 1.124 | 28.718 | 2.699 | 1.00 | 41.51 | C |
| ATOM | 137 | O | ALA | A | 277 | 1.832 | 28.631 | 3.708 | 1.00 | 41.21 | O |
| ATOM | 138 | N | GLN | A | 278 | −0.192 | 28.545 | 2.734 | 1.00 | 41.43 | N |
| ATOM | 139 | CA | GLN | A | 278 | −0.870 | 28.331 | 4.000 | 1.00 | 41.87 | C |
| ATOM | 140 | CB | GLN | A | 278 | −2.373 | 28.610 | 3.879 | 1.00 | 42.96 | C |
| ATOM | 141 | CG | GLN | A | 278 | −2.684 | 30.122 | 3.749 | 1.00 | 45.63 | C |
| ATOM | 142 | CD | GLN | A | 278 | −4.182 | 30.476 | 3.501 | 1.00 | 49.42 | C |
| ATOM | 143 | OE1 | GLN | A | 278 | −5.098 | 29.751 | 3.921 | 1.00 | 52.18 | O |
| ATOM | 144 | NE2 | GLN | A | 278 | −4.410 | 31.605 | 2.836 | 1.00 | 50.77 | N |
| ATOM | 145 | C | GLN | A | 278 | −0.570 | 26.945 | 4.521 | 1.00 | 40.99 | C |
| ATOM | 146 | O | GLN | A | 278 | −0.411 | 26.736 | 5.719 | 1.00 | 40.26 | O |
| ATOM | 147 | N | ASN | A | 279 | −0.449 | 25.993 | 3.621 | 1.00 | 40.42 | N |
| ATOM | 148 | CA | ASN | A | 279 | −0.117 | 24.635 | 4.021 | 1.00 | 40.51 | C |
| ATOM | 149 | CB | ASN | A | 279 | −0.075 | 23.755 | 2.795 | 1.00 | 41.05 | C |
| ATOM | 150 | CG | ASN | A | 279 | 0.276 | 22.322 | 3.120 | 1.00 | 44.76 | C |
| ATOM | 151 | OD1 | ASN | A | 279 | −0.554 | 21.573 | 3.640 | 1.00 | 48.82 | O |
| ATOM | 152 | ND2 | ASN | A | 279 | 1.522 | 21.922 | 2.818 | 1.00 | 51.37 | N |
| ATOM | 153 | C | ASN | A | 279 | 1.242 | 24.550 | 4.702 | 1.00 | 39.60 | C |
| ATOM | 154 | O | ASN | A | 279 | 1.411 | 23.920 | 5.746 | 1.00 | 40.22 | O |
| ATOM | 155 | N | ILE | A | 280 | 2.219 | 25.170 | 4.073 | 1.00 | 38.39 | N |
| ATOM | 156 | CA | ILE | A | 280 | 3.563 | 25.150 | 4.550 | 1.00 | 37.67 | C |
| ATOM | 157 | CB | ILE | A | 280 | 4.493 | 25.612 | 3.419 | 1.00 | 37.61 | C |
| ATOM | 158 | CG1 | ILE | A | 280 | 4.506 | 24.532 | 2.322 | 1.00 | 38.20 | C |
| ATOM | 159 | CD1 | ILE | A | 280 | 5.666 | 24.586 | 1.341 | 1.00 | 39.82 | C |
| ATOM | 160 | CG2 | ILE | A | 280 | 5.892 | 25.864 | 3.974 | 1.00 | 38.20 | C |
| ATOM | 161 | C | ILE | A | 280 | 3.697 | 25.986 | 5.823 | 1.00 | 36.72 | C |
| ATOM | 162 | O | ILE | A | 280 | 4.355 | 25.583 | 6.765 | 1.00 | 35.34 | O |
| ATOM | 163 | N | SER | A | 281 | 3.060 | 27.146 | 5.842 | 1.00 | 36.72 | N |
| ATOM | 164 | CA | SER | A | 281 | 3.018 | 27.979 | 7.043 | 1.00 | 36.93 | C |
| ATOM | 165 | CB | SER | A | 281 | 2.091 | 29.161 | 6.831 | 1.00 | 37.23 | C |
| ATOM | 166 | OG | SER | A | 281 | 2.681 | 30.069 | 5.909 | 1.00 | 38.94 | O |
| ATOM | 167 | C | SER | A | 281 | 2.543 | 27.219 | 8.237 | 1.00 | 36.20 | C |
| ATOM | 168 | O | SER | A | 281 | 3.131 | 27.298 | 9.295 | 1.00 | 35.96 | O |
| ATOM | 169 | N | LYS | A | 282 | 1.467 | 26.470 | 8.068 | 1.00 | 36.67 | N |
| ATOM | 170 | CA | LYS | A | 282 | 0.880 | 25.704 | 9.158 | 1.00 | 37.15 | C |
| ATOM | 171 | CB | LYS | A | 282 | −0.479 | 25.107 | 8.730 | 1.00 | 38.10 | C |
| ATOM | 172 | CG | LYS | A | 282 | −1.305 | 24.498 | 9.860 | 1.00 | 40.15 | C |
| ATOM | 173 | CD | LYS | A | 282 | −2.704 | 24.142 | 9.337 | 1.00 | 46.19 | C |
| ATOM | 174 | CE | LYS | A | 282 | −3.414 | 23.021 | 10.139 | 1.00 | 49.27 | C |
| ATOM | 175 | NZ | LYS | A | 282 | −3.455 | 21.732 | 9.376 | 1.00 | 51.98 | N |
| ATOM | 176 | C | LYS | A | 282 | 1.794 | 24.585 | 9.604 | 1.00 | 36.50 | C |
| ATOM | 177 | O | LYS | A | 282 | 1.940 | 24.307 | 10.798 | 1.00 | 35.79 | O |
| ATOM | 178 | N | SER | A | 283 | 2.409 | 23.916 | 8.643 | 1.00 | 35.63 | N |
| ATOM | 179 | CA | SER | A | 283 | 3.316 | 22.836 | 8.997 | 1.00 | 34.48 | C |
| ATOM | 180 | CB | SER | A | 283 | 3.848 | 22.181 | 7.744 | 1.00 | 34.45 | C |
| ATOM | 181 | OG | SER | A | 283 | 2.754 | 21.707 | 7.003 | 1.00 | 34.06 | O |
| ATOM | 182 | C | SER | A | 283 | 4.441 | 23.381 | 9.830 | 1.00 | 33.76 | C |
| ATOM | 183 | O | SER | A | 283 | 4.830 | 22.758 | 10.789 | 1.00 | 33.46 | O |
| ATOM | 184 | N | HIS | A | 284 | 4.943 | 24.552 | 9.471 | 1.00 | 33.35 | N |
| ATOM | 185 | CA | HIS | A | 284 | 6.007 | 25.195 | 10.224 | 1.00 | 34.42 | C |
| ATOM | 186 | CB | HIS | A | 284 | 6.402 | 26.460 | 9.513 | 1.00 | 34.00 | C |
| ATOM | 187 | CG | HIS | A | 284 | 7.306 | 27.355 | 10.288 | 1.00 | 34.20 | C |
| ATOM | 188 | ND1 | HIS | A | 284 | 8.677 | 27.240 | 10.249 | 1.00 | 32.90 | N |
| ATOM | 189 | CE1 | HIS | A | 284 | 9.221 | 28.201 | 10.968 | 1.00 | 33.90 | C |
| ATOM | 190 | NE2 | HIS | A | 284 | 8.249 | 28.946 | 11.472 | 1.00 | 35.01 | N |
| ATOM | 191 | CD2 | HIS | A | 284 | 7.042 | 28.448 | 11.045 | 1.00 | 36.29 | C |
| ATOM | 192 | C | HIS | A | 284 | 5.629 | 25.530 | 11.660 | 1.00 | 35.30 | C |
| ATOM | 193 | O | HIS | A | 284 | 6.391 | 25.244 | 12.589 | 1.00 | 34.75 | O |
| ATOM | 194 | N | LEU | A | 285 | 4.469 | 26.172 | 11.824 | 1.00 | 36.71 | N |
| ATOM | 195 | CA | LEU | A | 285 | 3.947 | 26.548 | 13.136 | 1.00 | 37.40 | C |
| ATOM | 196 | CB | LEU | A | 285 | 2.579 | 27.208 | 12.997 | 1.00 | 38.70 | C |
| ATOM | 197 | CG | LEU | A | 285 | 1.838 | 27.637 | 14.272 | 1.00 | 41.84 | C |
| ATOM | 198 | CD1 | LEU | A | 285 | 2.525 | 28.826 | 14.913 | 1.00 | 42.97 | C |
| ATOM | 199 | CD2 | LEU | A | 285 | 0.385 | 27.982 | 13.934 | 1.00 | 43.37 | C |
| ATOM | 200 | C | LEU | A | 285 | 3.846 | 25.351 | 14.046 | 1.00 | 37.27 | C |
| ATOM | 201 | O | LEU | A | 285 | 4.279 | 25.403 | 15.206 | 1.00 | 37.97 | O |
| ATOM | 202 | N | GLU | A | 286 | 3.369 | 24.237 | 13.510 | 1.00 | 36.60 | N |
| ATOM | 203 | CA | GLU | A | 286 | 3.134 | 23.058 | 14.337 | 1.00 | 36.96 | C |
| ATOM | 204 | CB | GLU | A | 286 | 1.988 | 22.229 | 13.722 | 1.00 | 37.46 | C |
| ATOM | 205 | CG | GLU | A | 286 | 0.751 | 23.091 | 13.462 | 1.00 | 42.20 | C |
| ATOM | 206 | CD | GLU | A | 286 | −0.487 | 22.289 | 13.142 | 1.00 | 48.25 | C |
| ATOM | 207 | OE1 | GLU | A | 286 | −0.343 | 21.219 | 12.510 | 1.00 | 52.12 | O |
| ATOM | 208 | OE2 | GLU | A | 286 | −1.601 | 22.726 | 13.534 | 1.00 | 52.12 | O |
| ATOM | 209 | C | GLU | A | 286 | 4.350 | 22.157 | 14.572 | 1.00 | 35.80 | C |
| ATOM | 210 | O | GLU | A | 286 | 4.231 | 21.190 | 15.303 | 1.00 | 34.55 | O |
| ATOM | 211 | N | THR | A | 287 | 5.485 | 22.439 | 13.931 | 1.00 | 34.46 | N |
| ATOM | 212 | CA | THR | A | 287 | 6.684 | 21.616 | 14.118 | 1.00 | 34.18 | C |
| ATOM | 213 | CB | THR | A | 287 | 7.154 | 20.957 | 12.814 | 1.00 | 33.50 | C |
| ATOM | 214 | OG1 | THR | A | 287 | 7.367 | 21.952 | 11.807 | 1.00 | 30.59 | O |
| ATOM | 215 | CG2 | THR | A | 287 | 6.122 | 20.050 | 12.250 | 1.00 | 34.40 | C |
| ATOM | 216 | C | THR | A | 287 | 7.879 | 22.382 | 14.670 | 1.00 | 34.80 | C |
| ATOM | 217 | O | THR | A | 287 | 8.965 | 21.849 | 14.681 | 1.00 | 35.10 | O |
| ATOM | 218 | N | CYS | A | 288 | 7.709 | 23.638 | 15.047 | 1.00 | 35.78 | N |
| ATOM | 219 | CA | CYS | A | 288 | 8.764 | 24.346 | 15.740 | 1.00 | 36.72 | C |
| ATOM | 220 | CB | CYS | A | 288 | 8.642 | 25.851 | 15.552 | 1.00 | 36.32 | C |
| ATOM | 221 | SG | CYS | A | 288 | 9.235 | 26.481 | 13.966 | 1.00 | 39.84 | S |
| ATOM | 222 | C | CYS | A | 288 | 8.682 | 23.996 | 17.230 | 1.00 | 36.71 | C |
| ATOM | 223 | O | CYS | A | 288 | 7.602 | 23.895 | 17.792 | 1.00 | 36.87 | O |
| ATOM | 224 | N | GLN | A | 289 | 9.830 | 23.812 | 17.866 | 1.00 | 37.19 | N |
| ATOM | 225 | CA | GLN | A | 289 | 9.862 | 23.516 | 19.278 | 1.00 | 37.25 | C |
| ATOM | 226 | CB | GLN | A | 289 | 11.279 | 23.153 | 19.694 | 1.00 | 37.24 | C |
| ATOM | 227 | CG | GLN | A | 289 | 11.409 | 22.864 | 21.168 | 1.00 | 38.95 | C |
| ATOM | 228 | CD | GLN | A | 289 | 12.661 | 22.072 | 21.551 | 1.00 | 40.39 | C |
| ATOM | 229 | OE1 | GLN | A | 289 | 13.694 | 22.091 | 20.847 | 1.00 | 39.04 | O |
| ATOM | 230 | NE2 | GLN | A | 289 | 12.567 | 21.369 | 22.682 | 1.00 | 38.81 | N |
| ATOM | 231 | C | GLN | A | 289 | 9.288 | 24.683 | 20.141 | 1.00 | 37.72 | C |
| ATOM | 232 | O | GLN | A | 289 | 8.603 | 24.432 | 21.124 | 1.00 | 37.80 | O |
| ATOM | 233 | N | TYR | A | 290 | 9.519 | 25.935 | 19.744 | 1.00 | 38.28 | N |
| ATOM | 234 | CA | TYR | A | 290 | 9.072 | 27.105 | 20.501 | 1.00 | 38.43 | C |
| ATOM | 235 | CB | TYR | A | 290 | 10.268 | 27.813 | 21.152 | 1.00 | 38.66 | C |
| ATOM | 236 | CG | TYR | A | 290 | 11.241 | 26.912 | 21.906 | 1.00 | 37.22 | C |
| ATOM | 237 | CD1 | TYR | A | 290 | 12.438 | 26.540 | 21.332 | 1.00 | 35.94 | C |
| ATOM | 238 | CE1 | TYR | A | 290 | 13.339 | 25.731 | 21.989 | 1.00 | 34.88 | C |
| ATOM | 239 | CZ | TYR | A | 290 | 13.061 | 25.293 | 23.244 | 1.00 | 35.86 | C |
| ATOM | 240 | OH | TYR | A | 290 | 13.983 | 24.491 | 23.870 | 1.00 | 34.34 | O |
| ATOM | 241 | CE2 | TYR | A | 290 | 11.872 | 25.660 | 23.866 | 1.00 | 36.55 | C |
| ATOM | 242 | CD2 | TYR | A | 290 | 10.978 | 26.487 | 23.193 | 1.00 | 35.75 | C |
| ATOM | 243 | C | TYR | A | 290 | 8.329 | 28.158 | 19.650 | 1.00 | 39.65 | C |
| ATOM | 244 | O | TYR | A | 290 | 8.582 | 28.309 | 18.443 | 1.00 | 39.27 | O |
| ATOM | 245 | N | LEU | A | 291 | 7.443 | 28.924 | 20.296 | 1.00 | 40.18 | N |
| ATOM | 246 | CA | LEU | A | 291 | 6.740 | 29.985 | 19.611 | 1.00 | 40.92 | C |
| ATOM | 247 | CB | LEU | A | 291 | 5.444 | 30.367 | 20.327 | 1.00 | 41.69 | C |
| ATOM | 248 | CG | LEU | A | 291 | 4.371 | 29.301 | 20.591 | 1.00 | 43.47 | C |
| ATOM | 249 | CD1 | LEU | A | 291 | 3.295 | 29.850 | 21.586 | 1.00 | 45.13 | C |
| ATOM | 250 | CD2 | LEU | A | 291 | 3.716 | 28.890 | 19.300 | 1.00 | 43.40 | C |
| ATOM | 251 | C | LEU | A | 291 | 7.635 | 31.202 | 19.487 | 1.00 | 41.12 | C |
| ATOM | 252 | O | LEU | A | 291 | 8.382 | 31.554 | 20.393 | 1.00 | 40.66 | O |
| ATOM | 253 | N | ARG | A | 292 | 7.508 | 31.876 | 18.356 | 1.00 | 41.62 | N |
| ATOM | 254 | CA | ARG | A | 292 | 8.317 | 33.034 | 18.065 | 1.00 | 42.34 | C |
| ATOM | 255 | CB | ARG | A | 292 | 7.830 | 33.695 | 16.766 | 1.00 | 42.90 | C |
| ATOM | 256 | CG | ARG | A | 292 | 8.712 | 34.814 | 16.241 | 1.00 | 45.07 | C |
| ATOM | 257 | CD | ARG | A | 292 | 10.053 | 34.331 | 15.752 | 1.00 | 48.94 | C |
| ATOM | 258 | NE | ARG | A | 292 | 10.873 | 35.373 | 15.134 | 1.00 | 50.47 | N |
| ATOM | 259 | CZ | ARG | A | 292 | 10.786 | 35.746 | 13.871 | 1.00 | 54.17 | C |
| ATOM | 260 | NH1 | ARG | A | 292 | 9.874 | 35.206 | 13.051 | 1.00 | 55.75 | N |
| ATOM | 261 | NH2 | ARG | A | 292 | 11.605 | 36.684 | 13.420 | 1.00 | 55.53 | N |
| ATOM | 262 | C | ARG | A | 292 | 8.206 | 34.005 | 19.199 | 1.00 | 42.41 | C |
| ATOM | 263 | O | ARG | A | 292 | 9.213 | 34.496 | 19.732 | 1.00 | 41.10 | O |
| ATOM | 264 | N | GLU | A | 293 | 6.958 | 34.287 | 19.564 | 1.00 | 43.20 | N |
| ATOM | 265 | CA | GLU | A | 293 | 6.675 | 35.284 | 20.586 | 1.00 | 43.83 | C |
| ATOM | 266 | CB | GLU | A | 293 | 5.164 | 35.519 | 20.679 | 1.00 | 44.63 | C |
| ATOM | 267 | CG | GLU | A | 293 | 4.562 | 36.144 | 19.410 | 1.00 | 47.94 | C |
| ATOM | 268 | CD | GLU | A | 293 | 4.517 | 35.201 | 18.194 | 1.00 | 51.44 | C |
| ATOM | 269 | OE1 | GLU | A | 293 | 4.477 | 33.955 | 18.392 | 1.00 | 52.38 | O |
| ATOM | 270 | OE2 | GLU | A | 293 | 4.516 | 35.711 | 17.033 | 1.00 | 52.22 | O |
| ATOM | 271 | C | GLU | A | 293 | 7.299 | 34.887 | 21.928 | 1.00 | 43.00 | C |
| ATOM | 272 | O | GLU | A | 293 | 7.878 | 35.732 | 22.598 | 1.00 | 43.39 | O |
| ATOM | 273 | N | GLU | A | 294 | 7.207 | 33.613 | 22.303 | 1.00 | 42.66 | N |
| ATOM | 274 | CA | GLU | A | 294 | 7.870 | 33.111 | 23.522 | 1.00 | 42.27 | C |
| ATOM | 275 | CB | GLU | A | 294 | 7.845 | 31.564 | 23.632 | 1.00 | 43.09 | C |
| ATOM | 276 | CG | GLU | A | 294 | 6.544 | 30.813 | 23.909 | 1.00 | 45.45 | C |
| ATOM | 277 | CD | GLU | A | 294 | 6.529 | 29.398 | 23.262 | 1.00 | 47.89 | C |
| ATOM | 278 | OE1 | GLU | A | 294 | 7.355 | 28.479 | 23.591 | 1.00 | 42.98 | O |
| ATOM | 279 | OE2 | GLU | A | 294 | 5.666 | 29.204 | 22.369 | 1.00 | 50.76 | O |
| ATOM | 280 | C | GLU | A | 294 | 9.364 | 33.469 | 23.488 | 1.00 | 40.96 | C |
| ATOM | 281 | O | GLU | A | 294 | 9.919 | 33.993 | 24.447 | 1.00 | 40.53 | O |
| ATOM | 282 | N | LEU | A | 295 | 10.017 | 33.106 | 22.388 | 1.00 | 39.12 | N |
| ATOM | 283 | CA | LEU | A | 295 | 11.462 | 33.297 | 22.239 | 1.00 | 38.55 | C |
| ATOM | 284 | CB | LEU | A | 295 | 11.959 | 32.681 | 20.918 | 1.00 | 38.19 | C |
| ATOM | 285 | CG | LEU | A | 295 | 11.815 | 31.163 | 20.806 | 1.00 | 38.68 | C |
| ATOM | 286 | CD1 | LEU | A | 295 | 11.654 | 30.733 | 19.372 | 1.00 | 40.29 | C |
| ATOM | 287 | CD2 | LEU | A | 295 | 13.003 | 30.450 | 21.445 | 1.00 | 38.56 | C |
| ATOM | 288 | C | LEU | A | 295 | 11.866 | 34.759 | 22.318 | 1.00 | 37.73 | C |
| ATOM | 289 | O | LEU | A | 295 | 12.884 | 35.075 | 22.907 | 1.00 | 36.93 | O |
| ATOM | 290 | N | GLN | A | 296 | 11.052 | 35.640 | 21.749 | 1.00 | 38.09 | N |
| ATOM | 291 | CA | GLN | A | 296 | 11.323 | 37.083 | 21.755 | 1.00 | 39.08 | C |
| ATOM | 292 | CB | GLN | A | 296 | 10.390 | 37.802 | 20.775 | 1.00 | 39.49 | C |
| ATOM | 293 | CG | GLN | A | 296 | 10.709 | 37.498 | 19.284 | 1.00 | 43.93 | C |
| ATOM | 294 | CD | GLN | A | 296 | 9.671 | 38.037 | 18.263 | 1.00 | 47.60 | C |
| ATOM | 295 | OE1 | GLN | A | 296 | 8.471 | 38.104 | 18.537 | 1.00 | 50.71 | O |
| ATOM | 296 | NE2 | GLN | A | 296 | 10.152 | 38.397 | 17.079 | 1.00 | 49.60 | N |
| ATOM | 297 | C | GLN | A | 296 | 11.203 | 37.728 | 23.148 | 1.00 | 38.56 | C |
| ATOM | 298 | O | GLN | A | 296 | 11.898 | 38.696 | 23.461 | 1.00 | 38.91 | O |
| ATOM | 299 | N | GLN | A | 297 | 10.351 | 37.176 | 23.988 | 1.00 | 38.53 | N |
| ATOM | 300 | CA | GLN | A | 297 | 10.104 | 37.771 | 25.297 | 1.00 | 38.64 | C |
| ATOM | 301 | CB | GLN | A | 297 | 8.692 | 37.402 | 25.783 | 1.00 | 38.82 | C |
| ATOM | 302 | CG | GLN | A | 297 | 7.621 | 38.365 | 25.195 | 1.00 | 42.59 | C |
| ATOM | 303 | CD | GLN | A | 297 | 6.170 | 37.850 | 25.288 | 1.00 | 48.07 | C |
| ATOM | 304 | OE1 | GLN | A | 297 | 5.797 | 37.151 | 26.239 | 1.00 | 50.31 | O |
| ATOM | 305 | NE2 | GLN | A | 297 | 5.356 | 38.199 | 24.289 | 1.00 | 51.73 | N |
| ATOM | 306 | C | GLN | A | 297 | 11.177 | 37.428 | 26.347 | 1.00 | 37.73 | C |
| ATOM | 307 | O | GLN | A | 297 | 11.210 | 38.040 | 27.413 | 1.00 | 37.76 | O |
| ATOM | 308 | N | ILE | A | 298 | 12.045 | 36.452 | 26.059 | 1.00 | 36.25 | N |
| ATOM | 309 | CA | ILE | A | 298 | 13.087 | 36.084 | 27.007 | 1.00 | 34.28 | C |
| ATOM | 310 | CB | ILE | A | 298 | 12.920 | 34.651 | 27.472 | 1.00 | 34.23 | C |
| ATOM | 311 | CG1 | ILE | A | 298 | 12.999 | 33.705 | 26.282 | 1.00 | 34.17 | C |
| ATOM | 312 | CD1 | ILE | A | 298 | 13.013 | 32.277 | 26.693 | 1.00 | 37.43 | C |
| ATOM | 313 | CG2 | ILE | A | 298 | 11.588 | 34.413 | 28.205 | 1.00 | 36.82 | C |
| ATOM | 314 | C | ILE | A | 298 | 14.520 | 36.278 | 26.479 | 1.00 | 32.82 | C |
| ATOM | 315 | O | ILE | A | 298 | 15.421 | 35.612 | 26.959 | 1.00 | 32.45 | O |
| ATOM | 316 | N | THR | A | 299 | 14.748 | 37.175 | 25.518 | 1.00 | 32.29 | N |
| ATOM | 317 | CA | THR | A | 299 | 16.110 | 37.434 | 25.027 | 1.00 | 32.00 | C |
| ATOM | 318 | CB | THR | A | 299 | 16.149 | 38.398 | 23.832 | 1.00 | 32.50 | C |
| ATOM | 319 | OG1 | THR | A | 299 | 15.244 | 39.478 | 24.047 | 1.00 | 34.44 | O |
| ATOM | 320 | CG2 | THR | A | 299 | 15.653 | 37.762 | 22.528 | 1.00 | 33.01 | C |
| ATOM | 321 | C | THR | A | 299 | 17.000 | 38.007 | 26.104 | 1.00 | 31.05 | C |
| ATOM | 322 | O | THR | A | 299 | 18.212 | 37.917 | 26.015 | 1.00 | 30.68 | O |
| ATOM | 323 | N | TRP | A | 300 | 16.412 | 38.607 | 27.132 | 1.00 | 30.79 | N |
| ATOM | 324 | CA | TRP | A | 300 | 17.218 | 39.162 | 28.237 | 1.00 | 29.80 | C |
| ATOM | 325 | CB | TRP | A | 300 | 16.413 | 40.155 | 29.094 | 1.00 | 29.57 | C |
| ATOM | 326 | CG | TRP | A | 300 | 15.189 | 39.547 | 29.664 | 1.00 | 27.22 | C |
| ATOM | 327 | CD1 | TRP | A | 300 | 13.988 | 39.423 | 29.044 | 1.00 | 25.70 | C |
| ATOM | 328 | NE1 | TRP | A | 300 | 13.096 | 38.775 | 29.862 | 1.00 | 24.08 | N |
| ATOM | 329 | CE2 | TRP | A | 300 | 13.727 | 38.462 | 31.033 | 1.00 | 24.93 | C |
| ATOM | 330 | CD2 | TRP | A | 300 | 15.045 | 38.926 | 30.936 | 1.00 | 23.62 | C |
| ATOM | 331 | CE3 | TRP | A | 300 | 15.884 | 38.761 | 32.030 | 1.00 | 26.31 | C |
| ATOM | 332 | CZ3 | TRP | A | 300 | 15.415 | 38.153 | 33.128 | 1.00 | 27.39 | C |
| ATOM | 333 | CH2 | TRP | A | 300 | 14.083 | 37.700 | 33.201 | 1.00 | 28.78 | C |
| ATOM | 334 | CZ2 | TRP | A | 300 | 13.231 | 37.857 | 32.168 | 1.00 | 24.74 | C |
| ATOM | 335 | C | TRP | A | 300 | 17.803 | 38.099 | 29.155 | 1.00 | 29.45 | C |
| ATOM | 336 | O | TRP | A | 300 | 18.751 | 38.365 | 29.906 | 1.00 | 30.38 | O |
| ATOM | 337 | N | GLN | A | 301 | 17.231 | 36.897 | 29.103 | 1.00 | 29.27 | N |
| ATOM | 338 | CA | GLN | A | 301 | 17.695 | 35.781 | 29.923 | 1.00 | 29.27 | C |
| ATOM | 339 | CB | GLN | A | 301 | 16.616 | 34.701 | 30.038 | 1.00 | 28.84 | C |
| ATOM | 340 | CG | GLN | A | 301 | 15.419 | 35.171 | 30.791 | 1.00 | 30.22 | C |
| ATOM | 341 | CD | GLN | A | 301 | 14.251 | 34.170 | 30.897 | 1.00 | 33.05 | C |
| ATOM | 342 | OE1 | GLN | A | 301 | 14.340 | 33.032 | 30.464 | 1.00 | 32.79 | O |
| ATOM | 343 | NE2 | GLN | A | 301 | 13.144 | 34.633 | 31.479 | 1.00 | 34.45 | N |
| ATOM | 344 | C | GLN | A | 301 | 18.964 | 35.186 | 29.327 | 1.00 | 29.50 | C |
| ATOM | 345 | O | GLN | A | 301 | 18.926 | 34.117 | 28.725 | 1.00 | 30.22 | O |
| ATOM | 346 | N | THR | A | 302 | 20.081 | 35.884 | 29.484 | 1.00 | 29.33 | N |
| ATOM | 347 | CA | THR | A | 302 | 21.372 | 35.407 | 29.007 | 1.00 | 29.03 | C |
| ATOM | 348 | CB | THR | A | 302 | 22.271 | 36.588 | 28.567 | 1.00 | 29.13 | C |
| ATOM | 349 | OG1 | THR | A | 302 | 22.307 | 37.572 | 29.603 | 1.00 | 28.27 | O |
| ATOM | 350 | CG2 | THR | A | 302 | 21.695 | 37.317 | 27.362 | 1.00 | 29.18 | C |
| ATOM | 351 | C | THR | A | 302 | 22.024 | 34.662 | 30.165 | 1.00 | 29.23 | C |
| ATOM | 352 | O | THR | A | 302 | 21.615 | 34.836 | 31.308 | 1.00 | 29.31 | O |
| ATOM | 353 | N | PHE | A | 303 | 22.987 | 33.791 | 29.874 | 1.00 | 29.05 | N |
| ATOM | 354 | CA | PHE | A | 303 | 23.704 | 33.101 | 30.929 | 1.00 | 29.57 | C |
| ATOM | 355 | CB | PHE | A | 303 | 24.678 | 32.033 | 30.380 | 1.00 | 29.12 | C |
| ATOM | 356 | CG | PHE | A | 303 | 23.998 | 30.815 | 29.837 | 1.00 | 27.07 | C |
| ATOM | 357 | CD1 | PHE | A | 303 | 23.798 | 30.691 | 28.475 | 1.00 | 27.05 | C |
| ATOM | 358 | CE1 | PHE | A | 303 | 23.156 | 29.630 | 27.957 | 1.00 | 27.11 | C |
| ATOM | 359 | CZ | PHE | A | 303 | 22.699 | 28.639 | 28.795 | 1.00 | 27.76 | C |
| ATOM | 360 | CE2 | PHE | A | 303 | 22.884 | 28.746 | 30.156 | 1.00 | 27.13 | C |
| ATOM | 361 | CD2 | PHE | A | 303 | 23.530 | 29.833 | 30.669 | 1.00 | 26.38 | C |
| ATOM | 362 | C | PHE | A | 303 | 24.461 | 34.134 | 31.780 | 1.00 | 30.22 | C |
| ATOM | 363 | O | PHE | A | 303 | 24.913 | 35.153 | 31.291 | 1.00 | 30.40 | O |
| ATOM | 364 | N | LEU | A | 304 | 24.598 | 33.854 | 33.053 | 1.00 | 31.25 | N |
| ATOM | 365 | CA | LEU | A | 304 | 25.332 | 34.733 | 33.954 | 1.00 | 32.65 | C |
| ATOM | 366 | CB | LEU | A | 304 | 24.930 | 34.475 | 35.390 | 1.00 | 32.80 | C |
| ATOM | 367 | CG | LEU | A | 304 | 23.457 | 34.647 | 35.722 | 1.00 | 33.60 | C |
| ATOM | 368 | CD1 | LEU | A | 304 | 23.213 | 34.101 | 37.076 | 1.00 | 35.11 | C |
| ATOM | 369 | CD2 | LEU | A | 304 | 23.051 | 36.084 | 35.695 | 1.00 | 35.33 | C |
| ATOM | 370 | C | LEU | A | 304 | 26.800 | 34.424 | 33.797 | 1.00 | 34.30 | C |
| ATOM | 371 | O | LEU | A | 304 | 27.171 | 33.345 | 33.332 | 1.00 | 32.86 | O |
| ATOM | 372 | N | GLN | A | 305 | 27.647 | 35.360 | 34.195 | 1.00 | 36.15 | N |
| ATOM | 373 | CA | GLN | A | 305 | 29.077 | 35.180 | 34.006 | 1.00 | 38.54 | C |
| ATOM | 374 | CB | GLN | A | 305 | 29.852 | 36.381 | 34.584 | 1.00 | 39.68 | C |
| ATOM | 375 | CG | GLN | A | 305 | 31.293 | 36.596 | 34.029 | 1.00 | 42.48 | C |
| ATOM | 376 | CD | GLN | A | 305 | 31.436 | 36.533 | 32.488 | 1.00 | 46.39 | C |
| ATOM | 377 | OE1 | GLN | A | 305 | 30.636 | 37.118 | 31.720 | 1.00 | 46.08 | O |
| ATOM | 378 | NE2 | GLN | A | 305 | 32.497 | 35.850 | 32.041 | 1.00 | 48.27 | N |
| ATOM | 379 | C | GLN | A | 305 | 29.584 | 33.832 | 34.575 | 1.00 | 38.86 | C |
| ATOM | 380 | O | GLN | A | 305 | 30.428 | 33.194 | 33.968 | 1.00 | 39.34 | O |
| ATOM | 381 | N | GLU | A | 306 | 29.031 | 33.391 | 35.698 | 1.00 | 39.71 | N |
| ATOM | 382 | CA | GLU | A | 306 | 29.451 | 32.155 | 36.350 | 1.00 | 39.99 | C |
| ATOM | 383 | CB | GLU | A | 306 | 28.756 | 31.992 | 37.705 | 1.00 | 41.15 | C |
| ATOM | 384 | CG | GLU | A | 306 | 29.089 | 33.078 | 38.735 | 1.00 | 47.39 | C |
| ATOM | 385 | CD | GLU | A | 306 | 28.167 | 33.080 | 39.980 | 1.00 | 55.43 | C |
| ATOM | 386 | OE1 | GLU | A | 306 | 27.643 | 31.999 | 40.357 | 1.00 | 59.46 | O |
| ATOM | 387 | OE2 | GLU | A | 306 | 27.962 | 34.167 | 40.612 | 1.00 | 60.68 | O |
| ATOM | 388 | C | GLU | A | 306 | 29.132 | 30.952 | 35.472 | 1.00 | 38.37 | C |
| ATOM | 389 | O | GLU | A | 306 | 29.926 | 30.022 | 35.353 | 1.00 | 38.01 | O |
| ATOM | 390 | N | GLU | A | 307 | 27.943 | 30.967 | 34.889 | 1.00 | 36.34 | N |
| ATOM | 391 | CA | GLU | A | 307 | 27.514 | 29.927 | 33.988 | 1.00 | 34.63 | C |
| ATOM | 392 | CB | GLU | A | 307 | 26.032 | 30.117 | 33.642 | 1.00 | 35.14 | C |
| ATOM | 393 | CG | GLU | A | 307 | 25.062 | 30.047 | 34.823 | 1.00 | 34.93 | C |
| ATOM | 394 | CD | GLU | A | 307 | 23.620 | 30.319 | 34.412 | 1.00 | 36.18 | C |
| ATOM | 395 | OE1 | GLU | A | 307 | 22.785 | 29.382 | 34.543 | 1.00 | 36.82 | O |
| ATOM | 396 | OE2 | GLU | A | 307 | 23.312 | 31.467 | 33.946 | 1.00 | 32.29 | O |
| ATOM | 397 | C | GLU | A | 307 | 28.395 | 29.957 | 32.716 | 1.00 | 33.42 | C |
| ATOM | 398 | O | GLU | A | 307 | 28.752 | 28.907 | 32.177 | 1.00 | 30.28 | O |
| ATOM | 399 | N | ILE | A | 308 | 28.778 | 31.149 | 32.264 | 1.00 | 32.73 | N |
| ATOM | 400 | CA | ILE | A | 308 | 29.593 | 31.251 | 31.062 | 1.00 | 33.52 | C |
| ATOM | 401 | CB | ILE | A | 308 | 29.723 | 32.701 | 30.595 | 1.00 | 33.62 | C |
| ATOM | 402 | CG1 | ILE | A | 308 | 28.384 | 33.198 | 30.061 | 1.00 | 34.47 | C |
| ATOM | 403 | CD1 | ILE | A | 308 | 28.385 | 34.618 | 29.697 | 1.00 | 34.55 | C |
| ATOM | 404 | CG2 | ILE | A | 308 | 30.758 | 32.864 | 29.484 | 1.00 | 33.46 | C |
| ATOM | 405 | C | ILE | A | 308 | 30.933 | 30.564 | 31.324 | 1.00 | 34.15 | C |
| ATOM | 406 | O | ILE | A | 308 | 31.373 | 29.709 | 30.560 | 1.00 | 33.82 | O |
| ATOM | 407 | N | GLU | A | 309 | 31.549 | 30.899 | 32.447 | 1.00 | 35.66 | N |
| ATOM | 408 | CA | GLU | A | 309 | 32.813 | 30.284 | 32.858 | 1.00 | 36.34 | C |
| ATOM | 409 | CB | GLU | A | 309 | 33.280 | 30.879 | 34.199 | 1.00 | 37.37 | C |
| ATOM | 410 | CG | GLU | A | 309 | 33.860 | 32.290 | 34.048 | 1.00 | 41.64 | C |
| ATOM | 411 | CD | GLU | A | 309 | 33.687 | 33.171 | 35.295 | 1.00 | 46.42 | C |
| ATOM | 412 | OE1 | GLU | A | 309 | 33.419 | 32.615 | 36.387 | 1.00 | 49.14 | O |
| ATOM | 413 | OE2 | GLU | A | 309 | 33.805 | 34.426 | 35.174 | 1.00 | 51.22 | O |
| ATOM | 414 | C | GLU | A | 309 | 32.718 | 28.766 | 32.941 | 1.00 | 35.17 | C |
| ATOM | 415 | O | GLU | A | 309 | 33.580 | 28.072 | 32.431 | 1.00 | 35.55 | O |
| ATOM | 416 | N | ASN | A | 310 | 31.679 | 28.249 | 33.578 | 1.00 | 34.21 | N |
| ATOM | 417 | CA | ASN | A | 310 | 31.478 | 26.817 | 33.603 | 1.00 | 34.07 | C |
| ATOM | 418 | CB | ASN | A | 310 | 30.168 | 26.511 | 34.282 | 1.00 | 35.39 | C |
| ATOM | 419 | CG | ASN | A | 310 | 29.871 | 25.036 | 34.346 | 1.00 | 38.20 | C |
| ATOM | 420 | OD1 | ASN | A | 310 | 30.608 | 24.260 | 34.994 | 1.00 | 45.99 | O |
| ATOM | 421 | ND2 | ASN | A | 310 | 28.778 | 24.629 | 33.712 | 1.00 | 42.62 | N |
| ATOM | 422 | C | ASN | A | 310 | 31.488 | 26.202 | 32.212 | 1.00 | 33.34 | C |
| ATOM | 423 | O | ASN | A | 310 | 32.171 | 25.193 | 31.987 | 1.00 | 33.73 | O |
| ATOM | 424 | N | TYR | A | 311 | 30.780 | 26.795 | 31.247 | 1.00 | 31.41 | N |
| ATOM | 425 | CA | TYR | A | 311 | 30.793 | 26.215 | 29.919 | 1.00 | 30.92 | C |
| ATOM | 426 | CB | TYR | A | 311 | 29.821 | 26.904 | 28.959 | 1.00 | 30.60 | C |
| ATOM | 427 | CG | TYR | A | 311 | 28.362 | 26.519 | 29.117 | 1.00 | 29.73 | C |
| ATOM | 428 | CD1 | TYR | A | 311 | 27.421 | 27.465 | 29.445 | 1.00 | 28.73 | C |
| ATOM | 429 | CE1 | TYR | A | 311 | 26.090 | 27.149 | 29.590 | 1.00 | 29.50 | C |
| ATOM | 430 | CZ | TYR | A | 311 | 25.671 | 25.875 | 29.403 | 1.00 | 29.74 | C |
| ATOM | 431 | OH | TYR | A | 311 | 24.332 | 25.610 | 29.557 | 1.00 | 32.23 | O |
| ATOM | 432 | CE2 | TYR | A | 311 | 26.578 | 24.897 | 29.056 | 1.00 | 29.37 | C |
| ATOM | 433 | CD2 | TYR | A | 311 | 27.925 | 25.224 | 28.915 | 1.00 | 29.13 | C |
| ATOM | 434 | C | TYR | A | 311 | 32.192 | 26.310 | 29.367 | 1.00 | 31.46 | C |
| ATOM | 435 | O | TYR | A | 311 | 32.635 | 25.434 | 28.636 | 1.00 | 30.86 | O |
| ATOM | 436 | N | GLN | A | 312 | 32.890 | 27.398 | 29.688 | 1.00 | 32.56 | N |
| ATOM | 437 | CA | GLN | A | 312 | 34.251 | 27.554 | 29.209 | 1.00 | 33.54 | C |
| ATOM | 438 | CB | GLN | A | 312 | 34.706 | 29.010 | 29.300 | 1.00 | 33.50 | C |
| ATOM | 439 | CG | GLN | A | 312 | 34.082 | 29.859 | 28.239 | 1.00 | 33.49 | C |
| ATOM | 440 | CD | GLN | A | 312 | 34.392 | 31.302 | 28.461 | 1.00 | 34.18 | C |
| ATOM | 441 | OE1 | GLN | A | 312 | 34.841 | 31.667 | 29.542 | 1.00 | 35.47 | O |
| ATOM | 442 | NE2 | GLN | A | 312 | 34.172 | 32.127 | 27.449 | 1.00 | 31.68 | N |
| ATOM | 443 | C | GLN | A | 312 | 35.202 | 26.603 | 29.928 | 1.00 | 34.41 | C |
| ATOM | 444 | O | GLN | A | 312 | 36.250 | 26.270 | 29.394 | 1.00 | 34.43 | O |
| ATOM | 445 | N | ASN | A | 313 | 34.834 | 26.151 | 31.116 | 1.00 | 35.17 | N |
| ATOM | 446 | CA | ASN | A | 313 | 35.680 | 25.192 | 31.827 | 1.00 | 36.70 | C |
| ATOM | 447 | CB | ASN | A | 313 | 35.383 | 25.209 | 33.333 | 1.00 | 37.66 | C |
| ATOM | 448 | CG | ASN | A | 313 | 35.992 | 26.423 | 34.051 | 1.00 | 41.22 | C |
| ATOM | 449 | OD1 | ASN | A | 313 | 36.882 | 27.096 | 33.522 | 1.00 | 42.54 | O |
| ATOM | 450 | ND2 | ASN | A | 313 | 35.478 | 26.717 | 35.265 | 1.00 | 44.69 | N |
| ATOM | 451 | C | ASN | A | 313 | 35.531 | 23.745 | 31.310 | 1.00 | 36.84 | C |
| ATOM | 452 | O | ASN | A | 313 | 36.405 | 22.889 | 31.568 | 1.00 | 36.22 | O |
| ATOM | 453 | N | LYS | A | 314 | 34.412 | 23.459 | 30.619 | 1.00 | 36.29 | N |
| ATOM | 454 | CA | LYS | A | 314 | 34.166 | 22.111 | 30.110 | 1.00 | 35.35 | C |
| ATOM | 455 | CB | LYS | A | 314 | 32.761 | 21.940 | 29.527 | 1.00 | 35.14 | C |
| ATOM | 456 | CG | LYS | A | 314 | 31.628 | 22.032 | 30.526 | 1.00 | 35.45 | C |
| ATOM | 457 | CD | LYS | A | 314 | 30.290 | 21.978 | 29.787 | 1.00 | 38.14 | C |
| ATOM | 458 | CE | LYS | A | 314 | 29.085 | 22.009 | 30.733 | 1.00 | 39.70 | C |
| ATOM | 459 | NZ | LYS | A | 314 | 29.206 | 21.049 | 31.917 | 1.00 | 44.38 | N |
| ATOM | 460 | C | LYS | A | 314 | 35.186 | 21.752 | 29.052 | 1.00 | 35.29 | C |
| ATOM | 461 | O | LYS | A | 314 | 35.676 | 22.605 | 28.307 | 1.00 | 35.12 | O |
| ATOM | 462 | N | GLN | A | 315 | 35.484 | 20.452 | 29.000 | 1.00 | 34.85 | N |
| ATOM | 463 | CA | GLN | A | 315 | 36.430 | 19.906 | 28.048 | 1.00 | 34.64 | C |
| ATOM | 464 | CB | GLN | A | 315 | 36.771 | 18.469 | 28.417 | 1.00 | 34.90 | C |
| ATOM | 465 | CG | GLN | A | 315 | 37.502 | 18.260 | 29.733 | 1.00 | 39.29 | C |
| ATOM | 466 | CD | GLN | A | 315 | 38.919 | 18.804 | 29.697 | 1.00 | 44.94 | C |
| ATOM | 467 | OE1 | GLN | A | 315 | 39.685 | 18.490 | 28.787 | 1.00 | 50.63 | O |
| ATOM | 468 | NE2 | GLN | A | 315 | 39.264 | 19.634 | 30.677 | 1.00 | 47.61 | N |
| ATOM | 469 | C | GLN | A | 315 | 35.846 | 19.884 | 26.647 | 1.00 | 33.86 | C |
| ATOM | 470 | O | GLN | A | 315 | 34.631 | 19.748 | 26.460 | 1.00 | 32.99 | O |
| ATOM | 471 | N | ARG | A | 316 | 36.729 | 19.965 | 25.664 | 1.00 | 32.95 | N |
| ATOM | 472 | CA | ARG | A | 316 | 36.315 | 19.977 | 24.292 | 1.00 | 32.86 | C |
| ATOM | 473 | CB | ARG | A | 316 | 37.519 | 19.829 | 23.385 | 1.00 | 33.76 | C |
| ATOM | 474 | CG | ARG | A | 316 | 37.205 | 20.195 | 21.947 | 1.00 | 38.32 | C |
| ATOM | 475 | CD | ARG | A | 316 | 38.414 | 20.275 | 21.047 | 1.00 | 43.87 | C |
| ATOM | 476 | NE | ARG | A | 316 | 38.022 | 20.301 | 19.640 | 1.00 | 49.79 | N |
| ATOM | 477 | CZ | ARG | A | 316 | 38.804 | 19.922 | 18.624 | 1.00 | 53.81 | C |
| ATOM | 478 | NH1 | ARG | A | 316 | 40.036 | 19.479 | 18.847 | 1.00 | 55.22 | N |
| ATOM | 479 | NH2 | ARG | A | 316 | 38.347 | 19.990 | 17.381 | 1.00 | 55.07 | N |
| ATOM | 480 | C | ARG | A | 316 | 35.289 | 18.895 | 23.966 | 1.00 | 31.22 | C |
| ATOM | 481 | O | ARG | A | 316 | 34.241 | 19.187 | 23.405 | 1.00 | 28.96 | O |
| ATOM | 482 | N | GLU | A | 317 | 35.575 | 17.649 | 24.327 | 1.00 | 30.76 | N |
| ATOM | 483 | CA | GLU | A | 317 | 34.680 | 16.574 | 23.930 | 1.00 | 30.63 | C |
| ATOM | 484 | CB | GLU | A | 317 | 35.315 | 15.181 | 24.082 | 1.00 | 31.32 | C |
| ATOM | 485 | CG | GLU | A | 317 | 35.547 | 14.697 | 25.484 | 1.00 | 33.23 | C |
| ATOM | 486 | CD | GLU | A | 317 | 36.850 | 15.194 | 26.090 | 1.00 | 38.72 | C |
| ATOM | 487 | OE1 | GLU | A | 317 | 37.349 | 14.502 | 27.024 | 1.00 | 44.77 | O |
| ATOM | 488 | OE2 | GLU | A | 317 | 37.365 | 16.259 | 25.674 | 1.00 | 34.98 | O |
| ATOM | 489 | C | GLU | A | 317 | 33.351 | 16.695 | 24.640 | 1.00 | 29.33 | C |
| ATOM | 490 | O | GLU | A | 317 | 32.344 | 16.314 | 24.099 | 1.00 | 30.11 | O |
| ATOM | 491 | N | VAL | A | 318 | 33.365 | 17.256 | 25.829 | 1.00 | 27.92 | N |
| ATOM | 492 | CA | VAL | A | 318 | 32.173 | 17.464 | 26.628 | 1.00 | 27.86 | C |
| ATOM | 493 | CB | VAL | A | 318 | 32.529 | 17.835 | 28.082 | 1.00 | 28.20 | C |
| ATOM | 494 | CG1 | VAL | A | 318 | 31.275 | 18.177 | 28.870 | 1.00 | 28.77 | C |
| ATOM | 495 | CG2 | VAL | A | 318 | 33.263 | 16.646 | 28.741 | 1.00 | 29.97 | C |
| ATOM | 496 | C | VAL | A | 318 | 31.244 | 18.528 | 26.013 | 1.00 | 27.21 | C |
| ATOM | 497 | O | VAL | A | 318 | 30.048 | 18.287 | 25.886 | 1.00 | 26.50 | O |
| ATOM | 498 | N | MET | A | 319 | 31.799 | 19.670 | 25.603 | 1.00 | 26.29 | N |
| ATOM | 499 | CA | MET | A | 319 | 31.018 | 20.716 | 24.961 | 1.00 | 26.24 | C |
| ATOM | 500 | CB | MET | A | 319 | 31.852 | 22.004 | 24.831 | 1.00 | 27.30 | C |
| ATOM | 501 | CG | MET | A | 319 | 31.050 | 23.268 | 24.597 | 1.00 | 28.90 | C |
| ATOM | 502 | SD | MET | A | 319 | 29.794 | 23.654 | 25.828 | 1.00 | 33.36 | S |
| ATOM | 503 | CE | MET | A | 319 | 28.848 | 24.807 | 24.932 | 1.00 | 31.15 | C |
| ATOM | 504 | C | MET | A | 319 | 30.472 | 20.228 | 23.615 | 1.00 | 25.53 | C |
| ATOM | 505 | O | MET | A | 319 | 29.325 | 20.447 | 23.281 | 1.00 | 25.99 | O |
| ATOM | 506 | N | TRP | A | 320 | 31.267 | 19.511 | 22.857 | 1.00 | 25.05 | N |
| ATOM | 507 | CA | TRP | A | 320 | 30.790 | 18.939 | 21.621 | 1.00 | 24.82 | C |
| ATOM | 508 | CB | TRP | A | 320 | 31.923 | 18.183 | 20.921 | 1.00 | 24.80 | C |
| ATOM | 509 | CG | TRP | A | 320 | 32.634 | 18.949 | 19.867 | 1.00 | 27.31 | C |
| ATOM | 510 | CD1 | TRP | A | 320 | 33.705 | 19.786 | 20.039 | 1.00 | 30.43 | C |
| ATOM | 511 | NE1 | TRP | A | 320 | 34.100 | 20.301 | 18.831 | 1.00 | 31.02 | N |
| ATOM | 512 | CE2 | TRP | A | 320 | 33.268 | 19.826 | 17.854 | 1.00 | 28.25 | C |
| ATOM | 513 | CD2 | TRP | A | 320 | 32.338 | 18.969 | 18.470 | 1.00 | 28.17 | C |
| ATOM | 514 | CE3 | TRP | A | 320 | 31.357 | 18.366 | 17.675 | 1.00 | 29.23 | C |
| ATOM | 515 | CZ3 | TRP | A | 320 | 31.356 | 18.606 | 16.320 | 1.00 | 28.97 | C |
| ATOM | 516 | CH2 | TRP | A | 320 | 32.293 | 19.478 | 15.739 | 1.00 | 28.19 | C |
| ATOM | 517 | CZ2 | TRP | A | 320 | 33.260 | 20.086 | 16.492 | 1.00 | 28.61 | C |
| ATOM | 518 | C | TRP | A | 320 | 29.594 | 17.984 | 21.832 | 1.00 | 24.11 | C |
| ATOM | 519 | O | TRP | A | 320 | 28.638 | 18.021 | 21.080 | 1.00 | 23.54 | O |
| ATOM | 520 | N | GLN | A | 321 | 29.644 | 17.118 | 22.832 | 1.00 | 24.43 | N |
| ATOM | 521 | CA | GLN | A | 321 | 28.544 | 16.178 | 23.043 | 1.00 | 24.39 | C |
| ATOM | 522 | CB | GLN | A | 321 | 28.836 | 15.179 | 24.168 | 1.00 | 24.76 | C |
| ATOM | 523 | CG | GLN | A | 321 | 27.876 | 14.013 | 24.140 | 1.00 | 24.84 | C |
| ATOM | 524 | CD | GLN | A | 321 | 27.120 | 13.800 | 25.417 | 1.00 | 26.37 | C |
| ATOM | 525 | OE1 | GLN | A | 321 | 27.146 | 14.650 | 26.271 | 1.00 | 29.09 | O |
| ATOM | 526 | NE2 | GLN | A | 321 | 26.444 | 12.627 | 25.556 | 1.00 | 26.55 | N |
| ATOM | 527 | C | GLN | A | 321 | 27.249 | 16.900 | 23.359 | 1.00 | 24.19 | C |
| ATOM | 528 | O | GLN | A | 321 | 26.180 | 16.559 | 22.832 | 1.00 | 24.08 | O |
| ATOM | 529 | N | LEU | A | 322 | 27.368 | 17.924 | 24.178 | 1.00 | 24.78 | N |
| ATOM | 530 | CA | LEU | A | 322 | 26.236 | 18.733 | 24.579 | 1.00 | 25.69 | C |
| ATOM | 531 | CB | LEU | A | 322 | 26.644 | 19.732 | 25.683 | 1.00 | 26.07 | C |
| ATOM | 532 | CG | LEU | A | 322 | 25.622 | 20.738 | 26.235 | 1.00 | 27.41 | C |
| ATOM | 533 | CD1 | LEU | A | 322 | 24.406 | 20.074 | 26.909 | 1.00 | 29.40 | C |
| ATOM | 534 | CD2 | LEU | A | 322 | 26.277 | 21.639 | 27.190 | 1.00 | 30.22 | C |
| ATOM | 535 | C | LEU | A | 322 | 25.615 | 19.430 | 23.374 | 1.00 | 26.06 | C |
| ATOM | 536 | O | LEU | A | 322 | 24.406 | 19.285 | 23.125 | 1.00 | 26.90 | O |
| ATOM | 537 | N | CYS | A | 323 | 26.420 | 20.140 | 22.587 | 1.00 | 25.58 | N |
| ATOM | 538 | CA | CYS | A | 323 | 25.893 | 20.774 | 21.384 | 1.00 | 25.11 | C |
| ATOM | 539 | CB | CYS | A | 323 | 26.996 | 21.554 | 20.686 | 1.00 | 25.72 | C |
| ATOM | 540 | SG | CYS | A | 323 | 27.607 | 22.944 | 21.675 | 1.00 | 29.20 | S |
| ATOM | 541 | C | CYS | A | 323 | 25.246 | 19.776 | 20.419 | 1.00 | 25.04 | C |
| ATOM | 542 | O | CYS | A | 323 | 24.215 | 20.057 | 19.816 | 1.00 | 23.70 | O |
| ATOM | 543 | N | ALA | A | 324 | 25.846 | 18.604 | 20.272 | 1.00 | 24.13 | N |
| ATOM | 544 | CA | ALA | A | 324 | 25.275 | 17.625 | 19.398 | 1.00 | 23.43 | C |
| ATOM | 545 | CB | ALA | A | 324 | 26.226 | 16.462 | 19.222 | 1.00 | 24.05 | C |
| ATOM | 546 | C | ALA | A | 324 | 23.913 | 17.141 | 19.912 | 1.00 | 23.43 | C |
| ATOM | 547 | O | ALA | A | 324 | 23.011 | 16.938 | 19.125 | 1.00 | 22.57 | O |
| ATOM | 548 | N | ILE | A | 325 | 23.776 | 16.932 | 21.218 | 1.00 | 23.59 | N |
| ATOM | 549 | CA | ILE | A | 325 | 22.482 | 16.591 | 21.791 | 1.00 | 24.38 | C |
| ATOM | 550 | CB | ILE | A | 325 | 22.556 | 16.407 | 23.323 | 1.00 | 24.71 | C |
| ATOM | 551 | CG1 | ILE | A | 325 | 23.293 | 15.136 | 23.709 | 1.00 | 25.02 | C |
| ATOM | 552 | CD1 | ILE | A | 325 | 23.649 | 15.149 | 25.190 | 1.00 | 27.28 | C |
| ATOM | 553 | CG2 | ILE | A | 325 | 21.158 | 16.398 | 23.942 | 1.00 | 22.26 | C |
| ATOM | 554 | C | ILE | A | 325 | 21.479 | 17.705 | 21.501 | 1.00 | 24.86 | C |
| ATOM | 555 | O | ILE | A | 325 | 20.384 | 17.453 | 21.044 | 1.00 | 24.56 | O |
| ATOM | 556 | N | LYS | A | 326 | 21.856 | 18.940 | 21.772 | 1.00 | 25.56 | N |
| ATOM | 557 | CA | LYS | A | 326 | 20.926 | 20.047 | 21.580 | 1.00 | 26.26 | C |
| ATOM | 558 | CB | LYS | A | 326 | 21.489 | 21.318 | 22.184 | 1.00 | 26.21 | C |
| ATOM | 559 | CG | LYS | A | 326 | 21.741 | 21.248 | 23.681 | 1.00 | 29.37 | C |
| ATOM | 560 | CD | LYS | A | 326 | 20.479 | 20.914 | 24.433 | 1.00 | 33.36 | C |
| ATOM | 561 | CE | LYS | A | 326 | 20.725 | 20.802 | 25.926 | 1.00 | 36.77 | C |
| ATOM | 562 | NZ | LYS | A | 326 | 19.694 | 19.921 | 26.567 | 1.00 | 37.66 | N |
| ATOM | 563 | C | LYS | A | 326 | 20.520 | 20.209 | 20.094 | 1.00 | 26.40 | C |
| ATOM | 564 | O | LYS | A | 326 | 19.348 | 20.386 | 19.803 | 1.00 | 25.16 | O |
| ATOM | 565 | N | ILE | A | 327 | 21.480 | 20.110 | 19.175 | 1.00 | 26.86 | N |
| ATOM | 566 | CA | ILE | A | 327 | 21.208 | 20.174 | 17.740 | 1.00 | 28.16 | C |
| ATOM | 567 | CB | ILE | A | 327 | 22.511 | 20.235 | 16.930 | 1.00 | 29.15 | C |
| ATOM | 568 | CG1 | ILE | A | 327 | 22.666 | 21.619 | 16.335 | 1.00 | 35.09 | C |
| ATOM | 569 | CD1 | ILE | A | 327 | 23.951 | 21.810 | 15.538 | 1.00 | 39.84 | C |
| ATOM | 570 | CG2 | ILE | A | 327 | 22.465 | 19.314 | 15.745 | 1.00 | 31.87 | C |
| ATOM | 571 | C | ILE | A | 327 | 20.378 | 19.015 | 17.250 | 1.00 | 27.66 | C |
| ATOM | 572 | O | ILE | A | 327 | 19.544 | 19.152 | 16.361 | 1.00 | 27.77 | O |
| ATOM | 573 | N | THR | A | 328 | 20.599 | 17.855 | 17.811 | 1.00 | 27.88 | N |
| ATOM | 574 | CA | THR | A | 328 | 19.810 | 16.726 | 17.415 | 1.00 | 28.75 | C |
| ATOM | 575 | CB | THR | A | 328 | 20.328 | 15.479 | 18.028 | 1.00 | 29.15 | C |
| ATOM | 576 | OG1 | THR | A | 328 | 21.665 | 15.267 | 17.581 | 1.00 | 28.97 | O |
| ATOM | 577 | CG2 | THR | A | 328 | 19.547 | 14.287 | 17.518 | 1.00 | 30.09 | C |
| ATOM | 578 | C | THR | A | 328 | 18.343 | 16.927 | 17.776 | 1.00 | 28.80 | C |
| ATOM | 579 | O | THR | A | 328 | 17.496 | 16.567 | 16.986 | 1.00 | 27.42 | O |
| ATOM | 580 | N | GLU | A | 329 | 18.051 | 17.530 | 18.928 | 1.00 | 29.14 | N |
| ATOM | 581 | CA | GLU | A | 329 | 16.658 | 17.852 | 19.278 | 1.00 | 30.60 | C |
| ATOM | 582 | CB | GLU | A | 329 | 16.546 | 18.559 | 20.643 | 1.00 | 31.73 | C |
| ATOM | 583 | CG | GLU | A | 329 | 17.198 | 17.863 | 21.820 | 1.00 | 37.45 | C |
| ATOM | 584 | CD | GLU | A | 329 | 17.178 | 18.684 | 23.122 | 1.00 | 43.70 | C |
| ATOM | 585 | OE1 | GLU | A | 329 | 16.578 | 19.802 | 23.159 | 1.00 | 42.77 | O |
| ATOM | 586 | OE2 | GLU | A | 329 | 17.756 | 18.174 | 24.135 | 1.00 | 49.00 | O |
| ATOM | 587 | C | GLU | A | 329 | 15.985 | 18.751 | 18.215 | 1.00 | 29.00 | C |
| ATOM | 588 | O | GLU | A | 329 | 14.864 | 18.502 | 17.776 | 1.00 | 28.96 | O |
| ATOM | 589 | N | ALA | A | 330 | 16.648 | 19.829 | 17.825 | 1.00 | 27.92 | N |
| ATOM | 590 | CA | ALA | A | 330 | 16.105 | 20.694 | 16.793 | 1.00 | 27.01 | C |
| ATOM | 591 | CB | ALA | A | 330 | 16.981 | 21.881 | 16.610 | 1.00 | 27.09 | C |
| ATOM | 592 | C | ALA | A | 330 | 15.900 | 19.967 | 15.447 | 1.00 | 27.24 | C |
| ATOM | 593 | O | ALA | A | 330 | 14.911 | 20.208 | 14.753 | 1.00 | 27.51 | O |
| ATOM | 594 | N | ILE | A | 331 | 16.837 | 19.109 | 15.070 | 1.00 | 26.90 | N |
| ATOM | 595 | CA | ILE | A | 331 | 16.695 | 18.308 | 13.860 | 1.00 | 27.25 | C |
| ATOM | 596 | CB | ILE | A | 331 | 17.973 | 17.520 | 13.585 | 1.00 | 26.89 | C |
| ATOM | 597 | CG1 | ILE | A | 331 | 19.052 | 18.507 | 13.149 | 1.00 | 27.96 | C |
| ATOM | 598 | CD1 | ILE | A | 331 | 20.406 | 17.877 | 12.923 | 1.00 | 29.48 | C |
| ATOM | 599 | CG2 | ILE | A | 331 | 17.766 | 16.414 | 12.484 | 1.00 | 26.39 | C |
| ATOM | 600 | C | ILE | A | 331 | 15.470 | 17.390 | 13.885 | 1.00 | 27.69 | C |
| ATOM | 601 | O | ILE | A | 331 | 14.810 | 17.205 | 12.859 | 1.00 | 27.06 | O |
| ATOM | 602 | N | GLN | A | 332 | 15.160 | 16.830 | 15.041 | 1.00 | 28.59 | N |
| ATOM | 603 | CA | GLN | A | 332 | 13.987 | 15.988 | 15.167 | 1.00 | 30.50 | C |
| ATOM | 604 | CB | GLN | A | 332 | 13.849 | 15.439 | 16.579 | 1.00 | 31.71 | C |
| ATOM | 605 | CG | GLN | A | 332 | 14.526 | 14.109 | 16.681 | 1.00 | 36.53 | C |
| ATOM | 606 | CD | GLN | A | 332 | 14.827 | 13.693 | 18.084 | 1.00 | 43.18 | C |
| ATOM | 607 | OE1 | GLN | A | 332 | 14.295 | 14.274 | 19.043 | 1.00 | 46.43 | O |
| ATOM | 608 | NE2 | GLN | A | 332 | 15.717 | 12.680 | 18.228 | 1.00 | 46.39 | N |
| ATOM | 609 | C | GLN | A | 332 | 12.736 | 16.742 | 14.763 | 1.00 | 30.16 | C |
| ATOM | 610 | O | GLN | A | 332 | 11.879 | 16.192 | 14.086 | 1.00 | 29.89 | O |
| ATOM | 611 | N | TYR | A | 333 | 12.666 | 18.012 | 15.142 | 1.00 | 29.91 | N |
| ATOM | 612 | CA | TYR | A | 333 | 11.548 | 18.863 | 14.773 | 1.00 | 29.79 | C |
| ATOM | 613 | CB | TYR | A | 333 | 11.552 | 20.152 | 15.632 | 1.00 | 30.02 | C |
| ATOM | 614 | CG | TYR | A | 333 | 11.052 | 19.929 | 17.038 | 1.00 | 30.98 | C |
| ATOM | 615 | CD1 | TYR | A | 333 | 11.928 | 19.789 | 18.095 | 1.00 | 32.18 | C |
| ATOM | 616 | CE1 | TYR | A | 333 | 11.460 | 19.555 | 19.372 | 1.00 | 34.65 | C |
| ATOM | 617 | CZ | TYR | A | 333 | 10.097 | 19.474 | 19.605 | 1.00 | 35.79 | C |
| ATOM | 618 | OH | TYR | A | 333 | 9.635 | 19.256 | 20.875 | 1.00 | 40.22 | O |
| ATOM | 619 | CE2 | TYR | A | 333 | 9.207 | 19.621 | 18.590 | 1.00 | 34.47 | C |
| ATOM | 620 | CD2 | TYR | A | 333 | 9.687 | 19.858 | 17.305 | 1.00 | 34.75 | C |
| ATOM | 621 | C | TYR | A | 333 | 11.543 | 19.187 | 13.272 | 1.00 | 29.21 | C |
| ATOM | 622 | O | TYR | A | 333 | 10.498 | 19.330 | 12.658 | 1.00 | 29.48 | O |
| ATOM | 623 | N | VAL | A | 334 | 12.711 | 19.337 | 12.683 | 1.00 | 28.49 | N |
| ATOM | 624 | CA | VAL | A | 334 | 12.781 | 19.602 | 11.247 | 1.00 | 28.13 | C |
| ATOM | 625 | CB | VAL | A | 334 | 14.204 | 20.060 | 10.872 | 1.00 | 28.00 | C |
| ATOM | 626 | CG1 | VAL | A | 334 | 14.470 | 19.953 | 9.349 | 1.00 | 28.89 | C |
| ATOM | 627 | CG2 | VAL | A | 334 | 14.441 | 21.427 | 11.401 | 1.00 | 26.70 | C |
| ATOM | 628 | C | VAL | A | 334 | 12.347 | 18.349 | 10.439 | 1.00 | 27.92 | C |
| ATOM | 629 | O | VAL | A | 334 | 11.770 | 18.465 | 9.378 | 1.00 | 26.84 | O |
| ATOM | 630 | N | VAL | A | 335 | 12.612 | 17.158 | 10.948 | 1.00 | 28.87 | N |
| ATOM | 631 | CA | VAL | A | 335 | 12.151 | 15.943 | 10.276 | 1.00 | 30.06 | C |
| ATOM | 632 | CB | VAL | A | 335 | 12.737 | 14.699 | 10.894 | 1.00 | 30.00 | C |
| ATOM | 633 | CG1 | VAL | A | 335 | 12.072 | 13.487 | 10.339 | 1.00 | 32.57 | C |
| ATOM | 634 | CG2 | VAL | A | 335 | 14.210 | 14.592 | 10.597 | 1.00 | 30.15 | C |
| ATOM | 635 | C | VAL | A | 335 | 10.588 | 15.881 | 10.266 | 1.00 | 30.90 | C |
| ATOM | 636 | O | VAL | A | 335 | 9.984 | 15.468 | 9.263 | 1.00 | 29.27 | O |
| ATOM | 637 | N | GLU | A | 336 | 9.953 | 16.356 | 11.344 | 1.00 | 31.20 | N |
| ATOM | 638 | CA | GLU | A | 336 | 8.489 | 16.425 | 11.393 | 1.00 | 32.50 | C |
| ATOM | 639 | CB | GLU | A | 336 | 7.961 | 16.735 | 12.812 | 1.00 | 33.25 | C |
| ATOM | 640 | CG | GLU | A | 336 | 8.283 | 15.671 | 13.862 | 1.00 | 36.91 | C |
| ATOM | 641 | CD | GLU | A | 336 | 7.627 | 14.299 | 13.616 | 1.00 | 44.18 | C |
| ATOM | 642 | OE1 | GLU | A | 336 | 6.463 | 14.240 | 13.123 | 1.00 | 48.70 | O |
| ATOM | 643 | OE2 | GLU | A | 336 | 8.276 | 13.256 | 13.928 | 1.00 | 47.83 | O |
| ATOM | 644 | C | GLU | A | 336 | 7.980 | 17.440 | 10.380 | 1.00 | 31.88 | C |
| ATOM | 645 | O | GLU | A | 336 | 6.994 | 17.194 | 9.697 | 1.00 | 32.26 | O |
| ATOM | 646 | N | PHE | A | 337 | 8.654 | 18.569 | 10.246 | 1.00 | 31.25 | N |
| ATOM | 647 | CA | PHE | A | 337 | 8.315 | 19.488 | 9.189 | 1.00 | 31.26 | C |
| ATOM | 648 | CB | PHE | A | 337 | 9.270 | 20.670 | 9.251 | 1.00 | 31.46 | C |
| ATOM | 649 | CG | PHE | A | 337 | 9.017 | 21.746 | 8.229 | 1.00 | 30.00 | C |
| ATOM | 650 | CD1 | PHE | A | 337 | 7.842 | 22.467 | 8.237 | 1.00 | 29.18 | C |
| ATOM | 651 | CE1 | PHE | A | 337 | 7.643 | 23.484 | 7.357 | 1.00 | 29.25 | C |
| ATOM | 652 | CZ | PHE | A | 337 | 8.620 | 23.811 | 6.418 | 1.00 | 30.20 | C |
| ATOM | 653 | CE2 | PHE | A | 337 | 9.799 | 23.094 | 6.381 | 1.00 | 30.76 | C |
| ATOM | 654 | CD2 | PHE | A | 337 | 9.988 | 22.059 | 7.287 | 1.00 | 31.21 | C |
| ATOM | 655 | C | PHE | A | 337 | 8.364 | 18.826 | 7.779 | 1.00 | 32.11 | C |
| ATOM | 656 | O | PHE | A | 337 | 7.402 | 18.929 | 7.011 | 1.00 | 31.39 | O |
| ATOM | 657 | N | ALA | A | 338 | 9.479 | 18.176 | 7.438 | 1.00 | 32.85 | N |
| ATOM | 658 | CA | ALA | A | 338 | 9.618 | 17.536 | 6.134 | 1.00 | 34.45 | C |
| ATOM | 659 | CB | ALA | A | 338 | 10.936 | 16.787 | 6.029 | 1.00 | 34.40 | C |
| ATOM | 660 | C | ALA | A | 338 | 8.448 | 16.593 | 5.849 | 1.00 | 35.78 | C |
| ATOM | 661 | O | ALA | A | 338 | 7.848 | 16.667 | 4.806 | 1.00 | 35.41 | O |
| ATOM | 662 | N | LYS | A | 339 | 8.127 | 15.725 | 6.790 | 1.00 | 37.72 | N |
| ATOM | 663 | CA | LYS | A | 339 | 7.058 | 14.776 | 6.598 | 1.00 | 39.80 | C |
| ATOM | 664 | CB | LYS | A | 339 | 6.761 | 14.056 | 7.886 | 1.00 | 40.02 | C |
| ATOM | 665 | CG | LYS | A | 339 | 7.901 | 13.158 | 8.291 | 1.00 | 42.45 | C |
| ATOM | 666 | CD | LYS | A | 339 | 7.440 | 11.942 | 9.028 | 1.00 | 44.60 | C |
| ATOM | 667 | CE | LYS | A | 339 | 7.206 | 12.224 | 10.454 | 1.00 | 46.22 | C |
| ATOM | 668 | NZ | LYS | A | 339 | 7.415 | 10.966 | 11.174 | 1.00 | 46.80 | N |
| ATOM | 669 | C | LYS | A | 339 | 5.777 | 15.391 | 6.072 | 1.00 | 41.25 | C |
| ATOM | 670 | O | LYS | A | 339 | 5.091 | 14.789 | 5.275 | 1.00 | 41.74 | O |
| ATOM | 671 | N | ARG | A | 340 | 5.485 | 16.605 | 6.497 | 1.00 | 42.74 | N |
| ATOM | 672 | CA | ARG | A | 340 | 4.266 | 17.275 | 6.127 | 1.00 | 43.70 | C |
| ATOM | 673 | CB | ARG | A | 340 | 3.887 | 18.223 | 7.229 | 1.00 | 43.78 | C |
| ATOM | 674 | CG | ARG | A | 340 | 3.550 | 17.533 | 8.494 | 1.00 | 45.03 | C |
| ATOM | 675 | CD | ARG | A | 340 | 3.273 | 18.483 | 9.594 | 1.00 | 46.53 | C |
| ATOM | 676 | NE | ARG | A | 340 | 3.333 | 17.821 | 10.887 | 1.00 | 49.07 | N |
| ATOM | 677 | CZ | ARG | A | 340 | 2.578 | 18.165 | 11.915 | 1.00 | 51.42 | C |
| ATOM | 678 | NH1 | ARG | A | 340 | 1.697 | 19.167 | 11.779 | 1.00 | 53.86 | N |
| ATOM | 679 | NH2 | ARG | A | 340 | 2.691 | 17.510 | 13.063 | 1.00 | 50.56 | N |
| ATOM | 680 | C | ARG | A | 340 | 4.355 | 18.086 | 4.858 | 1.00 | 44.39 | C |
| ATOM | 681 | O | ARG | A | 340 | 3.381 | 18.710 | 4.473 | 1.00 | 44.75 | O |
| ATOM | 682 | N | ILE | A | 341 | 5.516 | 18.136 | 4.231 | 1.00 | 44.93 | N |
| ATOM | 683 | CA | ILE | A | 341 | 5.626 | 18.907 | 3.015 | 1.00 | 45.79 | C |
| ATOM | 684 | CB | ILE | A | 341 | 7.073 | 19.340 | 2.776 | 1.00 | 45.44 | C |
| ATOM | 685 | CG1 | ILE | A | 341 | 7.446 | 20.384 | 3.818 | 1.00 | 45.69 | C |
| ATOM | 686 | CD1 | ILE | A | 341 | 8.901 | 20.710 | 3.847 | 1.00 | 46.77 | C |
| ATOM | 687 | CG2 | ILE | A | 341 | 7.240 | 19.881 | 1.357 | 1.00 | 45.67 | C |
| ATOM | 688 | C | ILE | A | 341 | 5.080 | 18.035 | 1.895 | 1.00 | 46.85 | C |
| ATOM | 689 | O | ILE | A | 341 | 5.483 | 16.883 | 1.738 | 1.00 | 46.56 | O |
| ATOM | 690 | N | ASP | A | 342 | 4.148 | 18.593 | 1.129 | 1.00 | 48.25 | N |
| ATOM | 691 | CA | ASP | A | 342 | 3.476 | 17.821 | 0.093 | 1.00 | 49.01 | C |
| ATOM | 692 | CB | ASP | A | 342 | 2.306 | 18.611 | −0.490 | 1.00 | 50.45 | C |
| ATOM | 693 | CG | ASP | A | 342 | 1.152 | 18.788 | 0.493 | 1.00 | 53.95 | C |
| ATOM | 694 | OD1 | ASP | A | 342 | 0.695 | 17.786 | 1.084 | 1.00 | 58.87 | O |
| ATOM | 695 | OD2 | ASP | A | 342 | 0.636 | 19.914 | 0.713 | 1.00 | 59.55 | O |
| ATOM | 696 | C | ASP | A | 342 | 4.424 | 17.453 | −1.031 | 1.00 | 48.01 | C |
| ATOM | 697 | O | ASP | A | 342 | 4.927 | 18.316 | −1.747 | 1.00 | 47.92 | O |
| ATOM | 698 | N | GLY | A | 343 | 4.642 | 16.155 | −1.185 | 1.00 | 46.69 | N |
| ATOM | 699 | CA | GLY | A | 343 | 5.544 | 15.642 | −2.199 | 1.00 | 45.52 | C |
| ATOM | 700 | C | GLY | A | 343 | 6.710 | 14.888 | −1.596 | 1.00 | 43.87 | C |
| ATOM | 701 | O | GLY | A | 343 | 7.268 | 13.970 | −2.205 | 1.00 | 43.67 | O |
| ATOM | 702 | N | PHE | A | 344 | 7.045 | 15.232 | −0.365 | 1.00 | 41.81 | N |
| ATOM | 703 | CA | PHE | A | 344 | 8.221 | 14.673 | 0.266 | 1.00 | 40.97 | C |
| ATOM | 704 | CB | PHE | A | 344 | 8.588 | 15.453 | 1.533 | 1.00 | 40.64 | C |
| ATOM | 705 | CG | PHE | A | 344 | 9.850 | 14.977 | 2.170 | 1.00 | 38.48 | C |
| ATOM | 706 | CD1 | PHE | A | 344 | 11.081 | 15.500 | 1.814 | 1.00 | 36.83 | C |
| ATOM | 707 | CE1 | PHE | A | 344 | 12.245 | 15.020 | 2.396 | 1.00 | 37.61 | C |
| ATOM | 708 | CZ | PHE | A | 344 | 12.190 | 14.000 | 3.311 | 1.00 | 36.20 | C |
| ATOM | 709 | CE2 | PHE | A | 344 | 10.961 | 13.456 | 3.658 | 1.00 | 36.79 | C |
| ATOM | 710 | CD2 | PHE | A | 344 | 9.809 | 13.939 | 3.084 | 1.00 | 36.78 | C |
| ATOM | 711 | C | PHE | A | 344 | 7.999 | 13.204 | 0.543 | 1.00 | 41.48 | C |
| ATOM | 712 | O | PHE | A | 344 | 8.864 | 12.366 | 0.254 | 1.00 | 41.42 | O |
| ATOM | 713 | N | MET | A | 345 | 6.814 | 12.881 | 1.056 | 1.00 | 42.09 | N |
| ATOM | 714 | CA | MET | A | 345 | 6.447 | 11.536 | 1.370 | 1.00 | 43.10 | C |
| ATOM | 715 | CB | MET | A | 345 | 5.247 | 11.496 | 2.326 | 1.00 | 43.84 | C |
| ATOM | 716 | CG | MET | A | 345 | 5.590 | 11.871 | 3.775 | 1.00 | 46.08 | C |
| ATOM | 717 | SD | MET | A | 345 | 7.102 | 11.080 | 4.441 | 1.00 | 50.98 | S |
| ATOM | 718 | CE | MET | A | 345 | 6.721 | 9.279 | 4.354 | 1.00 | 52.61 | C |
| ATOM | 719 | C | MET | A | 345 | 6.187 | 10.673 | 0.109 | 1.00 | 43.55 | C |
| ATOM | 720 | O | MET | A | 345 | 6.190 | 9.427 | 0.184 | 1.00 | 42.43 | O |
| ATOM | 721 | N | GLU | A | 346 | 5.974 | 11.311 | −1.040 | 1.00 | 44.36 | N |
| ATOM | 722 | CA | GLU | A | 346 | 5.839 | 10.568 | −2.291 | 1.00 | 45.64 | C |
| ATOM | 723 | CB | GLU | A | 346 | 5.107 | 11.384 | −3.339 | 1.00 | 46.00 | C |
| ATOM | 724 | CG | GLU | A | 346 | 3.616 | 11.455 | −3.132 | 1.00 | 49.03 | C |
| ATOM | 725 | CD | GLU | A | 346 | 2.989 | 12.662 | −3.821 | 1.00 | 54.25 | C |
| ATOM | 726 | OE1 | GLU | A | 346 | 3.726 | 13.555 | −4.328 | 1.00 | 56.30 | O |
| ATOM | 727 | OE2 | GLU | A | 346 | 1.740 | 12.718 | −3.852 | 1.00 | 58.05 | O |
| ATOM | 728 | C | GLU | A | 346 | 7.182 | 10.143 | −2.901 | 1.00 | 45.79 | C |
| ATOM | 729 | O | GLU | A | 346 | 7.209 | 9.327 | −3.826 | 1.00 | 46.33 | O |
| ATOM | 730 | N | LEU | A | 347 | 8.288 | 10.691 | −2.411 | 1.00 | 45.30 | N |
| ATOM | 731 | CA | LEU | A | 347 | 9.593 | 10.361 | −2.983 | 1.00 | 44.95 | C |
| ATOM | 732 | CB | LEU | A | 347 | 10.618 | 11.445 | −2.650 | 1.00 | 44.65 | C |
| ATOM | 733 | CG | LEU | A | 347 | 10.252 | 12.868 | −3.077 | 1.00 | 44.22 | C |
| ATOM | 734 | CD1 | LEU | A | 347 | 11.065 | 13.905 | −2.341 | 1.00 | 43.57 | C |
| ATOM | 735 | CD2 | LEU | A | 347 | 10.447 | 13.049 | −4.576 | 1.00 | 44.47 | C |
| ATOM | 736 | C | LEU | A | 347 | 10.086 | 9.010 | −2.469 | 1.00 | 44.60 | C |
| ATOM | 737 | O | LEU | A | 347 | 9.634 | 8.530 | −1.426 | 1.00 | 43.99 | O |
| ATOM | 738 | N | CYS | A | 348 | 11.007 | 8.392 | −3.205 | 1.00 | 44.39 | N |
| ATOM | 739 | CA | CYS | A | 348 | 11.599 | 7.132 | −2.754 | 1.00 | 44.78 | C |
| ATOM | 740 | CB | CYS | A | 348 | 12.511 | 6.526 | −3.835 | 1.00 | 44.94 | C |
| ATOM | 741 | SG | CYS | A | 348 | 13.860 | 7.612 | −4.385 | 1.00 | 48.13 | S |
| ATOM | 742 | C | CYS | A | 348 | 12.388 | 7.401 | −1.474 | 1.00 | 43.97 | C |
| ATOM | 743 | O | CYS | A | 348 | 12.982 | 8.475 | −1.326 | 1.00 | 42.90 | O |
| ATOM | 744 | N | GLN | A | 349 | 12.401 | 6.427 | −0.567 | 1.00 | 43.70 | N |
| ATOM | 745 | CA | GLN | A | 349 | 13.096 | 6.569 | 0.708 | 1.00 | 44.41 | C |
| ATOM | 746 | CB | GLN | A | 349 | 13.178 | 5.250 | 1.487 | 1.00 | 45.15 | C |
| ATOM | 747 | CG | GLN | A | 349 | 13.587 | 5.479 | 2.949 | 1.00 | 47.77 | C |
| ATOM | 748 | CD | GLN | A | 349 | 13.747 | 4.207 | 3.723 | 1.00 | 51.04 | C |
| ATOM | 749 | OE1 | GLN | A | 349 | 13.880 | 3.124 | 3.139 | 1.00 | 53.73 | O |
| ATOM | 750 | NE2 | GLN | A | 349 | 13.761 | 4.325 | 5.040 | 1.00 | 53.95 | N |
| ATOM | 751 | C | GLN | A | 349 | 14.499 | 7.124 | 0.495 | 1.00 | 43.84 | C |
| ATOM | 752 | O | GLN | A | 349 | 15.017 | 7.862 | 1.324 | 1.00 | 43.52 | O |
| ATOM | 753 | N | ASN | A | 350 | 15.106 | 6.753 | −0.623 | 1.00 | 43.26 | N |
| ATOM | 754 | CA | ASN | A | 350 | 16.451 | 7.175 | −0.929 | 1.00 | 43.16 | C |
| ATOM | 755 | CB | ASN | A | 350 | 16.936 | 6.460 | −2.175 | 1.00 | 43.98 | C |
| ATOM | 756 | CG | ASN | A | 350 | 16.970 | 4.940 | −1.994 | 1.00 | 48.26 | C |
| ATOM | 757 | OD1 | ASN | A | 350 | 18.058 | 4.340 | −1.904 | 1.00 | 52.77 | O |
| ATOM | 758 | ND2 | ASN | A | 350 | 15.773 | 4.305 | −1.934 | 1.00 | 51.57 | N |
| ATOM | 759 | C | ASN | A | 350 | 16.563 | 8.686 | −1.116 | 1.00 | 40.98 | C |
| ATOM | 760 | O | ASN | A | 350 | 17.499 | 9.296 | −0.637 | 1.00 | 40.25 | O |
| ATOM | 761 | N | ASP | A | 351 | 15.638 | 9.266 | −1.860 | 1.00 | 39.10 | N |
| ATOM | 762 | CA | ASP | A | 351 | 15.643 | 10.696 | −2.065 | 1.00 | 38.32 | C |
| ATOM | 763 | CB | ASP | A | 351 | 14.794 | 11.100 | −3.253 | 1.00 | 38.60 | C |
| ATOM | 764 | CG | ASP | A | 351 | 15.504 | 10.847 | −4.590 | 1.00 | 41.32 | C |
| ATOM | 765 | OD1 | ASP | A | 351 | 16.656 | 10.309 | −4.587 | 1.00 | 42.06 | O |
| ATOM | 766 | OD2 | ASP | A | 351 | 14.977 | 11.143 | −5.686 | 1.00 | 42.98 | O |
| ATOM | 767 | C | ASP | A | 351 | 15.194 | 11.404 | −0.783 | 1.00 | 36.92 | C |
| ATOM | 768 | O | ASP | A | 351 | 15.721 | 12.435 | −0.464 | 1.00 | 35.52 | O |
| ATOM | 769 | N | GLN | A | 352 | 14.279 | 10.813 | −0.023 | 1.00 | 35.97 | N |
| ATOM | 770 | CA | GLN | A | 352 | 13.905 | 11.377 | 1.265 | 1.00 | 35.61 | C |
| ATOM | 771 | CB | GLN | A | 352 | 12.875 | 10.478 | 1.950 | 1.00 | 35.48 | C |
| ATOM | 772 | CG | GLN | A | 352 | 11.497 | 10.599 | 1.322 | 1.00 | 37.95 | C |
| ATOM | 773 | CD | GLN | A | 352 | 10.561 | 9.498 | 1.737 | 1.00 | 40.62 | C |
| ATOM | 774 | OE1 | GLN | A | 352 | 10.668 | 8.974 | 2.839 | 1.00 | 40.95 | O |
| ATOM | 775 | NE2 | GLN | A | 352 | 9.649 | 9.123 | 0.848 | 1.00 | 44.80 | N |
| ATOM | 776 | C | GLN | A | 352 | 15.154 | 11.554 | 2.137 | 1.00 | 34.57 | C |
| ATOM | 777 | O | GLN | A | 352 | 15.388 | 12.611 | 2.720 | 1.00 | 32.40 | O |
| ATOM | 778 | N | ILE | A | 353 | 15.970 | 10.508 | 2.184 | 1.00 | 33.88 | N |
| ATOM | 779 | CA | ILE | A | 353 | 17.178 | 10.487 | 3.002 | 1.00 | 34.01 | C |
| ATOM | 780 | CB | ILE | A | 353 | 17.772 | 9.049 | 2.998 | 1.00 | 33.98 | C |
| ATOM | 781 | CG1 | ILE | A | 353 | 16.805 | 8.112 | 3.715 | 1.00 | 36.61 | C |
| ATOM | 782 | CD1 | ILE | A | 353 | 17.075 | 6.631 | 3.477 | 1.00 | 38.26 | C |
| ATOM | 783 | CG2 | ILE | A | 353 | 19.101 | 9.002 | 3.715 | 1.00 | 34.10 | C |
| ATOM | 784 | C | ILE | A | 353 | 18.225 | 11.498 | 2.548 | 1.00 | 32.99 | C |
| ATOM | 785 | O | ILE | A | 353 | 18.821 | 12.187 | 3.364 | 1.00 | 32.18 | O |
| ATOM | 786 | N | VAL | A | 354 | 18.466 | 11.563 | 1.246 | 1.00 | 31.86 | N |
| ATOM | 787 | CA | VAL | A | 354 | 19.433 | 12.515 | 0.698 | 1.00 | 31.38 | C |
| ATOM | 788 | CB | VAL | A | 354 | 19.569 | 12.320 | −0.821 | 1.00 | 31.50 | C |
| ATOM | 789 | CG1 | VAL | A | 354 | 20.148 | 13.516 | −1.458 | 1.00 | 31.80 | C |
| ATOM | 790 | CG2 | VAL | A | 354 | 20.388 | 11.039 | −1.149 | 1.00 | 32.45 | C |
| ATOM | 791 | C | VAL | A | 354 | 19.066 | 13.988 | 0.998 | 1.00 | 29.81 | C |
| ATOM | 792 | O | VAL | A | 354 | 19.939 | 14.794 | 1.317 | 1.00 | 29.46 | O |
| ATOM | 793 | N | LEU | A | 355 | 17.780 | 14.316 | 0.901 | 1.00 | 28.52 | N |
| ATOM | 794 | CA | LEU | A | 355 | 17.317 | 15.673 | 1.175 | 1.00 | 27.57 | C |
| ATOM | 795 | CB | LEU | A | 355 | 15.867 | 15.859 | 0.755 | 1.00 | 27.23 | C |
| ATOM | 796 | CG | LEU | A | 355 | 15.593 | 15.830 | −0.741 | 1.00 | 27.16 | C |
| ATOM | 797 | CD1 | LEU | A | 355 | 14.104 | 15.935 | −0.931 | 1.00 | 28.72 | C |
| ATOM | 798 | CD2 | LEU | A | 355 | 16.337 | 16.878 | −1.514 | 1.00 | 26.89 | C |
| ATOM | 799 | C | LEU | A | 355 | 17.435 | 16.028 | 2.642 | 1.00 | 26.38 | C |
| ATOM | 800 | O | LEU | A | 355 | 17.724 | 17.171 | 2.969 | 1.00 | 24.91 | O |
| ATOM | 801 | N | LEU | A | 356 | 17.184 | 15.067 | 3.522 | 1.00 | 26.87 | N |
| ATOM | 802 | CA | LEU | A | 356 | 17.330 | 15.291 | 4.962 | 1.00 | 28.02 | C |
| ATOM | 803 | CB | LEU | A | 356 | 16.567 | 14.232 | 5.788 | 1.00 | 28.75 | C |
| ATOM | 804 | CG | LEU | A | 356 | 15.046 | 14.442 | 5.700 | 1.00 | 31.27 | C |
| ATOM | 805 | CD1 | LEU | A | 356 | 14.317 | 13.319 | 6.329 | 1.00 | 33.71 | C |
| ATOM | 806 | CD2 | LEU | A | 356 | 14.608 | 15.740 | 6.323 | 1.00 | 31.98 | C |
| ATOM | 807 | C | LEU | A | 356 | 18.785 | 15.340 | 5.404 | 1.00 | 27.73 | C |
| ATOM | 808 | O | LEU | A | 356 | 19.138 | 16.160 | 6.234 | 1.00 | 27.81 | O |
| ATOM | 809 | N | LYS | A | 357 | 19.631 | 14.463 | 4.891 | 1.00 | 28.00 | N |
| ATOM | 810 | CA | LYS | A | 357 | 21.038 | 14.523 | 5.263 | 1.00 | 29.47 | C |
| ATOM | 811 | CB | LYS | A | 357 | 21.875 | 13.426 | 4.613 | 1.00 | 29.39 | C |
| ATOM | 812 | CG | LYS | A | 357 | 21.562 | 12.033 | 5.037 | 1.00 | 33.92 | C |
| ATOM | 813 | CD | LYS | A | 357 | 22.586 | 11.034 | 4.457 | 1.00 | 37.74 | C |
| ATOM | 814 | CE | LYS | A | 357 | 22.683 | 9.804 | 5.331 | 1.00 | 41.03 | C |
| ATOM | 815 | NZ | LYS | A | 357 | 23.255 | 8.598 | 4.607 | 1.00 | 43.01 | N |
| ATOM | 816 | C | LYS | A | 357 | 21.600 | 15.853 | 4.830 | 1.00 | 29.89 | C |
| ATOM | 817 | O | LYS | A | 357 | 22.382 | 16.444 | 5.542 | 1.00 | 30.98 | O |
| ATOM | 818 | N | ALA | A | 358 | 21.207 | 16.338 | 3.657 | 1.00 | 30.12 | N |
| ATOM | 819 | CA | ALA | A | 358 | 21.750 | 17.612 | 3.181 | 1.00 | 30.98 | C |
| ATOM | 820 | CB | ALA | A | 358 | 21.753 | 17.654 | 1.658 | 1.00 | 31.73 | C |
| ATOM | 821 | C | ALA | A | 358 | 21.056 | 18.857 | 3.702 | 1.00 | 30.81 | C |
| ATOM | 822 | O | ALA | A | 358 | 21.686 | 19.893 | 3.875 | 1.00 | 33.30 | O |
| ATOM | 823 | N | GLY | A | 359 | 19.765 | 18.777 | 3.940 | 1.00 | 29.32 | N |
| ATOM | 824 | CA | GLY | A | 359 | 19.040 | 19.938 | 4.331 | 1.00 | 28.50 | C |
| ATOM | 825 | C | GLY | A | 359 | 18.606 | 20.119 | 5.764 | 1.00 | 27.82 | C |
| ATOM | 826 | O | GLY | A | 359 | 18.236 | 21.223 | 6.110 | 1.00 | 27.60 | O |
| ATOM | 827 | N | SER | A | 360 | 18.630 | 19.089 | 6.591 | 1.00 | 26.28 | N |
| ATOM | 828 | CA | SER | A | 360 | 18.064 | 19.239 | 7.905 | 1.00 | 26.79 | C |
| ATOM | 829 | CB | SER | A | 360 | 17.984 | 17.889 | 8.631 | 1.00 | 27.01 | C |
| ATOM | 830 | OG | SER | A | 360 | 19.287 | 17.372 | 8.858 | 1.00 | 30.95 | O |
| ATOM | 831 | C | SER | A | 360 | 18.786 | 20.315 | 8.699 | 1.00 | 26.61 | C |
| ATOM | 832 | O | SER | A | 360 | 18.139 | 21.164 | 9.310 | 1.00 | 26.71 | O |
| ATOM | 833 | N | LEU | A | 361 | 20.116 | 20.345 | 8.635 | 1.00 | 26.46 | N |
| ATOM | 834 | CA | LEU | A | 361 | 20.887 | 21.373 | 9.338 | 1.00 | 26.75 | C |
| ATOM | 835 | CB | LEU | A | 361 | 22.380 | 21.042 | 9.306 | 1.00 | 26.79 | C |
| ATOM | 836 | CG | LEU | A | 361 | 23.250 | 21.762 | 10.334 | 1.00 | 28.75 | C |
| ATOM | 837 | CD1 | LEU | A | 361 | 22.803 | 21.528 | 11.780 | 1.00 | 28.83 | C |
| ATOM | 838 | CD2 | LEU | A | 361 | 24.747 | 21.342 | 10.154 | 1.00 | 30.56 | C |
| ATOM | 839 | C | LEU | A | 361 | 20.660 | 22.765 | 8.782 | 1.00 | 26.67 | C |
| ATOM | 840 | O | LEU | A | 361 | 20.593 | 23.740 | 9.545 | 1.00 | 26.63 | O |
| ATOM | 841 | N | GLU | A | 362 | 20.550 | 22.885 | 7.468 | 1.00 | 25.93 | N |
| ATOM | 842 | CA | GLU | A | 362 | 20.258 | 24.192 | 6.875 | 1.00 | 26.57 | C |
| ATOM | 843 | CB | GLU | A | 362 | 20.158 | 24.122 | 5.357 | 1.00 | 26.98 | C |
| ATOM | 844 | CG | GLU | A | 362 | 21.329 | 23.433 | 4.674 | 1.00 | 31.21 | C |
| ATOM | 845 | CD | GLU | A | 362 | 21.206 | 23.396 | 3.149 | 1.00 | 37.21 | C |
| ATOM | 846 | OE1 | GLU | A | 362 | 22.086 | 22.796 | 2.478 | 1.00 | 41.59 | O |
| ATOM | 847 | OE2 | GLU | A | 362 | 20.212 | 23.932 | 2.619 | 1.00 | 43.73 | O |
| ATOM | 848 | C | GLU | A | 362 | 18.931 | 24.726 | 7.430 | 1.00 | 25.89 | C |
| ATOM | 849 | O | GLU | A | 362 | 18.823 | 25.921 | 7.697 | 1.00 | 24.74 | O |
| ATOM | 850 | N | VAL | A | 363 | 17.920 | 23.868 | 7.592 | 1.00 | 25.19 | N |
| ATOM | 851 | CA | VAL | A | 363 | 16.647 | 24.362 | 8.112 | 1.00 | 26.09 | C |
| ATOM | 852 | CB | VAL | A | 363 | 15.494 | 23.356 | 7.990 | 1.00 | 25.99 | C |
| ATOM | 853 | CG1 | VAL | A | 363 | 14.256 | 23.894 | 8.658 | 1.00 | 27.49 | C |
| ATOM | 854 | CG2 | VAL | A | 363 | 15.182 | 23.075 | 6.538 | 1.00 | 27.46 | C |
| ATOM | 855 | C | VAL | A | 363 | 16.815 | 24.781 | 9.574 | 1.00 | 25.70 | C |
| ATOM | 856 | O | VAL | A | 363 | 16.210 | 25.732 | 10.020 | 1.00 | 25.15 | O |
| ATOM | 857 | N | VAL | A | 364 | 17.682 | 24.091 | 10.308 | 1.00 | 26.29 | N |
| ATOM | 858 | CA | VAL | A | 364 | 17.942 | 24.451 | 11.690 | 1.00 | 25.82 | C |
| ATOM | 859 | CB | VAL | A | 364 | 18.867 | 23.435 | 12.363 | 1.00 | 26.43 | C |
| ATOM | 860 | CG1 | VAL | A | 364 | 19.315 | 23.910 | 13.716 | 1.00 | 25.43 | C |
| ATOM | 861 | CG2 | VAL | A | 364 | 18.171 | 22.105 | 12.538 | 1.00 | 27.04 | C |
| ATOM | 862 | C | VAL | A | 364 | 18.528 | 25.865 | 11.747 | 1.00 | 26.05 | C |
| ATOM | 863 | O | VAL | A | 364 | 18.110 | 26.692 | 12.570 | 1.00 | 25.23 | O |
| ATOM | 864 | N | PHE | A | 365 | 19.491 | 26.162 | 10.877 | 1.00 | 25.43 | N |
| ATOM | 865 | CA | PHE | A | 365 | 20.098 | 27.491 | 10.897 | 1.00 | 25.79 | C |
| ATOM | 866 | CB | PHE | A | 365 | 21.372 | 27.506 | 10.102 | 1.00 | 26.05 | C |
| ATOM | 867 | CG | PHE | A | 365 | 22.482 | 26.755 | 10.743 | 1.00 | 26.79 | C |
| ATOM | 868 | CD1 | PHE | A | 365 | 22.830 | 26.990 | 12.046 | 1.00 | 28.32 | C |
| ATOM | 869 | CE1 | PHE | A | 365 | 23.869 | 26.329 | 12.628 | 1.00 | 29.90 | C |
| ATOM | 870 | CZ | PHE | A | 365 | 24.601 | 25.430 | 11.894 | 1.00 | 31.06 | C |
| ATOM | 871 | CE2 | PHE | A | 365 | 24.290 | 25.215 | 10.614 | 1.00 | 30.47 | C |
| ATOM | 872 | CD2 | PHE | A | 365 | 23.226 | 25.886 | 10.024 | 1.00 | 29.26 | C |
| ATOM | 873 | C | PHE | A | 365 | 19.157 | 28.591 | 10.418 | 1.00 | 26.05 | C |
| ATOM | 874 | O | PHE | A | 365 | 19.265 | 29.745 | 10.846 | 1.00 | 26.08 | O |
| ATOM | 875 | N | ILE | A | 366 | 18.213 | 28.242 | 9.554 | 1.00 | 26.60 | N |
| ATOM | 876 | CA | ILE | A | 366 | 17.183 | 29.194 | 9.178 | 1.00 | 27.03 | C |
| ATOM | 877 | CB | ILE | A | 366 | 16.365 | 28.719 | 7.970 | 1.00 | 27.55 | C |
| ATOM | 878 | CG1 | ILE | A | 366 | 17.273 | 28.657 | 6.731 | 1.00 | 27.31 | C |
| ATOM | 879 | CD1 | ILE | A | 366 | 16.641 | 28.018 | 5.520 | 1.00 | 26.80 | C |
| ATOM | 880 | CG2 | ILE | A | 366 | 15.179 | 29.670 | 7.720 | 1.00 | 27.78 | C |
| ATOM | 881 | C | ILE | A | 366 | 16.325 | 29.422 | 10.404 | 1.00 | 26.64 | C |
| ATOM | 882 | O | ILE | A | 366 | 16.186 | 30.523 | 10.833 | 1.00 | 27.09 | O |
| ATOM | 883 | N | ARG | A | 367 | 15.792 | 28.375 | 11.009 | 1.00 | 27.45 | N |
| ATOM | 884 | CA | ARG | A | 367 | 14.990 | 28.542 | 12.229 | 1.00 | 27.84 | C |
| ATOM | 885 | CB | ARG | A | 367 | 14.500 | 27.204 | 12.733 | 1.00 | 27.21 | C |
| ATOM | 886 | CG | ARG | A | 367 | 13.501 | 26.572 | 11.840 | 1.00 | 27.21 | C |
| ATOM | 887 | CD | ARG | A | 367 | 12.989 | 25.262 | 12.377 | 1.00 | 26.26 | C |
| ATOM | 888 | NE | ARG | A | 367 | 11.797 | 24.825 | 11.687 | 1.00 | 27.92 | N |
| ATOM | 889 | CZ | ARG | A | 367 | 11.017 | 23.827 | 12.095 | 1.00 | 30.29 | C |
| ATOM | 890 | NH1 | ARG | A | 367 | 11.308 | 23.140 | 13.190 | 1.00 | 30.75 | N |
| ATOM | 891 | NH2 | ARG | A | 367 | 9.933 | 23.528 | 11.412 | 1.00 | 31.09 | N |
| ATOM | 892 | C | ARG | A | 367 | 15.723 | 29.268 | 13.367 | 1.00 | 28.32 | C |
| ATOM | 893 | O | ARG | A | 367 | 15.104 | 29.904 | 14.228 | 1.00 | 28.51 | O |
| ATOM | 894 | N | MET | A | 368 | 17.038 | 29.172 | 13.374 | 1.00 | 29.28 | N |
| ATOM | 895 | CA | MET | A | 368 | 17.832 | 29.887 | 14.359 | 1.00 | 30.61 | C |
| ATOM | 896 | CB | MET | A | 368 | 19.311 | 29.754 | 14.039 | 1.00 | 30.32 | C |
| ATOM | 897 | CG | MET | A | 368 | 20.188 | 30.279 | 15.100 | 1.00 | 30.51 | C |
| ATOM | 898 | SD | MET | A | 368 | 21.905 | 29.966 | 14.792 | 1.00 | 31.75 | S |
| ATOM | 899 | CE | MET | A | 368 | 22.107 | 30.986 | 13.388 | 1.00 | 33.23 | C |
| ATOM | 900 | C | MET | A | 368 | 17.484 | 31.372 | 14.427 | 1.00 | 31.27 | C |
| ATOM | 901 | O | MET | A | 368 | 17.590 | 31.982 | 15.481 | 1.00 | 31.35 | O |
| ATOM | 902 | N | CYS | A | 369 | 17.074 | 31.963 | 13.328 | 1.00 | 32.36 | N |
| ATOM | 903 | CA | CYS | A | 369 | 16.775 | 33.385 | 13.375 | 1.00 | 34.85 | C |
| ATOM | 904 | CB | CYS | A | 369 | 16.614 | 34.005 | 11.987 | 1.00 | 35.08 | C |
| ATOM | 905 | SG | CYS | A | 369 | 15.048 | 33.719 | 11.195 | 1.00 | 41.49 | S |
| ATOM | 906 | C | CYS | A | 369 | 15.601 | 33.738 | 14.286 | 1.00 | 34.66 | C |
| ATOM | 907 | O | CYS | A | 369 | 15.556 | 34.844 | 14.785 | 1.00 | 36.34 | O |
| ATOM | 908 | N | ARG | A | 370 | 14.705 | 32.794 | 14.569 | 1.00 | 33.99 | N |
| ATOM | 909 | CA | ARG | A | 370 | 13.589 | 33.045 | 15.460 | 1.00 | 32.77 | C |
| ATOM | 910 | CB | ARG | A | 370 | 12.559 | 31.933 | 15.319 | 1.00 | 32.42 | C |
| ATOM | 911 | CG | ARG | A | 370 | 12.173 | 31.608 | 13.890 | 1.00 | 32.81 | C |
| ATOM | 912 | CD | ARG | A | 370 | 11.507 | 30.270 | 13.790 | 1.00 | 33.89 | C |
| ATOM | 913 | NE | ARG | A | 370 | 10.153 | 30.290 | 14.304 | 1.00 | 32.17 | N |
| ATOM | 914 | CZ | ARG | A | 370 | 9.752 | 29.738 | 15.412 | 1.00 | 32.53 | C |
| ATOM | 915 | NH1 | ARG | A | 370 | 10.598 | 29.098 | 16.193 | 1.00 | 35.61 | N |
| ATOM | 916 | NH2 | ARG | A | 370 | 8.472 | 29.815 | 15.748 | 1.00 | 33.49 | N |
| ATOM | 917 | C | ARG | A | 370 | 14.051 | 33.036 | 16.912 | 1.00 | 32.83 | C |
| ATOM | 918 | O | ARG | A | 370 | 13.322 | 33.465 | 17.817 | 1.00 | 32.92 | O |
| ATOM | 919 | N | ALA | A | 371 | 15.241 | 32.488 | 17.131 | 1.00 | 31.07 | N |
| ATOM | 920 | CA | ALA | A | 371 | 15.757 | 32.294 | 18.455 | 1.00 | 29.60 | C |
| ATOM | 921 | CB | ALA | A | 371 | 15.928 | 30.792 | 18.720 | 1.00 | 29.76 | C |
| ATOM | 922 | C | ALA | A | 371 | 17.077 | 33.001 | 18.554 | 1.00 | 28.59 | C |
| ATOM | 923 | O | ALA | A | 371 | 17.964 | 32.561 | 19.226 | 1.00 | 27.57 | O |
| ATOM | 924 | N | PHE | A | 372 | 17.198 | 34.127 | 17.888 | 1.00 | 28.91 | N |
| ATOM | 925 | CA | PHE | A | 372 | 18.452 | 34.836 | 17.848 | 1.00 | 28.90 | C |
| ATOM | 926 | CB | PHE | A | 372 | 19.079 | 34.660 | 16.480 | 1.00 | 28.04 | C |
| ATOM | 927 | CG | PHE | A | 372 | 20.485 | 35.112 | 16.406 | 1.00 | 27.18 | C |
| ATOM | 928 | CD1 | PHE | A | 372 | 21.515 | 34.231 | 16.614 | 1.00 | 28.47 | C |
| ATOM | 929 | CE1 | PHE | A | 372 | 22.829 | 34.661 | 16.546 | 1.00 | 30.76 | C |
| ATOM | 930 | CZ | PHE | A | 372 | 23.107 | 36.004 | 16.312 | 1.00 | 28.92 | C |
| ATOM | 931 | CE2 | PHE | A | 372 | 22.085 | 36.873 | 16.119 | 1.00 | 28.65 | C |
| ATOM | 932 | CD2 | PHE | A | 372 | 20.781 | 36.429 | 16.158 | 1.00 | 27.09 | C |
| ATOM | 933 | C | PHE | A | 372 | 18.202 | 36.304 | 18.127 | 1.00 | 30.17 | C |
| ATOM | 934 | O | PHE | A | 372 | 17.312 | 36.889 | 17.543 | 1.00 | 30.94 | O |
| ATOM | 935 | N | ASP | A | 373 | 19.005 | 36.885 | 19.000 | 1.00 | 31.57 | N |
| ATOM | 936 | CA | ASP | A | 373 | 18.910 | 38.293 | 19.376 | 1.00 | 33.31 | C |
| ATOM | 937 | CB | ASP | A | 373 | 19.058 | 38.438 | 20.900 | 1.00 | 33.82 | C |
| ATOM | 938 | CG | ASP | A | 373 | 18.989 | 39.902 | 21.381 | 1.00 | 36.02 | C |
| ATOM | 939 | OD1 | ASP | A | 373 | 18.985 | 40.818 | 20.530 | 1.00 | 41.04 | O |
| ATOM | 940 | OD2 | ASP | A | 373 | 18.972 | 40.227 | 22.591 | 1.00 | 36.84 | O |
| ATOM | 941 | C | ASP | A | 373 | 20.020 | 39.049 | 18.649 | 1.00 | 34.95 | C |
| ATOM | 942 | O | ASP | A | 373 | 21.198 | 39.063 | 19.064 | 1.00 | 34.64 | O |
| ATOM | 943 | N | SER | A | 374 | 19.636 | 39.678 | 17.553 | 1.00 | 36.75 | N |
| ATOM | 944 | CA | SER | A | 374 | 20.585 | 40.409 | 16.717 | 1.00 | 39.28 | C |
| ATOM | 945 | CB | SER | A | 374 | 19.866 | 40.916 | 15.474 | 1.00 | 39.26 | C |
| ATOM | 946 | OG | SER | A | 374 | 20.836 | 41.201 | 14.487 | 1.00 | 43.88 | O |
| ATOM | 947 | C | SER | A | 374 | 21.325 | 41.581 | 17.378 | 1.00 | 39.94 | C |
| ATOM | 948 | O | SER | A | 374 | 22.515 | 41.766 | 17.173 | 1.00 | 40.56 | O |
| ATOM | 949 | N | GLN | A | 375 | 20.626 | 42.366 | 18.186 | 1.00 | 41.01 | N |
| ATOM | 950 | CA | GLN | A | 375 | 21.239 | 43.516 | 18.810 | 1.00 | 41.65 | C |
| ATOM | 951 | CB | GLN | A | 375 | 20.197 | 44.362 | 19.565 | 1.00 | 42.91 | C |
| ATOM | 952 | CG | GLN | A | 375 | 18.913 | 44.675 | 18.767 | 1.00 | 47.27 | C |
| ATOM | 953 | CD | GLN | A | 375 | 18.116 | 45.864 | 19.353 | 1.00 | 53.49 | C |
| ATOM | 954 | OE1 | GLN | A | 375 | 18.626 | 46.997 | 19.399 | 1.00 | 57.17 | O |
| ATOM | 955 | NE2 | GLN | A | 375 | 16.877 | 45.608 | 19.788 | 1.00 | 54.72 | N |
| ATOM | 956 | C | GLN | A | 375 | 22.344 | 43.098 | 19.762 | 1.00 | 40.86 | C |
| ATOM | 957 | O | GLN | A | 375 | 23.365 | 43.773 | 19.858 | 1.00 | 41.50 | O |
| ATOM | 958 | N | ASN | A | 376 | 22.155 | 41.987 | 20.463 | 1.00 | 39.20 | N |
| ATOM | 959 | CA | ASN | A | 376 | 23.137 | 41.552 | 21.441 | 1.00 | 38.16 | C |
| ATOM | 960 | CB | ASN | A | 376 | 22.438 | 41.264 | 22.766 | 1.00 | 38.40 | C |
| ATOM | 961 | CG | ASN | A | 376 | 21.791 | 42.527 | 23.387 | 1.00 | 40.33 | C |
| ATOM | 962 | OD1 | ASN | A | 376 | 22.488 | 43.368 | 23.935 | 1.00 | 41.58 | O |
| ATOM | 963 | ND2 | ASN | A | 376 | 20.452 | 42.644 | 23.301 | 1.00 | 40.49 | N |
| ATOM | 964 | C | ASN | A | 376 | 24.019 | 40.360 | 21.002 | 1.00 | 36.51 | C |
| ATOM | 965 | O | ASN | A | 376 | 24.852 | 39.906 | 21.769 | 1.00 | 37.34 | O |
| ATOM | 966 | N | ASN | A | 377 | 23.840 | 39.859 | 19.789 | 1.00 | 33.92 | N |
| ATOM | 967 | CA | ASN | A | 377 | 24.632 | 38.727 | 19.284 | 1.00 | 32.39 | C |
| ATOM | 968 | CB | ASN | A | 377 | 26.094 | 39.138 | 19.079 | 1.00 | 31.52 | C |
| ATOM | 969 | CG | ASN | A | 377 | 26.772 | 38.334 | 17.993 | 1.00 | 31.73 | C |
| ATOM | 970 | OD1 | ASN | A | 377 | 26.142 | 37.995 | 16.998 | 1.00 | 33.03 | O |
| ATOM | 971 | ND2 | ASN | A | 377 | 28.050 | 38.007 | 18.176 | 1.00 | 27.90 | N |
| ATOM | 972 | C | ASN | A | 377 | 24.550 | 37.477 | 20.194 | 1.00 | 31.00 | C |
| ATOM | 973 | O | ASN | A | 377 | 25.560 | 37.005 | 20.708 | 1.00 | 31.17 | O |
| ATOM | 974 | N | THR | A | 378 | 23.336 | 36.976 | 20.410 | 1.00 | 29.28 | N |
| ATOM | 975 | CA | THR | A | 378 | 23.110 | 35.831 | 21.282 | 1.00 | 28.19 | C |
| ATOM | 976 | CB | THR | A | 378 | 22.643 | 36.241 | 22.727 | 1.00 | 29.06 | C |
| ATOM | 977 | OG1 | THR | A | 378 | 21.395 | 36.949 | 22.674 | 1.00 | 27.46 | O |
| ATOM | 978 | CG2 | THR | A | 378 | 23.663 | 37.127 | 23.462 | 1.00 | 28.26 | C |
| ATOM | 979 | C | THR | A | 378 | 22.070 | 34.895 | 20.708 | 1.00 | 26.71 | C |
| ATOM | 980 | O | THR | A | 378 | 21.139 | 35.318 | 20.011 | 1.00 | 26.15 | O |
| ATOM | 981 | N | VAL | A | 379 | 22.231 | 33.615 | 21.032 | 1.00 | 26.22 | N |
| ATOM | 982 | CA | VAL | A | 379 | 21.361 | 32.557 | 20.517 | 1.00 | 24.86 | C |
| ATOM | 983 | CB | VAL | A | 379 | 22.149 | 31.671 | 19.525 | 1.00 | 24.33 | C |
| ATOM | 984 | CG1 | VAL | A | 379 | 23.217 | 30.881 | 20.210 | 1.00 | 24.14 | C |
| ATOM | 985 | CG2 | VAL | A | 379 | 21.210 | 30.747 | 18.777 | 1.00 | 24.47 | C |
| ATOM | 986 | C | VAL | A | 379 | 20.815 | 31.711 | 21.637 | 1.00 | 24.23 | C |
| ATOM | 987 | O | VAL | A | 379 | 21.507 | 31.421 | 22.559 | 1.00 | 24.32 | O |
| ATOM | 988 | N | TYR | A | 380 | 19.569 | 31.283 | 21.519 | 1.00 | 24.94 | N |
| ATOM | 989 | CA | TYR | A | 380 | 18.905 | 30.450 | 22.506 | 1.00 | 25.03 | C |
| ATOM | 990 | CB | TYR | A | 380 | 17.410 | 30.313 | 22.141 | 1.00 | 24.86 | C |
| ATOM | 991 | CG | TYR | A | 380 | 16.514 | 29.758 | 23.215 | 1.00 | 27.20 | C |
| ATOM | 992 | CD1 | TYR | A | 380 | 16.614 | 30.197 | 24.535 | 1.00 | 32.61 | C |
| ATOM | 993 | CE1 | TYR | A | 380 | 15.799 | 29.705 | 25.521 | 1.00 | 32.61 | C |
| ATOM | 994 | CZ | TYR | A | 380 | 14.852 | 28.746 | 25.210 | 1.00 | 35.82 | C |
| ATOM | 995 | OH | TYR | A | 380 | 14.038 | 28.236 | 26.193 | 1.00 | 38.69 | O |
| ATOM | 996 | CE2 | TYR | A | 380 | 14.742 | 28.277 | 23.938 | 1.00 | 34.16 | C |
| ATOM | 997 | CD2 | TYR | A | 380 | 15.589 | 28.791 | 22.939 | 1.00 | 32.66 | C |
| ATOM | 998 | C | TYR | A | 380 | 19.589 | 29.086 | 22.523 | 1.00 | 25.13 | C |
| ATOM | 999 | O | TYR | A | 380 | 19.647 | 28.416 | 21.514 | 1.00 | 25.44 | O |
| ATOM | 1000 | N | PHE | A | 381 | 20.127 | 28.698 | 23.675 | 1.00 | 25.96 | N |
| ATOM | 1001 | CA | PHE | A | 381 | 20.902 | 27.470 | 23.824 | 1.00 | 25.59 | C |
| ATOM | 1002 | CB | PHE | A | 381 | 22.328 | 27.773 | 23.471 | 1.00 | 25.60 | C |
| ATOM | 1003 | CG | PHE | A | 381 | 23.263 | 26.709 | 23.828 | 1.00 | 25.28 | C |
| ATOM | 1004 | CD1 | PHE | A | 381 | 23.306 | 25.561 | 23.106 | 1.00 | 26.68 | C |
| ATOM | 1005 | CE1 | PHE | A | 381 | 24.190 | 24.559 | 23.454 | 1.00 | 29.89 | C |
| ATOM | 1006 | CZ | PHE | A | 381 | 25.049 | 24.733 | 24.537 | 1.00 | 28.60 | C |
| ATOM | 1007 | CE2 | PHE | A | 381 | 25.007 | 25.865 | 25.234 | 1.00 | 28.13 | C |
| ATOM | 1008 | CD2 | PHE | A | 381 | 24.107 | 26.851 | 24.895 | 1.00 | 27.85 | C |
| ATOM | 1009 | C | PHE | A | 381 | 20.879 | 26.914 | 25.251 | 1.00 | 26.62 | C |
| ATOM | 1010 | O | PHE | A | 381 | 21.215 | 27.604 | 26.209 | 1.00 | 26.25 | O |
| ATOM | 1011 | N | ASP | A | 382 | 20.481 | 25.664 | 25.416 | 1.00 | 27.37 | N |
| ATOM | 1012 | CA | ASP | A | 382 | 20.441 | 25.075 | 26.766 | 1.00 | 28.62 | C |
| ATOM | 1013 | CB | ASP | A | 382 | 21.885 | 24.760 | 27.242 | 1.00 | 28.80 | C |
| ATOM | 1014 | CG | ASP | A | 382 | 21.936 | 23.653 | 28.283 | 1.00 | 31.74 | C |
| ATOM | 1015 | OD1 | ASP | A | 382 | 20.973 | 22.859 | 28.374 | 1.00 | 34.71 | O |
| ATOM | 1016 | OD2 | ASP | A | 382 | 22.911 | 23.477 | 29.045 | 1.00 | 34.98 | O |
| ATOM | 1017 | C | ASP | A | 382 | 19.705 | 25.888 | 27.853 | 1.00 | 28.15 | C |
| ATOM | 1018 | O | ASP | A | 382 | 20.150 | 25.923 | 28.997 | 1.00 | 29.36 | O |
| ATOM | 1019 | N | GLY | A | 383 | 18.564 | 26.485 | 27.514 | 1.00 | 28.44 | N |
| ATOM | 1020 | CA | GLY | A | 383 | 17.770 | 27.238 | 28.488 | 1.00 | 28.32 | C |
| ATOM | 1021 | C | GLY | A | 383 | 17.958 | 28.758 | 28.594 | 1.00 | 27.86 | C |
| ATOM | 1022 | O | GLY | A | 383 | 17.144 | 29.476 | 29.196 | 1.00 | 29.11 | O |
| ATOM | 1023 | N | LYS | A | 384 | 19.032 | 29.289 | 28.033 | 1.00 | 26.62 | N |
| ATOM | 1024 | CA | LYS | A | 384 | 19.241 | 30.731 | 28.108 | 1.00 | 26.42 | C |
| ATOM | 1025 | CB | LYS | A | 384 | 20.185 | 31.068 | 29.267 | 1.00 | 26.08 | C |
| ATOM | 1026 | CG | LYS | A | 384 | 19.627 | 30.747 | 30.653 | 1.00 | 28.51 | C |
| ATOM | 1027 | CD | LYS | A | 384 | 20.486 | 31.424 | 31.751 | 1.00 | 29.39 | C |
| ATOM | 1028 | CE | LYS | A | 384 | 20.028 | 31.086 | 33.196 | 1.00 | 30.70 | C |
| ATOM | 1029 | NZ | LYS | A | 384 | 20.910 | 31.746 | 34.288 | 1.00 | 29.22 | N |
| ATOM | 1030 | C | LYS | A | 384 | 19.797 | 31.259 | 26.791 | 1.00 | 25.94 | C |
| ATOM | 1031 | O | LYS | A | 384 | 20.097 | 30.466 | 25.900 | 1.00 | 26.76 | O |
| ATOM | 1032 | N | TYR | A | 385 | 19.974 | 32.578 | 26.672 | 1.00 | 24.63 | N |
| ATOM | 1033 | CA | TYR | A | 385 | 20.572 | 33.145 | 25.466 | 1.00 | 24.57 | C |
| ATOM | 1034 | CB | TYR | A | 385 | 19.948 | 34.488 | 25.115 | 1.00 | 24.03 | C |
| ATOM | 1035 | CG | TYR | A | 385 | 18.685 | 34.353 | 24.311 | 1.00 | 24.39 | C |
| ATOM | 1036 | CD1 | TYR | A | 385 | 17.468 | 34.095 | 24.917 | 1.00 | 26.02 | C |
| ATOM | 1037 | CE1 | TYR | A | 385 | 16.320 | 34.000 | 24.163 | 1.00 | 26.31 | C |
| ATOM | 1038 | CZ | TYR | A | 385 | 16.411 | 34.121 | 22.799 | 1.00 | 28.74 | C |
| ATOM | 1039 | OH | TYR | A | 385 | 15.321 | 33.976 | 21.988 | 1.00 | 27.24 | O |
| ATOM | 1040 | CE2 | TYR | A | 385 | 17.628 | 34.353 | 22.195 | 1.00 | 26.89 | C |
| ATOM | 1041 | CD2 | TYR | A | 385 | 18.723 | 34.458 | 22.944 | 1.00 | 24.65 | C |
| ATOM | 1042 | C | TYR | A | 385 | 22.101 | 33.213 | 25.671 | 1.00 | 24.69 | C |
| ATOM | 1043 | O | TYR | A | 385 | 22.590 | 33.626 | 26.720 | 1.00 | 25.62 | O |
| ATOM | 1044 | N | ALA | A | 386 | 22.825 | 32.741 | 24.671 | 1.00 | 24.40 | N |
| ATOM | 1045 | CA | ALA | A | 386 | 24.254 | 32.515 | 24.738 | 1.00 | 24.38 | C |
| ATOM | 1046 | CB | ALA | A | 386 | 24.514 | 31.107 | 24.385 | 1.00 | 23.79 | C |
| ATOM | 1047 | C | ALA | A | 386 | 25.008 | 33.368 | 23.745 | 1.00 | 25.29 | C |
| ATOM | 1048 | O | ALA | A | 386 | 24.623 | 33.438 | 22.579 | 1.00 | 26.61 | O |
| ATOM | 1049 | N | SER | A | 387 | 26.085 | 33.976 | 24.209 | 1.00 | 25.98 | N |
| ATOM | 1050 | CA | SER | A | 387 | 27.031 | 34.711 | 23.369 | 1.00 | 26.98 | C |
| ATOM | 1051 | CB | SER | A | 387 | 27.840 | 35.628 | 24.236 | 1.00 | 26.78 | C |
| ATOM | 1052 | OG | SER | A | 387 | 28.416 | 34.847 | 25.267 | 1.00 | 30.72 | O |
| ATOM | 1053 | C | SER | A | 387 | 27.985 | 33.696 | 22.787 | 1.00 | 27.67 | C |
| ATOM | 1054 | O | SER | A | 387 | 27.984 | 32.548 | 23.233 | 1.00 | 26.96 | O |
| ATOM | 1055 | N | PRO | A | 388 | 28.776 | 34.084 | 21.785 | 1.00 | 28.55 | N |
| ATOM | 1056 | CA | PRO | A | 388 | 29.788 | 33.193 | 21.216 | 1.00 | 29.40 | C |
| ATOM | 1057 | CB | PRO | A | 388 | 30.510 | 34.088 | 20.180 | 1.00 | 29.67 | C |
| ATOM | 1058 | CG | PRO | A | 388 | 29.538 | 35.118 | 19.839 | 1.00 | 29.41 | C |
| ATOM | 1059 | CD | PRO | A | 388 | 28.781 | 35.393 | 21.107 | 1.00 | 29.03 | C |
| ATOM | 1060 | C | PRO | A | 388 | 30.775 | 32.659 | 22.250 | 1.00 | 30.29 | C |
| ATOM | 1061 | O | PRO | A | 388 | 31.346 | 31.581 | 22.052 | 1.00 | 30.55 | O |
| ATOM | 1062 | N | ASP | A | 389 | 30.972 | 33.389 | 23.338 | 1.00 | 31.61 | N |
| ATOM | 1063 | CA | ASP | A | 389 | 31.864 | 32.951 | 24.406 | 1.00 | 32.74 | C |
| ATOM | 1064 | CB | ASP | A | 389 | 31.882 | 33.986 | 25.510 | 1.00 | 34.07 | C |
| ATOM | 1065 | CG | ASP | A | 389 | 32.640 | 35.207 | 25.127 | 1.00 | 39.93 | C |
| ATOM | 1066 | OD1 | ASP | A | 389 | 33.325 | 35.175 | 24.070 | 1.00 | 47.04 | O |
| ATOM | 1067 | OD2 | ASP | A | 389 | 32.601 | 36.259 | 25.813 | 1.00 | 46.60 | O |
| ATOM | 1068 | C | ASP | A | 389 | 31.484 | 31.614 | 25.044 | 1.00 | 31.53 | C |
| ATOM | 1069 | O | ASP | A | 389 | 32.359 | 30.893 | 25.528 | 1.00 | 31.63 | O |
| ATOM | 1070 | N | VAL | A | 390 | 30.189 | 31.317 | 25.069 | 1.00 | 29.87 | N |
| ATOM | 1071 | CA | VAL | A | 390 | 29.667 | 30.079 | 25.616 | 1.00 | 29.20 | C |
| ATOM | 1072 | CB | VAL | A | 390 | 28.119 | 30.083 | 25.572 | 1.00 | 29.33 | C |
| ATOM | 1073 | CG1 | VAL | A | 390 | 27.579 | 28.705 | 25.643 | 1.00 | 29.50 | C |
| ATOM | 1074 | CG2 | VAL | A | 390 | 27.582 | 30.907 | 26.704 | 1.00 | 29.08 | C |
| ATOM | 1075 | C | VAL | A | 390 | 30.183 | 28.865 | 24.870 | 1.00 | 28.35 | C |
| ATOM | 1076 | O | VAL | A | 390 | 30.307 | 27.811 | 25.440 | 1.00 | 28.58 | O |
| ATOM | 1077 | N | PHE | A | 391 | 30.551 | 29.038 | 23.610 | 1.00 | 28.30 | N |
| ATOM | 1078 | CA | PHE | A | 391 | 31.038 | 27.943 | 22.785 | 1.00 | 27.98 | C |
| ATOM | 1079 | CB | PHE | A | 391 | 30.365 | 28.034 | 21.431 | 1.00 | 27.73 | C |
| ATOM | 1080 | CG | PHE | A | 391 | 28.882 | 28.039 | 21.514 | 1.00 | 26.60 | C |
| ATOM | 1081 | CD1 | PHE | A | 391 | 28.180 | 29.233 | 21.549 | 1.00 | 25.13 | C |
| ATOM | 1082 | CE1 | PHE | A | 391 | 26.814 | 29.228 | 21.640 | 1.00 | 24.90 | C |
| ATOM | 1083 | CZ | PHE | A | 391 | 26.129 | 28.025 | 21.674 | 1.00 | 24.94 | C |
| ATOM | 1084 | CE2 | PHE | A | 391 | 26.812 | 26.837 | 21.650 | 1.00 | 24.26 | C |
| ATOM | 1085 | CD2 | PHE | A | 391 | 28.180 | 26.840 | 21.574 | 1.00 | 25.61 | C |
| ATOM | 1086 | C | PHE | A | 391 | 32.566 | 27.838 | 22.571 | 1.00 | 28.31 | C |
| ATOM | 1087 | O | PHE | A | 391 | 33.018 | 27.054 | 21.729 | 1.00 | 27.05 | O |
| ATOM | 1088 | N | LYS | A | 392 | 33.328 | 28.579 | 23.356 | 1.00 | 29.26 | N |
| ATOM | 1089 | CA | LYS | A | 392 | 34.795 | 28.578 | 23.281 | 1.00 | 31.34 | C |
| ATOM | 1090 | CB | LYS | A | 392 | 35.366 | 29.495 | 24.377 | 1.00 | 32.61 | C |
| ATOM | 1091 | CG | LYS | A | 392 | 36.861 | 29.771 | 24.281 | 1.00 | 35.79 | C |
| ATOM | 1092 | CD | LYS | A | 392 | 37.301 | 30.620 | 25.474 | 1.00 | 39.49 | C |
| ATOM | 1093 | CE | LYS | A | 392 | 38.826 | 30.579 | 25.699 | 1.00 | 41.94 | C |
| ATOM | 1094 | NZ | LYS | A | 392 | 39.185 | 31.071 | 27.075 | 1.00 | 42.28 | N |
| ATOM | 1095 | C | LYS | A | 392 | 35.456 | 27.202 | 23.366 | 1.00 | 31.54 | C |
| ATOM | 1096 | O | LYS | A | 392 | 36.322 | 26.898 | 22.567 | 1.00 | 31.50 | O |
| ATOM | 1097 | N | SER | A | 393 | 35.008 | 26.343 | 24.288 | 1.00 | 32.67 | N |
| ATOM | 1098 | CA | SER | A | 393 | 35.571 | 25.003 | 24.431 | 1.00 | 32.44 | C |
| ATOM | 1099 | CB | SER | A | 393 | 34.989 | 24.270 | 25.624 | 1.00 | 32.78 | C |
| ATOM | 1100 | OG | SER | A | 393 | 35.056 | 25.069 | 26.784 | 1.00 | 35.03 | O |
| ATOM | 1101 | C | SER | A | 393 | 35.400 | 24.110 | 23.240 | 1.00 | 32.29 | C |
| ATOM | 1102 | O | SER | A | 393 | 36.048 | 23.062 | 23.169 | 1.00 | 32.31 | O |
| ATOM | 1103 | N | LEU | A | 394 | 34.534 | 24.465 | 22.303 | 1.00 | 32.26 | N |
| ATOM | 1104 | CA | LEU | A | 394 | 34.436 | 23.653 | 21.121 | 1.00 | 31.95 | C |
| ATOM | 1105 | CB | LEU | A | 394 | 33.304 | 24.091 | 20.195 | 1.00 | 32.22 | C |
| ATOM | 1106 | CG | LEU | A | 394 | 31.829 | 23.898 | 20.560 | 1.00 | 30.03 | C |
| ATOM | 1107 | CD1 | LEU | A | 394 | 30.964 | 24.445 | 19.463 | 1.00 | 27.50 | C |
| ATOM | 1108 | CD2 | LEU | A | 394 | 31.547 | 22.458 | 20.809 | 1.00 | 31.13 | C |
| ATOM | 1109 | C | LEU | A | 394 | 35.720 | 23.720 | 20.316 | 1.00 | 32.56 | C |
| ATOM | 1110 | O | LEU | A | 394 | 35.948 | 22.892 | 19.493 | 1.00 | 32.76 | O |
| ATOM | 1111 | N | GLY | A | 395 | 36.520 | 24.749 | 20.498 | 1.00 | 35.25 | N |
| ATOM | 1112 | CA | GLY | A | 395 | 37.717 | 24.928 | 19.696 | 1.00 | 36.37 | C |
| ATOM | 1113 | C | GLY | A | 395 | 37.426 | 25.284 | 18.243 | 1.00 | 37.83 | C |
| ATOM | 1114 | O | GLY | A | 395 | 38.249 | 24.980 | 17.367 | 1.00 | 38.76 | O |
| ATOM | 1115 | N | CYS | A | 396 | 36.300 | 25.969 | 17.988 | 1.00 | 38.87 | N |
| ATOM | 1116 | CA | CYS | A | 396 | 35.877 | 26.363 | 16.626 | 1.00 | 39.61 | C |
| ATOM | 1117 | CB | CYS | A | 396 | 34.603 | 25.638 | 16.231 | 1.00 | 40.04 | C |
| ATOM | 1118 | SG | CYS | A | 396 | 34.493 | 23.892 | 16.532 | 1.00 | 47.12 | S |
| ATOM | 1119 | C | CYS | A | 396 | 35.483 | 27.836 | 16.522 | 1.00 | 38.76 | C |
| ATOM | 1120 | O | CYS | A | 396 | 34.364 | 28.140 | 16.063 | 1.00 | 37.44 | O |
| ATOM | 1121 | N | GLU | A | 397 | 36.359 | 28.747 | 16.928 | 1.00 | 38.45 | N |
| ATOM | 1122 | CA | GLU | A | 397 | 35.995 | 30.158 | 16.905 | 1.00 | 39.15 | C |
| ATOM | 1123 | CB | GLU | A | 397 | 37.123 | 31.064 | 17.392 | 1.00 | 39.98 | C |
| ATOM | 1124 | CG | GLU | A | 397 | 37.786 | 30.611 | 18.702 | 1.00 | 45.20 | C |
| ATOM | 1125 | CD | GLU | A | 397 | 37.249 | 31.254 | 19.991 | 1.00 | 51.31 | C |
| ATOM | 1126 | OE1 | GLU | A | 397 | 36.045 | 31.041 | 20.323 | 1.00 | 55.26 | O |
| ATOM | 1127 | OE2 | GLU | A | 397 | 38.046 | 31.920 | 20.717 | 1.00 | 53.17 | O |
| ATOM | 1128 | C | GLU | A | 397 | 35.487 | 30.605 | 15.520 | 1.00 | 37.80 | C |
| ATOM | 1129 | O | GLU | A | 397 | 34.511 | 31.296 | 15.465 | 1.00 | 37.56 | O |
| ATOM | 1130 | N | ASP | A | 398 | 36.118 | 30.197 | 14.423 | 1.00 | 37.26 | N |
| ATOM | 1131 | CA | ASP | A | 398 | 35.685 | 30.640 | 13.091 | 1.00 | 37.80 | C |
| ATOM | 1132 | CB | ASP | A | 398 | 36.695 | 30.253 | 12.000 | 1.00 | 39.29 | C |
| ATOM | 1133 | CG | ASP | A | 398 | 38.008 | 31.019 | 12.097 | 1.00 | 42.86 | C |
| ATOM | 1134 | OD1 | ASP | A | 398 | 38.097 | 32.024 | 12.844 | 1.00 | 48.94 | O |
| ATOM | 1135 | OD2 | ASP | A | 398 | 39.018 | 30.660 | 11.450 | 1.00 | 47.39 | O |
| ATOM | 1136 | C | ASP | A | 398 | 34.305 | 30.103 | 12.676 | 1.00 | 35.53 | C |
| ATOM | 1137 | O | ASP | A | 398 | 33.472 | 30.848 | 12.182 | 1.00 | 35.67 | O |
| ATOM | 1138 | N | PHE | A | 399 | 34.075 | 28.814 | 12.852 | 1.00 | 33.02 | N |
| ATOM | 1139 | CA | PHE | A | 399 | 32.755 | 28.264 | 12.574 | 1.00 | 31.62 | C |
| ATOM | 1140 | CB | PHE | A | 399 | 32.757 | 26.754 | 12.735 | 1.00 | 31.05 | C |
| ATOM | 1141 | CG | PHE | A | 399 | 31.399 | 26.151 | 12.729 | 1.00 | 30.78 | C |
| ATOM | 1142 | CD1 | PHE | A | 399 | 30.702 | 26.027 | 11.567 | 1.00 | 30.86 | C |
| ATOM | 1143 | CE1 | PHE | A | 399 | 29.437 | 25.502 | 11.561 | 1.00 | 31.41 | C |
| ATOM | 1144 | CZ | PHE | A | 399 | 28.856 | 25.092 | 12.742 | 1.00 | 31.17 | C |
| ATOM | 1145 | CE2 | PHE | A | 399 | 29.526 | 25.243 | 13.905 | 1.00 | 30.20 | C |
| ATOM | 1146 | CD2 | PHE | A | 399 | 30.797 | 25.777 | 13.906 | 1.00 | 29.57 | C |
| ATOM | 1147 | C | PHE | A | 399 | 31.665 | 28.932 | 13.466 | 1.00 | 30.38 | C |
| ATOM | 1148 | O | PHE | A | 399 | 30.567 | 29.240 | 13.014 | 1.00 | 30.06 | O |
| ATOM | 1149 | N | ILE | A | 400 | 31.975 | 29.187 | 14.718 | 1.00 | 29.09 | N |
| ATOM | 1150 | CA | ILE | A | 400 | 30.988 | 29.793 | 15.584 | 1.00 | 28.21 | C |
| ATOM | 1151 | CB | ILE | A | 400 | 31.453 | 29.709 | 17.022 | 1.00 | 27.49 | C |
| ATOM | 1152 | CG1 | ILE | A | 400 | 31.426 | 28.255 | 17.501 | 1.00 | 28.01 | C |
| ATOM | 1153 | CD1 | ILE | A | 400 | 30.013 | 27.629 | 17.525 | 1.00 | 27.35 | C |
| ATOM | 1154 | CG2 | ILE | A | 400 | 30.557 | 30.501 | 17.930 | 1.00 | 28.61 | C |
| ATOM | 1155 | C | ILE | A | 400 | 30.717 | 31.213 | 15.132 | 1.00 | 28.30 | C |
| ATOM | 1156 | O | ILE | A | 400 | 29.538 | 31.652 | 15.105 | 1.00 | 27.29 | O |
| ATOM | 1157 | N | SER | A | 401 | 31.774 | 31.949 | 14.758 | 1.00 | 27.64 | N |
| ATOM | 1158 | CA | SER | A | 401 | 31.552 | 33.329 | 14.298 | 1.00 | 28.85 | C |
| ATOM | 1159 | CB | SER | A | 401 | 32.850 | 34.152 | 14.224 | 1.00 | 28.93 | C |
| ATOM | 1160 | OG | SER | A | 401 | 33.622 | 33.761 | 13.129 | 1.00 | 35.10 | O |
| ATOM | 1161 | C | SER | A | 401 | 30.736 | 33.397 | 12.997 | 1.00 | 27.77 | C |
| ATOM | 1162 | O | SER | A | 401 | 29.938 | 34.283 | 12.818 | 1.00 | 28.99 | O |
| ATOM | 1163 | N | PHE | A | 402 | 30.910 | 32.423 | 12.133 | 1.00 | 27.14 | N |
| ATOM | 1164 | CA | PHE | A | 402 | 30.144 | 32.276 | 10.923 | 1.00 | 27.41 | C |
| ATOM | 1165 | CB | PHE | A | 402 | 30.800 | 31.145 | 10.137 | 1.00 | 27.67 | C |
| ATOM | 1166 | CG | PHE | A | 402 | 30.303 | 30.940 | 8.733 | 1.00 | 30.03 | C |
| ATOM | 1167 | CD1 | PHE | A | 402 | 29.486 | 31.840 | 8.091 | 1.00 | 31.94 | C |
| ATOM | 1168 | CE1 | PHE | A | 402 | 29.061 | 31.618 | 6.813 | 1.00 | 30.13 | C |
| ATOM | 1169 | CZ | PHE | A | 402 | 29.438 | 30.512 | 6.124 | 1.00 | 30.57 | C |
| ATOM | 1170 | CE2 | PHE | A | 402 | 30.266 | 29.595 | 6.712 | 1.00 | 33.09 | C |
| ATOM | 1171 | CD2 | PHE | A | 402 | 30.704 | 29.815 | 8.030 | 1.00 | 34.00 | C |
| ATOM | 1172 | C | PHE | A | 402 | 28.657 | 31.992 | 11.290 | 1.00 | 26.84 | C |
| ATOM | 1173 | O | PHE | A | 402 | 27.733 | 32.571 | 10.717 | 1.00 | 25.94 | O |
| ATOM | 1174 | N | VAL | A | 403 | 28.416 | 31.141 | 12.280 | 1.00 | 26.02 | N |
| ATOM | 1175 | CA | VAL | A | 403 | 27.035 | 30.855 | 12.669 | 1.00 | 25.17 | C |
| ATOM | 1176 | CB | VAL | A | 403 | 26.967 | 29.787 | 13.797 | 1.00 | 24.18 | C |
| ATOM | 1177 | CG1 | VAL | A | 403 | 25.606 | 29.750 | 14.394 | 1.00 | 25.35 | C |
| ATOM | 1178 | CG2 | VAL | A | 403 | 27.294 | 28.446 | 13.255 | 1.00 | 24.23 | C |
| ATOM | 1179 | C | VAL | A | 403 | 26.339 | 32.131 | 13.158 | 1.00 | 24.80 | C |
| ATOM | 1180 | O | VAL | A | 403 | 25.194 | 32.422 | 12.831 | 1.00 | 24.70 | O |
| ATOM | 1181 | N | PHE | A | 404 | 27.011 | 32.868 | 13.998 | 1.00 | 24.99 | N |
| ATOM | 1182 | CA | PHE | A | 404 | 26.398 | 34.058 | 14.547 | 1.00 | 25.96 | C |
| ATOM | 1183 | CB | PHE | A | 404 | 27.212 | 34.579 | 15.719 | 1.00 | 26.02 | C |
| ATOM | 1184 | CG | PHE | A | 404 | 26.912 | 33.880 | 17.037 | 1.00 | 25.96 | C |
| ATOM | 1185 | CD1 | PHE | A | 404 | 27.378 | 32.614 | 17.296 | 1.00 | 26.60 | C |
| ATOM | 1186 | CE1 | PHE | A | 404 | 27.087 | 31.962 | 18.535 | 1.00 | 27.71 | C |
| ATOM | 1187 | CZ | PHE | A | 404 | 26.358 | 32.572 | 19.475 | 1.00 | 26.31 | C |
| ATOM | 1188 | CE2 | PHE | A | 404 | 25.881 | 33.846 | 19.230 | 1.00 | 29.49 | C |
| ATOM | 1189 | CD2 | PHE | A | 404 | 26.178 | 34.501 | 18.010 | 1.00 | 28.52 | C |
| ATOM | 1190 | C | PHE | A | 404 | 26.232 | 35.121 | 13.488 | 1.00 | 26.66 | C |
| ATOM | 1191 | O | PHE | A | 404 | 25.278 | 35.891 | 13.515 | 1.00 | 27.41 | O |
| ATOM | 1192 | N | GLU | A | 405 | 27.151 | 35.166 | 12.537 | 1.00 | 27.79 | N |
| ATOM | 1193 | CA | GLU | A | 405 | 27.078 | 36.165 | 11.487 | 1.00 | 27.80 | C |
| ATOM | 1194 | CB | GLU | A | 405 | 28.339 | 36.163 | 10.623 | 1.00 | 28.35 | C |
| ATOM | 1195 | CG | GLU | A | 405 | 28.147 | 37.016 | 9.384 | 1.00 | 31.23 | C |
| ATOM | 1196 | CD | GLU | A | 405 | 29.446 | 37.528 | 8.769 | 1.00 | 35.82 | C |
| ATOM | 1197 | OE1 | GLU | A | 405 | 30.552 | 37.009 | 9.096 | 1.00 | 37.08 | O |
| ATOM | 1198 | OE2 | GLU | A | 405 | 29.334 | 38.448 | 7.937 | 1.00 | 35.95 | O |
| ATOM | 1199 | C | GLU | A | 405 | 25.861 | 35.864 | 10.629 | 1.00 | 27.96 | C |
| ATOM | 1200 | O | GLU | A | 405 | 25.126 | 36.773 | 10.228 | 1.00 | 25.82 | O |
| ATOM | 1201 | N | PHE | A | 406 | 25.658 | 34.576 | 10.373 | 1.00 | 27.97 | N |
| ATOM | 1202 | CA | PHE | A | 406 | 24.508 | 34.148 | 9.629 | 1.00 | 29.23 | C |
| ATOM | 1203 | CB | PHE | A | 406 | 24.549 | 32.656 | 9.326 | 1.00 | 28.50 | C |
| ATOM | 1204 | CG | PHE | A | 406 | 23.459 | 32.229 | 8.434 | 1.00 | 29.33 | C |
| ATOM | 1205 | CD1 | PHE | A | 406 | 23.588 | 32.396 | 7.058 | 1.00 | 31.26 | C |
| ATOM | 1206 | CE1 | PHE | A | 406 | 22.574 | 32.049 | 6.214 | 1.00 | 29.99 | C |
| ATOM | 1207 | CZ | PHE | A | 406 | 21.389 | 31.531 | 6.737 | 1.00 | 30.86 | C |
| ATOM | 1208 | CE2 | PHE | A | 406 | 21.253 | 31.340 | 8.104 | 1.00 | 30.00 | C |
| ATOM | 1209 | CD2 | PHE | A | 406 | 22.277 | 31.698 | 8.949 | 1.00 | 27.04 | C |
| ATOM | 1210 | C | PHE | A | 406 | 23.204 | 34.511 | 10.367 | 1.00 | 30.08 | C |
| ATOM | 1211 | O | PHE | A | 406 | 22.238 | 34.936 | 9.746 | 1.00 | 29.57 | O |
| ATOM | 1212 | N | GLY | A | 407 | 23.186 | 34.337 | 11.681 | 1.00 | 31.28 | N |
| ATOM | 1213 | CA | GLY | A | 407 | 22.009 | 34.643 | 12.485 | 1.00 | 31.96 | C |
| ATOM | 1214 | C | GLY | A | 407 | 21.699 | 36.116 | 12.401 | 1.00 | 33.32 | C |
| ATOM | 1215 | O | GLY | A | 407 | 20.581 | 36.539 | 12.135 | 1.00 | 32.80 | O |
| ATOM | 1216 | N | LYS | A | 408 | 22.729 | 36.915 | 12.577 | 1.00 | 35.20 | N |
| ATOM | 1217 | CA | LYS | A | 408 | 22.586 | 38.344 | 12.430 | 1.00 | 36.71 | C |
| ATOM | 1218 | CB | LYS | A | 408 | 23.931 | 38.993 | 12.688 | 1.00 | 37.55 | C |
| ATOM | 1219 | CG | LYS | A | 408 | 23.845 | 40.473 | 12.958 | 1.00 | 40.89 | C |
| ATOM | 1220 | CD | LYS | A | 408 | 25.125 | 41.000 | 13.605 | 1.00 | 44.55 | C |
| ATOM | 1221 | CE | LYS | A | 408 | 25.143 | 40.750 | 15.121 | 1.00 | 46.16 | C |
| ATOM | 1222 | NZ | LYS | A | 408 | 26.310 | 41.466 | 15.786 | 1.00 | 47.26 | N |
| ATOM | 1223 | C | LYS | A | 408 | 22.090 | 38.756 | 11.033 | 1.00 | 37.33 | C |
| ATOM | 1224 | O | LYS | A | 408 | 21.233 | 39.637 | 10.881 | 1.00 | 37.08 | O |
| ATOM | 1225 | N | SER | A | 409 | 22.610 | 38.106 | 10.005 | 1.00 | 37.62 | N |
| ATOM | 1226 | CA | SER | A | 409 | 22.258 | 38.499 | 8.649 | 1.00 | 38.03 | C |
| ATOM | 1227 | CB | SER | A | 409 | 23.258 | 37.906 | 7.681 | 1.00 | 37.87 | C |
| ATOM | 1228 | OG | SER | A | 409 | 22.671 | 37.759 | 6.421 | 1.00 | 43.04 | O |
| ATOM | 1229 | C | SER | A | 409 | 20.816 | 38.139 | 8.273 | 1.00 | 37.59 | C |
| ATOM | 1230 | O | SER | A | 409 | 20.146 | 38.891 | 7.596 | 1.00 | 37.65 | O |
| ATOM | 1231 | N | LEU | A | 410 | 20.330 | 36.994 | 8.730 | 1.00 | 38.00 | N |
| ATOM | 1232 | CA | LEU | A | 410 | 18.944 | 36.599 | 8.476 | 1.00 | 38.06 | C |
| ATOM | 1233 | CB | LEU | A | 410 | 18.771 | 35.108 | 8.728 | 1.00 | 37.93 | C |
| ATOM | 1234 | CG | LEU | A | 410 | 17.711 | 34.363 | 7.930 | 1.00 | 39.48 | C |
| ATOM | 1235 | CD1 | LEU | A | 410 | 17.866 | 34.566 | 6.431 | 1.00 | 40.64 | C |
| ATOM | 1236 | CD2 | LEU | A | 410 | 17.795 | 32.873 | 8.280 | 1.00 | 39.84 | C |
| ATOM | 1237 | C | LEU | A | 410 | 17.969 | 37.447 | 9.323 | 1.00 | 37.89 | C |
| ATOM | 1238 | O | LEU | A | 410 | 16.907 | 37.826 | 8.859 | 1.00 | 36.02 | O |
| ATOM | 1239 | N | CYS | A | 411 | 18.344 | 37.745 | 10.558 | 1.00 | 39.15 | N |
| ATOM | 1240 | CA | CYS | A | 411 | 17.552 | 38.634 | 11.416 | 1.00 | 40.62 | C |
| ATOM | 1241 | CB | CYS | A | 411 | 18.243 | 38.819 | 12.753 | 1.00 | 41.09 | C |
| ATOM | 1242 | SG | CYS | A | 411 | 18.069 | 37.449 | 13.893 | 1.00 | 41.89 | S |
| ATOM | 1243 | C | CYS | A | 411 | 17.371 | 40.029 | 10.822 | 1.00 | 41.43 | C |
| ATOM | 1244 | O | CYS | A | 411 | 16.301 | 40.596 | 10.880 | 1.00 | 41.63 | O |
| ATOM | 1245 | N | SER | A | 412 | 18.433 | 40.579 | 10.253 | 1.00 | 42.39 | N |
| ATOM | 1246 | CA | SER | A | 412 | 18.381 | 41.897 | 9.641 | 1.00 | 43.17 | C |
| ATOM | 1247 | CB | SER | A | 412 | 19.756 | 42.278 | 9.067 | 1.00 | 42.83 | C |
| ATOM | 1248 | OG | SER | A | 412 | 19.897 | 41.811 | 7.730 | 1.00 | 43.89 | O |
| ATOM | 1249 | C | SER | A | 412 | 17.325 | 42.003 | 8.534 | 1.00 | 44.19 | C |
| ATOM | 1250 | O | SER | A | 412 | 17.030 | 43.119 | 8.072 | 1.00 | 45.40 | O |
| ATOM | 1251 | N | MET | A | 413 | 16.771 | 40.880 | 8.085 | 1.00 | 43.86 | N |
| ATOM | 1252 | CA | MET | A | 413 | 15.732 | 40.913 | 7.065 | 1.00 | 44.53 | C |
| ATOM | 1253 | CB | MET | A | 413 | 15.874 | 39.740 | 6.102 | 1.00 | 44.68 | C |
| ATOM | 1254 | CG | MET | A | 413 | 17.179 | 39.724 | 5.375 | 1.00 | 45.77 | C |
| ATOM | 1255 | SD | MET | A | 413 | 17.293 | 38.227 | 4.484 | 1.00 | 52.29 | S |
| ATOM | 1256 | CE | MET | A | 413 | 19.021 | 38.123 | 4.084 | 1.00 | 52.15 | C |
| ATOM | 1257 | C | MET | A | 413 | 14.319 | 40.920 | 7.666 | 1.00 | 44.82 | C |
| ATOM | 1258 | O | MET | A | 413 | 13.354 | 41.154 | 6.948 | 1.00 | 45.27 | O |
| ATOM | 1259 | N | HIS | A | 414 | 14.207 | 40.686 | 8.969 | 1.00 | 44.42 | N |
| ATOM | 1260 | CA | HIS | A | 414 | 12.917 | 40.694 | 9.651 | 1.00 | 45.37 | C |
| ATOM | 1261 | CB | HIS | A | 414 | 12.369 | 42.124 | 9.831 | 1.00 | 46.54 | C |
| ATOM | 1262 | CG | HIS | A | 414 | 13.414 | 43.131 | 10.202 | 1.00 | 49.03 | C |
| ATOM | 1263 | ND1 | HIS | A | 414 | 13.905 | 43.252 | 11.484 | 1.00 | 52.00 | N |
| ATOM | 1264 | CE1 | HIS | A | 414 | 14.830 | 44.198 | 11.511 | 1.00 | 52.74 | C |
| ATOM | 1265 | NE2 | HIS | A | 414 | 14.956 | 44.694 | 10.290 | 1.00 | 53.78 | N |
| ATOM | 1266 | CD2 | HIS | A | 414 | 14.078 | 44.046 | 9.452 | 1.00 | 51.02 | C |
| ATOM | 1267 | C | HIS | A | 414 | 11.920 | 39.867 | 8.867 | 1.00 | 44.54 | C |
| ATOM | 1268 | O | HIS | A | 414 | 10.905 | 40.380 | 8.397 | 1.00 | 45.13 | O |
| ATOM | 1269 | N | LEU | A | 415 | 12.221 | 38.586 | 8.712 | 1.00 | 43.37 | N |
| ATOM | 1270 | CA | LEU | A | 415 | 11.345 | 37.696 | 7.992 | 1.00 | 42.05 | C |
| ATOM | 1271 | CB | LEU | A | 415 | 12.098 | 36.448 | 7.563 | 1.00 | 42.25 | C |
| ATOM | 1272 | CG | LEU | A | 415 | 13.274 | 36.739 | 6.635 | 1.00 | 42.37 | C |
| ATOM | 1273 | CD1 | LEU | A | 415 | 14.213 | 35.584 | 6.615 | 1.00 | 43.50 | C |
| ATOM | 1274 | CD2 | LEU | A | 415 | 12.736 | 37.013 | 5.254 | 1.00 | 42.77 | C |
| ATOM | 1275 | C | LEU | A | 415 | 10.200 | 37.311 | 8.889 | 1.00 | 41.21 | C |
| ATOM | 1276 | O | LEU | A | 415 | 10.379 | 37.116 | 10.073 | 1.00 | 40.43 | O |
| ATOM | 1277 | N | THR | A | 416 | 9.019 | 37.191 | 8.310 | 1.00 | 40.22 | N |
| ATOM | 1278 | CA | THR | A | 416 | 7.852 | 36.771 | 9.050 | 1.00 | 39.80 | C |
| ATOM | 1279 | CB | THR | A | 416 | 6.587 | 37.152 | 8.269 | 1.00 | 39.84 | C |
| ATOM | 1280 | OG1 | THR | A | 416 | 6.623 | 36.538 | 6.978 | 1.00 | 39.74 | O |
| ATOM | 1281 | CG2 | THR | A | 416 | 6.546 | 38.636 | 7.950 | 1.00 | 39.98 | C |
| ATOM | 1282 | C | THR | A | 416 | 7.890 | 35.270 | 9.203 | 1.00 | 39.01 | C |
| ATOM | 1283 | O | THR | A | 416 | 8.698 | 34.609 | 8.553 | 1.00 | 38.71 | O |
| ATOM | 1284 | N | GLU | A | 417 | 7.004 | 34.728 | 10.035 | 1.00 | 38.46 | N |
| ATOM | 1285 | CA | GLU | A | 417 | 6.925 | 33.289 | 10.241 | 1.00 | 38.70 | C |
| ATOM | 1286 | CB | GLU | A | 417 | 5.879 | 32.940 | 11.318 | 1.00 | 38.85 | C |
| ATOM | 1287 | CG | GLU | A | 417 | 6.350 | 33.068 | 12.768 | 1.00 | 41.11 | C |
| ATOM | 1288 | CD | GLU | A | 417 | 7.583 | 32.225 | 13.115 | 1.00 | 41.56 | C |
| ATOM | 1289 | OE1 | GLU | A | 417 | 7.436 | 31.070 | 13.527 | 1.00 | 40.66 | O |
| ATOM | 1290 | OE2 | GLU | A | 417 | 8.707 | 32.744 | 13.014 | 1.00 | 44.69 | O |
| ATOM | 1291 | C | GLU | A | 417 | 6.599 | 32.558 | 8.921 | 1.00 | 38.40 | C |
| ATOM | 1292 | O | GLU | A | 417 | 7.140 | 31.493 | 8.629 | 1.00 | 37.21 | O |
| ATOM | 1293 | N | ASP | A | 418 | 5.717 | 33.122 | 8.117 | 1.00 | 38.52 | N |
| ATOM | 1294 | CA | ASP | A | 418 | 5.382 | 32.489 | 6.845 | 1.00 | 39.36 | C |
| ATOM | 1295 | CB | ASP | A | 418 | 4.134 | 33.115 | 6.221 | 1.00 | 40.08 | C |
| ATOM | 1296 | CG | ASP | A | 418 | 2.858 | 32.783 | 7.007 | 1.00 | 44.36 | C |
| ATOM | 1297 | OD1 | ASP | A | 418 | 2.931 | 31.961 | 7.953 | 1.00 | 50.10 | O |
| ATOM | 1298 | OD2 | ASP | A | 418 | 1.736 | 33.282 | 6.742 | 1.00 | 48.86 | O |
| ATOM | 1299 | C | ASP | A | 418 | 6.540 | 32.531 | 5.865 | 1.00 | 38.10 | C |
| ATOM | 1300 | O | ASP | A | 418 | 6.712 | 31.634 | 5.057 | 1.00 | 37.14 | O |
| ATOM | 1301 | N | GLU | A | 419 | 7.311 | 33.602 | 5.925 | 1.00 | 37.67 | N |
| ATOM | 1302 | CA | GLU | A | 419 | 8.479 | 33.730 | 5.074 | 1.00 | 37.99 | C |
| ATOM | 1303 | CB | GLU | A | 419 | 9.062 | 35.165 | 5.163 | 1.00 | 38.22 | C |
| ATOM | 1304 | CG | GLU | A | 419 | 8.269 | 36.175 | 4.336 | 1.00 | 40.90 | C |
| ATOM | 1305 | CD | GLU | A | 419 | 8.530 | 37.629 | 4.682 | 1.00 | 41.76 | C |
| ATOM | 1306 | OE1 | GLU | A | 419 | 9.193 | 37.895 | 5.685 | 1.00 | 43.26 | O |
| ATOM | 1307 | OE2 | GLU | A | 419 | 8.059 | 38.512 | 3.938 | 1.00 | 45.04 | O |
| ATOM | 1308 | C | GLU | A | 419 | 9.519 | 32.657 | 5.459 | 1.00 | 36.84 | C |
| ATOM | 1309 | O | GLU | A | 419 | 10.106 | 32.012 | 4.590 | 1.00 | 36.16 | O |
| ATOM | 1310 | N | ILE | A | 420 | 9.708 | 32.453 | 6.760 | 1.00 | 35.93 | N |
| ATOM | 1311 | CA | ILE | A | 420 | 10.695 | 31.488 | 7.233 | 1.00 | 35.74 | C |
| ATOM | 1312 | CB | ILE | A | 420 | 10.899 | 31.607 | 8.740 | 1.00 | 36.17 | C |
| ATOM | 1313 | CG1 | ILE | A | 420 | 11.712 | 32.873 | 9.008 | 1.00 | 38.07 | C |
| ATOM | 1314 | CD1 | ILE | A | 420 | 11.838 | 33.228 | 10.464 | 1.00 | 39.66 | C |
| ATOM | 1315 | CG2 | ILE | A | 420 | 11.617 | 30.377 | 9.287 | 1.00 | 36.15 | C |
| ATOM | 1316 | C | ILE | A | 420 | 10.311 | 30.085 | 6.856 | 1.00 | 34.34 | C |
| ATOM | 1317 | O | ILE | A | 420 | 11.156 | 29.282 | 6.517 | 1.00 | 33.17 | O |
| ATOM | 1318 | N | ALA | A | 421 | 9.013 | 29.813 | 6.914 | 1.00 | 33.91 | N |
| ATOM | 1319 | CA | ALA | A | 421 | 8.459 | 28.535 | 6.558 | 1.00 | 32.70 | C |
| ATOM | 1320 | CB | ALA | A | 421 | 7.024 | 28.543 | 6.813 | 1.00 | 33.68 | C |
| ATOM | 1321 | C | ALA | A | 421 | 8.676 | 28.194 | 5.084 | 1.00 | 32.56 | C |
| ATOM | 1322 | O | ALA | A | 421 | 9.065 | 27.064 | 4.769 | 1.00 | 31.53 | O |
| ATOM | 1323 | N | LEU | A | 422 | 8.384 | 29.140 | 4.188 | 1.00 | 31.71 | N |
| ATOM | 1324 | CA | LEU | A | 422 | 8.508 | 28.871 | 2.773 | 1.00 | 32.27 | C |
| ATOM | 1325 | CB | LEU | A | 422 | 7.779 | 29.923 | 1.948 | 1.00 | 33.13 | C |
| ATOM | 1326 | CG | LEU | A | 422 | 6.261 | 29.927 | 2.141 | 1.00 | 37.47 | C |
| ATOM | 1327 | CD1 | LEU | A | 422 | 5.611 | 30.995 | 1.361 | 1.00 | 40.39 | C |
| ATOM | 1328 | CD2 | LEU | A | 422 | 5.677 | 28.631 | 1.696 | 1.00 | 40.84 | C |
| ATOM | 1329 | C | LEU | A | 422 | 9.972 | 28.770 | 2.367 | 1.00 | 31.63 | C |
| ATOM | 1330 | O | LEU | A | 422 | 10.319 | 28.037 | 1.452 | 1.00 | 31.16 | O |
| ATOM | 1331 | N | PHE | A | 423 | 10.828 | 29.525 | 3.046 | 1.00 | 31.46 | N |
| ATOM | 1332 | CA | PHE | A | 423 | 12.247 | 29.487 | 2.758 | 1.00 | 31.86 | C |
| ATOM | 1333 | CB | PHE | A | 423 | 12.957 | 30.654 | 3.414 | 1.00 | 32.18 | C |
| ATOM | 1334 | CG | PHE | A | 423 | 14.396 | 30.795 | 3.015 | 1.00 | 32.59 | C |
| ATOM | 1335 | CD1 | PHE | A | 423 | 14.800 | 30.673 | 1.696 | 1.00 | 34.16 | C |
| ATOM | 1336 | CE1 | PHE | A | 423 | 16.128 | 30.853 | 1.357 | 1.00 | 34.47 | C |
| ATOM | 1337 | CZ | PHE | A | 423 | 17.056 | 31.142 | 2.336 | 1.00 | 33.24 | C |
| ATOM | 1338 | CE2 | PHE | A | 423 | 16.654 | 31.246 | 3.647 | 1.00 | 34.02 | C |
| ATOM | 1339 | CD2 | PHE | A | 423 | 15.342 | 31.079 | 3.977 | 1.00 | 33.39 | C |
| ATOM | 1340 | C | PHE | A | 423 | 12.852 | 28.159 | 3.239 | 1.00 | 31.47 | C |
| ATOM | 1341 | O | PHE | A | 423 | 13.743 | 27.612 | 2.592 | 1.00 | 31.65 | O |
| ATOM | 1342 | N | SER | A | 424 | 12.379 | 27.678 | 4.387 | 1.00 | 30.47 | N |
| ATOM | 1343 | CA | SER | A | 424 | 12.730 | 26.364 | 4.874 | 1.00 | 29.56 | C |
| ATOM | 1344 | CB | SER | A | 424 | 12.038 | 26.069 | 6.182 | 1.00 | 28.62 | C |
| ATOM | 1345 | OG | SER | A | 424 | 12.536 | 26.902 | 7.175 | 1.00 | 30.82 | O |
| ATOM | 1346 | C | SER | A | 424 | 12.337 | 25.283 | 3.897 | 1.00 | 28.62 | C |
| ATOM | 1347 | O | SER | A | 424 | 13.121 | 24.391 | 3.610 | 1.00 | 28.23 | O |
| ATOM | 1348 | N | ALA | A | 425 | 11.106 | 25.324 | 3.433 | 1.00 | 28.24 | N |
| ATOM | 1349 | CA | ALA | A | 425 | 10.643 | 24.333 | 2.465 | 1.00 | 28.61 | C |
| ATOM | 1350 | CB | ALA | A | 425 | 9.139 | 24.429 | 2.249 | 1.00 | 28.26 | C |
| ATOM | 1351 | C | ALA | A | 425 | 11.389 | 24.386 | 1.123 | 1.00 | 29.02 | C |
| ATOM | 1352 | O | ALA | A | 425 | 11.530 | 23.372 | 0.434 | 1.00 | 30.06 | O |
| ATOM | 1353 | N | PHE | A | 426 | 11.873 | 25.567 | 0.778 | 1.00 | 30.03 | N |
| ATOM | 1354 | CA | PHE | A | 426 | 12.611 | 25.820 | −0.451 | 1.00 | 31.03 | C |
| ATOM | 1355 | CB | PHE | A | 426 | 12.751 | 27.342 | −0.637 | 1.00 | 30.64 | C |
| ATOM | 1356 | CG | PHE | A | 426 | 13.479 | 27.713 | −1.875 | 1.00 | 33.04 | C |
| ATOM | 1357 | CD1 | PHE | A | 426 | 12.835 | 27.731 | −3.094 | 1.00 | 32.88 | C |
| ATOM | 1358 | CE1 | PHE | A | 426 | 13.524 | 28.021 | −4.238 | 1.00 | 34.86 | C |
| ATOM | 1359 | CZ | PHE | A | 426 | 14.872 | 28.293 | −4.189 | 1.00 | 34.01 | C |
| ATOM | 1360 | CE2 | PHE | A | 426 | 15.515 | 28.287 | −2.986 | 1.00 | 34.91 | C |
| ATOM | 1361 | CD2 | PHE | A | 426 | 14.817 | 28.006 | −1.834 | 1.00 | 35.40 | C |
| ATOM | 1362 | C | PHE | A | 426 | 14.009 | 25.142 | −0.447 | 1.00 | 31.56 | C |
| ATOM | 1363 | O | PHE | A | 426 | 14.328 | 24.361 | −1.329 | 1.00 | 31.28 | O |
| ATOM | 1364 | N | VAL | A | 427 | 14.846 | 25.426 | 0.557 | 1.00 | 32.26 | N |
| ATOM | 1365 | CA | VAL | A | 427 | 16.204 | 24.824 | 0.597 | 1.00 | 33.10 | C |
| ATOM | 1366 | CB | VAL | A | 427 | 17.168 | 25.602 | 1.524 | 1.00 | 33.53 | C |
| ATOM | 1367 | CG1 | VAL | A | 427 | 17.205 | 27.072 | 1.140 | 1.00 | 34.66 | C |
| ATOM | 1368 | CG2 | VAL | A | 427 | 16.772 | 25.463 | 2.953 | 1.00 | 35.57 | C |
| ATOM | 1369 | C | VAL | A | 427 | 16.186 | 23.370 | 0.987 | 1.00 | 32.54 | C |
| ATOM | 1370 | O | VAL | A | 427 | 17.182 | 22.637 | 0.881 | 1.00 | 32.60 | O |
| ATOM | 1371 | N | LEU | A | 428 | 15.030 | 22.933 | 1.433 | 1.00 | 33.17 | N |
| ATOM | 1372 | CA | LEU | A | 428 | 14.895 | 21.543 | 1.788 | 1.00 | 33.15 | C |
| ATOM | 1373 | CB | LEU | A | 428 | 13.754 | 21.382 | 2.751 | 1.00 | 33.88 | C |
| ATOM | 1374 | CG | LEU | A | 428 | 13.731 | 20.007 | 3.346 | 1.00 | 35.63 | C |
| ATOM | 1375 | CD1 | LEU | A | 428 | 14.751 | 19.913 | 4.473 | 1.00 | 36.29 | C |
| ATOM | 1376 | CD2 | LEU | A | 428 | 12.345 | 19.712 | 3.830 | 1.00 | 40.57 | C |
| ATOM | 1377 | C | LEU | A | 428 | 14.640 | 20.688 | 0.563 | 1.00 | 32.72 | C |
| ATOM | 1378 | O | LEU | A | 428 | 15.188 | 19.577 | 0.444 | 1.00 | 32.28 | O |
| ATOM | 1379 | N | MET | A | 429 | 13.762 | 21.145 | −0.315 | 1.00 | 33.59 | N |
| ATOM | 1380 | CA | MET | A | 429 | 13.441 | 20.428 | −1.521 | 1.00 | 34.98 | C |
| ATOM | 1381 | CB | MET | A | 429 | 11.977 | 20.670 | −1.871 | 1.00 | 35.55 | C |
| ATOM | 1382 | CG | MET | A | 429 | 11.036 | 20.479 | −0.714 | 1.00 | 38.33 | C |
| ATOM | 1383 | SD | MET | A | 429 | 10.845 | 18.793 | −0.205 | 1.00 | 43.35 | S |
| ATOM | 1384 | CE | MET | A | 429 | 10.460 | 18.095 | −1.842 | 1.00 | 40.58 | C |
| ATOM | 1385 | C | MET | A | 429 | 14.293 | 20.979 | −2.639 | 1.00 | 35.57 | C |
| ATOM | 1386 | O | MET | A | 429 | 13.772 | 21.578 | −3.556 | 1.00 | 35.78 | O |
| ATOM | 1387 | N | SER | A | 430 | 15.602 | 20.813 | −2.559 | 1.00 | 36.04 | N |
| ATOM | 1388 | CA | SER | A | 430 | 16.465 | 21.298 | −3.608 | 1.00 | 37.23 | C |
| ATOM | 1389 | CB | SER | A | 430 | 17.782 | 21.792 | −3.060 | 1.00 | 37.79 | C |
| ATOM | 1390 | OG | SER | A | 430 | 17.672 | 22.092 | −1.698 | 1.00 | 40.76 | O |
| ATOM | 1391 | C | SER | A | 430 | 16.760 | 20.144 | −4.507 | 1.00 | 37.02 | C |
| ATOM | 1392 | O | SER | A | 430 | 17.088 | 19.054 | −4.034 | 1.00 | 37.47 | O |
| ATOM | 1393 | N | ALA | A | 431 | 16.683 | 20.375 | −5.800 | 1.00 | 37.33 | N |
| ATOM | 1394 | CA | ALA | A | 431 | 16.869 | 19.301 | −6.749 | 1.00 | 38.02 | C |
| ATOM | 1395 | CB | ALA | A | 431 | 16.086 | 19.581 | −7.999 | 1.00 | 38.37 | C |
| ATOM | 1396 | C | ALA | A | 431 | 18.332 | 19.088 | −7.071 | 1.00 | 39.47 | C |
| ATOM | 1397 | O | ALA | A | 431 | 18.675 | 18.155 | −7.801 | 1.00 | 40.70 | O |
| ATOM | 1398 | N | ASP | A | 432 | 19.219 | 19.916 | −6.528 | 1.00 | 40.30 | N |
| ATOM | 1399 | CA | ASP | A | 432 | 20.619 | 19.731 | −6.853 | 1.00 | 41.30 | C |
| ATOM | 1400 | CB | ASP | A | 432 | 21.304 | 21.041 | −7.201 | 1.00 | 42.25 | C |
| ATOM | 1401 | CG | ASP | A | 432 | 21.026 | 22.093 | −6.217 | 1.00 | 45.95 | C |
| ATOM | 1402 | OD1 | ASP | A | 432 | 21.919 | 22.947 | −6.001 | 1.00 | 48.76 | O |
| ATOM | 1403 | OD2 | ASP | A | 432 | 19.930 | 22.144 | −5.610 | 1.00 | 53.78 | O |
| ATOM | 1404 | C | ASP | A | 432 | 21.425 | 18.985 | −5.818 | 1.00 | 40.17 | C |
| ATOM | 1405 | O | ASP | A | 432 | 22.623 | 18.843 | −5.968 | 1.00 | 42.18 | O |
| ATOM | 1406 | N | ARG | A | 433 | 20.810 | 18.440 | −4.800 | 1.00 | 38.39 | N |
| ATOM | 1407 | CA | ARG | A | 433 | 21.597 | 17.625 | −3.915 | 1.00 | 37.16 | C |
| ATOM | 1408 | CB | ARG | A | 433 | 20.744 | 17.081 | −2.804 | 1.00 | 36.78 | C |
| ATOM | 1409 | CG | ARG | A | 433 | 19.976 | 18.117 | −2.084 | 1.00 | 37.06 | C |
| ATOM | 1410 | CD | ARG | A | 433 | 20.810 | 19.149 | −1.420 | 1.00 | 36.88 | C |
| ATOM | 1411 | NE | ARG | A | 433 | 19.938 | 19.958 | −0.589 | 1.00 | 38.16 | N |
| ATOM | 1412 | CZ | ARG | A | 433 | 20.335 | 20.952 | 0.180 | 1.00 | 37.36 | C |
| ATOM | 1413 | NH1 | ARG | A | 433 | 21.611 | 21.301 | 0.238 | 1.00 | 34.55 | N |
| ATOM | 1414 | NH2 | ARG | A | 433 | 19.443 | 21.633 | 0.885 | 1.00 | 38.38 | N |
| ATOM | 1415 | C | ARG | A | 433 | 22.145 | 16.450 | −4.731 | 1.00 | 37.08 | C |
| ATOM | 1416 | O | ARG | A | 433 | 21.441 | 15.925 | −5.593 | 1.00 | 35.43 | O |
| ATOM | 1417 | N | SER | A | 434 | 23.370 | 16.022 | −4.430 | 1.00 | 36.72 | N |
| ATOM | 1418 | CA | SER | A | 434 | 23.963 | 14.877 | −5.105 | 1.00 | 36.76 | C |
| ATOM | 1419 | CB | SER | A | 434 | 25.390 | 14.603 | −4.593 | 1.00 | 36.85 | C |
| ATOM | 1420 | OG | SER | A | 434 | 26.176 | 15.757 | −4.604 | 1.00 | 38.46 | O |
| ATOM | 1421 | C | SER | A | 434 | 23.196 | 13.630 | −4.782 | 1.00 | 36.80 | C |
| ATOM | 1422 | O | SER | A | 434 | 22.660 | 13.481 | −3.670 | 1.00 | 35.82 | O |
| ATOM | 1423 | N | TRP | A | 435 | 23.206 | 12.720 | −5.754 | 1.00 | 36.33 | N |
| ATOM | 1424 | CA | TRP | A | 435 | 22.706 | 11.376 | −5.614 | 1.00 | 36.22 | C |
| ATOM | 1425 | CB | TRP | A | 435 | 23.314 | 10.715 | −4.376 | 1.00 | 36.57 | C |
| ATOM | 1426 | CG | TRP | A | 435 | 24.778 | 10.979 | −4.204 | 1.00 | 38.75 | C |
| ATOM | 1427 | CD1 | TRP | A | 435 | 25.379 | 11.502 | −3.115 | 1.00 | 40.89 | C |
| ATOM | 1428 | NE1 | TRP | A | 435 | 26.733 | 11.585 | −3.315 | 1.00 | 41.15 | N |
| ATOM | 1429 | CE2 | TRP | A | 435 | 27.026 | 11.109 | −4.560 | 1.00 | 39.69 | C |
| ATOM | 1430 | CD2 | TRP | A | 435 | 25.825 | 10.717 | −5.147 | 1.00 | 38.76 | C |
| ATOM | 1431 | CE3 | TRP | A | 435 | 25.862 | 10.172 | −6.425 | 1.00 | 40.76 | C |
| ATOM | 1432 | CZ3 | TRP | A | 435 | 27.074 | 10.049 | −7.063 | 1.00 | 41.43 | C |
| ATOM | 1433 | CH2 | TRP | A | 435 | 28.250 | 10.450 | −6.451 | 1.00 | 41.35 | C |
| ATOM | 1434 | CZ2 | TRP | A | 435 | 28.249 | 10.976 | −5.195 | 1.00 | 41.84 | C |
| ATOM | 1435 | C | TRP | A | 435 | 21.193 | 11.264 | −5.575 | 1.00 | 36.43 | C |
| ATOM | 1436 | O | TRP | A | 435 | 20.674 | 10.269 | −5.110 | 1.00 | 35.92 | O |
| ATOM | 1437 | N | LEU | A | 436 | 20.483 | 12.272 | −6.048 | 1.00 | 37.43 | N |
| ATOM | 1438 | CA | LEU | A | 436 | 19.013 | 12.185 | −6.139 | 1.00 | 38.64 | C |
| ATOM | 1439 | CB | LEU | A | 436 | 18.419 | 13.585 | −6.194 | 1.00 | 38.27 | C |
| ATOM | 1440 | CG | LEU | A | 436 | 18.387 | 14.376 | −4.902 | 1.00 | 37.94 | C |
| ATOM | 1441 | CD1 | LEU | A | 436 | 17.844 | 15.752 | −5.190 | 1.00 | 38.90 | C |
| ATOM | 1442 | CD2 | LEU | A | 436 | 17.547 | 13.667 | −3.885 | 1.00 | 37.76 | C |
| ATOM | 1443 | C | LEU | A | 436 | 18.531 | 11.436 | −7.394 | 1.00 | 40.22 | C |
| ATOM | 1444 | O | LEU | A | 436 | 19.022 | 11.691 | −8.497 | 1.00 | 40.43 | O |
| ATOM | 1445 | N | GLN | A | 437 | 17.542 | 10.562 | −7.241 | 1.00 | 42.27 | N |
| ATOM | 1446 | CA | GLN | A | 437 | 16.981 | 9.825 | −8.370 | 1.00 | 43.95 | C |
| ATOM | 1447 | CB | GLN | A | 437 | 16.334 | 8.537 | −7.896 | 1.00 | 44.38 | C |
| ATOM | 1448 | CG | GLN | A | 437 | 17.247 | 7.710 | −7.059 | 1.00 | 46.96 | C |
| ATOM | 1449 | CD | GLN | A | 437 | 16.647 | 6.389 | −6.653 | 1.00 | 50.03 | C |
| ATOM | 1450 | OE1 | GLN | A | 437 | 15.546 | 6.043 | −7.084 | 1.00 | 54.83 | O |
| ATOM | 1451 | NE2 | GLN | A | 437 | 17.370 | 5.640 | −5.826 | 1.00 | 50.03 | N |
| ATOM | 1452 | C | GLN | A | 437 | 15.958 | 10.644 | −9.136 | 1.00 | 44.68 | C |
| ATOM | 1453 | O | GLN | A | 437 | 16.051 | 10.788 | −10.346 | 1.00 | 45.04 | O |
| ATOM | 1454 | N | GLU | A | 438 | 15.000 | 11.221 | −8.433 | 1.00 | 45.84 | N |
| ATOM | 1455 | CA | GLU | A | 438 | 13.933 | 11.977 | −9.093 | 1.00 | 46.74 | C |
| ATOM | 1456 | CB | GLU | A | 438 | 12.628 | 11.716 | −8.360 | 1.00 | 47.49 | C |
| ATOM | 1457 | CG | GLU | A | 438 | 12.433 | 10.254 | −8.005 | 1.00 | 49.80 | C |
| ATOM | 1458 | CD | GLU | A | 438 | 11.318 | 10.066 | −7.011 | 1.00 | 54.24 | C |
| ATOM | 1459 | OE1 | GLU | A | 438 | 10.145 | 10.256 | −7.410 | 1.00 | 55.09 | O |
| ATOM | 1460 | OE2 | GLU | A | 438 | 11.625 | 9.744 | −5.832 | 1.00 | 58.98 | O |
| ATOM | 1461 | C | GLU | A | 438 | 14.169 | 13.475 | −9.154 | 1.00 | 46.63 | C |
| ATOM | 1462 | O | GLU | A | 438 | 13.399 | 14.242 | −8.586 | 1.00 | 47.40 | O |
| ATOM | 1463 | N | LYS | A | 439 | 15.192 | 13.897 | −9.887 | 1.00 | 46.50 | N |
| ATOM | 1464 | CA | LYS | A | 439 | 15.535 | 15.311 | −9.995 | 1.00 | 45.87 | C |
| ATOM | 1465 | CB | LYS | A | 439 | 16.834 | 15.521 | −10.794 | 1.00 | 46.05 | C |
| ATOM | 1466 | CG | LYS | A | 439 | 18.096 | 14.931 | −10.113 | 1.00 | 48.48 | C |
| ATOM | 1467 | CD | LYS | A | 439 | 19.417 | 15.775 | −10.247 | 1.00 | 51.35 | C |
| ATOM | 1468 | CE | LYS | A | 439 | 20.205 | 15.702 | −8.875 | 1.00 | 54.04 | C |
| ATOM | 1469 | NZ | LYS | A | 439 | 21.624 | 16.236 | −8.775 | 1.00 | 53.95 | N |
| ATOM | 1470 | C | LYS | A | 439 | 14.405 | 16.129 | −10.609 | 1.00 | 45.17 | C |
| ATOM | 1471 | O | LYS | A | 439 | 14.149 | 17.248 | −10.179 | 1.00 | 44.73 | O |
| ATOM | 1472 | N | VAL | A | 440 | 13.723 | 15.582 | −11.607 | 1.00 | 44.46 | N |
| ATOM | 1473 | CA | VAL | A | 440 | 12.665 | 16.324 | −12.279 | 1.00 | 43.79 | C |
| ATOM | 1474 | CB | VAL | A | 440 | 12.234 | 15.633 | −13.601 | 1.00 | 44.85 | C |
| ATOM | 1475 | CG1 | VAL | A | 440 | 10.895 | 16.199 | −14.096 | 1.00 | 43.93 | C |
| ATOM | 1476 | CG2 | VAL | A | 440 | 13.359 | 15.762 | −14.686 | 1.00 | 44.89 | C |
| ATOM | 1477 | C | VAL | A | 440 | 11.437 | 16.574 | −11.385 | 1.00 | 42.76 | C |
| ATOM | 1478 | O | VAL | A | 440 | 10.908 | 17.667 | −11.371 | 1.00 | 42.48 | O |
| ATOM | 1479 | N | LYS | A | 441 | 10.981 | 15.582 | −10.638 | 1.00 | 42.10 | N |
| ATOM | 1480 | CA | LYS | A | 441 | 9.842 | 15.811 | −9.737 | 1.00 | 42.12 | C |
| ATOM | 1481 | CB | LYS | A | 441 | 9.337 | 14.469 | −9.167 | 1.00 | 42.37 | C |
| ATOM | 1482 | CG | LYS | A | 441 | 8.268 | 14.551 | −8.058 | 1.00 | 44.18 | C |
| ATOM | 1483 | CD | LYS | A | 441 | 7.770 | 13.145 | −7.635 | 1.00 | 46.69 | C |
| ATOM | 1484 | CE | LYS | A | 441 | 7.415 | 13.029 | −6.135 | 1.00 | 48.06 | C |
| ATOM | 1485 | NZ | LYS | A | 441 | 6.374 | 13.988 | −5.640 | 1.00 | 50.40 | N |
| ATOM | 1486 | C | LYS | A | 441 | 10.222 | 16.812 | −8.621 | 1.00 | 41.30 | C |
| ATOM | 1487 | O | LYS | A | 441 | 9.493 | 17.751 | −8.337 | 1.00 | 41.41 | O |
| ATOM | 1488 | N | ILE | A | 442 | 11.384 | 16.639 | −8.016 | 1.00 | 40.14 | N |
| ATOM | 1489 | CA | ILE | A | 442 | 11.792 | 17.530 | −6.947 | 1.00 | 39.47 | C |
| ATOM | 1490 | CB | ILE | A | 442 | 13.092 | 17.036 | −6.347 | 1.00 | 39.22 | C |
| ATOM | 1491 | CG1 | ILE | A | 442 | 12.817 | 15.709 | −5.642 | 1.00 | 37.84 | C |
| ATOM | 1492 | CD1 | ILE | A | 442 | 14.038 | 14.971 | −5.155 | 1.00 | 36.84 | C |
| ATOM | 1493 | CG2 | ILE | A | 442 | 13.694 | 18.109 | −5.421 | 1.00 | 38.41 | C |
| ATOM | 1494 | C | ILE | A | 442 | 11.924 | 18.939 | −7.475 | 1.00 | 40.04 | C |
| ATOM | 1495 | O | ILE | A | 442 | 11.519 | 19.907 | −6.828 | 1.00 | 39.89 | O |
| ATOM | 1496 | N | GLU | A | 443 | 12.474 | 19.068 | −8.671 | 1.00 | 40.56 | N |
| ATOM | 1497 | CA | GLU | A | 443 | 12.583 | 20.380 | −9.305 | 1.00 | 41.62 | C |
| ATOM | 1498 | CB | GLU | A | 443 | 13.325 | 20.252 | −10.625 | 1.00 | 42.43 | C |
| ATOM | 1499 | CG | GLU | A | 443 | 13.473 | 21.556 | −11.384 | 1.00 | 46.01 | C |
| ATOM | 1500 | CD | GLU | A | 443 | 14.586 | 22.409 | −10.832 | 1.00 | 52.43 | C |
| ATOM | 1501 | OE1 | GLU | A | 443 | 15.520 | 21.840 | −10.224 | 1.00 | 58.50 | O |
| ATOM | 1502 | OE2 | GLU | A | 443 | 14.540 | 23.649 | −10.991 | 1.00 | 56.77 | O |
| ATOM | 1503 | C | GLU | A | 443 | 11.221 | 21.041 | −9.531 | 1.00 | 41.19 | C |
| ATOM | 1504 | O | GLU | A | 443 | 11.051 | 22.217 | −9.257 | 1.00 | 41.75 | O |
| ATOM | 1505 | N | LYS | A | 444 | 10.241 | 20.309 | −10.027 | 1.00 | 41.34 | N |
| ATOM | 1506 | CA | LYS | A | 444 | 8.918 | 20.906 | −10.222 | 1.00 | 41.91 | C |
| ATOM | 1507 | CB | LYS | A | 444 | 7.954 | 19.921 | −10.887 | 1.00 | 42.50 | C |
| ATOM | 1508 | CG | LYS | A | 444 | 8.359 | 19.402 | −12.287 | 1.00 | 46.11 | C |
| ATOM | 1509 | CD | LYS | A | 444 | 8.622 | 20.539 | −13.249 | 1.00 | 50.76 | C |
| ATOM | 1510 | CE | LYS | A | 444 | 9.166 | 20.064 | −14.593 | 1.00 | 53.57 | C |
| ATOM | 1511 | NZ | LYS | A | 444 | 10.041 | 21.141 | −15.193 | 1.00 | 55.91 | N |
| ATOM | 1512 | C | LYS | A | 444 | 8.333 | 21.367 | −8.869 | 1.00 | 41.11 | C |
| ATOM | 1513 | O | LYS | A | 444 | 7.669 | 22.385 | −8.787 | 1.00 | 40.91 | O |
| ATOM | 1514 | N | LEU | A | 445 | 8.564 | 20.599 | −7.811 | 1.00 | 40.57 | N |
| ATOM | 1515 | CA | LEU | A | 445 | 8.100 | 20.993 | −6.475 | 1.00 | 40.29 | C |
| ATOM | 1516 | CB | LEU | A | 445 | 8.291 | 19.823 | −5.510 | 1.00 | 40.73 | C |
| ATOM | 1517 | CG | LEU | A | 445 | 7.471 | 18.577 | −5.846 | 1.00 | 43.76 | C |
| ATOM | 1518 | CD1 | LEU | A | 445 | 8.070 | 17.339 | −5.224 | 1.00 | 46.83 | C |
| ATOM | 1519 | CD2 | LEU | A | 445 | 6.006 | 18.699 | −5.405 | 1.00 | 45.67 | C |
| ATOM | 1520 | C | LEU | A | 445 | 8.802 | 22.264 | −5.932 | 1.00 | 39.05 | C |
| ATOM | 1521 | O | LEU | A | 445 | 8.162 | 23.148 | −5.355 | 1.00 | 38.44 | O |
| ATOM | 1522 | N | GLN | A | 446 | 10.113 | 22.379 | −6.131 | 1.00 | 37.84 | N |
| ATOM | 1523 | CA | GLN | A | 446 | 10.804 | 23.553 | −5.630 | 1.00 | 37.19 | C |
| ATOM | 1524 | CB | GLN | A | 446 | 12.322 | 23.443 | −5.770 | 1.00 | 36.98 | C |
| ATOM | 1525 | CG | GLN | A | 446 | 13.058 | 24.617 | −5.128 | 1.00 | 38.75 | C |
| ATOM | 1526 | CD | GLN | A | 446 | 14.563 | 24.588 | −5.312 | 1.00 | 40.58 | C |
| ATOM | 1527 | OE1 | GLN | A | 446 | 15.065 | 24.583 | −6.428 | 1.00 | 42.73 | O |
| ATOM | 1528 | NE2 | GLN | A | 446 | 15.284 | 24.599 | −4.206 | 1.00 | 41.51 | N |
| ATOM | 1529 | C | GLN | A | 446 | 10.312 | 24.808 | −6.332 | 1.00 | 36.39 | C |
| ATOM | 1530 | O | GLN | A | 446 | 10.218 | 25.880 | −5.715 | 1.00 | 35.15 | O |
| ATOM | 1531 | N | GLN | A | 447 | 10.037 | 24.680 | −7.626 | 1.00 | 36.58 | N |
| ATOM | 1532 | CA | GLN | A | 447 | 9.547 | 25.810 | −8.415 | 1.00 | 37.09 | C |
| ATOM | 1533 | CB | GLN | A | 447 | 9.316 | 25.428 | −9.872 | 1.00 | 37.88 | C |
| ATOM | 1534 | CG | GLN | A | 447 | 10.545 | 25.058 | −10.659 | 1.00 | 42.37 | C |
| ATOM | 1535 | CD | GLN | A | 447 | 10.176 | 24.683 | −12.104 | 1.00 | 49.26 | C |
| ATOM | 1536 | OE1 | GLN | A | 447 | 9.144 | 25.147 | −12.634 | 1.00 | 53.32 | O |
| ATOM | 1537 | NE2 | GLN | A | 447 | 10.993 | 23.831 | −12.729 | 1.00 | 52.11 | N |
| ATOM | 1538 | C | GLN | A | 447 | 8.231 | 26.343 | −7.845 | 1.00 | 35.60 | C |
| ATOM | 1539 | O | GLN | A | 447 | 8.075 | 27.518 | −7.726 | 1.00 | 33.62 | O |
| ATOM | 1540 | N | LYS | A | 448 | 7.305 | 25.461 | −7.508 | 1.00 | 35.56 | N |
| ATOM | 1541 | CA | LYS | A | 448 | 6.055 | 25.897 | −6.903 | 1.00 | 36.26 | C |
| ATOM | 1542 | CB | LYS | A | 448 | 5.125 | 24.707 | −6.684 | 1.00 | 36.75 | C |
| ATOM | 1543 | CG | LYS | A | 448 | 4.434 | 24.265 | −7.968 | 1.00 | 41.05 | C |
| ATOM | 1544 | CD | LYS | A | 448 | 3.642 | 22.962 | −7.752 | 1.00 | 45.62 | C |
| ATOM | 1545 | CE | LYS | A | 448 | 2.734 | 22.623 | −8.941 | 1.00 | 47.75 | C |
| ATOM | 1546 | NZ | LYS | A | 448 | 1.655 | 21.655 | −8.507 | 1.00 | 51.38 | N |
| ATOM | 1547 | C | LYS | A | 448 | 6.304 | 26.615 | −5.584 | 1.00 | 35.43 | C |
| ATOM | 1548 | O | LYS | A | 448 | 5.762 | 27.697 | −5.345 | 1.00 | 34.04 | O |
| ATOM | 1549 | N | ILE | A | 449 | 7.118 | 25.997 | −4.720 | 1.00 | 35.23 | N |
| ATOM | 1550 | CA | ILE | A | 449 | 7.422 | 26.574 | −3.428 | 1.00 | 35.08 | C |
| ATOM | 1551 | CB | ILE | A | 449 | 8.400 | 25.663 | −2.647 | 1.00 | 35.31 | C |
| ATOM | 1552 | CG1 | ILE | A | 449 | 7.662 | 24.412 | −2.135 | 1.00 | 34.62 | C |
| ATOM | 1553 | CD1 | ILE | A | 449 | 8.565 | 23.272 | −1.666 | 1.00 | 35.68 | C |
| ATOM | 1554 | CG2 | ILE | A | 449 | 9.036 | 26.439 | −1.508 | 1.00 | 34.70 | C |
| ATOM | 1555 | C | ILE | A | 449 | 8.009 | 27.950 | −3.648 | 1.00 | 35.67 | C |
| ATOM | 1556 | O | ILE | A | 449 | 7.663 | 28.891 | −2.954 | 1.00 | 36.39 | O |
| ATOM | 1557 | N | GLN | A | 450 | 8.863 | 28.092 | −4.648 | 1.00 | 36.15 | N |
| ATOM | 1558 | CA | GLN | A | 450 | 9.491 | 29.383 | −4.898 | 1.00 | 37.22 | C |
| ATOM | 1559 | CB | GLN | A | 450 | 10.624 | 29.245 | −5.929 | 1.00 | 37.71 | C |
| ATOM | 1560 | CG | GLN | A | 450 | 11.440 | 30.525 | −6.143 | 1.00 | 39.93 | C |
| ATOM | 1561 | CD | GLN | A | 450 | 12.459 | 30.381 | −7.271 | 1.00 | 44.24 | C |
| ATOM | 1562 | OE1 | GLN | A | 450 | 13.107 | 29.339 | −7.409 | 1.00 | 46.56 | O |
| ATOM | 1563 | NE2 | GLN | A | 450 | 12.603 | 31.421 | −8.071 | 1.00 | 46.99 | N |
| ATOM | 1564 | C | GLN | A | 450 | 8.497 | 30.453 | −5.351 | 1.00 | 37.23 | C |
| ATOM | 1565 | O | GLN | A | 450 | 8.668 | 31.629 | −5.033 | 1.00 | 36.57 | O |
| ATOM | 1566 | N | LEU | A | 451 | 7.460 | 30.067 | −6.095 | 1.00 | 37.47 | N |
| ATOM | 1567 | CA | LEU | A | 451 | 6.491 | 31.055 | −6.548 | 1.00 | 38.17 | C |
| ATOM | 1568 | CB | LEU | A | 451 | 5.602 | 30.496 | −7.658 | 1.00 | 39.23 | C |
| ATOM | 1569 | CG | LEU | A | 451 | 6.299 | 30.228 | −8.992 | 1.00 | 40.16 | C |
| ATOM | 1570 | CD1 | LEU | A | 451 | 5.417 | 29.356 | −9.874 | 1.00 | 42.60 | C |
| ATOM | 1571 | CD2 | LEU | A | 451 | 6.671 | 31.543 | −9.709 | 1.00 | 40.87 | C |
| ATOM | 1572 | C | LEU | A | 451 | 5.675 | 31.479 | −5.350 | 1.00 | 38.38 | C |
| ATOM | 1573 | O | LEU | A | 451 | 5.328 | 32.670 | −5.177 | 1.00 | 37.56 | O |
| ATOM | 1574 | N | ALA | A | 452 | 5.396 | 30.500 | −4.499 | 1.00 | 38.51 | N |
| ATOM | 1575 | CA | ALA | A | 452 | 4.683 | 30.763 | −3.261 | 1.00 | 39.16 | C |
| ATOM | 1576 | CB | ALA | A | 452 | 4.352 | 29.452 | −2.566 | 1.00 | 39.25 | C |
| ATOM | 1577 | C | ALA | A | 452 | 5.492 | 31.668 | −2.335 | 1.00 | 39.16 | C |
| ATOM | 1578 | O | ALA | A | 452 | 4.960 | 32.541 | −1.667 | 1.00 | 38.45 | O |
| ATOM | 1579 | N | LEU | A | 453 | 6.793 | 31.461 | −2.302 | 1.00 | 39.99 | N |
| ATOM | 1580 | CA | LEU | A | 453 | 7.650 | 32.284 | −1.454 | 1.00 | 40.16 | C |
| ATOM | 1581 | CB | LEU | A | 453 | 9.078 | 31.735 | −1.455 | 1.00 | 39.61 | C |
| ATOM | 1582 | CG | LEU | A | 453 | 10.108 | 32.594 | −0.724 | 1.00 | 38.75 | C |
| ATOM | 1583 | CD1 | LEU | A | 453 | 9.785 | 32.702 | 0.737 | 1.00 | 37.68 | C |
| ATOM | 1584 | CD2 | LEU | A | 453 | 11.487 | 31.994 | −0.924 | 1.00 | 39.28 | C |
| ATOM | 1585 | C | LEU | A | 453 | 7.649 | 33.727 | −1.936 | 1.00 | 41.01 | C |
| ATOM | 1586 | O | LEU | A | 453 | 7.559 | 34.652 | −1.143 | 1.00 | 40.74 | O |
| ATOM | 1587 | N | GLN | A | 454 | 7.784 | 33.912 | −3.245 | 1.00 | 43.21 | N |
| ATOM | 1588 | CA | GLN | A | 454 | 7.778 | 35.242 | −3.857 | 1.00 | 44.37 | C |
| ATOM | 1589 | CB | GLN | A | 454 | 8.018 | 35.097 | −5.353 | 1.00 | 44.93 | C |
| ATOM | 1590 | CG | GLN | A | 454 | 8.139 | 36.413 | −6.092 | 1.00 | 47.00 | C |
| ATOM | 1591 | CD | GLN | A | 454 | 8.484 | 36.232 | −7.549 | 1.00 | 50.13 | C |
| ATOM | 1592 | OE1 | GLN | A | 454 | 7.937 | 35.355 | −8.224 | 1.00 | 51.04 | O |
| ATOM | 1593 | NE2 | GLN | A | 454 | 9.397 | 37.062 | −8.044 | 1.00 | 52.53 | N |
| ATOM | 1594 | C | GLN | A | 454 | 6.438 | 35.937 | −3.580 | 1.00 | 45.15 | C |
| ATOM | 1595 | O | GLN | A | 454 | 6.385 | 37.105 | −3.193 | 1.00 | 45.17 | O |
| ATOM | 1596 | N | HIS | A | 455 | 5.363 | 35.186 | −3.735 | 1.00 | 46.23 | N |
| ATOM | 1597 | CA | HIS | A | 455 | 4.029 | 35.654 | −3.418 | 1.00 | 47.70 | C |
| ATOM | 1598 | CB | HIS | A | 455 | 3.064 | 34.479 | −3.557 | 1.00 | 47.88 | C |
| ATOM | 1599 | CG | HIS | A | 455 | 1.659 | 34.782 | −3.164 | 1.00 | 50.63 | C |
| ATOM | 1600 | ND1 | HIS | A | 455 | 0.939 | 35.826 | −3.706 | 1.00 | 52.70 | N |
| ATOM | 1601 | CE1 | HIS | A | 455 | −0.272 | 35.843 | −3.172 | 1.00 | 53.57 | C |
| ATOM | 1602 | NE2 | HIS | A | 455 | −0.363 | 34.844 | −2.309 | 1.00 | 53.78 | N |
| ATOM | 1603 | CD2 | HIS | A | 455 | 0.833 | 34.167 | −2.283 | 1.00 | 52.74 | C |
| ATOM | 1604 | C | HIS | A | 455 | 3.974 | 36.276 | −2.018 | 1.00 | 48.21 | C |
| ATOM | 1605 | O | HIS | A | 455 | 3.561 | 37.425 | −1.852 | 1.00 | 48.61 | O |
| ATOM | 1606 | N | VAL | A | 456 | 4.436 | 35.544 | −1.017 | 1.00 | 48.96 | N |
| ATOM | 1607 | CA | VAL | A | 456 | 4.409 | 36.035 | 0.369 | 1.00 | 49.83 | C |
| ATOM | 1608 | CB | VAL | A | 456 | 4.656 | 34.884 | 1.353 | 1.00 | 49.91 | C |
| ATOM | 1609 | CG1 | VAL | A | 456 | 4.882 | 35.394 | 2.759 | 1.00 | 50.35 | C |
| ATOM | 1610 | CG2 | VAL | A | 456 | 3.498 | 33.899 | 1.325 | 1.00 | 50.07 | C |
| ATOM | 1611 | C | VAL | A | 456 | 5.437 | 37.101 | 0.684 | 1.00 | 50.39 | C |
| ATOM | 1612 | O | VAL | A | 456 | 5.262 | 37.865 | 1.625 | 1.00 | 50.47 | O |
| ATOM | 1613 | N | LEU | A | 457 | 6.529 | 37.119 | −0.062 | 1.00 | 51.74 | N |
| ATOM | 1614 | CA | LEU | A | 457 | 7.538 | 38.140 | 0.143 | 1.00 | 53.31 | C |
| ATOM | 1615 | CB | LEU | A | 457 | 8.801 | 37.829 | −0.648 | 1.00 | 53.04 | C |
| ATOM | 1616 | CG | LEU | A | 457 | 9.664 | 36.711 | −0.065 | 1.00 | 52.30 | C |
| ATOM | 1617 | CD1 | LEU | A | 457 | 10.705 | 36.350 | −1.081 | 1.00 | 51.56 | C |
| ATOM | 1618 | CD2 | LEU | A | 457 | 10.296 | 37.172 | 1.254 | 1.00 | 50.93 | C |
| ATOM | 1619 | C | LEU | A | 457 | 6.979 | 39.480 | −0.294 | 1.00 | 55.43 | C |
| ATOM | 1620 | O | LEU | A | 457 | 7.071 | 40.456 | 0.437 | 1.00 | 54.68 | O |
| ATOM | 1621 | N | GLN | A | 458 | 6.372 | 39.516 | −1.480 | 1.00 | 58.58 | N |
| ATOM | 1622 | CA | GLN | A | 458 | 5.836 | 40.764 | −2.020 | 1.00 | 61.31 | C |
| ATOM | 1623 | CB | GLN | A | 458 | 5.596 | 40.661 | −3.538 | 1.00 | 61.66 | C |
| ATOM | 1624 | CG | GLN | A | 458 | 4.556 | 39.646 | −4.008 | 1.00 | 63.40 | C |
| ATOM | 1625 | CD | GLN | A | 458 | 4.816 | 39.189 | −5.449 | 1.00 | 65.86 | C |
| ATOM | 1626 | OE1 | GLN | A | 458 | 5.793 | 39.622 | −6.069 | 1.00 | 67.19 | O |
| ATOM | 1627 | NE2 | GLN | A | 458 | 3.949 | 38.313 | −5.978 | 1.00 | 66.76 | N |
| ATOM | 1628 | C | GLN | A | 458 | 4.591 | 41.256 | −1.265 | 1.00 | 63.26 | C |
| ATOM | 1629 | O | GLN | A | 458 | 4.268 | 42.434 | −1.282 | 1.00 | 63.33 | O |
| ATOM | 1630 | N | LYS | A | 459 | 3.929 | 40.359 | −0.560 | 1.00 | 65.71 | N |
| ATOM | 1631 | CA | LYS | A | 459 | 2.770 | 40.732 | 0.228 | 1.00 | 67.66 | C |
| ATOM | 1632 | CB | LYS | A | 459 | 2.241 | 39.477 | 0.894 | 1.00 | 67.68 | C |
| ATOM | 1633 | CG | LYS | A | 459 | 1.061 | 39.617 | 1.825 | 1.00 | 67.62 | C |
| ATOM | 1634 | CD | LYS | A | 459 | 0.638 | 38.243 | 2.332 | 1.00 | 67.67 | C |
| ATOM | 1635 | CE | LYS | A | 459 | −0.042 | 37.427 | 1.241 | 1.00 | 67.92 | C |
| ATOM | 1636 | NZ | LYS | A | 459 | −0.160 | 35.983 | 1.597 | 1.00 | 68.58 | N |
| ATOM | 1637 | C | LYS | A | 459 | 3.116 | 41.776 | 1.290 | 1.00 | 69.97 | C |
| ATOM | 1638 | O | LYS | A | 459 | 2.285 | 42.618 | 1.648 | 1.00 | 70.29 | O |
| ATOM | 1639 | N | ASN | A | 460 | 4.361 | 41.747 | 1.755 | 1.00 | 72.42 | N |
| ATOM | 1640 | CA | ASN | A | 460 | 4.802 | 42.571 | 2.871 | 1.00 | 74.25 | C |
| ATOM | 1641 | CB | ASN | A | 460 | 5.508 | 41.681 | 3.896 | 1.00 | 74.44 | C |
| ATOM | 1642 | CG | ASN | A | 460 | 4.624 | 40.534 | 4.390 | 1.00 | 74.77 | C |
| ATOM | 1643 | OD1 | ASN | A | 460 | 3.556 | 40.762 | 4.964 | 1.00 | 74.61 | O |
| ATOM | 1644 | ND2 | ASN | A | 460 | 5.071 | 39.292 | 4.165 | 1.00 | 74.61 | N |
| ATOM | 1645 | C | ASN | A | 460 | 5.731 | 43.721 | 2.501 | 1.00 | 76.15 | C |
| ATOM | 1646 | O | ASN | A | 460 | 5.676 | 44.785 | 3.123 | 1.00 | 76.33 | O |
| ATOM | 1647 | N | HIS | A | 461 | 6.582 | 43.529 | 1.496 | 1.00 | 78.30 | N |
| ATOM | 1648 | CA | HIS | A | 461 | 7.564 | 44.550 | 1.141 | 1.00 | 79.89 | C |
| ATOM | 1649 | CB | HIS | A | 461 | 8.846 | 44.307 | 1.932 | 1.00 | 80.20 | C |
| ATOM | 1650 | CG | HIS | A | 461 | 8.629 | 44.208 | 3.407 | 1.00 | 81.84 | C |
| ATOM | 1651 | ND1 | HIS | A | 461 | 8.597 | 45.312 | 4.234 | 1.00 | 84.02 | N |
| ATOM | 1652 | CE1 | HIS | A | 461 | 8.377 | 44.919 | 5.478 | 1.00 | 84.23 | C |
| ATOM | 1653 | NE2 | HIS | A | 461 | 8.253 | 43.603 | 5.484 | 1.00 | 83.90 | N |
| ATOM | 1654 | CD2 | HIS | A | 461 | 8.406 | 43.135 | 4.201 | 1.00 | 83.33 | C |
| ATOM | 1655 | C | HIS | A | 461 | 7.907 | 44.600 | −0.344 | 1.00 | 80.88 | C |
| ATOM | 1656 | O | HIS | A | 461 | 9.067 | 44.431 | −0.721 | 1.00 | 80.77 | O |
| ATOM | 1657 | N | ARG | A | 462 | 6.912 | 44.841 | −1.191 | 1.00 | 82.27 | N |
| ATOM | 1658 | CA | ARG | A | 462 | 7.194 | 45.051 | −2.605 | 1.00 | 83.28 | C |
| ATOM | 1659 | CB | ARG | A | 462 | 5.903 | 45.330 | −3.393 | 1.00 | 83.78 | C |
| ATOM | 1660 | CG | ARG | A | 462 | 5.303 | 44.070 | −4.042 | 1.00 | 85.00 | C |
| ATOM | 1661 | CD | ARG | A | 462 | 3.898 | 44.230 | −4.665 | 1.00 | 86.49 | C |
| ATOM | 1662 | NE | ARG | A | 462 | 3.505 | 43.033 | −5.417 | 1.00 | 87.52 | N |
| ATOM | 1663 | CZ | ARG | A | 462 | 2.281 | 42.778 | −5.865 | 1.00 | 88.16 | C |
| ATOM | 1664 | NH1 | ARG | A | 462 | 1.283 | 43.636 | −5.655 | 1.00 | 88.54 | N |
| ATOM | 1665 | NH2 | ARG | A | 462 | 2.056 | 41.653 | −6.532 | 1.00 | 87.86 | N |
| ATOM | 1666 | C | ARG | A | 462 | 8.191 | 46.222 | −2.687 | 1.00 | 83.63 | C |
| ATOM | 1667 | O | ARG | A | 462 | 8.541 | 46.699 | −3.770 | 1.00 | 83.73 | O |
| ATOM | 1668 | N | GLU | A | 463 | 8.649 | 46.649 | −1.506 | 1.00 | 83.89 | N |
| ATOM | 1669 | CA | GLU | A | 463 | 9.624 | 47.723 | −1.335 | 1.00 | 83.96 | C |
| ATOM | 1670 | CB | GLU | A | 463 | 9.847 | 47.970 | 0.156 | 1.00 | 84.16 | C |
| ATOM | 1671 | CG | GLU | A | 463 | 10.746 | 46.939 | 0.821 | 1.00 | 84.97 | C |
| ATOM | 1672 | CD | GLU | A | 463 | 10.741 | 47.071 | 2.326 | 1.00 | 85.96 | C |
| ATOM | 1673 | OE1 | GLU | A | 463 | 10.286 | 48.126 | 2.808 | 1.00 | 87.00 | O |
| ATOM | 1674 | OE2 | GLU | A | 463 | 11.182 | 46.126 | 3.020 | 1.00 | 86.69 | O |
| ATOM | 1675 | C | GLU | A | 463 | 10.980 | 47.413 | −1.965 | 1.00 | 83.47 | C |
| ATOM | 1676 | O | GLU | A | 463 | 11.752 | 48.325 | −2.276 | 1.00 | 83.54 | O |
| ATOM | 1677 | N | ASP | A | 464 | 11.274 | 46.125 | −2.115 | 1.00 | 82.69 | N |
| ATOM | 1678 | CA | ASP | A | 464 | 12.532 | 45.673 | −2.691 | 1.00 | 82.00 | C |
| ATOM | 1679 | CB | ASP | A | 464 | 13.576 | 45.440 | −1.582 | 1.00 | 82.03 | C |
| ATOM | 1680 | CG | ASP | A | 464 | 14.138 | 46.743 | −1.000 | 1.00 | 82.58 | C |
| ATOM | 1681 | OD1 | ASP | A | 464 | 13.981 | 47.814 | −1.626 | 1.00 | 82.80 | O |
| ATOM | 1682 | OD2 | ASP | A | 464 | 14.766 | 46.795 | 0.079 | 1.00 | 82.96 | O |
| ATOM | 1683 | C | ASP | A | 464 | 12.251 | 44.380 | −3.467 | 1.00 | 81.10 | C |
| ATOM | 1684 | O | ASP | A | 464 | 11.103 | 44.121 | −3.868 | 1.00 | 81.10 | O |
| ATOM | 1685 | N | GLY | A | 465 | 13.305 | 43.599 | −3.701 | 1.00 | 79.69 | N |
| ATOM | 1686 | CA | GLY | A | 465 | 13.210 | 42.292 | −4.339 | 1.00 | 78.42 | C |
| ATOM | 1687 | C | GLY | A | 465 | 14.125 | 41.358 | −3.567 | 1.00 | 77.09 | C |
| ATOM | 1688 | O | GLY | A | 465 | 15.055 | 40.776 | −4.128 | 1.00 | 76.83 | O |
| ATOM | 1689 | N | ILE | A | 466 | 13.839 | 41.229 | −2.269 | 1.00 | 75.34 | N |
| ATOM | 1690 | CA | ILE | A | 466 | 14.687 | 40.507 | −1.318 | 1.00 | 73.67 | C |
| ATOM | 1691 | CB | ILE | A | 466 | 14.195 | 40.728 | 0.123 | 1.00 | 73.83 | C |
| ATOM | 1692 | CG1 | ILE | A | 466 | 13.734 | 42.163 | 0.325 | 1.00 | 74.49 | C |
| ATOM | 1693 | CD1 | ILE | A | 466 | 13.216 | 42.442 | 1.714 | 1.00 | 75.46 | C |
| ATOM | 1694 | CG2 | ILE | A | 466 | 15.312 | 40.453 | 1.110 | 1.00 | 74.24 | C |
| ATOM | 1695 | C | ILE | A | 466 | 14.756 | 39.023 | −1.579 | 1.00 | 71.72 | C |
| ATOM | 1696 | O | ILE | A | 466 | 15.601 | 38.329 | −1.009 | 1.00 | 71.07 | O |
| ATOM | 1697 | N | LEU | A | 467 | 13.860 | 38.535 | −2.425 | 1.00 | 69.76 | N |
| ATOM | 1698 | CA | LEU | A | 467 | 13.872 | 37.134 | −2.791 | 1.00 | 68.62 | C |
| ATOM | 1699 | CB | LEU | A | 467 | 12.849 | 36.853 | −3.889 | 1.00 | 68.53 | C |
| ATOM | 1700 | CG | LEU | A | 467 | 12.722 | 35.400 | −4.353 | 1.00 | 68.87 | C |
| ATOM | 1701 | CD1 | LEU | A | 467 | 12.138 | 34.497 | −3.294 | 1.00 | 69.10 | C |
| ATOM | 1702 | CD2 | LEU | A | 467 | 11.854 | 35.347 | −5.579 | 1.00 | 70.26 | C |
| ATOM | 1703 | C | LEU | A | 467 | 15.273 | 36.746 | −3.247 | 1.00 | 67.31 | C |
| ATOM | 1704 | O | LEU | A | 467 | 15.733 | 35.642 | −2.961 | 1.00 | 67.18 | O |
| ATOM | 1705 | N | THR | A | 468 | 15.954 | 37.660 | −3.934 | 1.00 | 65.58 | N |
| ATOM | 1706 | CA | THR | A | 468 | 17.285 | 37.382 | −4.452 | 1.00 | 64.62 | C |
| ATOM | 1707 | CB | THR | A | 468 | 17.695 | 38.399 | −5.584 | 1.00 | 64.88 | C |
| ATOM | 1708 | OG1 | THR | A | 468 | 17.784 | 39.733 | −5.065 | 1.00 | 64.25 | O |
| ATOM | 1709 | CG2 | THR | A | 468 | 16.631 | 38.493 | −6.680 | 1.00 | 64.06 | C |
| ATOM | 1710 | C | THR | A | 468 | 18.316 | 37.367 | −3.331 | 1.00 | 63.63 | C |
| ATOM | 1711 | O | THR | A | 468 | 19.200 | 36.515 | −3.298 | 1.00 | 63.53 | O |
| ATOM | 1712 | N | LYS | A | 469 | 18.215 | 38.324 | −2.420 | 1.00 | 62.39 | N |
| ATOM | 1713 | CA | LYS | A | 469 | 19.088 | 38.367 | −1.252 | 1.00 | 61.38 | C |
| ATOM | 1714 | CB | LYS | A | 469 | 18.797 | 39.647 | −0.484 | 1.00 | 62.13 | C |
| ATOM | 1715 | CG | LYS | A | 469 | 19.486 | 39.829 | 0.860 | 1.00 | 62.89 | C |
| ATOM | 1716 | CD | LYS | A | 469 | 19.256 | 41.310 | 1.276 | 1.00 | 64.52 | C |
| ATOM | 1717 | CE | LYS | A | 469 | 19.505 | 41.608 | 2.756 | 1.00 | 65.74 | C |
| ATOM | 1718 | NZ | LYS | A | 469 | 18.942 | 42.951 | 3.178 | 1.00 | 66.28 | N |
| ATOM | 1719 | C | LYS | A | 469 | 18.854 | 37.133 | −0.355 | 1.00 | 59.69 | C |
| ATOM | 1720 | O | LYS | A | 469 | 19.766 | 36.645 | 0.317 | 1.00 | 59.53 | O |
| ATOM | 1721 | N | LEU | A | 470 | 17.627 | 36.633 | −0.360 | 1.00 | 57.33 | N |
| ATOM | 1722 | CA | LEU | A | 470 | 17.292 | 35.439 | 0.387 | 1.00 | 56.01 | C |
| ATOM | 1723 | CB | LEU | A | 470 | 15.771 | 35.324 | 0.527 | 1.00 | 55.44 | C |
| ATOM | 1724 | CG | LEU | A | 470 | 15.242 | 34.622 | 1.770 | 1.00 | 54.97 | C |
| ATOM | 1725 | CD1 | LEU | A | 470 | 15.881 | 35.167 | 3.018 | 1.00 | 54.08 | C |
| ATOM | 1726 | CD2 | LEU | A | 470 | 13.721 | 34.701 | 1.859 | 1.00 | 56.05 | C |
| ATOM | 1727 | C | LEU | A | 470 | 17.861 | 34.213 | −0.331 | 1.00 | 55.43 | C |
| ATOM | 1728 | O | LEU | A | 470 | 18.522 | 33.382 | 0.281 | 1.00 | 54.90 | O |
| ATOM | 1729 | N | ILE | A | 471 | 17.624 | 34.111 | −1.635 | 1.00 | 54.68 | N |
| ATOM | 1730 | CA | ILE | A | 471 | 18.120 | 32.986 | −2.412 | 1.00 | 54.43 | C |
| ATOM | 1731 | CB | ILE | A | 471 | 17.538 | 33.016 | −3.836 | 1.00 | 54.76 | C |
| ATOM | 1732 | CG1 | ILE | A | 471 | 16.097 | 32.526 | −3.792 | 1.00 | 55.40 | C |
| ATOM | 1733 | CD1 | ILE | A | 471 | 15.438 | 32.478 | −5.119 | 1.00 | 56.34 | C |
| ATOM | 1734 | CG2 | ILE | A | 471 | 18.372 | 32.144 | −4.798 | 1.00 | 55.26 | C |
| ATOM | 1735 | C | ILE | A | 471 | 19.631 | 33.016 | −2.433 | 1.00 | 53.73 | C |
| ATOM | 1736 | O | ILE | A | 471 | 20.281 | 31.996 | −2.616 | 1.00 | 53.69 | O |
| ATOM | 1737 | N | CYS | A | 472 | 20.189 | 34.198 | −2.239 | 1.00 | 52.86 | N |
| ATOM | 1738 | CA | CYS | A | 472 | 21.625 | 34.347 | −2.120 | 1.00 | 52.63 | C |
| ATOM | 1739 | CB | CYS | A | 472 | 21.979 | 35.829 | −1.990 | 1.00 | 52.92 | C |
| ATOM | 1740 | SG | CYS | A | 472 | 22.874 | 36.495 | −3.399 | 1.00 | 58.34 | S |
| ATOM | 1741 | C | CYS | A | 472 | 22.157 | 33.604 | −0.886 | 1.00 | 50.97 | C |
| ATOM | 1742 | O | CYS | A | 472 | 23.290 | 33.104 | −0.901 | 1.00 | 50.65 | O |
| ATOM | 1743 | N | LYS | A | 473 | 21.348 | 33.555 | 0.179 | 1.00 | 48.54 | N |
| ATOM | 1744 | CA | LYS | A | 473 | 21.758 | 32.936 | 1.439 | 1.00 | 47.29 | C |
| ATOM | 1745 | CB | LYS | A | 473 | 20.754 | 33.227 | 2.562 | 1.00 | 47.39 | C |
| ATOM | 1746 | CG | LYS | A | 473 | 20.619 | 34.712 | 2.930 | 1.00 | 49.59 | C |
| ATOM | 1747 | CD | LYS | A | 473 | 21.832 | 35.243 | 3.708 | 1.00 | 52.10 | C |
| ATOM | 1748 | CE | LYS | A | 473 | 22.062 | 36.733 | 3.414 | 1.00 | 54.59 | C |
| ATOM | 1749 | NZ | LYS | A | 473 | 23.470 | 37.017 | 2.981 | 1.00 | 56.43 | N |
| ATOM | 1750 | C | LYS | A | 473 | 21.875 | 31.450 | 1.277 | 1.00 | 45.26 | C |
| ATOM | 1751 | O | LYS | A | 473 | 22.631 | 30.804 | 1.995 | 1.00 | 45.05 | O |
| ATOM | 1752 | N | VAL | A | 474 | 21.122 | 30.919 | 0.327 | 1.00 | 43.23 | N |
| ATOM | 1753 | CA | VAL | A | 474 | 21.136 | 29.518 | 0.102 | 1.00 | 42.19 | C |
| ATOM | 1754 | CB | VAL | A | 474 | 20.387 | 29.116 | −1.145 | 1.00 | 42.59 | C |
| ATOM | 1755 | CG1 | VAL | A | 474 | 20.645 | 27.663 | −1.449 | 1.00 | 42.42 | C |
| ATOM | 1756 | CG2 | VAL | A | 474 | 18.910 | 29.383 | −0.957 | 1.00 | 42.39 | C |
| ATOM | 1757 | C | VAL | A | 474 | 22.546 | 29.055 | −0.055 | 1.00 | 41.04 | C |
| ATOM | 1758 | O | VAL | A | 474 | 22.919 | 28.042 | 0.503 | 1.00 | 39.72 | O |
| ATOM | 1759 | N | SER | A | 475 | 23.345 | 29.780 | −0.812 | 1.00 | 39.27 | N |
| ATOM | 1760 | CA | SER | A | 475 | 24.718 | 29.353 | −0.979 | 1.00 | 38.63 | C |
| ATOM | 1761 | CB | SER | A | 475 | 25.402 | 30.157 | −2.070 | 1.00 | 38.92 | C |
| ATOM | 1762 | OG | SER | A | 475 | 26.788 | 29.921 | −2.029 | 1.00 | 40.43 | O |
| ATOM | 1763 | C | SER | A | 475 | 25.490 | 29.483 | 0.336 | 1.00 | 37.50 | C |
| ATOM | 1764 | O | SER | A | 475 | 26.430 | 28.759 | 0.576 | 1.00 | 36.17 | O |
| ATOM | 1765 | N | THR | A | 476 | 25.118 | 30.425 | 1.192 | 1.00 | 36.92 | N |
| ATOM | 1766 | CA | THR | A | 476 | 25.854 | 30.597 | 2.443 | 1.00 | 36.78 | C |
| ATOM | 1767 | CB | THR | A | 476 | 25.460 | 31.898 | 3.133 | 1.00 | 37.27 | C |
| ATOM | 1768 | OG1 | THR | A | 476 | 25.293 | 32.946 | 2.166 | 1.00 | 39.90 | O |
| ATOM | 1769 | CG2 | THR | A | 476 | 26.543 | 32.416 | 4.043 | 1.00 | 37.41 | C |
| ATOM | 1770 | C | THR | A | 476 | 25.588 | 29.423 | 3.378 | 1.00 | 36.02 | C |
| ATOM | 1771 | O | THR | A | 476 | 26.489 | 28.981 | 4.084 | 1.00 | 35.49 | O |
| ATOM | 1772 | N | LEU | A | 477 | 24.355 | 28.916 | 3.360 | 1.00 | 35.62 | N |
| ATOM | 1773 | CA | LEU | A | 477 | 23.939 | 27.801 | 4.200 | 1.00 | 35.50 | C |
| ATOM | 1774 | CB | LEU | A | 477 | 22.457 | 27.515 | 4.019 | 1.00 | 35.72 | C |
| ATOM | 1775 | CG | LEU | A | 477 | 21.480 | 28.292 | 4.875 | 1.00 | 35.56 | C |
| ATOM | 1776 | CD1 | LEU | A | 477 | 20.057 | 28.141 | 4.321 | 1.00 | 36.36 | C |
| ATOM | 1777 | CD2 | LEU | A | 477 | 21.584 | 27.800 | 6.279 | 1.00 | 33.36 | C |
| ATOM | 1778 | C | LEU | A | 477 | 24.670 | 26.538 | 3.890 | 1.00 | 35.75 | C |
| ATOM | 1779 | O | LEU | A | 477 | 24.966 | 25.754 | 4.792 | 1.00 | 35.71 | O |
| ATOM | 1780 | N | ARG | A | 478 | 24.941 | 26.311 | 2.610 | 1.00 | 35.96 | N |
| ATOM | 1781 | CA | ARG | A | 478 | 25.659 | 25.124 | 2.183 | 1.00 | 36.68 | C |
| ATOM | 1782 | CB | ARG | A | 478 | 25.606 | 24.989 | 0.666 | 1.00 | 37.49 | C |
| ATOM | 1783 | CG | ARG | A | 478 | 24.216 | 24.645 | 0.185 | 1.00 | 39.62 | C |
| ATOM | 1784 | CD | ARG | A | 478 | 24.110 | 24.375 | −1.293 | 1.00 | 41.13 | C |
| ATOM | 1785 | NE | ARG | A | 478 | 22.727 | 24.112 | −1.658 | 1.00 | 42.25 | N |
| ATOM | 1786 | CZ | ARG | A | 478 | 22.342 | 23.503 | −2.763 | 1.00 | 43.78 | C |
| ATOM | 1787 | NH1 | ARG | A | 478 | 23.246 | 23.088 | −3.641 | 1.00 | 45.06 | N |
| ATOM | 1788 | NH2 | ARG | A | 478 | 21.045 | 23.312 | −3.003 | 1.00 | 42.39 | N |
| ATOM | 1789 | C | ARG | A | 478 | 27.093 | 25.179 | 2.666 | 1.00 | 36.22 | C |
| ATOM | 1790 | O | ARG | A | 478 | 27.655 | 24.170 | 3.056 | 1.00 | 36.31 | O |
| ATOM | 1791 | N | ALA | A | 479 | 27.690 | 26.361 | 2.645 | 1.00 | 35.43 | N |
| ATOM | 1792 | CA | ALA | A | 479 | 29.045 | 26.502 | 3.144 | 1.00 | 34.83 | C |
| ATOM | 1793 | CB | ALA | A | 479 | 29.634 | 27.829 | 2.714 | 1.00 | 34.35 | C |
| ATOM | 1794 | C | ALA | A | 479 | 29.029 | 26.382 | 4.674 | 1.00 | 34.50 | C |
| ATOM | 1795 | O | ALA | A | 479 | 29.923 | 25.803 | 5.254 | 1.00 | 33.03 | O |
| ATOM | 1796 | N | LEU | A | 480 | 28.011 | 26.924 | 5.330 | 1.00 | 34.39 | N |
| ATOM | 1797 | CA | LEU | A | 480 | 27.931 | 26.756 | 6.768 | 1.00 | 35.19 | C |
| ATOM | 1798 | CB | LEU | A | 480 | 26.787 | 27.598 | 7.348 | 1.00 | 35.91 | C |
| ATOM | 1799 | CG | LEU | A | 480 | 26.747 | 27.805 | 8.845 | 1.00 | 35.84 | C |
| ATOM | 1800 | CD1 | LEU | A | 480 | 28.051 | 28.355 | 9.382 | 1.00 | 37.04 | C |
| ATOM | 1801 | CD2 | LEU | A | 480 | 25.590 | 28.734 | 9.189 | 1.00 | 36.87 | C |
| ATOM | 1802 | C | LEU | A | 480 | 27.787 | 25.276 | 7.138 | 1.00 | 35.48 | C |
| ATOM | 1803 | O | LEU | A | 480 | 28.525 | 24.768 | 7.999 | 1.00 | 36.43 | O |
| ATOM | 1804 | N | CYS | A | 481 | 26.886 | 24.557 | 6.482 | 1.00 | 34.90 | N |
| ATOM | 1805 | CA | CYS | A | 481 | 26.721 | 23.150 | 6.768 | 1.00 | 35.75 | C |
| ATOM | 1806 | CB | CYS | A | 481 | 25.408 | 22.617 | 6.151 | 1.00 | 35.51 | C |
| ATOM | 1807 | SG | CYS | A | 481 | 23.970 | 23.492 | 6.880 | 1.00 | 39.57 | S |
| ATOM | 1808 | C | CYS | A | 481 | 27.955 | 22.339 | 6.343 | 1.00 | 35.95 | C |
| ATOM | 1809 | O | CYS | A | 481 | 28.248 | 21.278 | 6.914 | 1.00 | 35.58 | O |
| ATOM | 1810 | N | GLY | A | 482 | 28.690 | 22.846 | 5.359 | 1.00 | 35.86 | N |
| ATOM | 1811 | CA | GLY | A | 482 | 29.909 | 22.197 | 4.922 | 1.00 | 35.86 | C |
| ATOM | 1812 | C | GLY | A | 482 | 30.978 | 22.309 | 5.983 | 1.00 | 35.56 | C |
| ATOM | 1813 | O | GLY | A | 482 | 31.689 | 21.375 | 6.268 | 1.00 | 35.07 | O |
| ATOM | 1814 | N | ARG | A | 483 | 31.111 | 23.464 | 6.587 | 1.00 | 36.33 | N |
| ATOM | 1815 | CA | ARG | A | 483 | 32.099 | 23.572 | 7.623 | 1.00 | 37.63 | C |
| ATOM | 1816 | CB | ARG | A | 483 | 32.250 | 25.000 | 8.046 | 1.00 | 38.73 | C |
| ATOM | 1817 | CG | ARG | A | 483 | 32.770 | 25.873 | 6.900 | 1.00 | 43.48 | C |
| ATOM | 1818 | CD | ARG | A | 483 | 34.089 | 26.567 | 7.220 | 1.00 | 48.75 | C |
| ATOM | 1819 | NE | ARG | A | 483 | 34.129 | 27.928 | 6.699 | 1.00 | 52.27 | N |
| ATOM | 1820 | CZ | ARG | A | 483 | 34.148 | 29.016 | 7.463 | 1.00 | 56.41 | C |
| ATOM | 1821 | NH1 | ARG | A | 483 | 34.138 | 28.914 | 8.793 | 1.00 | 55.60 | N |
| ATOM | 1822 | NH2 | ARG | A | 483 | 34.177 | 30.224 | 6.894 | 1.00 | 59.68 | N |
| ATOM | 1823 | C | ARG | A | 483 | 31.767 | 22.661 | 8.813 | 1.00 | 37.14 | C |
| ATOM | 1824 | O | ARG | A | 483 | 32.662 | 22.026 | 9.401 | 1.00 | 36.58 | O |
| ATOM | 1825 | N | HIS | A | 484 | 30.486 | 22.567 | 9.161 | 1.00 | 36.27 | N |
| ATOM | 1826 | CA | HIS | A | 484 | 30.119 | 21.712 | 10.270 | 1.00 | 35.26 | C |
| ATOM | 1827 | CB | HIS | A | 484 | 28.615 | 21.655 | 10.449 | 1.00 | 34.25 | C |
| ATOM | 1828 | CG | HIS | A | 484 | 28.161 | 20.599 | 11.394 | 1.00 | 31.44 | C |
| ATOM | 1829 | ND1 | HIS | A | 484 | 27.481 | 19.481 | 10.975 | 1.00 | 26.89 | N |
| ATOM | 1830 | CE1 | HIS | A | 484 | 27.178 | 18.740 | 12.032 | 1.00 | 29.68 | C |
| ATOM | 1831 | NE2 | HIS | A | 484 | 27.689 | 19.307 | 13.111 | 1.00 | 26.36 | N |
| ATOM | 1832 | CD2 | HIS | A | 484 | 28.293 | 20.484 | 12.740 | 1.00 | 29.62 | C |
| ATOM | 1833 | C | HIS | A | 484 | 30.656 | 20.333 | 10.013 | 1.00 | 36.35 | C |
| ATOM | 1834 | O | HIS | A | 484 | 31.264 | 19.740 | 10.893 | 1.00 | 35.93 | O |
| ATOM | 1835 | N | THR | A | 485 | 30.457 | 19.818 | 8.805 | 1.00 | 38.13 | N |
| ATOM | 1836 | CA | THR | A | 485 | 30.881 | 18.466 | 8.520 | 1.00 | 39.75 | C |
| ATOM | 1837 | CB | THR | A | 485 | 30.421 | 18.032 | 7.111 | 1.00 | 40.58 | C |
| ATOM | 1838 | OG1 | THR | A | 485 | 29.011 | 17.726 | 7.102 | 1.00 | 42.13 | O |
| ATOM | 1839 | CG2 | THR | A | 485 | 31.050 | 16.685 | 6.702 | 1.00 | 41.01 | C |
| ATOM | 1840 | C | THR | A | 485 | 32.404 | 18.340 | 8.663 | 1.00 | 40.64 | C |
| ATOM | 1841 | O | THR | A | 485 | 32.903 | 17.312 | 9.092 | 1.00 | 41.11 | O |
| ATOM | 1842 | N | GLU | A | 486 | 33.136 | 19.380 | 8.297 | 1.00 | 41.10 | N |
| ATOM | 1843 | CA | GLU | A | 486 | 34.583 | 19.329 | 8.343 | 1.00 | 41.77 | C |
| ATOM | 1844 | CB | GLU | A | 486 | 35.200 | 20.533 | 7.597 | 1.00 | 42.58 | C |
| ATOM | 1845 | CG | GLU | A | 486 | 35.297 | 20.311 | 6.080 | 1.00 | 45.90 | C |
| ATOM | 1846 | CD | GLU | A | 486 | 35.265 | 21.577 | 5.215 | 1.00 | 48.00 | C |
| ATOM | 1847 | OE1 | GLU | A | 486 | 34.972 | 21.411 | 4.013 | 1.00 | 50.12 | O |
| ATOM | 1848 | OE2 | GLU | A | 486 | 35.538 | 22.715 | 5.690 | 1.00 | 50.93 | O |
| ATOM | 1849 | C | GLU | A | 486 | 35.048 | 19.311 | 9.775 | 1.00 | 40.94 | C |
| ATOM | 1850 | O | GLU | A | 486 | 35.970 | 18.573 | 10.146 | 1.00 | 40.53 | O |
| ATOM | 1851 | N | LYS | A | 487 | 34.445 | 20.161 | 10.584 | 1.00 | 39.75 | N |
| ATOM | 1852 | CA | LYS | A | 487 | 34.828 | 20.215 | 11.976 | 1.00 | 38.97 | C |
| ATOM | 1853 | CB | LYS | A | 487 | 34.122 | 21.369 | 12.697 | 1.00 | 39.27 | C |
| ATOM | 1854 | CG | LYS | A | 487 | 34.760 | 22.719 | 12.395 | 1.00 | 42.48 | C |
| ATOM | 1855 | CD | LYS | A | 487 | 36.275 | 22.575 | 12.241 | 1.00 | 45.26 | C |
| ATOM | 1856 | CE | LYS | A | 487 | 36.953 | 23.908 | 12.119 | 1.00 | 47.44 | C |
| ATOM | 1857 | NZ | LYS | A | 487 | 37.048 | 24.550 | 13.468 | 1.00 | 50.31 | N |
| ATOM | 1858 | C | LYS | A | 487 | 34.509 | 18.896 | 12.637 | 1.00 | 38.04 | C |
| ATOM | 1859 | O | LYS | A | 487 | 35.306 | 18.390 | 13.405 | 1.00 | 37.61 | O |
| ATOM | 1860 | N | LEU | A | 488 | 33.354 | 18.329 | 12.317 | 1.00 | 37.23 | N |
| ATOM | 1861 | CA | LEU | A | 488 | 32.937 | 17.098 | 12.946 | 1.00 | 37.18 | C |
| ATOM | 1862 | CB | LEU | A | 488 | 31.502 | 16.755 | 12.561 | 1.00 | 36.30 | C |
| ATOM | 1863 | CG | LEU | A | 488 | 31.030 | 15.382 | 12.993 | 1.00 | 36.25 | C |
| ATOM | 1864 | CD1 | LEU | A | 488 | 31.060 | 15.184 | 14.496 | 1.00 | 37.06 | C |
| ATOM | 1865 | CD2 | LEU | A | 488 | 29.636 | 15.172 | 12.465 | 1.00 | 37.46 | C |
| ATOM | 1866 | C | LEU | A | 488 | 33.885 | 15.955 | 12.593 | 1.00 | 37.99 | C |
| ATOM | 1867 | O | LEU | A | 488 | 34.238 | 15.147 | 13.466 | 1.00 | 37.01 | O |
| ATOM | 1868 | N | MET | A | 489 | 34.263 | 15.851 | 11.317 | 1.00 | 38.60 | N |
| ATOM | 1869 | CA | MET | A | 489 | 35.240 | 14.828 | 10.932 | 1.00 | 40.07 | C |
| ATOM | 1870 | CB | MET | A | 489 | 35.412 | 14.694 | 9.409 | 1.00 | 40.81 | C |
| ATOM | 1871 | CG | MET | A | 489 | 34.178 | 14.171 | 8.704 | 1.00 | 44.15 | C |
| ATOM | 1872 | SD | MET | A | 489 | 33.194 | 12.913 | 9.641 | 1.00 | 54.88 | S |
| ATOM | 1873 | CE | MET | A | 489 | 33.908 | 11.239 | 9.132 | 1.00 | 55.79 | C |
| ATOM | 1874 | C | MET | A | 489 | 36.572 | 15.107 | 11.636 | 1.00 | 39.24 | C |
| ATOM | 1875 | O | MET | A | 489 | 37.216 | 14.197 | 12.086 | 1.00 | 38.99 | O |
| ATOM | 1876 | N | ALA | A | 490 | 36.960 | 16.356 | 11.813 | 1.00 | 39.22 | N |
| ATOM | 1877 | CA | ALA | A | 490 | 38.226 | 16.606 | 12.512 | 1.00 | 39.46 | C |
| ATOM | 1878 | CB | ALA | A | 490 | 38.623 | 18.062 | 12.412 | 1.00 | 38.63 | C |
| ATOM | 1879 | C | ALA | A | 490 | 38.152 | 16.166 | 13.997 | 1.00 | 40.06 | C |
| ATOM | 1880 | O | ALA | A | 490 | 39.131 | 15.669 | 14.568 | 1.00 | 40.68 | O |
| ATOM | 1881 | N | PHE | A | 491 | 36.979 | 16.351 | 14.602 | 1.00 | 39.40 | N |
| ATOM | 1882 | CA | PHE | A | 491 | 36.756 | 15.996 | 15.982 | 1.00 | 38.41 | C |
| ATOM | 1883 | CB | PHE | A | 491 | 35.449 | 16.640 | 16.482 | 1.00 | 37.82 | C |
| ATOM | 1884 | CG | PHE | A | 491 | 35.055 | 16.218 | 17.871 | 1.00 | 34.23 | C |
| ATOM | 1885 | CD1 | PHE | A | 491 | 35.515 | 16.907 | 18.979 | 1.00 | 31.46 | C |
| ATOM | 1886 | CE1 | PHE | A | 491 | 35.168 | 16.507 | 20.263 | 1.00 | 31.14 | C |
| ATOM | 1887 | CZ | PHE | A | 491 | 34.329 | 15.411 | 20.445 | 1.00 | 29.45 | C |
| ATOM | 1888 | CE2 | PHE | A | 491 | 33.873 | 14.724 | 19.340 | 1.00 | 32.47 | C |
| ATOM | 1889 | CD2 | PHE | A | 491 | 34.233 | 15.134 | 18.058 | 1.00 | 31.03 | C |
| ATOM | 1890 | C | PHE | A | 491 | 36.695 | 14.484 | 16.112 | 1.00 | 39.01 | C |
| ATOM | 1891 | O | PHE | A | 491 | 37.183 | 13.910 | 17.079 | 1.00 | 38.94 | O |
| ATOM | 1892 | N | LYS | A | 492 | 36.077 | 13.831 | 15.153 | 1.00 | 39.53 | N |
| ATOM | 1893 | CA | LYS | A | 492 | 35.941 | 12.401 | 15.226 | 1.00 | 40.55 | C |
| ATOM | 1894 | CB | LYS | A | 492 | 34.934 | 11.958 | 14.192 | 1.00 | 40.93 | C |
| ATOM | 1895 | CG | LYS | A | 492 | 34.730 | 10.473 | 14.114 | 1.00 | 42.40 | C |
| ATOM | 1896 | CD | LYS | A | 492 | 33.670 | 10.118 | 13.098 | 1.00 | 44.01 | C |
| ATOM | 1897 | CE | LYS | A | 492 | 33.493 | 8.628 | 12.999 | 1.00 | 44.96 | C |
| ATOM | 1898 | NZ | LYS | A | 492 | 32.414 | 8.271 | 12.044 | 1.00 | 47.85 | N |
| ATOM | 1899 | C | LYS | A | 492 | 37.283 | 11.668 | 15.031 | 1.00 | 41.46 | C |
| ATOM | 1900 | O | LYS | A | 492 | 37.425 | 10.516 | 15.389 | 1.00 | 41.69 | O |
| ATOM | 1901 | N | ALA | A | 493 | 38.275 | 12.336 | 14.476 | 1.00 | 42.47 | N |
| ATOM | 1902 | CA | ALA | A | 493 | 39.570 | 11.696 | 14.290 | 1.00 | 43.19 | C |
| ATOM | 1903 | CB | ALA | A | 493 | 40.374 | 12.445 | 13.226 | 1.00 | 43.14 | C |
| ATOM | 1904 | C | ALA | A | 493 | 40.340 | 11.640 | 15.612 | 1.00 | 43.40 | C |
| ATOM | 1905 | O | ALA | A | 493 | 41.244 | 10.812 | 15.802 | 1.00 | 44.12 | O |
| ATOM | 1906 | N | ILE | A | 494 | 39.979 | 12.516 | 16.531 | 1.00 | 42.66 | N |
| ATOM | 1907 | CA | ILE | A | 494 | 40.651 | 12.589 | 17.806 | 1.00 | 42.35 | C |
| ATOM | 1908 | CB | ILE | A | 494 | 40.873 | 14.048 | 18.163 | 1.00 | 42.63 | C |
| ATOM | 1909 | CG1 | ILE | A | 494 | 41.824 | 14.686 | 17.155 | 1.00 | 44.49 | C |
| ATOM | 1910 | CD1 | ILE | A | 494 | 41.756 | 16.198 | 17.142 | 1.00 | 44.88 | C |
| ATOM | 1911 | CG2 | ILE | A | 494 | 41.451 | 14.167 | 19.560 | 1.00 | 44.25 | C |
| ATOM | 1912 | C | ILE | A | 494 | 39.882 | 11.920 | 18.942 | 1.00 | 41.61 | C |
| ATOM | 1913 | O | ILE | A | 494 | 40.485 | 11.551 | 19.953 | 1.00 | 41.78 | O |
| ATOM | 1914 | N | TYR | A | 495 | 38.566 | 11.796 | 18.796 | 1.00 | 40.39 | N |
| ATOM | 1915 | CA | TYR | A | 495 | 37.722 | 11.225 | 19.830 | 1.00 | 39.98 | C |
| ATOM | 1916 | CB | TYR | A | 495 | 36.916 | 12.324 | 20.513 | 1.00 | 39.79 | C |
| ATOM | 1917 | CG | TYR | A | 495 | 37.731 | 13.430 | 21.115 | 1.00 | 38.81 | C |
| ATOM | 1918 | CD1 | TYR | A | 495 | 37.949 | 14.585 | 20.415 | 1.00 | 38.27 | C |
| ATOM | 1919 | CE1 | TYR | A | 495 | 38.685 | 15.600 | 20.932 | 1.00 | 38.45 | C |
| ATOM | 1920 | CZ | TYR | A | 495 | 39.209 | 15.508 | 22.194 | 1.00 | 39.44 | C |
| ATOM | 1921 | OH | TYR | A | 495 | 39.947 | 16.582 | 22.669 | 1.00 | 40.01 | O |
| ATOM | 1922 | CE2 | TYR | A | 495 | 39.013 | 14.366 | 22.941 | 1.00 | 38.56 | C |
| ATOM | 1923 | CD2 | TYR | A | 495 | 38.260 | 13.329 | 22.402 | 1.00 | 38.69 | C |
| ATOM | 1924 | C | TYR | A | 495 | 36.747 | 10.209 | 19.257 | 1.00 | 40.43 | C |
| ATOM | 1925 | O | TYR | A | 495 | 35.548 | 10.290 | 19.483 | 1.00 | 40.56 | O |
| ATOM | 1926 | N | PRO | A | 496 | 37.255 | 9.201 | 18.567 | 1.00 | 40.98 | N |
| ATOM | 1927 | CA | PRO | A | 496 | 36.379 | 8.261 | 17.871 | 1.00 | 40.62 | C |
| ATOM | 1928 | CB | PRO | A | 496 | 37.344 | 7.229 | 17.288 | 1.00 | 41.12 | C |
| ATOM | 1929 | CG | PRO | A | 496 | 38.747 | 7.789 | 17.496 | 1.00 | 41.49 | C |
| ATOM | 1930 | CD | PRO | A | 496 | 38.682 | −8.848 | 18.486 | 1.00 | 40.68 | C |
| ATOM | 1931 | C | PRO | A | 496 | 35.392 | 7.567 | 18.796 | 1.00 | 40.79 | C |
| ATOM | 1932 | O | PRO | A | 496 | 34.263 | 7.261 | 18.404 | 1.00 | 41.69 | O |
| ATOM | 1933 | N | ASP | A | 497 | 35.814 | 7.254 | 20.008 | 1.00 | 40.89 | N |
| ATOM | 1934 | CA | ASP | A | 497 | 34.937 | 6.528 | 20.913 | 1.00 | 40.80 | C |
| ATOM | 1935 | CB | ASP | A | 497 | 35.745 | 5.792 | 21.988 | 1.00 | 41.84 | C |
| ATOM | 1936 | CG | ASP | A | 497 | 36.300 | 4.475 | 21.485 | 1.00 | 45.64 | C |
| ATOM | 1937 | OD1 | ASP | A | 497 | 36.821 | 3.696 | 22.320 | 1.00 | 53.60 | O |
| ATOM | 1938 | OD2 | ASP | A | 497 | 36.249 | 4.124 | 20.282 | 1.00 | 48.70 | O |
| ATOM | 1939 | C | ASP | A | 497 | 33.902 | 7.409 | 21.579 | 1.00 | 39.07 | C |
| ATOM | 1940 | O | ASP | A | 497 | 32.847 | 6.919 | 21.956 | 1.00 | 38.09 | O |
| ATOM | 1941 | N | ILE | A | 498 | 34.211 | 8.697 | 21.744 | 1.00 | 38.14 | N |
| ATOM | 1942 | CA | ILE | A | 498 | 33.230 | 9.640 | 22.273 | 1.00 | 37.33 | C |
| ATOM | 1943 | CB | ILE | A | 498 | 33.864 | 11.039 | 22.491 | 1.00 | 38.00 | C |
| ATOM | 1944 | CG1 | ILE | A | 498 | 34.902 | 11.018 | 23.627 | 1.00 | 39.27 | C |
| ATOM | 1945 | CD1 | ILE | A | 498 | 34.375 | 10.559 | 24.947 | 1.00 | 40.90 | C |
| ATOM | 1946 | CG2 | ILE | A | 498 | 32.797 | 12.090 | 22.770 | 1.00 | 37.86 | C |
| ATOM | 1947 | C | ILE | A | 498 | 32.092 | 9.696 | 21.272 | 1.00 | 36.02 | C |
| ATOM | 1948 | O | ILE | A | 498 | 30.929 | 9.572 | 21.625 | 1.00 | 35.72 | O |
| ATOM | 1949 | N | VAL | A | 499 | 32.429 | 9.814 | 20.002 | 1.00 | 35.39 | N |
| ATOM | 1950 | CA | VAL | A | 499 | 31.400 | 9.915 | 18.994 | 1.00 | 35.49 | C |
| ATOM | 1951 | CB | VAL | A | 499 | 31.969 | 10.182 | 17.580 | 1.00 | 34.87 | C |
| ATOM | 1952 | CG1 | VAL | A | 499 | 30.868 | 10.168 | 16.543 | 1.00 | 34.58 | C |
| ATOM | 1953 | CG2 | VAL | A | 499 | 32.686 | 11.485 | 17.542 | 1.00 | 34.93 | C |
| ATOM | 1954 | C | VAL | A | 499 | 30.598 | 8.647 | 18.975 | 1.00 | 35.88 | C |
| ATOM | 1955 | O | VAL | A | 499 | 29.384 | 8.684 | 18.933 | 1.00 | 36.02 | O |
| ATOM | 1956 | N | ARG | A | 500 | 31.278 | 7.515 | 19.004 | 1.00 | 36.41 | N |
| ATOM | 1957 | CA | ARG | A | 500 | 30.600 | 6.237 | 18.880 | 1.00 | 36.86 | C |
| ATOM | 1958 | CB | ARG | A | 500 | 31.629 | 5.129 | 18.714 | 1.00 | 37.54 | C |
| ATOM | 1959 | CG | ARG | A | 500 | 31.058 | 3.739 | 18.596 | 1.00 | 41.50 | C |
| ATOM | 1960 | CD | ARG | A | 500 | 32.137 | 2.639 | 18.598 | 1.00 | 46.56 | C |
| ATOM | 1961 | NE | ARG | A | 500 | 31.814 | 1.588 | 19.576 | 1.00 | 51.65 | N |
| ATOM | 1962 | CZ | ARG | A | 500 | 32.514 | 1.331 | 20.688 | 1.00 | 52.82 | C |
| ATOM | 1963 | NH1 | ARG | A | 500 | 33.613 | 2.013 | 20.978 | 1.00 | 53.74 | N |
| ATOM | 1964 | NH2 | ARG | A | 500 | 32.117 | 0.370 | 21.508 | 1.00 | 54.03 | N |
| ATOM | 1965 | C | ARG | A | 500 | 29.724 | 5.950 | 20.066 | 1.00 | 36.07 | C |
| ATOM | 1966 | O | ARG | A | 500 | 28.570 | 5.586 | 19.884 | 1.00 | 36.22 | O |
| ATOM | 1967 | N | LEU | A | 501 | 30.247 | 6.133 | 21.277 | 1.00 | 35.25 | N |
| ATOM | 1968 | CA | LEU | A | 501 | 29.493 | 5.780 | 22.495 | 1.00 | 35.10 | C |
| ATOM | 1969 | CB | LEU | A | 501 | 30.444 | 5.244 | 23.602 | 1.00 | 35.42 | C |
| ATOM | 1970 | CG | LEU | A | 501 | 31.187 | 3.915 | 23.312 | 1.00 | 38.90 | C |
| ATOM | 1971 | CD1 | LEU | A | 501 | 32.025 | 3.417 | 24.493 | 1.00 | 41.10 | C |
| ATOM | 1972 | CD2 | LEU | A | 501 | 30.219 | 2.807 | 22.904 | 1.00 | 40.70 | C |
| ATOM | 1973 | C | LEU | A | 501 | 28.609 | 6.885 | 23.083 | 1.00 | 33.61 | C |
| ATOM | 1974 | O | LEU | A | 501 | 27.660 | 6.590 | 23.788 | 1.00 | 32.60 | O |
| ATOM | 1975 | N | HIS | A | 502 | 28.891 | 8.152 | 22.799 | 1.00 | 33.08 | N |
| ATOM | 1976 | CA | HIS | A | 502 | 28.121 | 9.226 | 23.475 | 1.00 | 32.46 | C |
| ATOM | 1977 | CB | HIS | A | 502 | 29.049 | 9.936 | 24.480 | 1.00 | 32.21 | C |
| ATOM | 1978 | CG | HIS | A | 502 | 29.690 | 8.993 | 25.454 | 1.00 | 32.45 | C |
| ATOM | 1979 | ND1 | HIS | A | 502 | 28.998 | 8.440 | 26.515 | 1.00 | 34.45 | N |
| ATOM | 1980 | CE1 | HIS | A | 502 | 29.796 | 7.616 | 27.175 | 1.00 | 33.23 | C |
| ATOM | 1981 | NE2 | HIS | A | 502 | 30.978 | 7.610 | 26.582 | 1.00 | 32.06 | N |
| ATOM | 1982 | CD2 | HIS | A | 502 | 30.940 | 8.457 | 25.499 | 1.00 | 32.50 | C |
| ATOM | 1983 | C | HIS | A | 502 | 27.392 | 10.237 | 22.575 | 1.00 | 31.35 | C |
| ATOM | 1984 | O | HIS | A | 502 | 26.671 | 11.084 | 23.064 | 1.00 | 32.31 | O |
| ATOM | 1985 | N | PHE | A | 503 | 27.572 | 10.171 | 21.269 | 1.00 | 30.30 | N |
| ATOM | 1986 | CA | PHE | A | 503 | 26.839 | 11.071 | 20.401 | 1.00 | 29.71 | C |
| ATOM | 1987 | CB | PHE | A | 503 | 27.636 | 11.366 | 19.123 | 1.00 | 28.59 | C |
| ATOM | 1988 | CG | PHE | A | 503 | 28.589 | 12.527 | 19.253 | 1.00 | 27.67 | C |
| ATOM | 1989 | CD1 | PHE | A | 503 | 29.265 | 12.774 | 20.443 | 1.00 | 28.62 | C |
| ATOM | 1990 | CE1 | PHE | A | 503 | 30.132 | 13.869 | 20.575 | 1.00 | 24.57 | C |
| ATOM | 1991 | CZ | PHE | A | 503 | 30.313 | 14.715 | 19.523 | 1.00 | 23.93 | C |
| ATOM | 1992 | CE2 | PHE | A | 503 | 29.635 | 14.481 | 18.322 | 1.00 | 27.80 | C |
| ATOM | 1993 | CD2 | PHE | A | 503 | 28.783 | 13.402 | 18.194 | 1.00 | 27.49 | C |
| ATOM | 1994 | C | PHE | A | 503 | 25.465 | 10.483 | 20.086 | 1.00 | 29.88 | C |
| ATOM | 1995 | O | PHE | A | 503 | 25.301 | 9.290 | 20.004 | 1.00 | 29.58 | O |
| ATOM | 1996 | N | PRO | A | 504 | 24.472 | 11.327 | 19.887 | 1.00 | 30.28 | N |
| ATOM | 1997 | CA | PRO | A | 504 | 23.131 | 10.838 | 19.561 | 1.00 | 30.42 | C |
| ATOM | 1998 | CB | PRO | A | 504 | 22.305 | 12.107 | 19.435 | 1.00 | 30.02 | C |
| ATOM | 1999 | CG | PRO | A | 504 | 23.129 | 13.163 | 20.095 | 1.00 | 30.50 | C |
| ATOM | 2000 | CD | PRO | A | 504 | 24.559 | 12.793 | 19.900 | 1.00 | 29.70 | C |
| ATOM | 2001 | C | PRO | A | 504 | 23.112 | 10.094 | 18.232 | 1.00 | 30.68 | C |
| ATOM | 2002 | O | PRO | A | 504 | 23.698 | 10.533 | 17.261 | 1.00 | 29.79 | O |
| ATOM | 2003 | N | PRO | A | 505 | 22.410 | 8.973 | 18.199 | 1.00 | 31.99 | N |
| ATOM | 2004 | CA | PRO | A | 505 | 22.330 | 8.126 | 16.997 | 1.00 | 32.55 | C |
| ATOM | 2005 | CB | PRO | A | 505 | 21.334 | 7.055 | 17.397 | 1.00 | 32.91 | C |
| ATOM | 2006 | CG | PRO | A | 505 | 21.520 | 6.943 | 18.876 | 1.00 | 33.39 | C |
| ATOM | 2007 | CD | PRO | A | 505 | 21.710 | 8.384 | 19.347 | 1.00 | 32.17 | C |
| ATOM | 2008 | C | PRO | A | 505 | 21.866 | 8.820 | 15.736 | 1.00 | 32.64 | C |
| ATOM | 2009 | O | PRO | A | 505 | 22.416 | 8.540 | 14.662 | 1.00 | 33.22 | O |
| ATOM | 2010 | N | LEU | A | 506 | 20.888 | 9.709 | 15.843 | 1.00 | 32.69 | N |
| ATOM | 2011 | CA | LEU | A | 506 | 20.388 | 10.427 | 14.673 | 1.00 | 32.07 | C |
| ATOM | 2012 | CB | LEU | A | 506 | 19.117 | 11.204 | 15.015 | 1.00 | 32.44 | C |
| ATOM | 2013 | CG | LEU | A | 506 | 18.566 | 12.012 | 13.840 | 1.00 | 31.16 | C |
| ATOM | 2014 | CD1 | LEU | A | 506 | 18.156 | 11.092 | 12.722 | 1.00 | 33.03 | C |
| ATOM | 2015 | CD2 | LEU | A | 506 | 17.398 | 12.836 | 14.302 | 1.00 | 32.49 | C |
| ATOM | 2016 | C | LEU | A | 506 | 21.425 | 11.369 | 14.091 | 1.00 | 32.28 | C |
| ATOM | 2017 | O | LEU | A | 506 | 21.491 | 11.528 | 12.893 | 1.00 | 32.61 | O |
| ATOM | 2018 | N | TYR | A | 507 | 22.210 | 12.012 | 14.949 | 1.00 | 32.83 | N |
| ATOM | 2019 | CA | TYR | A | 507 | 23.327 | 12.879 | 14.533 | 1.00 | 32.73 | C |
| ATOM | 2020 | CB | TYR | A | 507 | 23.927 | 13.589 | 15.762 | 1.00 | 32.18 | C |
| ATOM | 2021 | CG | TYR | A | 507 | 25.025 | 14.643 | 15.516 | 1.00 | 30.21 | C |
| ATOM | 2022 | CD1 | TYR | A | 507 | 24.733 | 15.980 | 15.458 | 1.00 | 27.51 | C |
| ATOM | 2023 | CE1 | TYR | A | 507 | 25.715 | 16.908 | 15.284 | 1.00 | 28.26 | C |
| ATOM | 2024 | CZ | TYR | A | 507 | 27.020 | 16.500 | 15.176 | 1.00 | 29.93 | C |
| ATOM | 2025 | OH | TYR | A | 507 | 28.032 | 17.414 | 14.997 | 1.00 | 30.78 | O |
| ATOM | 2026 | CE2 | TYR | A | 507 | 27.336 | 15.180 | 15.239 | 1.00 | 28.89 | C |
| ATOM | 2027 | CD2 | TYR | A | 507 | 26.365 | 14.273 | 15.423 | 1.00 | 29.57 | C |
| ATOM | 2028 | C | TYR | A | 507 | 24.405 | 12.054 | 13.802 | 1.00 | 33.67 | C |
| ATOM | 2029 | O | TYR | A | 507 | 24.928 | 12.468 | 12.770 | 1.00 | 32.52 | O |
| ATOM | 2030 | N | LYS | A | 508 | 24.730 | 10.895 | 14.351 | 1.00 | 35.19 | N |
| ATOM | 2031 | CA | LYS | A | 508 | 25.674 | 9.979 | 13.703 | 1.00 | 36.84 | C |
| ATOM | 2032 | CB | LYS | A | 508 | 25.931 | 8.740 | 14.548 | 1.00 | 36.89 | C |
| ATOM | 2033 | CG | LYS | A | 508 | 26.837 | 8.988 | 15.763 | 1.00 | 38.78 | C |
| ATOM | 2034 | CD | LYS | A | 508 | 27.446 | 7.686 | 16.296 | 1.00 | 39.11 | C |
| ATOM | 2035 | CE | LYS | A | 508 | 26.536 | 7.042 | 17.265 | 1.00 | 41.23 | C |
| ATOM | 2036 | NZ | LYS | A | 508 | 27.053 | 5.702 | 17.721 | 1.00 | 42.04 | N |
| ATOM | 2037 | C | LYS | A | 508 | 25.165 | 9.560 | 12.332 | 1.00 | 37.51 | C |
| ATOM | 2038 | O | LYS | A | 508 | 25.908 | 9.643 | 11.386 | 1.00 | 37.79 | O |
| ATOM | 2039 | N | GLU | A | 509 | 23.893 | 9.165 | 12.232 | 1.00 | 38.63 | N |
| ATOM | 2040 | CA | GLU | A | 509 | 23.313 | 8.734 | 10.968 | 1.00 | 39.31 | C |
| ATOM | 2041 | CB | GLU | A | 509 | 21.863 | 8.281 | 11.131 | 1.00 | 39.58 | C |
| ATOM | 2042 | CG | GLU | A | 509 | 21.670 | 6.932 | 11.815 | 1.00 | 44.44 | C |
| ATOM | 2043 | CD | GLU | A | 509 | 20.198 | 6.514 | 11.993 | 1.00 | 48.40 | C |
| ATOM | 2044 | OE1 | GLU | A | 509 | 19.956 | 5.642 | 12.854 | 1.00 | 52.15 | O |
| ATOM | 2045 | OE2 | GLU | A | 509 | 19.285 | 7.028 | 11.285 | 1.00 | 50.58 | O |
| ATOM | 2046 | C | GLU | A | 509 | 23.333 | 9.841 | 9.937 | 1.00 | 39.53 | C |
| ATOM | 2047 | O | GLU | A | 509 | 23.504 | 9.571 | 8.745 | 1.00 | 39.45 | O |
| ATOM | 2048 | N | LEU | A | 510 | 23.126 | 11.083 | 10.379 | 1.00 | 39.18 | N |
| ATOM | 2049 | CA | LEU | A | 510 | 23.067 | 12.201 | 9.456 | 1.00 | 39.07 | C |
| ATOM | 2050 | CB | LEU | A | 510 | 22.226 | 13.338 | 10.040 | 1.00 | 39.33 | C |
| ATOM | 2051 | CG | LEU | A | 510 | 20.725 | 13.091 | 10.200 | 1.00 | 39.95 | C |
| ATOM | 2052 | CD1 | LEU | A | 510 | 20.091 | 14.296 | 10.800 | 1.00 | 41.52 | C |
| ATOM | 2053 | CD2 | LEU | A | 510 | 20.058 | 12.768 | 8.899 | 1.00 | 40.94 | C |
| ATOM | 2054 | C | LEU | A | 510 | 24.401 | 12.783 | 9.030 | 1.00 | 38.89 | C |
| ATOM | 2055 | O | LEU | A | 510 | 24.503 | 13.334 | 7.943 | 1.00 | 39.03 | O |
| ATOM | 2056 | N | PHE | A | 511 | 25.427 | 12.680 | 9.854 | 1.00 | 38.62 | N |
| ATOM | 2057 | CA | PHE | A | 511 | 26.619 | 13.464 | 9.591 | 1.00 | 38.51 | C |
| ATOM | 2058 | CB | PHE | A | 511 | 26.736 | 14.569 | 10.646 | 1.00 | 38.47 | C |
| ATOM | 2059 | CG | PHE | A | 511 | 25.542 | 15.483 | 10.711 | 1.00 | 39.17 | C |
| ATOM | 2060 | CD1 | PHE | A | 511 | 24.848 | 15.663 | 11.910 | 1.00 | 37.90 | C |
| ATOM | 2061 | CE1 | PHE | A | 511 | 23.762 | 16.508 | 11.985 | 1.00 | 37.16 | C |
| ATOM | 2062 | CZ | PHE | A | 511 | 23.324 | 17.165 | 10.866 | 1.00 | 39.02 | C |
| ATOM | 2063 | CE2 | PHE | A | 511 | 24.005 | 16.998 | 9.653 | 1.00 | 39.88 | C |
| ATOM | 2064 | CD2 | PHE | A | 511 | 25.113 | 16.167 | 9.587 | 1.00 | 40.32 | C |
| ATOM | 2065 | C | PHE | A | 511 | 27.915 | 12.682 | 9.565 | 1.00 | 38.68 | C |
| ATOM | 2066 | O | PHE | A | 511 | 28.923 | 13.297 | 9.211 | 1.00 | 38.22 | O |
| ATOM | 2067 | OXT | PHE | A | 511 | 27.963 | 11.489 | 9.895 | 1.00 | 39.77 | O |
| ATOM | 2068 | C65 | CHS | L | 1 | 29.670 | 21.352 | 16.280 | 1.00 | 40.20 | C |
| ATOM | 2069 | C63 | CHS | L | 1 | 28.173 | 21.713 | 16.502 | 1.00 | 35.49 | C |
| ATOM | 2070 | C69 | CHS | L | 1 | 27.552 | 20.583 | 17.354 | 1.00 | 36.38 | C |
| ATOM | 2071 | C60 | CHS | L | 1 | 28.076 | 23.102 | 17.181 | 1.00 | 33.50 | C |
| ATOM | 2072 | C57 | CHS | L | 1 | 26.755 | 23.921 | 17.026 | 1.00 | 29.77 | C |
| ATOM | 2073 | C54 | CHS | L | 1 | 26.543 | 24.885 | 18.224 | 1.00 | 26.05 | C |
| ATOM | 2074 | C48 | CHS | L | 1 | 25.339 | 25.868 | 18.122 | 1.00 | 27.06 | C |
| ATOM | 2075 | C50 | CHS | L | 1 | 25.416 | 26.630 | 16.786 | 1.00 | 30.39 | C |
| ATOM | 2076 | C38 | CHS | L | 1 | 23.999 | 25.113 | 18.108 | 1.00 | 27.35 | C |
| ATOM | 2077 | C35 | CHS | L | 1 | 23.898 | 24.161 | 19.333 | 1.00 | 25.32 | C |
| ATOM | 2078 | C29 | CHS | L | 1 | 22.637 | 25.885 | 18.088 | 1.00 | 25.81 | C |
| ATOM | 2079 | C25 | CHS | L | 1 | 22.075 | 26.442 | 16.760 | 1.00 | 25.48 | C |
| ATOM | 2080 | C40 | CHS | L | 1 | 22.674 | 27.065 | 19.089 | 1.00 | 24.66 | C |
| ATOM | 2081 | C30 | CHS | L | 1 | 21.683 | 24.733 | 18.480 | 1.00 | 24.66 | C |
| ATOM | 2082 | C32 | CHS | L | 1 | 22.378 | 23.988 | 19.639 | 1.00 | 25.34 | C |
| ATOM | 2083 | C18 | CHS | L | 1 | 20.267 | 25.269 | 18.823 | 1.00 | 24.65 | C |
| ATOM | 2084 | C15 | CHS | L | 1 | 19.389 | 24.126 | 19.379 | 1.00 | 27.21 | C |
| ATOM | 2085 | C20 | CHS | L | 1 | 19.656 | 25.910 | 17.559 | 1.00 | 25.94 | C |
| ATOM | 2086 | C23 | CHS | L | 1 | 20.616 | 26.987 | 16.956 | 1.00 | 25.70 | C |
| ATOM | 2087 | C22 | CHS | L | 1 | 18.195 | 26.423 | 17.788 | 1.00 | 25.96 | C |
| ATOM | 2088 | C44 | CHS | L | 1 | 18.216 | 27.828 | 18.461 | 1.00 | 23.41 | C |
| ATOM | 2089 | C12 | CHS | L | 1 | 17.391 | 25.437 | 18.627 | 1.00 | 27.66 | C |
| ATOM | 2090 | C9 | CHS | L | 1 | 15.878 | 25.602 | 18.705 | 1.00 | 27.52 | C |
| ATOM | 2091 | C13 | CHS | L | 1 | 17.928 | 24.439 | 19.343 | 1.00 | 28.47 | C |
| ATOM | 2092 | C1 | CHS | L | 1 | 17.457 | 26.540 | 16.432 | 1.00 | 24.95 | C |
| ATOM | 2093 | C4 | CHS | L | 1 | 15.966 | 26.932 | 16.596 | 1.00 | 25.61 | C |
| ATOM | 2094 | C7 | CHS | L | 1 | 15.184 | 25.850 | 17.366 | 1.00 | 27.22 | C |
| ATOM | 2095 | O6 | CHS | L | 1 | 13.884 | 26.323 | 17.783 | 1.00 | 33.31 | O |
| ATOM | 2096 | S1 | CHS | L | 1 | 12.600 | 26.109 | 16.995 | 1.00 | 36.30 | S |
| ATOM | 2097 | O3 | CHS | L | 1 | 11.492 | 26.689 | 17.749 | 1.00 | 35.79 | O |
| ATOM | 2098 | O2 | CHS | L | 1 | 12.386 | 24.736 | 16.612 | 1.00 | 34.23 | O |
| ATOM | 2099 | O4 | CHS | L | 1 | 12.791 | 26.916 | 15.768 | 1.00 | 31.69 | O |
| ATOM | 2100 | O | HOH | V | 1 | 34.374 | 18.778 | 31.636 | 1.00 | 24.18 | O |
| ATOM | 2101 | O | HOH | V | 2 | 13.751 | 22.827 | 14.717 | 1.00 | 25.05 | O |
| ATOM | 2102 | O | HOH | V | 3 | 17.585 | 18.928 | 1.035 | 1.00 | 25.61 | O |
| ATOM | 2103 | O | HOH | V | 4 | 19.468 | 24.062 | 23.227 | 1.00 | 25.84 | O |
| ATOM | 2104 | O | HOH | V | 5 | 28.242 | 16.854 | 27.596 | 1.00 | 28.05 | O |
| ATOM | 2105 | O | HOH | V | 6 | 26.219 | 37.914 | 34.970 | 1.00 | 28.23 | O |
| ATOM | 2106 | O | HOH | V | 7 | 25.424 | 34.243 | 27.348 | 1.00 | 29.16 | O |
| ATOM | 2107 | O | HOH | V | 8 | 37.940 | 8.815 | 22.096 | 1.00 | 29.22 | O |
| ATOM | 2108 | O | HOH | V | 9 | 34.532 | 27.583 | 19.498 | 1.00 | 30.78 | O |
| ATOM | 2109 | O | HOH | V | 10 | 36.533 | 26.810 | 13.236 | 1.00 | 30.95 | O |
| ATOM | 2110 | O | HOH | V | 11 | 19.929 | 34.372 | 33.559 | 1.00 | 31.12 | O |
| ATOM | 2111 | O | HOH | V | 12 | 30.464 | 36.924 | 16.913 | 1.00 | 32.33 | O |
| ATOM | 2112 | O | HOH | V | 13 | 39.694 | 19.997 | 26.277 | 1.00 | 32.46 | O |
| ATOM | 2113 | O | HOH | V | 14 | 10.618 | 25.915 | 9.224 | 1.00 | 33.09 | O |
| ATOM | 2114 | O | HOH | V | 15 | 19.089 | 15.060 | 21.458 | 1.00 | 33.35 | O |
| ATOM | 2115 | O | HOH | V | 16 | 19.982 | 38.502 | 24.574 | 1.00 | 33.56 | O |
| ATOM | 2116 | O | HOH | V | 17 | 29.188 | 18.773 | 31.889 | 1.00 | 34.82 | O |
| ATOM | 2117 | O | HOH | V | 18 | 32.816 | 26.367 | 25.939 | 1.00 | 35.28 | O |
| ATOM | 2118 | O | HOH | V | 19 | 21.757 | 13.531 | 27.380 | 1.00 | 35.38 | O |
| ATOM | 2119 | O | HOH | V | 20 | 21.923 | 18.531 | 7.233 | 1.00 | 35.96 | O |
| ATOM | 2120 | O | HOH | V | 21 | 33.494 | 32.602 | 17.864 | 1.00 | 36.22 | O |
| ATOM | 2121 | O | HOH | V | 22 | 29.529 | 39.171 | 20.916 | 1.00 | 36.73 | O |
| ATOM | 2122 | O | HOH | V | 23 | 26.956 | 19.335 | 8.080 | 1.00 | 37.09 | O |
| ATOM | 2123 | O | HOH | V | 24 | 17.942 | 26.092 | 21.769 | 1.00 | 37.19 | O |
| ATOM | 2124 | O | HOH | V | 25 | 30.129 | 36.843 | 14.123 | 1.00 | 37.60 | O |
| ATOM | 2125 | O | HOH | V | 26 | 26.979 | 35.078 | 38.228 | 1.00 | 37.66 | O |
| ATOM | 2126 | O | HOH | V | 27 | 11.702 | 12.320 | −11.449 | 1.00 | 37.83 | O |
| ATOM | 2127 | O | HOH | V | 28 | 24.019 | 40.426 | 26.459 | 1.00 | 38.20 | O |
| ATOM | 2128 | O | HOH | V | 29 | 32.889 | 38.167 | 8.976 | 1.00 | 38.30 | O |
| ATOM | 2129 | O | HOH | V | 30 | 26.368 | 37.122 | 27.563 | 1.00 | 38.38 | O |
| ATOM | 2130 | O | HOH | V | 31 | 26.038 | 37.728 | 37.444 | 1.00 | 38.65 | O |
| ATOM | 2131 | O | HOH | V | 32 | −0.184 | 22.111 | 6.895 | 1.00 | 38.91 | O |
| ATOM | 2132 | O | HOH | V | 33 | 24.132 | 19.988 | −1.108 | 1.00 | 39.60 | O |
| ATOM | 2133 | O | HOH | V | 34 | 17.228 | 26.642 | 24.728 | 1.00 | 39.66 | O |
| ATOM | 2134 | O | HOH | V | 35 | 24.430 | 17.401 | −1.616 | 1.00 | 40.07 | O |
| ATOM | 2135 | O | HOH | V | 36 | 14.466 | 37.465 | 9.931 | 1.00 | 40.09 | O |
| ATOM | 2136 | O | HOH | V | 37 | 38.590 | 23.562 | 23.909 | 1.00 | 40.15 | O |
| ATOM | 2137 | O | HOH | V | 38 | 20.064 | 26.866 | 31.634 | 1.00 | 40.16 | O |
| ATOM | 2138 | O | HOH | V | 39 | 5.285 | 36.507 | 11.739 | 1.00 | 40.86 | O |
| ATOM | 2139 | O | HOH | V | 40 | 5.515 | 30.530 | 16.484 | 1.00 | 40.87 | O |
| ATOM | 2140 | O | HOH | V | 41 | 24.115 | 12.660 | 27.541 | 1.00 | 40.92 | O |
| ATOM | 2141 | O | HOH | V | 42 | 23.756 | 5.602 | 14.704 | 1.00 | 41.17 | O |
| ATOM | 2142 | O | HOH | V | 43 | 10.524 | 40.707 | 27.825 | 1.00 | 41.34 | O |
| ATOM | 2143 | O | HOH | V | 44 | 26.115 | 7.036 | 20.648 | 1.00 | 41.58 | O |
| ATOM | 2144 | O | HOH | V | 45 | 22.909 | 14.371 | 0.739 | 1.00 | 41.86 | O |
| ATOM | 2145 | O | HOH | V | 46 | 38.001 | 12.389 | 26.816 | 1.00 | 42.12 | O |
| ATOM | 2146 | O | HOH | V | 47 | 27.038 | 38.349 | 22.646 | 1.00 | 42.13 | O |
| ATOM | 2147 | O | HOH | V | 48 | 27.926 | 38.210 | 14.696 | 1.00 | 42.24 | O |
| ATOM | 2148 | O | HOH | V | 49 | 19.208 | 17.322 | 27.275 | 1.00 | 42.36 | O |
| ATOM | 2149 | O | HOH | V | 50 | 17.702 | 22.996 | 26.002 | 1.00 | 42.43 | O |
| ATOM | 2150 | O | HOH | V | 51 | 21.518 | 40.403 | 26.071 | 1.00 | 42.44 | O |
| ATOM | 2151 | O | HOH | V | 52 | 29.008 | 37.276 | 26.773 | 1.00 | 42.97 | O |
| ATOM | 2152 | O | HOH | V | 53 | 16.797 | 40.023 | 17.063 | 1.00 | 43.20 | O |
| ATOM | 2153 | O | HOH | V | 54 | 27.959 | 18.192 | 30.078 | 1.00 | 43.25 | O |
| ATOM | 2154 | O | HOH | V | 55 | 27.189 | 38.094 | 30.644 | 1.00 | 43.31 | O |
| ATOM | 2155 | O | HOH | V | 56 | 32.853 | 5.119 | 27.654 | 1.00 | 43.36 | O |
| ATOM | 2156 | O | HOH | V | 57 | 25.498 | 15.066 | 28.345 | 1.00 | 43.59 | O |
| ATOM | 2157 | O | HOH | V | 58 | 26.277 | 3.798 | 24.349 | 1.00 | 43.69 | O |
| ATOM | 2158 | O | HOH | V | 59 | 24.431 | 4.474 | 16.791 | 1.00 | 43.83 | O |
| ATOM | 2159 | O | HOH | V | 60 | 17.931 | 21.585 | 22.061 | 1.00 | 43.86 | O |
| ATOM | 2160 | O | HOH | V | 61 | 3.622 | 36.866 | 6.230 | 1.00 | 43.90 | O |
| ATOM | 2161 | O | HOH | V | 62 | 29.565 | 0.033 | 25.439 | 1.00 | 44.03 | O |
| ATOM | 2162 | O | HOH | V | 63 | 37.471 | 27.196 | 26.904 | 1.00 | 44.11 | O |
| ATOM | 2163 | O | HOH | V | 64 | 14.114 | 35.615 | 19.648 | 1.00 | 44.14 | O |
| ATOM | 2164 | O | HOH | V | 65 | 32.375 | 30.570 | 3.935 | 1.00 | 44.29 | O |
| ATOM | 2165 | O | HOH | V | 66 | 23.674 | 14.801 | −1.168 | 1.00 | 44.29 | O |
| ATOM | 2166 | O | HOH | V | 67 | 10.607 | 41.218 | 4.126 | 1.00 | 44.59 | O |
| ATOM | 2167 | O | HOH | V | 68 | 5.553 | 22.195 | −4.232 | 1.00 | 45.31 | O |
| ATOM | 2168 | O | HOH | V | 69 | 18.683 | 8.716 | −3.851 | 1.00 | 45.45 | O |
| ATOM | 2169 | O | HOH | V | 70 | 31.216 | 36.554 | 23.614 | 1.00 | 45.65 | O |
| ATOM | 2170 | O | HOH | V | 71 | 32.042 | 38.382 | 12.898 | 1.00 | 45.78 | O |
| ATOM | 2171 | O | HOH | V | 72 | 41.571 | 18.538 | 20.942 | 1.00 | 45.85 | O |
| ATOM | 2172 | O | HOH | V | 73 | 24.529 | 18.718 | 30.164 | 1.00 | 45.93 | O |
| ATOM | 2173 | O | HOH | V | 74 | 12.539 | 36.492 | 16.322 | 1.00 | 45.97 | O |
| ATOM | 2174 | O | HOH | V | 75 | 41.603 | 15.767 | 13.285 | 1.00 | 46.25 | O |
| ATOM | 2175 | O | HOH | V | 76 | 21.193 | 17.911 | 28.154 | 1.00 | 46.47 | O |
| ATOM | 2176 | O | HOH | V | 77 | 29.518 | 22.554 | 36.275 | 1.00 | 46.60 | O |
| ATOM | 2177 | O | HOH | V | 78 | 32.636 | 35.308 | 17.598 | 1.00 | 47.00 | O |
| ATOM | 2178 | O | HOH | V | 79 | 13.479 | 40.537 | 21.946 | 1.00 | 47.13 | O |
| ATOM | 2179 | O | HOH | V | 80 | 32.129 | 25.488 | 3.702 | 1.00 | 47.21 | O |
| ATOM | 2180 | O | HOH | V | 81 | 5.317 | 15.523 | 10.878 | 1.00 | 47.24 | O |
| ATOM | 2181 | O | HOH | V | 82 | 14.590 | 12.994 | −13.138 | 1.00 | 47.42 | O |
| ATOM | 2182 | O | HOH | V | 83 | 31.688 | 18.521 | 32.632 | 1.00 | 47.42 | O |
| ATOM | 2183 | O | HOH | V | 84 | 17.527 | 41.724 | 24.627 | 1.00 | 47.58 | O |
| ATOM | 2184 | O | HOH | V | 85 | 2.758 | 27.645 | −6.136 | 1.00 | 47.76 | O |
| ATOM | 2185 | O | HOH | V | 86 | 39.479 | 15.402 | 9.361 | 1.00 | 47.77 | O |
| ATOM | 2186 | O | HOH | V | 87 | 27.097 | 39.457 | 7.716 | 1.00 | 47.80 | O |
| ATOM | 2187 | O | HOH | V | 88 | 14.854 | 38.493 | 18.833 | 1.00 | 48.18 | O |
| ATOM | 2188 | O | HOH | V | 89 | 16.442 | 28.965 | 31.902 | 1.00 | 48.22 | O |
| ATOM | 2189 | O | HOH | V | 90 | 6.592 | 18.094 | 16.102 | 1.00 | 48.28 | O |
| ATOM | 2190 | O | HOH | V | 91 | 25.862 | 43.114 | 20.667 | 1.00 | 48.28 | O |
| ATOM | 2191 | O | HOH | V | 92 | 25.820 | 31.283 | 38.083 | 1.00 | 48.37 | O |
| ATOM | 2192 | O | HOH | V | 93 | 21.448 | 8.215 | −6.909 | 1.00 | 48.43 | O |
| ATOM | 2193 | O | HOH | V | 94 | 30.315 | 7.953 | 13.447 | 1.00 | 48.47 | O |
| ATOM | 2194 | O | HOH | V | 95 | 11.333 | 3.609 | −1.448 | 1.00 | 48.50 | O |
| ATOM | 2195 | O | HOH | V | 96 | 25.475 | 26.684 | −3.988 | 1.00 | 48.54 | O |
| ATOM | 2196 | O | HOH | V | 97 | 21.825 | 7.068 | 6.249 | 1.00 | 48.57 | O |
| ATOM | 2197 | O | HOH | V | 98 | 26.277 | 39.627 | 9.743 | 1.00 | 48.67 | O |
| ATOM | 2198 | O | HOH | V | 99 | 10.637 | 33.053 | 31.840 | 1.00 | 48.70 | O |
| ATOM | 2199 | O | HOH | V | 100 | 8.248 | 10.674 | 15.177 | 1.00 | 48.90 | O |
| ATOM | 2200 | O | HOH | V | 101 | 5.925 | 25.750 | 18.970 | 1.00 | 48.97 | O |
| ATOM | 2201 | O | HOH | V | 102 | 15.403 | 29.322 | −7.859 | 1.00 | 49.09 | O |
| ATOM | 2202 | O | HOH | V | 103 | 3.536 | 35.057 | 9.239 | 1.00 | 49.16 | O |
| ATOM | 2203 | O | HOH | V | 104 | 24.615 | 19.046 | 6.759 | 1.00 | 49.20 | O |
| ATOM | 2204 | O | HOH | V | 105 | 26.458 | 22.354 | 32.348 | 1.00 | 49.22 | O |
| ATOM | 2205 | O | HOH | V | 106 | 29.329 | 9.438 | 8.558 | 1.00 | 49.24 | O |
| ATOM | 2206 | O | HOH | V | 107 | 38.968 | 26.081 | 25.260 | 1.00 | 49.30 | O |
| ATOM | 2207 | O | HOH | V | 108 | 33.166 | 31.078 | 20.463 | 1.00 | 49.55 | O |
| ATOM | 2208 | O | HOH | V | 109 | 23.661 | 41.348 | 9.046 | 1.00 | 49.57 | O |
| ATOM | 2209 | O | HOH | V | 110 | −1.905 | 38.422 | −2.103 | 1.00 | 49.58 | O |
| ATOM | 2210 | O | HOH | V | 111 | 23.567 | 25.829 | 32.172 | 1.00 | 49.84 | O |
| ATOM | 2211 | O | HOH | V | 112 | 39.174 | 28.173 | 18.167 | 1.00 | 49.89 | O |
| ATOM | 2212 | O | HOH | V | 113 | 6.546 | 17.330 | −8.809 | 1.00 | 50.15 | O |
| ATOM | 2213 | O | HOH | V | 114 | 15.378 | 36.610 | 16.329 | 1.00 | 50.24 | O |
| ATOM | 2214 | O | HOH | V | 115 | 24.014 | 21.545 | 30.393 | 1.00 | 50.25 | O |
| ATOM | 2215 | O | HOH | V | 116 | 13.119 | 40.412 | 25.848 | 1.00 | 50.51 | O |
| ATOM | 2216 | O | HOH | V | 117 | 34.344 | 32.657 | 10.572 | 1.00 | 50.99 | O |
| ATOM | 2217 | O | HOH | V | 118 | 26.462 | 26.162 | 35.827 | 1.00 | 51.01 | O |
| ATOM | 2218 | O | HOH | V | 119 | 37.119 | 20.213 | 15.138 | 1.00 | 51.01 | O |
| ATOM | 2219 | O | HOH | V | 120 | 20.402 | 7.809 | 0.264 | 1.00 | 51.21 | O |
| ATOM | 2220 | O | HOH | V | 121 | 32.907 | 23.096 | 33.511 | 1.00 | 51.26 | O |
| ATOM | 2221 | O | HOH | V | 122 | 18.316 | 15.239 | 25.933 | 1.00 | 51.36 | O |
| ATOM | 2222 | O | HOH | V | 123 | 22.210 | 27.272 | 33.235 | 1.00 | 51.39 | O |
| ATOM | 2223 | O | HOH | V | 124 | 4.773 | 34.446 | −7.751 | 1.00 | 51.56 | O |
| ATOM | 2224 | O | HOH | V | 125 | −11.176 | 24.480 | −12.026 | 1.00 | 51.70 | O |
| ATOM | 2225 | O | HOH | V | 126 | 29.201 | 40.901 | 16.488 | 1.00 | 51.72 | O |
| ATOM | 2226 | O | HOH | V | 127 | −14.169 | 32.191 | −15.792 | 1.00 | 51.83 | O |
| ATOM | 2227 | O | HOH | V | 128 | 27.174 | 21.751 | 2.087 | 1.00 | 51.83 | O |
| ATOM | 2228 | O | HOH | V | 129 | 12.661 | 30.921 | 30.244 | 1.00 | 51.89 | O |
| ATOM | 2229 | O | HOH | V | 130 | 16.175 | 21.212 | 25.031 | 1.00 | 51.93 | O |
| ATOM | 2230 | O | HOH | V | 131 | 20.211 | 40.398 | 29.373 | 1.00 | 52.00 | O |
| ATOM | 2231 | O | HOH | V | 132 | −13.899 | 29.982 | −18.098 | 1.00 | 52.10 | O |
| ATOM | 2232 | O | HOH | V | 133 | 15.844 | 9.719 | 16.408 | 1.00 | 52.40 | O |
| ATOM | 2233 | O | HOH | V | 134 | 31.386 | 39.552 | 17.146 | 1.00 | 52.59 | O |
| ATOM | 2234 | O | HOH | V | 135 | 0.640 | 32.016 | 4.846 | 1.00 | 52.62 | O |
| ATOM | 2235 | O | HOH | V | 136 | 42.270 | 20.847 | 16.516 | 1.00 | 52.68 | O |
| ATOM | 2236 | O | HOH | V | 137 | 17.490 | 42.697 | 21.680 | 1.00 | 52.79 | O |
| ATOM | 2237 | O | HOH | V | 138 | 29.839 | 40.760 | 8.920 | 1.00 | 52.82 | O |
| ATOM | 2238 | O | HOH | V | 139 | 4.290 | 30.109 | 9.865 | 1.00 | 52.89 | O |
| ATOM | 2239 | O | HOH | V | 140 | 19.892 | 7.267 | −2.229 | 1.00 | 53.00 | O |
| ATOM | 2240 | O | HOH | V | 141 | 9.212 | 35.423 | 31.648 | 1.00 | 53.10 | O |
| ATOM | 2241 | O | HOH | V | 142 | 31.957 | 28.820 | 37.099 | 1.00 | 53.23 | O |
| ATOM | 2242 | O | HOH | V | 143 | 40.970 | 31.344 | 14.438 | 1.00 | 53.27 | O |
| ATOM | 2243 | O | HOH | V | 144 | 40.782 | 22.485 | 20.153 | 1.00 | 53.41 | O |
| ATOM | 2244 | O | HOH | V | 145 | 4.688 | 33.007 | −12.475 | 1.00 | 53.62 | O |
| ATOM | 2245 | O | HOH | V | 146 | 12.405 | 42.730 | −6.583 | 1.00 | 53.65 | O |
| ATOM | 2246 | O | HOH | V | 147 | 30.118 | 41.753 | 23.343 | 1.00 | 53.90 | O |
| ATOM | 2247 | O | HOH | V | 148 | 40.310 | 19.731 | 15.626 | 1.00 | 54.03 | O |
| ATOM | 2248 | O | HOH | V | 149 | 9.904 | 10.991 | −10.125 | 1.00 | 54.05 | O |
| ATOM | 2249 | O | HOH | V | 150 | 35.645 | 26.966 | 10.324 | 1.00 | 54.10 | O |
| ATOM | 2250 | O | HOH | V | 151 | 3.249 | 13.849 | 9.818 | 1.00 | 54.12 | O |
| ATOM | 2251 | O | HOH | V | 152 | 8.994 | 41.917 | 8.500 | 1.00 | 54.13 | O |
| ATOM | 2252 | O | HOH | V | 153 | 25.494 | 7.351 | 8.323 | 1.00 | 54.13 | O |
| ATOM | 2253 | O | HOH | V | 154 | −19.853 | 30.788 | −6.983 | 1.00 | 54.14 | O |
| ATOM | 2254 | O | HOH | V | 155 | 28.190 | 2.825 | 26.243 | 1.00 | 54.20 | O |
| ATOM | 2255 | O | HOH | V | 156 | 14.754 | 37.385 | 13.109 | 1.00 | 54.33 | O |
| ATOM | 2256 | O | HOH | V | 157 | 13.944 | 9.467 | −11.887 | 1.00 | 54.33 | O |
| ATOM | 2257 | O | HOH | V | 158 | 5.602 | 27.878 | 16.557 | 1.00 | 54.52 | O |
| ATOM | 2258 | O | HOH | V | 159 | 35.605 | 5.102 | 13.754 | 1.00 | 54.70 | O |
| ATOM | 2259 | O | HOH | V | 160 | 24.308 | 35.094 | −1.504 | 1.00 | 54.70 | O |
| ATOM | 2260 | O | HOH | V | 161 | 28.337 | 18.154 | 4.417 | 1.00 | 54.85 | O |
| ATOM | 2261 | O | HOH | V | 162 | 34.895 | 34.791 | 30.514 | 1.00 | 54.94 | O |
| ATOM | 2262 | O | HOH | V | 163 | 4.910 | 16.781 | 14.763 | 1.00 | 55.00 | O |
| ATOM | 2263 | O | HOH | V | 164 | 11.023 | 5.038 | 5.200 | 1.00 | 55.13 | O |
| ATOM | 2264 | O | HOH | V | 165 | 39.542 | 17.117 | 25.841 | 1.00 | 55.21 | O |
| ATOM | 2265 | O | HOH | V | 166 | 24.386 | 37.780 | −6.091 | 1.00 | 55.24 | O |
| ATOM | 2266 | O | HOH | V | 167 | 20.889 | 19.941 | 29.200 | 1.00 | 55.34 | O |
| ATOM | 2267 | O | HOH | V | 168 | 6.752 | 22.057 | −14.805 | 1.00 | 55.38 | O |
| ATOM | 2268 | O | HOH | V | 169 | 25.163 | 5.963 | 12.276 | 1.00 | 55.50 | O |
| ATOM | 2269 | O | HOH | V | 170 | 28.609 | 39.754 | 12.521 | 1.00 | 55.51 | O |
| ATOM | 2270 | O | HOH | V | 171 | 11.031 | 13.390 | 14.244 | 1.00 | 55.56 | O |
| ATOM | 2271 | O | HOH | V | 172 | 24.662 | 35.439 | 2.257 | 1.00 | 55.60 | O |
| ATOM | 2272 | O | HOH | V | 173 | 36.161 | 34.403 | 25.458 | 1.00 | 55.61 | O |
| ATOM | 2273 | O | HOH | V | 174 | 9.631 | 6.595 | 3.959 | 1.00 | 55.62 | O |
| ATOM | 2274 | O | HOH | V | 175 | 38.038 | 17.337 | 8.038 | 1.00 | 55.79 | O |
| ATOM | 2275 | O | HOH | V | 176 | 0.834 | 36.938 | 9.105 | 1.00 | 55.88 | O |
| ATOM | 2276 | O | HOH | V | 177 | 30.977 | 6.285 | 15.282 | 1.00 | 55.89 | O |
| ATOM | 2277 | O | HOH | V | 178 | 36.456 | 34.296 | 14.233 | 1.00 | 55.99 | O |
| ATOM | 2278 | O | HOH | V | 179 | −16.740 | 27.510 | −6.376 | 1.00 | 56.17 | O |
| ATOM | 2279 | O | HOH | V | 180 | 17.830 | 28.498 | −7.217 | 1.00 | 56.32 | O |
| ATOM | 2280 | O | HOH | V | 181 | 27.092 | 42.066 | 23.041 | 1.00 | 56.37 | O |
| ATOM | 2281 | O | HOH | V | 182 | 32.854 | 20.918 | 3.041 | 1.00 | 56.40 | O |
| ATOM | 2282 | O | HOH | V | 183 | 33.531 | 36.555 | 20.279 | 1.00 | 56.52 | O |
| ATOM | 2283 | O | HOH | V | 184 | 29.161 | 9.790 | 11.459 | 1.00 | 56.74 | O |
| ATOM | 2284 | O | HOH | V | 185 | 30.947 | 21.061 | 33.802 | 1.00 | 56.78 | O |
| ATOM | 2285 | O | HOH | V | 186 | 4.419 | 10.917 | 12.329 | 1.00 | 57.20 | O |
| ATOM | 2286 | O | HOH | V | 187 | 35.731 | 21.349 | 33.562 | 1.00 | 57.25 | O |
| ATOM | 2287 | O | HOH | V | 188 | 10.127 | 34.435 | −7.921 | 1.00 | 57.31 | O |
| ATOM | 2288 | O | HOH | V | 189 | 24.815 | 39.636 | 5.433 | 1.00 | 57.31 | O |
| ATOM | 2289 | O | HOH | V | 190 | 16.135 | 28.959 | −10.450 | 1.00 | 57.40 | O |
| ATOM | 2290 | O | HOH | V | 191 | 28.755 | 27.074 | −0.804 | 1.00 | 57.58 | O |
| ATOM | 2291 | O | HOH | V | 192 | 37.694 | 28.335 | 20.845 | 1.00 | 58.09 | O |
| ATOM | 2292 | O | HOH | V | 193 | 12.094 | 17.839 | 22.662 | 1.00 | 58.11 | O |
| ATOM | 2293 | O | HOH | V | 194 | 16.739 | 12.034 | −12.326 | 1.00 | 58.15 | O |
| ATOM | 2294 | O | HOH | V | 195 | 35.658 | 34.834 | 39.271 | 1.00 | 58.17 | O |
| ATOM | 2295 | O | HOH | V | 196 | 6.325 | 23.731 | −10.962 | 1.00 | 58.22 | O |
| ATOM | 2296 | O | HOH | V | 197 | 40.239 | 32.219 | 19.158 | 1.00 | 58.22 | O |
| ATOM | 2297 | O | HOH | V | 198 | 42.009 | 17.600 | 28.618 | 1.00 | 58.40 | O |
| ATOM | 2298 | O | HOH | V | 199 | 13.977 | 43.767 | 4.977 | 1.00 | 58.43 | O |
| ATOM | 2299 | O | HOH | V | 200 | 16.099 | 2.261 | −8.711 | 1.00 | 58.43 | O |
| ATOM | 2300 | O | HOH | V | 201 | 28.967 | 4.492 | 15.572 | 1.00 | 58.44 | O |
| ATOM | 2301 | O | HOH | V | 202 | −9.860 | 29.231 | −7.273 | 1.00 | 58.50 | O |
| ATOM | 2302 | O | HOH | V | 203 | 19.071 | 45.087 | 23.986 | 1.00 | 58.89 | O |
| ATOM | 2303 | O | HOH | V | 204 | 23.529 | 32.968 | −5.882 | 1.00 | 59.37 | O |
| ATOM | 2304 | O | HOH | V | 205 | 42.799 | 19.843 | 29.263 | 1.00 | 59.50 | O |
| ATOM | 2305 | O | HOH | V | 206 | 21.137 | 24.412 | 31.357 | 1.00 | 59.65 | O |
| ATOM | 2306 | O | HOH | V | 207 | 21.855 | 22.367 | 32.296 | 1.00 | 59.84 | O |
| ATOM | 2307 | O | HOH | V | 208 | 3.008 | 31.149 | 11.774 | 1.00 | 59.88 | O |
| ATOM | 2308 | O | HOH | V | 209 | 25.290 | 39.928 | 29.735 | 1.00 | 59.89 | O |
| ATOM | 2309 | O | HOH | V | 210 | 20.546 | 26.076 | −5.981 | 1.00 | 60.27 | O |
| ATOM | 2310 | O | HOH | V | 211 | 7.922 | 7.232 | −0.636 | 1.00 | 60.32 | O |
| ATOM | 2311 | O | HOH | V | 212 | 7.268 | 35.864 | −11.540 | 1.00 | 60.42 | O |
| ATOM | 2312 | O | HOH | V | 213 | 5.789 | 26.044 | −10.740 | 1.00 | 60.43 | O |
| ATOM | 2313 | O | HOH | V | 214 | 26.552 | 14.136 | −0.935 | 1.00 | 60.54 | O |
| ATOM | 2314 | O | HOH | V | 215 | 41.103 | 32.645 | 22.002 | 1.00 | 60.87 | O |
| ATOM | 2315 | O | HOH | V | 216 | 10.211 | 45.156 | 8.475 | 1.00 | 61.19 | O |
| ATOM | 2316 | O | HOH | V | 217 | 25.176 | 9.626 | 5.053 | 1.00 | 61.26 | O |
| ATOM | 2317 | O | HOH | V | 218 | 11.154 | 41.223 | 20.664 | 1.00 | 61.36 | O |
| ATOM | 2318 | O | HOH | V | 219 | 12.673 | 39.495 | 16.829 | 1.00 | 61.48 | O |
| ATOM | 2319 | O | HOH | V | 220 | 6.931 | 21.130 | 20.882 | 1.00 | 61.81 | O |
| ATOM | 2320 | O | HOH | V | 221 | 34.324 | 35.314 | 27.782 | 1.00 | 61.99 | O |
| ATOM | 2321 | O | HOH | V | 222 | 22.533 | 27.773 | −4.889 | 1.00 | 62.36 | O |
| ATOM | 2322 | O | HOH | V | 223 | 26.615 | 24.563 | −3.514 | 1.00 | 62.43 | O |
| ATOM | 2323 | O | HOH | V | 224 | 22.620 | 8.612 | −0.252 | 1.00 | 62.66 | O |
| ATOM | 2324 | O | HOH | V | 225 | 7.850 | 40.686 | 5.273 | 1.00 | 62.89 | O |
| ATOM | 2325 | O | HOH | V | 226 | 13.072 | 10.222 | 19.270 | 1.00 | 62.97 | O |
| ATOM | 2326 | O | HOH | V | 227 | 36.863 | 23.365 | 8.281 | 1.00 | 63.29 | O |
| ATOM | 2327 | O | HOH | V | 228 | 3.086 | 21.555 | 0.662 | 1.00 | 63.45 | O |
| ATOM | 2328 | O | HOH | V | 229 | 40.090 | 16.185 | 28.416 | 1.00 | 63.62 | O |
| ATOM | 2329 | O | HOH | V | 230 | 28.499 | 41.692 | 20.652 | 1.00 | 63.74 | O |
| ATOM | 2330 | O | HOH | V | 231 | 5.053 | 21.524 | −0.933 | 1.00 | 63.76 | O |
| ATOM | 2331 | O | HOH | V | 232 | 18.279 | 22.767 | −9.711 | 1.00 | 63.86 | O |
| ATOM | 2332 | O | HOH | V | 233 | 4.021 | 13.207 | 14.475 | 1.00 | 64.13 | O |
| ATOM | 2333 | O | HOH | V | 234 | 20.707 | 46.785 | 18.023 | 1.00 | 64.17 | O |
| ATOM | 2334 | O | HOH | V | 235 | 18.269 | 24.266 | −5.185 | 1.00 | 64.61 | O |
| ATOM | 2335 | O | HOH | V | 236 | −1.075 | 31.083 | 6.459 | 1.00 | 64.71 | O |
| ATOM | 2336 | O | HOH | V | 237 | 36.067 | 6.771 | 10.766 | 1.00 | 64.72 | O |
| ATOM | 2337 | O | HOH | V | 238 | 41.379 | 11.059 | 22.312 | 1.00 | 64.85 | O |
| ATOM | 2338 | O | HOH | V | 239 | 2.764 | 21.069 | −4.139 | 1.00 | 64.89 | O |
| ATOM | 2339 | O | HOH | V | 240 | 36.774 | 12.493 | 29.500 | 1.00 | 64.91 | O |
| ATOM | 2340 | O | HOH | V | 241 | 33.576 | 7.054 | 15.865 | 1.00 | 65.14 | O |
| ATOM | 2341 | O | HOH | V | 242 | 14.783 | 25.737 | 26.607 | 1.00 | 65.39 | O |
| ATOM | 2342 | O | HOH | V | 243 | 19.632 | 29.934 | −8.079 | 1.00 | 65.40 | O |
| ATOM | 2343 | O | HOH | V | 244 | 19.893 | 42.353 | 12.315 | 1.00 | 65.64 | O |
| ATOM | 2344 | O | HOH | V | 245 | 30.511 | 40.345 | 32.883 | 1.00 | 66.15 | O |
| ATOM | 2345 | O | HOH | V | 246 | 6.494 | 31.838 | −13.514 | 1.00 | 66.20 | O |
| ATOM | 2346 | O | HOH | V | 247 | 41.592 | 29.824 | 26.952 | 1.00 | 67.14 | O |
| ATOM | 2347 | O | HOH | V | 248 | 10.095 | 12.965 | −13.010 | 1.00 | 68.08 | O |
| ATOM | 2348 | O | HOH | V | 249 | 28.076 | 13.999 | 7.164 | 1.00 | 68.53 | O |
| ATOM | 2349 | O | HOH | V | 250 | 16.142 | 3.364 | 3.323 | 1.00 | 68.80 | O |
| ATOM | 2350 | O | HOH | V | 251 | 11.453 | 41.019 | −1.487 | 1.00 | 68.99 | O |
| ATOM | 2351 | O | HOH | V | 252 | 22.049 | 30.697 | 36.943 | 1.00 | 69.14 | O |
| ATOM | 2352 | O | HOH | V | 253 | 31.306 | 29.631 | 39.320 | 1.00 | 69.25 | O |
| ATOM | 2353 | O | HOH | V | 254 | 26.120 | 35.578 | 0.298 | 1.00 | 69.34 | O |
| ATOM | 2354 | O | HOH | V | 255 | 37.240 | 33.402 | 37.433 | 1.00 | 69.56 | O |
| ATOM | 2355 | O | HOH | V | 256 | 14.450 | 19.763 | 22.059 | 1.00 | 70.09 | O |

1. Crystalline LBD of RORα.

2. The crystalline LBD of RORα of claim 1 wherein said LBD of RORα is associated with a small molecule.

3. The crystalline LBD of RORα of claim 1 wherein said LBD of RORα is associated with a lipophilic substance.

4. The crystalline LBD of RORα of claim 2 wherein said lipophilic substance is cholesterol or a derivative of cholesterol.

5. The crystalline LBD of RORα of claim 1 wherein said crystal comprises a unit cell having the dimensions of a=55.9 Å±2 Å, b=49.9 Å±2 Å, c=60.7 Å±2 Å and β=98.70°±5°and space group P2(1).

6. The crystalline LBD of RORα of claim 1 wherein said crystalline LBD of RORα comprises the atomic structure coordinates of Table 8 or 9 or a part thereof.

7. A heavy atom derivative of a crystal according to claim 1.

8. A computer readable medium comprising a model embodying the structure of the LBD RORα comprising one or more sets of atomic coordinates in Table 8 or 9.

9. A method for identifying a substance binding to the LBD of RORα, comprising:
(a) contacting a candidate substance with a model embodying the structure of the LBD of RORα comprising one or more sets of atomic coordinates in Table 8 or 9;
(b) assessing the interaction of said candidate substance with said model, and;
(c) selecting a substance which is predicted to interact with the LED of RORα.

10. (canceled)

11. A method according to claim 9 wherein the substance interacts directly or indirectly with one or more amino acids selected from the group consisting of:
Cys321, Gln322, Tyr323, Leu328,Trp353, Cys356, Ala357, Lys359, Ile360, Glu362,Ala363, Val397, Phe398, Arg400, Met401, Arg403, Ala404, Val412, Tyr413, Phe414, Phe424, Leu427, Cys429, Phe432, Ile433, Val436, His517, Lys520 and Tyr540.

12. A method according to claim 9 wherein the substance interacts directly or indirectly with one or more amino acids selected from the group consisting of:
Gln322, Tyr323, Arg400 and Arg403.

13. A method according to claim 9 using a homologue of said model, wherein said homologue has a root mean square derivation from the backbone atoms of said amino acids of not more than 1.5 Å.

14. The method of claim 9 wherein the substance is a small molecule.

15. The method of claim 14 wherein said substance is cholesterol or a cholesterol derivative.

16. A method for identifying an agonist or antagonist that binds to the LBD of RORα comprising:
(a) selecting a potential compound by performing rational drug design with one or more sets of atomic coordinates set forth in Table 8 or 9, wherein said selecting is performed in conjunction with computer modeling;
(b) contacting the potential compound with a LBD of RORα and
(c) measuring the biological activity of RORα.

17. The method of claim 16 wherein said compound is designed to interact directly or indirectly with one or more amino acids selected from the group consisting of:
Cys321, Gln322, Tyr323, Leu328,Trp353, Cys356, Ala357, Lys359, Ile360, Glu362, Ala363, Val397, Phe398, Arg400, Met401, Arg403, Ala404, Val412, Tyr413, Phe414, Phe424, Leu427, Cys429, Phe432, Ile433, Val436, His517, Lys520 and Tyr540.

18. A method according to claim 17 wherein the substance interacts directly or indirectly with one or more amino acids selected from the group consisting of:
Gln322, Tyr323, Arg400 and Arg403.

19. The method of claim 18 wherein said compound is selected as RORα agonist if it stabilizes helix 12 of the LBD of RORα in the agonist position.

20. The method of claim 19 wherein said compound is selected as RORα antagonist if it destabilizes helix 12 of the LBD of RORα from the agonist position.

21. A pharmaceutical composition comprising a therapeutically effective amount of a compound stabilizing helix 12 of RORα in the agonist position and a pharmaceutically acceptable carrier.

22. A pharmaceutical composition comprising a therapeutically effective amount of a compound destabilizing helix 12 of RORα from the agonist position and a pharmaceutically acceptable carrier.

23. A method of screening for compounds interacting with RORα comprising:
(a) contacting RORα with a candidate compound,
(b) measuring interactions between the candidate compound and RORα in the absence of sterols, and
(c) selecting said compound if it interacts with RORα.

24. The method of claim 23 for the screening for compounds useful for the treatment of cholesterol related diseases.

25. The method of claim 23 for the screening for compounds useful for the treatment of endocrine disorders, atherosclerosis and cardiovascular diseases, metabolic diseases such as for instance obesity, inflammatory diseases, skin diseases, diseases related to the CNS, such as for instance Alzheimer disease and tumor related diseases.

26. Use of RORα in cellular screening assays for the identification of compounds useful for the treatment of cholesterol related diseases.

27. Use of RORα in cellular screening assays for the identification of compounds with endocrine disorders, atherosclerosis and cardiovascular diseases, metabolic diseases such as for instance obesity, inflammatory diseases, skin diseases, diseases related to the CNS, such as for instance Alzheimer disease and tumor related diseases.

28. A composition comprising LBD of RORα and cholesterol or a cholesterol derivative.

29. A composition according to claim 28 wherein said composition is crystallizable.

30. The crystalline LBD of RORα of claim 2 wherein said LBD of RORα is associated with a lipophilic substance.

31. The crystalline LBD of RORα of claim 2 wherein said crystal comprises a unit cell having the dimensions of a=55.9 Å±2 Å,b=49.9 Å±2 Å, c=60.7 Å±2 Å and β=98.7±5° and space groupP2(1).

32. The crystalline LBD of RORα of claim 3 wherein said crystal comprises a unit cell having the dimensions of a=55.9 Å±2 Å, b=49.9 Å±2 Å, c=60.7 Å±2 Å and β=98.7°±5° and space groupP2(1).

33. The crystalline LBD of RORα of claim 2 wherein said crystalline LBD of RORα comprises the atomic structure coordinates of Table 8 or 9 or a part thereof.

34. The crystalline LBD of RORα of claim 3 wherein said crystalline LBD of RORα comprises the atomic structure coordinates of Table 8 or 9 or a part thereof.

35. A heavy atom derivative of a crystal according to claim 2.

36. A heavy atom derivative of a crystal according to claim 3.

37. A heavy atom derivative of a crystal according to claim 31.

38. A heavy atom derivative of a crystal according to claim 32.

39. A heavy atom derivative of a crystal according to claim 33.

40. A heavy atom derivative of a crystal according to claim 34.
